(19) United States
(12) Patent Application Publication
(10) Pub. No.: US 2024/0398781 A1
(43) Pub. Date: Dec. 5, 2024

(54) SPIROCYCLIC CYCLIC MODULATORS OF CHOLESTEROL BIOSYNTHESIS AND THEIR USE FOR PROMOTING REMYELINATION

(71) Applicants: GENENTECH, INC., South San Francisco, CA (US); CONVELO THERAPEUTICS, INC., Cleveland, OH (US)

(72) Inventors: Marie-Gabrielle BRAUN, South San Francisco,, CA (US); Georgette CASTANEDO, South San Francisco, CA (US); William VERNIER, South San Francisco, CA (US); Matthew VOLGRAF, South San Francisco, CA (US); James Guy BREITENBUCHER, Valley View, OH (US); Brad LANG, Valley View, OH (US)

(21) Appl. No.: 18/670,608

(22) Filed: May 21, 2024

Related U.S. Application Data
(63) Continuation of application No. PCT/US2022/080357, filed on Nov. 22, 2022.
(60) Provisional application No. 63/282,354, filed on Nov. 23, 2021.

(51) Int. Cl. A61K 31/4545; A61K 31/407; A61K 31/4155; A61K 31/4178; A61K 31/438; A61K 31/4439; A61K 31/454; C07D 495/10

(52) U.S. Cl. CPC A61K 31/4545; A61K 31/407; A61K 31/4155; A61K 31/4178; A61K 31/438; A61K 31/4439; A61K 31/454; C07D 495/10

(57) ABSTRACT

The subject matter described herein is directed to myelin-promoting compounds of Formula I and pharmaceutical salts thereof, methods of preparing the compounds, pharmaceutical compositions comprising the compounds, and methods of administering the compounds for the treatment of disorders, such as myelin-related disorders.

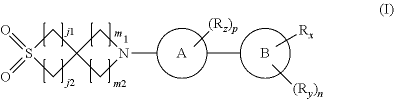

### CROSS REFERENCE TO RELATED APPLICATIONS

This application is a bypass continuation of international application no. PCT/US2022/080357 filed Nov. 22, 2022, which claims the benefit of priority to U.S. Provisional Patent Application No. 63/282,354, filed on Nov. 23, 2021, the contents of which is incorporated by reference herein in its entirety for all purposes.

### FIELD

The subject matter described herein is directed to myelin-promoting compounds of Formula I, methods of making the compounds, their pharmaceutical compositions, and their use in the treatment of myelin-related disorders.

### BACKGROUND

Myelin-related disorders are disorders that result in abnormalities of the myelin sheath (e.g., dysmyelination, demyelination and hypomyelination) in a subject's neural cells, e.g., CNS neurons including their axons. Loss or degradation of the myelin sheath in such disorders produces a slowing or cessation of nerve cell conduction. The resulting myelin related disorders are characterized by deficits in sensation, motor function, cognition, or other physiological functions. Myelin related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, and radiation-induced demyelination.

MS is the most common myelin-related disorder affecting several million people globally and is estimated to result in about 18,000 deaths per year. MS is a complex neurological disease characterized by deterioration of central nervous system (CNS) myelin. Myelin, composed in its majority by lipids (70% lipids, 30% protein), protects axons and makes saltatory conduction possible, which speeds axonal electric impulse. Demyelination of axons in chronic MS can result in axon degeneration and neuronal cell death. Additionally, MS destroys oligodendrocytes, the highly specialized CNS cells that generate and maintain myelin. A repair process, called remyelination, takes place in early phases of the disease, but over time, the oligodendrocytes are unable to completely rebuild and restore the myelin sheath. Repeated attacks lead to successively less effective remyelination, until a scar-like plaque is built up around the damaged axons. These scars are the origin of the symptoms.

At present, there is no cure for myelin-related disorders, and only a handful of disease-modifying therapies are available. Accordingly, there is a need for new therapeutic approaches to the treatment of myelin-related disorders, including the promotion of remyelination. The subject matter described herein addresses this unmet need.

### BRIEF SUMMARY

In certain embodiments, the subject matter described herein is directed to a compound of Formula I, or a pharmaceutically acceptable salt thereof.

In certain embodiments, the subject matter described herein is directed to a pharmaceutical composition comprising a compound of Formula I, or a pharmaceutically acceptable salt thereof, and a pharmaceutically acceptable excipient.

In certain embodiments, the subject matter described herein is directed to methods of treating a disorder in a subject in need thereof, wherein the disorder is a myelin-related disorder, comprising administering to the subject an effective amount of a compound of Formula I, or a pharmaceutically acceptable salt thereof; or a pharmaceutical composition comprising a compound of Formula I, or a pharmaceutically acceptable salt thereof, and a pharmaceutically acceptable excipient.

In certain embodiments, the subject matter described herein is directed to a compound of Formula I, or a pharmaceutically acceptable salt thereof, or a pharmaceutical composition comprising a pharmaceutically acceptable excipient and a compound of Formula I, or a pharmaceutically acceptable salt thereof, for use in treating a myelin-related disorder.

In certain embodiments, the subject matter described herein is directed to methods of promoting myelination in a subject in need thereof, comprising administering to the subject a therapeutically effective amount of a compound of Formula I, or a pharmaceutically acceptable salt thereof; or a pharmaceutical composition comprising a compound of Formula I, or a pharmaceutically acceptable salt thereof, a pharmaceutically acceptable excipient.

In certain embodiments, the subject matter described herein is directed to the use of a compound of Formula I, or a pharmaceutically acceptable salt thereof, or a pharmaceutical composition comprising a pharmaceutically acceptable excipient and a compound of Formula I, or a pharmaceutically acceptable salt thereof, in the manufacture of a medicament for treating a myelin-related disorder.

In certain embodiments, the subject matter described herein is directed to methods of preparing compounds of Formula I, or a pharmaceutically acceptable salt thereof.

Other embodiments are also described.

### DETAILED DESCRIPTION

Described herein are compounds of Formula I, methods of making the compounds, their pharmaceutical compositions, and their use in the treatment of myelin-related disorders. In some embodiments, the compounds provided herein are myelin-promoting.

Without wishing to be bound by theory, the enhancement and/or inducement of the accumulation of Δ8,9-unsaturated sterol intermediates of the cholesterol biosynthesis pathway in oligodendrocyte progenitor cells (OPCs) can induce oligodendrocyte generation. Enhancement and/or inducement of the accumulation of Δ8,9-unsaturated sterol intermediates can be provided, for example, by modulating and/or inhibiting the enzymes within the OPC cholesterol biosynthesis pathway that inhibit Δ8,9-unsaturated sterol intermediate accumulation and/or for which the Δ8,9-unsaturated sterol intermediates are substrates, as well as directly and/or indirectly administering Δ8,9-unsaturated sterol intermediates to the OPCs. Enhancement and/or inducement of the accumulation of Δ8,9-unsaturated sterol intermediates may promote OPC differentiation, survival, proliferation, and/or maturation, and it is thought this might treat disease and/or disorders in subjects where myelination is beneficial to the subject.

As such, in some embodiments, an agent, such as a compound of Formula I, or a pharmaceutically acceptable salt thereof, that can enhance and/or induce accumulation of Δ8,9-unsaturated sterol intermediates of the cholesterol biosynthesis pathway in OPCs can be administered to a subject, and/or to the OPCs, at an amount effective to promote and/or induce OPC differentiation, proliferation, and/or maturation, as well as oligodendrocyte generation. In certain embodiments, the agent, for example a compound of Formula I, or a pharmaceutically acceptable salt thereof, is a compound that inhibits enzyme-mediated synthesis of one or more sterol intermediates in the cholesterol biosynthesis pathway of the OPCs, and/or promotes accumulation of Δ8,9-unsaturated sterol intermediates.

In certain embodiments, the compound of Formula I, or a pharmaceutically acceptable salt thereof, can modulate and/or inhibit one or more enzyme-mediated conversion steps of the cholesterol biosynthises pathway, such as in the pathway from lanosterol to cholesterol, for example, between lanosterol and/or lathosterol; modulating and/or inhibiting one or more of these steps in OPCs may promote and/or induce oligodendrocyte generation. For example, in some embodiments, a compound of Formula I or pharmaceutically acceptable salt thereof can inhibit CYP51, sterol 14-reductase (TM7SF2 and/or LBR), SC4MOL, NSDHL, and/or emopamil binding protein (EBP) enzyme mediated synthesis of sterol intermediates in the cholesterol biosynthesis pathway. In certain embodiments, a compound of Formula I or pharmaceutically acceptable salt thereof can inhibit CYP51, sterol 14-reductase and/or EBP. In certain embodiments, the compound of Formula I or pharmaceutically acceptable salt thereof can inhibit EBP.

For example, in certain embodiments, the compound of Formula I, or pharmaceutically acceptable salt thereof, used in the methods described herein can inhibit enzyme mediated conversion of zymostenol to lathosterol through the inhibition of emopamil binding protein (EBP) isomerase enzyme activity. Alternatively, in certain embodiments, the compound of Formula I, or pharmaceutically acceptable salt thereof, used in the methods described herein can inhibit sterol C14 reductase enzyme activity or CYP51 enzyme activity in the cholesterol biosynthesis pathway.

Emopamil Binding Protein (EBP) is an enzyme responsible for one of the final steps in the production of cholesterol. Specifically, EBP converts zymostenol to lathosterol, where other enzymes then modify lathosterol to produce cholesterol. EBP is also referred to as Δ8-Δ7-sterol isomerase, 3-beta-hydroxysteroid-Delta(8),Delta(7)-isomerase, CDPX2, CHO2, CPX, or CPXD).

Without being bound by a particular theory, it is believed that compounds of Formula I or a pharmaceutically acceptable salt thereof can inhibit EBP mediated conversion of zymostenol to lathosterol in the cholesterol biosynthesis pathway of OPCs resulting in enhancement and/or inducement of the accumulation of Δ8,9-unsaturated sterol intermediates. In some embodiments, enhancement and/or inducement of the accumulation of Δ8,9-unsaturated sterol intermediates can promote OPC differentiation, survival, proliferation and/or maturation and treat disease and/or disorders in subjects where myelination or myelinization is beneficial to the subject. This mechanism of promoting myelination is distinct from the primary action of immunomodulatory agents that are often used to treat myelin-related disorders.

The presently disclosed subject matter will now be described more fully hereinafter. However, many modifications and other embodiments of the presently disclosed subject matter set forth herein may come to mind to one skilled in the art to which the presently disclosed subject matter pertains having the benefit of the teachings presented in the descriptions herein. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±50%. In certain other embodiments, the term “about” includes the indicated amount 20%. In certain other embodiments, the term “about” includes the indicated amount ±10%. In other embodiments, the term “about” includes the indicated amount ±5%. In certain other embodiments, the term “about” includes the indicated amount±1%. In certain other embodiments, the term “about” includes the indicated amount±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last-mentioned group contains the atom by which the moiety is attached to the rest of the molecule.

“Alkenyl” refers to an alkyl group containing at least one carbon-carbon double bond and, unless otherwise described, may have from 2 to 20 carbon atoms (i.e., C₂-C₂₀ alkenyl), 2 to 8 carbon atoms (i.e., C₂-C₈ alkenyl), 2 to 6 carbon atoms (i.e., C₂-C₆ alkenyl) or 2 to 4 carbon atoms (i.e., C₂-C₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond, unless otherwise described, may have from 2 to 20 carbon atoms (i.e., C₂-C₂₀ alkynyl), 2 to 8 carbon atoms (i.e., C₂-C₈ alkynyl), 2 to 6 carbon atoms (i.e., C₂-C₆ alkynyl) or 2 to 4 carbon atoms (i.e., C₂-C₄ alkynyl). The term “alkynyl” also includes those groups having one triple bond and one double bond.

“Alkoxy” refers to the group “alkyl-O—” (e.g., C₁-C₃ alkoxy or C₁-C₆ alkoxy). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkylthio” refers to the group “alkyl-S—”.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a heterocyclyl; which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of the point of attachment. If one or more aryl groups are fused with a heterocyclyl, the resulting ring system is heterocyclyl regardless of the point of attachment.

“Arylalkyl” or “Aralkyl” refers to the group “aryl-alkyl-”, such as (C₆-C₁₀ aryl)-C₁-C₃ alkyl. A non-limiting example of arylalkyl is benzyl.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein.

“Cycloalkyl” refers to a saturated or partially unsaturated cyclic alkyl group having a single ring or multiple rings which may include fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any moiety comprising a non-aromatic alkyl ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl. As used herein, “halocycloalkyl,” such as C₃-C₇ halocycloalkyl, refers to a C₃-C₇ cycloalkyl group that is substituted with one or more halogens.

“Cycloalkylalkyl” refers to the group “cycloalkyl-alkyl-”, such as (C₃-C₆ cycloalkyl)-C₁-C₃ alkyl.

“Guanidino” refers to —NR^{y}C(═NR^{z})(NR^{y}R^{z}), wherein each R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Hydrazino” refers to —NHNH₂.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro (fluorine), chloro (chlorine), bromo (bromine) or iodo (iodine).

“Haloalkyl” refers to an unbranched or branched alkyl group as defined above, wherein one or more (e.g., 1 to 6, or 1 to 3) hydrogen atoms are replaced by a halogen. For example, halo-C₁-C₃ alkyl refers to an alkyl group of 1 to 3 carbons wherein at least one hydrogen atom is replaced by a halogen. Halo-C₁-C₆ alkyl refers to an alkyl group of 1 to 6 carbons wherein at least one hydrogen atom is replaced by a halogen. Where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen. For example, halo-C₁-C₃ alkoxy refers to an alkoxy group of 1 to 3 carbons wherein at least one hydrogen atom is replaced by a halogen. Halo-C₁-C₆ alkoxy refers to an alkoxy group of 1 to 6 carbons wherein at least one hydrogen atom is replaced by a halogen. Non-limiting examples of haloalkoxy are —OCH₂CF₃, —OCF₂H, and —OCF₃.

“Hydroxyalkyl” refers to an alkyl group as defined above, wherein one or more (e.g., 1 to 6, or 1 to 3) hydrogen atoms are replaced by a hydroxy group (e.g., hydroxy-C₁-C₃-alkyl, hydroxy-C₁-C₆-alkyl). The term “hydroxy-C₁-C₃ alkyl” refers to a one to three carbon alkyl chain where one or more hydrogens on any carbon is replaced by a hydroxy group, in particular, one hydrogen on one carbon of the chain is replaced by a hydroxy group. The term “hydroxy-C₁-C₆ alkyl” refers to a one to six carbon alkyl chain where one or more hydrogens on any carbon is replaced by a hydroxy group, in particular, one hydrogen on one carbon of the chain is replaced by a hydroxy group. Non-limiting examples of hydroxyalkyl include —CH₂OH, —CH₂CH₂OH, and —C(CH₃)₂CH₂OH.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. In certain embodiments, the heteroalkyl can have 1 to 3 carbon atoms (e.g., C₁-C₃ heteroalkyl) or 1 to 6 carbon atoms (e.g., C₁-C₆ heteroalkyl), and one or more (e.g., 1, 2, or 3) heteroatoms or heteroatomic groups. The term “heteroalkyl” includes unbranched or branched saturated chain having carbon and heteroatoms. By way of example, 1, 2, or 3 carbon atoms of the alkyl group in the “heteroalkyl” may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein. In certain embodiments, heteroalkyl can have 1 to 20 carbon atoms, 1 to 15 carbon atoms, 1 to 12 carbon atoms, 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems (i.e., 9-10 membered heteroaryl), 5-10 membered ring systems (i.e., 5-10 membered heteroaryl), 5-7 membered ring systems (i.e., 5-7 membered heteroaryl), 5-6 membered ring systems (i.e., 5-6 membered heteroaryl), or 4-6 membered ring systems (i.e., 4-6 membered heteroaryl), each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic group, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”, such as (5- to 10-membered monocyclic heteroaryl)-C₁-C₃ alkyl.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro. Any non-aromatic ring containing at least one heteroatom is considered a heterocyclyl, regardless of the attachment (i.e., can be bound through a carbon atom or a heteroatom). Further, the term heterocyclyl is intended to encompass a moiety comprising any non-aromatic ring containing at least one heteroatom, which ring may be fused to an aryl or heteroaryl ring, regardless of the attachment to the remainder of the molecule. The term heterocyclyl is also intended to encompass a moiety comprising a cycloalkyl ring which is fused to a heteroaryl ring, regardless of the attachment to the remainder of the molecule. Additionally, the term heterocyclyl is intended to encompass a moiety comprising a cycloalkyl ring which is fused to a heterocyclyl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- to 6-ring atoms, it is also referred to herein as a 4- to 6-membered heterocyclyl. Also disclosed herein are 5- or 6-membered heterocyclyls, having 5 or 6 ring atoms, respectively, and 5- to 10-membered heterocyclyls, having 5 to 10 ring atoms. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, tetrahydropyranyl, thiomorpholinyl, thiamorpholinyl, 1-oxo-thiomorpholinyl and 1,1-dioxo-thiomorpholinyl. In certain embodiments, the term “heterocyclyl” can include “spiroheterocyclyl” when there are two positions for substitution on the same carbon atom, wherein at least one ring of the spiro system comprises at least one heteroatom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Oxo” refers to the group (═O).

“Cyano” refers to the group (—CN).

“N-oxide” refers to the group (—N⁺—O—).

“Thiol” refers to the group (—SH).

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. A non-limiting example of a sulfonyl group is —SO₂(C₁-C₆ alkyl), which is herein referred to as alkylsulfonyl. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5, 1 to 4, or 1 to 3) hydrogen atom is replaced by a bond to a non-hydrogen moiety. Unless otherwise described, such non-hydrogen moieties may include, but are not limited to, alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanidino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5, 1 to 4, or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl)substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure include, for example, those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), and mixture of isomers (such as a mixture of stereoisomers), of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use. Generally, such a material is not biologically or otherwise undesirable, e.g., the material may be incorporated into a pharmaceutical composition administered to a patient without causing any significant undesirable biological effects or interacting in a deleterious manner with any of the other components of the composition in which it is contained.

The term “pharmaceutically acceptable salt” of a given compound includes salts which are generally safe and not biologically or otherwise undesireable, and includes those which are acceptable for veterinary use as well as human pharmaceutical use. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. Solvates include hydrates.

Some of the compounds described herein may exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. Another example of a compound that has several tautomers is 1,4-thiazine. The tautomers are 1λ⁴,4-thiazine, 2H-1,4-thiazine, and 4H-1,4-thiazine, wherein only 1λ⁴,4-thiazine is aromatic.

The compounds described herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Treatment” or “treating” is an approach for obtaining beneficial or desired results including but not limited to clinical results. Beneficial or desired results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease or condition, that is, causing the regression of clinical symptoms (e.g., ameliorating the disease state, providing partial or total remission of the disease or condition, enhancing effect of another medication, delaying the progression of the disease, increasing the quality of life, and/or prolonging survival). Also encompassed by “treatment” or “treating” is a reduction of pathological consequence of demyelination.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, or deuterated analog thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one of ordinary skill in the art. The effective amount of a compound of the disclosure in such a therapeutic method is, for example, from about 0.01 mg/kg/day to about 1000 mg/kg/day, or from about 0.1 mg/kg/day to about 100 mg/kg/day.

The term “excipient” as used herein refers to an inert or inactive substance that may be used in the production of a drug or pharmaceutical composition, such as a tablet containing a compound as described herein (or pharmaceutically acceptable salt) as an active ingredient. Various substances may be embraced by the term excipient, including without limitation any substance used as a diluent, filler or extender, binder, disintegrant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Binders may include, e.g., carbomers, povidone, xanthan gum, etc.; coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch de, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g. dextrose, fructose de, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose de, sorbitol, sucrose de, etc.; and wet granulation agents include, e.g., calcium carbonate, maltodextrin, microcrystalline cellulose, etc. In some cases, the term “excipient” ecompassess pharmaceutically acceptable carriers.

Additional definitions may also be provided below as appropriate.

### II. Compounds

In certain embodiments, the subject matter described herein is directed to compounds of Formula I:

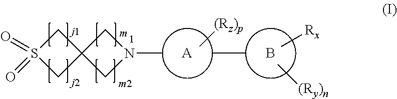

or pharmaceutically acceptable salts thereof, wherein,
j₁, j₂, and m₁ are each independently 1, 2, or 3;
m₂ is 0, 1, 2, or 3;
   wherein the sum of j₁ and j₂ and the sum of m₁ and m₂ are each no more than 5, and the total sum of j₁, j₂, m₁, and m₂ is no more than 9;
Ring A is

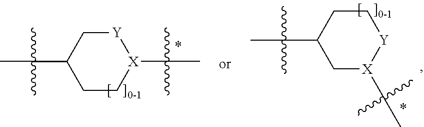

where [symbol] indicates the connection of the ring to the remainder of the molecule, wherein * indicates the connection of Ring A to Ring B;
   X is N or CH, Y is O or CH₂, wherein when X is N then Y is CH₂;
R_{z} if present, in each instance is independently selected from the group consisting of halogen, hydroxy, C₁-C₆ alkyl, halo-C₁-C₆ alkyl, C₁-C₆ alkoxy, halo-C₁-C₆ alkoxy, —CN, and —NRR′;
and, two R_{z}, together with the atom on Ring A to which each is attached, may form a bridge;
p is 0, 1, 2, or 3;
Ring B is phenyl, or 5- to 6-membered heteroaryl comprising one, two, or three heteroatoms independently selected from the group consisting of O, N, and S;
R_{y} if present, in each instance is independently selected from the group consisting of halogen, C₁-C₆ alkyl, halo-C₁-C₆ alkyl, hydroxy, C₁-C₆ alkoxy, halo-C₁-C₆ alkoxy, C₃-C₇ cycloalkyl, C₃-C₇ halocycloalkyl, —SO₂(C₁-C₆ alkyl), —CN, and —NRR′;
n is 0, 1, 2, 3, or 4;
Rₓ is selected from the group consisting of halogen, hydroxy, C₁-C₁₀ alkyl, halo-C₁-C₆ alkyl, C₁-C₆ alkoxy, halo-C₁-C₆ alkoxy, 5- to 7-membered heterocyclyl, C₃-C₇ cycloalkyl, C₆-C₁₀ aryl, 5- to 6-membered heteroaryl, —SO₂(C₁-C₆ alkyl), —CN, and —NRR′;
   wherein said heterocyclyl, cycloalkyl, aryl, or heteroaryl is substituted with (R_{xA})_{q};
      wherein q is 0, 1, 2, 3, 4, or 5; and
      R_{xA}, if present, in each instance is independently selected from the group consisting of halogen, C₁-C₆ alkyl, hydroxy, C₁-C₆ alkoxy, halo-C₁-C₆ alkoxy, halo-C₁-C₆ alkyl, —SO₂(C₁-C₆ alkyl), —CN, and —NR″R′″;
R, R′, R″, and R′″ are each independently H, C₁-C₆ alkyl, or halo-C₁-C₆ alkyl.

In certain embodiments, compounds include those of Formula I, or pharmaceutically acceptable salts thereof, where Ring B is phenyl or a 5- or 6-membered heteroaryl comprising one to three N. In certain embodiments, compounds include those of Formula I where Ring B is selected from the group consisting of phenyl, pyridinyl, pyrimidinyl, pyrrolyl, pyrazolyl, imidazolyl, triazolyl, pyridazinyl, and triazinyl, or pharmaceutically acceptable salts thereof. In certain embodiments, compounds include those of Formula I where Ring B is selected from the group consisting of phenyl, pyrazolyl, pyridinyl, triazolyl, and imidazolyl, or pharmaceutically acceptable salts thereof. In certain embodiments, compounds include those of Formula I where Ring B is selected from the group consisting of pyrazolyl, pyridinyl, triazolyl, and imidazolyl, or pharmaceutically acceptable salts thereof. In certain embodiments, compounds include those of Formula I where Ring B is a 5-membered heteroaryl comprising two or three N, or pharmaceutically acceptable salts thereof. In certain embodiments, compounds include those of Formula I, or pharmaceutically acceptable salts thereof, where Ring B is selected from the group consisting of pyrazolyl and triazolyl.

In certain embodiments, compounds include those of Formula I, or pharmaceutically acceptable salts thereof,
where Ring B is phenyl or a 5- or 6-membered heteroaryl comprising one to three N;
j₁, j₂, and m₁ are each independently 1, 2, or 3; and
m₂ is 0, 1, 2, or 3;
wherein the sum of j₁ and j₂ and the sum of m₁ and m₂ are each no more than 4, and the total sum of j₁, j₂, m₁, and m₂ is no more than 7.

In certain embodiments, compounds include those of Formula I, or pharmaceutically acceptable salts thereof, where Rₓ is selected from the group consisting of halogen, C₁-C₁₀ alkyl, halo-C₁-C₆ alkyl, C₁-C₆ alkoxy, halo-C₁-C₆ alkoxy, 5- to 7-membered heterocyclyl, C₃-C₇ cycloalkyl, and 5- to 6-membered heteroaryl;
wherein said heterocyclyl, cycloalkyl, or aryl is substituted with (R_{xA})_{q}, wherein q is an integer from 0 to 3; and
R_{xA}, if present, in each instance is independently selected from the group consisting of halogen, C₁-C₆ alkyl, halo-C₁-C₆ alkyl, C₁-C₆ alkoxy, and halo-C₁-C₆ alkoxy.

In certain embodiments, compounds include those of Formula I, or pharmaceutically acceptable salts thereof, where each R_{y}, if present, is independently selected from the group consisting of halogen, C₁-C₆ alkyl, halo-C₁-C₆ alkyl, C₁-C₆ alkoxy, halo-C₁-C₆ alkoxy, C₃-C₇ cycloalkyl, and C₃-C₇ halocycloalkyl. In certain embodiments, R_{y} is selected from the group consisting of halogen, C₃-C₇ cycloalkyl, and C₁-C₆ alkyl. In certain embodiments, R_{y} is C₃-C₇ cycloalkyl. In certain embodiments, R_{y} is selected from the group consisting of cyclopropyl, cyclobutyl, and cyclopentyl. In certain embodiments, R_{y} is C₁-C₆ alkyl. In certain embodiments, R_{y} is selected from the group consisting of methyl, ethyl, propyl, and butyl. In certain embodiments, R_{y} is propyl. In certain embodiments, R_{y} is isopropyl.

In some embodiments, of the compounds of Formula I, or pharmaceutically acceptable salts thereof, p is 0, 1, or 2. In some embodiments, p is 0. In certain embodiments, R_{z} if present, in each instance is independently selected from the group consisting of halogen, hydroxy, C₁-C₃ alkyl, halo-C₁-C₃ alkyl, C₁-C₃ alkoxy, halo-C₁-C₃ alkoxy, —CN, and —NRR′, wherein each R and R′ is independently H, C₁-C₃ alkyl, or halo-C₁-C₃ alkyl. In further embodiments, R_{z}, if present, in each instance is independently halogen, hydroxy, C₁-C₃ alkyl, or halo-C₁-C₃ alkyl.

In certain embodiments, compounds include those of Formula I, or pharmaceutically acceptable salts thereof, where the sum of m₁ and m₂ is not greater than 3.

In some embodiments of the compounds of Formula I, or pharmaceutically acceptable salts thereof, Ring A is

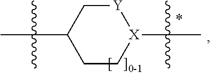

wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In other embodiments of the compounds of Formula I, or pharmaceutically acceptable salts thereof, Ring A is

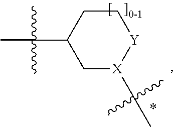

where wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In still further embodiments of the compounds of Formula I, or pharmaceutically acceptable salts thereof, m₁ and m₂ are independently 1 or 2; j₁ and j₂ are independently 1 or 2.

In certain embodiments of the compounds of Formula I, or pharmaceutically acceptable salts thereof, one of m₁ and m₂ is 1 and the other is 2; both of j₁ and j₂ are 1.

In other embodiments of the compounds of Formula I, or pharmaceutically acceptable salts thereof, both of m₁ and m₂ are 2; both of j₁ and j₂ are 1.

In certain embodiments of the compounds of Formula I, or pharmaceutically acceptable salts thereof, Ring A is selected from the group consisting of

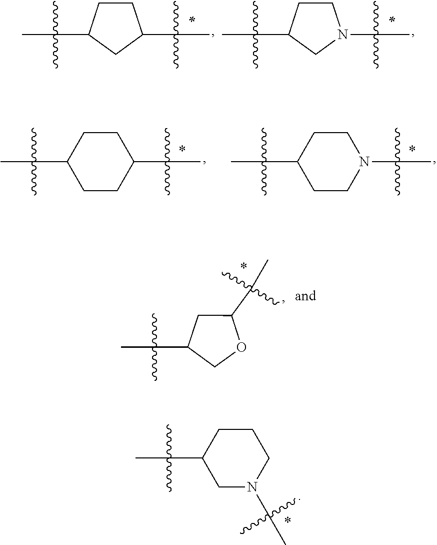 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

In some embodiments of Formula I, or pharmaceutically acceptable salts thereof, Ring A is:

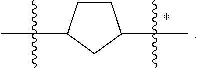 [SMILES: *C1CCC(*)C1]

In certain embodiments, m₁ and m₂ are independently 1 or 2; j₁ and j₂ are independently 1 or 2.

In some embodiments of Formula I, or pharmaceutically acceptable salts thereof, Ring A is:

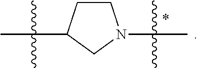 [SMILES: *C1CCN(*)C1]

In certain embodiments, m₁ and m₂ are independently 1 or 2; j₁ and j₂ are independently 1 or 2.

In some embodiments of Formula I, or pharmaceutically acceptable salts thereof, Ring A is:

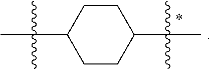 [SMILES: *C1CCC(*)CC1]

In certain embodiments, m₁ and m₂ are independently 1 or 2; j₁ and j₂ are independently 1 or 2.

In some embodiments of Formula I, or pharmaceutically acceptable salts thereof, Ring A is:

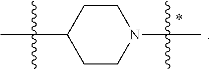 [SMILES: *C1CCN(*)CC1]

In certain embodiments, m₁ and m₂ are independently 1 or 2; j₁ and j₂ are independently 1 or 2.

In some embodiments of Formula I, or pharmaceutically acceptable salts thereof, Ring A is:

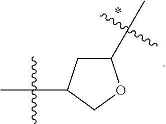 [SMILES: *C1COC(*)C1]

In certain embodiments, m₁ and m₂ are independently 1 or 2; j₁ and j₂ are independently 1 or 2.

In some embodiments of Formula I, or pharmaceutically acceptable salts thereof, Ring A is:

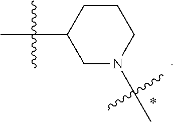 [SMILES: *C1CCCN(*)C1]

In certain embodiments, m₁ and m₂ are independently 1 or 2; j₁ and j₂ are independently 1 or 2.

In certain embodiments (Embodiment A), compounds of Formula I are of Formula Ia:

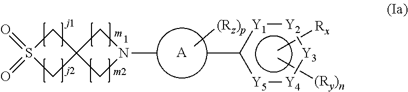

or pharmaceutically acceptable salts thereof, wherein Y₁, Y₂, Y₃, Y₄, and Y₅ are each independently selected from the group consisting of CH, C, N, S, and SH, wherein C and S are substituted with Rₓ or R_{y}, and wherein one, two, or three of Y₁, Y₂, Y₃, Y₄, and Y₅ can be N, SH, S—Rₓ, or S—R_{y}; and j₁, j₂, m₁, m₂, R_{z}, Rₓ, R_{y}, p, and n are as defined for Formula I.

In certain embodiments of the above Embodiment A, compounds include those of Formula Ia, or pharmaceutically acceptable salts thereof, where Y₁-Y₅ are each independently selected from the group consisting of CH, C, and N, wherein C is substituted with R_{y} or Rₓ. In certain embodiments, compounds include those of Formula Ia where one of Y₁, Y₂, Y₃, Y₄, and Y₅ is N; one is CRₓ; and the remainder are each independently CH or CR_{y}. In certain embodiments, compounds include those of Formula Ia where one of Y₁, Y₂, Y₃, Y₄, and Y₅ is CRₓ, and the remainder are each independently CH or CR_{y}. In certain embodiments, compounds include those of Formula Ia where Y₃ is C—Rₓ. In certain embodiments, compounds include those of Formula Ia where Y₁, Y₂, Y₄, and Y₅ are each CH and Y₃ is C—Rₓ. In certain embodiments, compounds include those of Formula Ia where Y₁, Y₃, and Y₅ are CH, Y₂ is N, and Y₄ is C—Rₓ. In certain embodiments, compounds include those of Formula Ia where Y₁ is N, Y₂, Y₄, and Y₅ are CH, and Y₃ is C—Rₓ. In certain embodiments, compounds include those of Formula Ia where Y₁ is C—R_{y}, Y₂ and Y₄ are CH, Y₅ is N, and Y₃ is C—Rₓ. In certain embodiments, compounds include those of Formula Ia where Y₁ is C—R_{y}, Y₂ and Y₅ are CH, Y₄ is N, and Y₃ is C—Rₓ. In certain embodiments, compounds include those of Formula Ia where Y₁ is C—R_{y}, Y₂ is N, Y₄ and Y₅ are CH, and Y₃ is C—Rₓ. In certain embodiments, R_{y} is selected from the group consisting of halogen, C₃-C₇ cycloalkyl, and C₁-C₆ alkyl. In certain embodiments, R_{y} is C₃-C₇ cycloalkyl. In certain embodiments, R_{y} is selected from the group consisting of cyclopropyl, cyclobutyl, and cyclopentyl. In certain embodiments, R_{y} is C₁-C₆ alkyl. In certain embodiments, R_{y} is selected from the group consisting of methyl, ethyl, propyl, and butyl. In certain embodiments, R_{y} is propyl. In certain embodiments, R_{y} is isopropyl.

In some embodiments of the above Embodiment A, compounds of Formula Ia, or pharmaceutically acceptable salts thereof, include those where p is 0, 1, or 2. In some embodiments, p is 0. In certain embodiments, R_{z} if present, in each instance is independently selected from the group consisting of halogen, hydroxy, C₁-C₃ alkyl, halo-C₁-C₃ alkyl, C₁-C₃ alkoxy, halo-C₁-C₃ alkoxy, —CN, and —NRR′, wherein each R and R′ is independently H, C₁-C₃ alkyl, or halo-C₁-C₃ alkyl. In further embodiments, R_{z}, if present, in each instance is independently halogen, hydroxy, C₁-C₃ alkyl, or halo-C₁-C₃ alkyl.

In some embodiments of the above Embodiment A, compounds of Formula Ia, or pharmaceutically acceptable salts thereof, include those where Ring A is

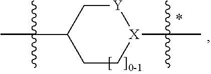

wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In other embodiments of the above Embodiment A, compounds of Formula Ia, or pharmaceutically acceptable salts thereof, include those where Ring A is

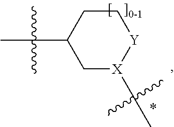

where wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In still further embodiments of the above Embodiment A, compounds of Formula Ia, or pharmaceutically acceptable salts thereof, include those where m₁ and m₂ are independently 1 or 2; j₁ and j₂ are independently 1 or 2.

In certain embodiments of the above Embodiment A, compounds of Formula Ia, or pharmaceutically acceptable salts thereof, include those where one of m₁ and m₂ is 1 and the other is 2; both of j₁ and j₂ are 1.

In other embodiments of the above Embodiment A, compounds of Formula Ia, or pharmaceutically acceptable salts thereof, include those where both of m₁ and m₂ are 2; both of j₁ and j₂ are 1.

In certain embodiments of the above Embodiment A, compounds of Formula Ia, or pharmaceutically acceptable salts thereof, include those where Ring A is selected from the group consisting of

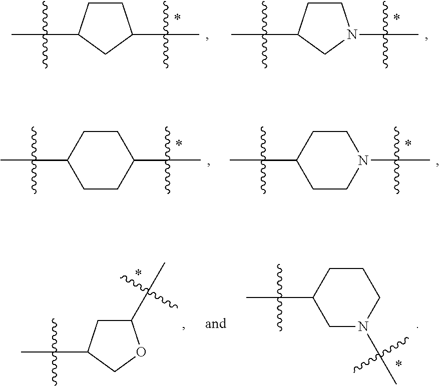 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

In some embodiments of the above Embodiment A, compounds of Formula Ia, or pharmaceutically acceptable salts thereof, include those where Ring A is:

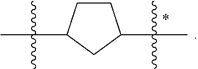 [SMILES: *C1CCC(*)C1]

In certain embodiments, m₁ and m₂ are independently 1 or 2; j₁ and j₂ are independently 1 or 2.

In some embodiments of the above Embodiment A, compounds of Formula Ia, or pharmaceutically acceptable salts thereof, include those where Ring A is:

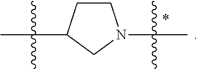 [SMILES: *C1CCN(*)C1]

In certain embodiments, m₁ and m₂ are independently 1 or 2; j₁ and j₂ are independently 1 or 2.

In some embodiments of the above Embodiment A, compounds of Formula Ia, or pharmaceutically acceptable salts thereof, include those where Ring A is:

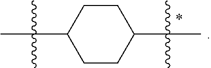 [SMILES: *C1CCC(*)CC1]

In certain embodiments, m₁ and m₂ are independently 1 or 2; j₁ and j₂ are independently 1 or 2.

In some embodiments of the above Embodiment A, compounds of Formula Ia, or pharmaceutically acceptable salts thereof, include those where Ring A is:

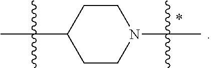 [SMILES: *C1CCN(*)CC1]

In certain embodiments, m₁ and m₂ are independently 1 or 2; j₁ and j₂ are independently 1 or 2.

In some embodiments of the above Embodiment A, compounds of Formula Ia, or pharmaceutically acceptable salts thereof, include those where Ring A is:

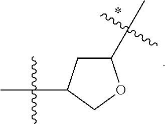 [SMILES: *C1COC(*)C1]

In certain embodiments, m₁ and m₂ are independently 1 or 2; j₁ and j₂ are independently 1 or 2.

In some embodiments of the above Embodiment A, compounds of Formula Ia, or pharmaceutically acceptable salts thereof, include those where Ring A is:

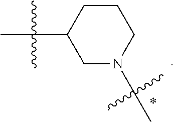 [SMILES: *C1CCCN(*)C1]

In certain embodiments, m₁ and m₂ are independently 1 or 2; j₁ and j₂ are independently 1 or 2.

In some embodiments of the above Embodiment A, compounds of Formula Ia, or pharmaceutically acceptable salts thereof, include those where Y₁, Y₂, Y₃, Y₄, and Y₅ are each independently N, C, or CH, wherein one or two of Y₁, Y₂, Y₃, Y₄, and Y₅ can be N;
n is 0 or 1;
R_{y} is C₃-C₅ cycloalkyl, halogen, or C₁-C₆ alkyl;
Rₓ is halo-C₁-C₆ alkyl;
p is 0;
Ring A is selected from the group consisting of

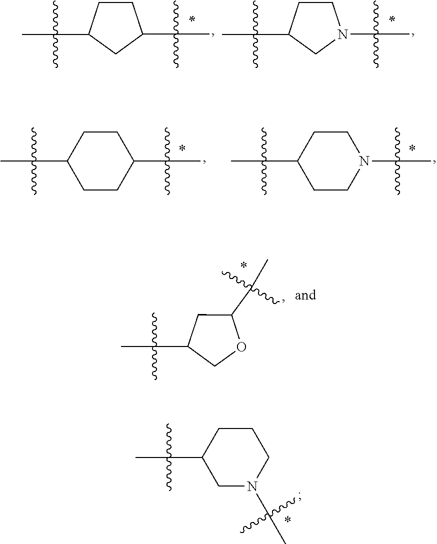 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

one of m₁ and m₂ is 2 and the other is 1; and
j₁ and j₂ are each 1.

In some embodiments of the above Embodiment A, such as, but not limited to, the above-described embodiment, compounds include those of Formula Ia, or pharmaceutically acceptable salts thereof, where one of Y₁, Y₂, Y₃, Y₄, and Y₅ is N. In certain embodiments, compounds include those where Rₓ is trifluoromethyl.

In certain embodiments (Embodiment B), compounds of Formula I are of Formula Ib:

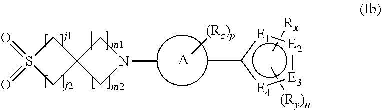

or pharmaceutically acceptable salts thereof, wherein E₁, E₂, E₃, and E₄ are each independently selected from the group consisting of CH, S, SH, C—Rₓ, C—R_{y}, N—Rₓ, N—R_{y}, N, O, NH, S—Rₓ, and S—R_{y}, wherein one, two, or three of E₁, E₂, E₃, or E₄ are S, SH, N, N—Rₓ, N—R_{y}, O, NH, S—Rₓ, or S—R_{y}; and j₁, j₂, m₁, m₂, R_{z}, Rₓ, R_{y}, p, and n are as defined for Formula I.

In certain embodiments of the above Embodiment B, compounds of Formula Ib, or pharmaceutically acceptable salts thereof, include those where E₂ is C—Rₓ. In certain embodiments, compounds include those of Formula Ib where E₃ is N, N—R_{y}, or CH. In certain embodiments, compounds include those of Formula Ib where n is 1. In still further embodiments, E₂ is C—Rₓ; and E₃ is N, N—R_{y}, or CH; and n is 1.

In certain embodiments of the above Embodiment B, compounds of Formula Ib, or pharmaceutically acceptable salts thereof, include those where R_{y} is selected from the group consisting of halogen, C₃-C₇ cycloalkyl, and C₁-C₆ alkyl. In certain embodiments, R_{y} is C₃-C₇ cycloalkyl. In certain embodiments, R_{y} is selected from the group consisting of cyclopropyl, cyclobutyl, and cyclopentyl. In certain embodiments, R_{y} is C₁-C₆ alkyl. In certain embodiments, R_{y} is selected from the group consisting of methyl, ethyl, propyl, and butyl. In certain embodiments, R_{y} is propyl. In certain embodiments, R_{y} is isopropyl.

In certain embodiments of the above Embodiment B, compounds of Formula Ib, or pharmaceutically acceptable salts thereof, include those where p is 0, 1, or 2. In some embodiments, p is 0. In certain embodiments, R_{z} if present, in each instance is independently selected from the group consisting of halogen, hydroxy, C₁-C₃ alkyl, halo-C₁-C₃ alkyl, C₁-C₃ alkoxy, halo-C₁-C₃ alkoxy, —CN, and —NRR′, wherein each R and R′ is independently H, C₁-C₃ alkyl, or halo-C₁-C₃ alkyl. In further embodiments, R_{z}, if present, in each instance is independently halogen, hydroxy, C₁-C₃ alkyl, or halo-C₁-C₃ alkyl.

In certain embodiments of the above Embodiment B, compounds of Formula Ib, or pharmaceutically acceptable salts thereof, include those where Ring A is

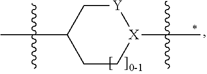

wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In certain embodiments of the above Embodiment B, compounds of Formula Ib, or pharmaceutically acceptable salts thereof, include those where Ring A is

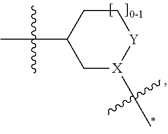

where wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In still further embodiments of the above Embodiment B, compounds of Formula Ib, or pharmaceutically acceptable salts thereof, include those where m₁ and m₂ are independently 1 or 2; and j₁ and j₂ are independently 1 or 2.

In certain embodiments of the above Embodiment B, compounds of Formula Ib, or pharmaceutically acceptable salts thereof, include those where one of m₁ and m₂ is 1 and the other is 2; and both of j₁ and j₂ are 1.

In other embodiments of the above Embodiment B, compounds of Formula Ib, or pharmaceutically acceptable salts thereof, include those where both of m₁ and m₂ are 2; both of j₁ and j₂ are 1.

In certain embodiments of the above Embodiment B, compounds of Formula Ib, or pharmaceutically acceptable salts thereof, include those where Ring A is selected from the group consisting of

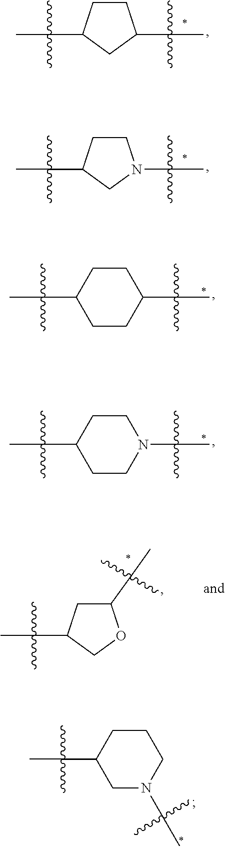 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

In some embodiments of the above Embodiment B, compounds of Formula Ib, or pharmaceutically acceptable salts thereof, include those where Ring A is:

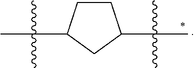 [SMILES: *C1CCC(*)C1]

In certain embodiments, m₁ and m₂ are independently 1 or 2; j₁ and j₂ are independently 1 or 2.

In some embodiments of the above Embodiment B, compounds of Formula Ib, or pharmaceutically acceptable salts thereof, include those where Ring A is:

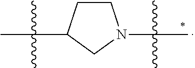 [SMILES: *C1CCN(*)C1]

In certain embodiments, m₁ and m₂ are independently 1 or 2; j₁ and j₂ are independently 1 or 2.

In some embodiments of the above Embodiment B, compounds of Formula Ib, or pharmaceutically acceptable salts thereof, include those where Ring A is:

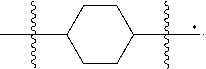 [SMILES: *C1CCC(*)CC1]

In certain embodiments, m₁ and m₂ are independently 1 or 2; j₁ and j₂ are independently 1 or 2.

In some embodiments of the above Embodiment B, compounds of Formula Ib, or pharmaceutically acceptable salts thereof, include those where Ring A is:

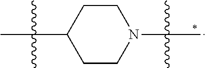 [SMILES: *C1CCN(*)CC1]

In certain embodiments, m₁ and m₂ are independently 1 or 2; j₁ and j₂ are independently 1 or 2.

In some embodiments of the above Embodiment B, compounds of Formula Ib, or pharmaceutically acceptable salts thereof, include those where Ring A is:

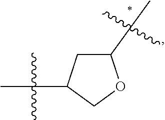 [SMILES: *C1COC(*)C1]

In certain embodiments, m₁ and m₂ are independently 1 or 2; j₁ and j₂ are independently 1 or 2.

In some embodiments of the above Embodiment B, compounds of Formula Ib, or pharmaceutically acceptable salts thereof, include those where Ring A is:

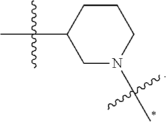 [SMILES: *C1CCCN(*)C1]

In certain embodiments, m₁ and m₂ are independently 1 or 2; j₁ and j₂ are independently 1 or 2.

In certain embodiments (Embodiment C), compounds of Formula I are of Formula Ic:

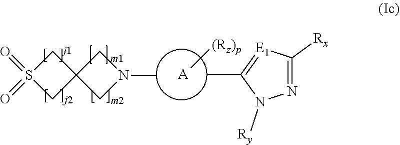

or pharmaceutically acceptable salts thereof, wherein E₁ is N or CH; and j₁, j₂, m₁, m₂, R_{z}, Rₓ, R_{y}, and p are as defined for Formula I.

In certain embodiments of the above Embodiment C, compounds of Formula Ic, or pharmaceutically acceptable salts thereof, include those where Rₓ is selected from the group consisting of halo-C₁-C₆ alkyl, C₃-C₅ cycloalkyl, phenyl, and 6-membered heteroaryl, wherein said cycloalkyl, phenyl, or heteroaryl is substituted with (R_{xA})_{q}. In certain embodiments, compounds include those where Rₓ is selected from the group consisting of trifluoromethyl, difluoromethyl, fluoromethyl, trifluoroethyl, difluoroethyl, and fluoroethyl. In certain embodiments, compounds include those where Rₓ is trifluoromethyl. In certain embodiments, compounds include those where Rₓ is cyclopropyl, and q is 0. In certain embodiments, R_{y} is selected from the group consisting of halogen, C₃-C₇ cycloalkyl, and C₁-C₆ alkyl. In certain embodiments, R_{y} is C₃-C₇ cycloalkyl. In certain embodiments, R_{y} is selected from the group consisting of cyclopropyl, cyclobutyl, and cyclopentyl. In certain embodiments, R_{y} is C₁-C₆ alkyl. In certain embodiments, R_{y} is selected from the group consisting of methyl, ethyl, propyl, and butyl. In certain embodiments, R_{y} is propyl. In certain embodiments, R_{y} is isopropyl.

In certain embodiments of the above Embodiment C, compounds of Formula Ic, or pharmaceutically acceptable salts thereof, include those where p is 0, 1, or 2. In some embodiments, p is 0. In certain embodiments, R_{z} if present, in each instance is independently selected from the group consisting of halogen, hydroxy, C₁-C₃ alkyl, halo-C₁-C₃ alkyl, C₁-C₃ alkoxy, halo-C₁-C₃ alkoxy, —CN, and —NRR′, wherein each R and R′ is independently H, C₁-C₃ alkyl, or halo-C₁-C₃ alkyl. In further embodiments, R_{z}, if present, in each instance is independently halogen, hydroxy, C₁-C₃ alkyl, or halo-C₁-C₃ alkyl.

In certain embodiments of the above Embodiment C, compounds of Formula Ic, or pharmaceutically acceptable salts thereof, include those where Ring A is

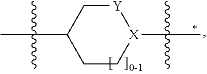

wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In certain embodiments of the above Embodiment C, compounds of Formula Ic, or pharmaceutically acceptable salts thereof, include those where Ring A is

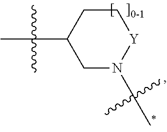

where wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In still further embodiments of the above Embodiment C, compounds of Formula Ic, or pharmaceutically acceptable salts thereof, include those where m₁ and m₂ are independently 1 or 2; j₁ and j₂ are independently 1 or 2.

In certain embodiments of the above Embodiment C, compounds of Formula Ic, or pharmaceutically acceptable salts thereof, include those where one of m₁ and m₂ is 1 and the other is 2; both of jᵢ and j₂ are 1.

In other embodiments of the above Embodiment C, compounds of Formula Ic, or pharmaceutically acceptable salts thereof, include those where both of m₁ and m₂ are 2; both of j₁ and j₂ are 1.

In certain embodiments of the above Embodiment C, compounds of Formula Ic, or pharmaceutically acceptable salts thereof, include those where Ring A is selected from the group consisting of

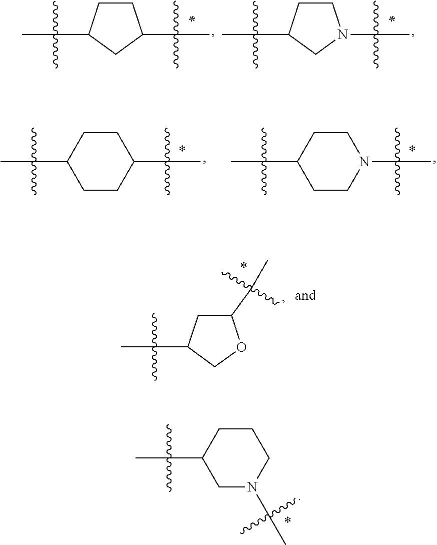 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

In some embodiments of the above Embodiment C, compounds of Formula Ic, or pharmaceutically acceptable salts thereof, include those where Ring A is:

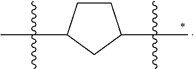 [SMILES: *C1CCC(*)C1]

In certain embodiments, m₁ and m₂ are independently 1 or 2; j₁ and j₂ are independently 1 or 2.

In some embodiments of the above Embodiment C, compounds of Formula Ic, or pharmaceutically acceptable salts thereof, include those where Ring A is:

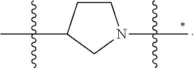 [SMILES: *C1CCN(*)C1]

In certain embodiments, m₁ and m₂ are independently 1 or 2; j₁ and j₂ are independently 1 or 2.

In some embodiments of the above Embodiment C, compounds of Formula Ic, or pharmaceutically acceptable salts thereof, include those where Ring A is:

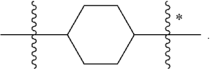 [SMILES: *C1CCC(*)CC1]

In certain embodiments, m₁ and m₂ are independently 1 or 2; j₁ and j₂ are independently 1 or 2.

In some embodiments of the above Embodiment C, compounds of Formula Ic, or pharmaceutically acceptable salts thereof, include those where Ring A is:

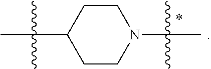 [SMILES: *C1CCN(*)CC1]

In certain embodiments, m₁ and m₂ are independently 1 or 2; j₁ and j₂ are independently 1 or 2.

In some embodiments of the above Embodiment C, compounds of Formula Ic, or pharmaceutically acceptable salts thereof, include those where Ring A is:

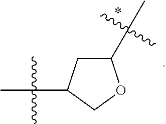 [SMILES: *C1COC(*)C1]

In certain embodiments, m₁ and m₂ are independently 1 or 2; j₁ and j₂ are independently 1 or 2.

In some embodiments of the above Embodiment C, compounds of Formula Ic, or pharmaceutically acceptable salts thereof, include those where Ring A is:

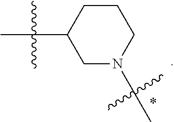 [SMILES: *C1CCCN(*)C1]

In certain embodiments, m₁ and m₂ are independently 1 or 2; j₁ and j₂ are independently 1 or 2.

In certain further embodiments of the above Embodiment C, compounds of Formula Ic, or pharmaceutically acceptable salts thereof, include those where
E₁ is CH or N;
R_{y} is C₁-C₆ alkyl or C₃-C₅ cycloalkyl;
Rₓ is selected from the group consisting of halo-C₁-C₆ alkyl, phenyl, C₃-C₅ cycloalkyl, and 5- or 6-membered heteroaryl; wherein said cycloalkyl, phenyl or heteroaryl is substituted with (R_{xA})_{q};
   wherein q is 0, 1, or 2; and
   R_{xA}, if present, is in each instance independently selected from the group consisting of halogen, C₁-C₆ alkyl, hydroxy, C₁-C₆ alkoxy, halo-C₁-C₆ alkoxy, and halo-C₁-C₆ alkyl;
p is 0;
Ring A is

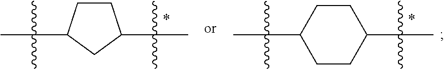 [SMILES: *C1CCC(*)C1 ; *C1CCC(*)CC1]

one of m₁ and m₂ is 2 and the other is 1, or m₁ and m₂ are each 2, or one of m₁ and m₂ is 3 and the other is 1, or m₁ and m₂ are each 1; and
j₁ and j₂ are each 1, or one of j₁ and j₂ is 2 and the other is 1, or j₁ and j₂ are each 2.

In certain aspects of the above Embodiment C, compounds of Formula Ic, or pharmaceutically acceptable salts thereof, include those where one of m₁ and m₂ is 2 and the other is 1; and j₁ and j₂ are each 1. In certain embodiments, compounds include those where R_{y} is selected from the group consisting of methyl, ethyl, propyl, cyclopropyl, cyclobutyl, and cyclopentyl. In certain embodiments, compounds include those where R_{y} is isopropyl or cyclopropyl. In certain embodiments, compounds include those where Rₓ is selected from the group consisting of halo-C₁-C₆ alkyl, phenyl, 6-membered heteroaryl, and cyclopropyl, wherein said phenyl, 6-membered heteroaryl, or cyclopropyl is substituted with (R_{xA})_{q}. In certain embodiments, compounds include those where Rₓ is trifluoromethyl. In certain embodiments, compounds include those where Rₓ is cyclopropyl, wherein q is 0. In certain embodiments, compounds include those where Rₓ is pyridinyl substituted with (R_{xA})_{q}, wherein q is 1. In certain embodiments, compounds include those where R_{xA} is halo-C₁-C₆ alkyl or halo-C₁-C₆ alkoxy. In certain embodiments, compounds include those where R_{xA} is trifluoromethyl or trifluoromethoxy.

In some embodiments of the compounds of Formula I, Ia, Ib, or Ic, or a pharmaceutically acceptable salt of any of the foregoing, Rₓ is selected from the group consisting of halogen, C₁-C₁₀ alkyl, halo-C₁-C₆ alkyl, C₁-C₆ alkoxy, halo-C₁-C₆ alkoxy, 5- to 7-membered heterocyclyl, C₃-C₇ cycloalkyl, and 5- to 6-membered heteroaryl;
wherein said heterocyclyl, cycloalkyl, or aryl is substituted with (R_{xA})_{q}, wherein q is an integer from 0 to 3; and
R_{xA}, if present, in each instance is independently selected from the group consisting of halogen, C₁-C₆ alkyl, halo-C₁-C₆ alkyl, C₁-C₆ alkoxy, and halo-C₁-C₆ alkoxy.

In certain embodiments (Embodiment D), compounds of Formula I are of Formula Id:

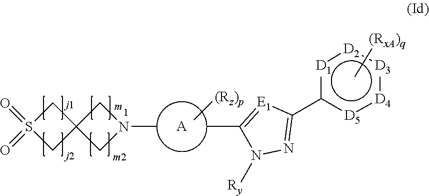

or pharmaceutically acceptable salts thereof, wherein D₁, D₂, D₃, D₄, and D₅ are each independently N, C when bound to R_{xA}, or CH, wherein up to three of D₁, D₂, D₃, D₄, and D₅ are N; and q is 1 or 2; and j₁, j₂, m₁, m₂, R_{z}, R_{y}, and p are as defined for Formula I.

In certain embodiments of the above Embodiment D, compounds of Formula Id, or pharmaceutically acceptable salts thereof, include those where D₃ or D₄ is C—R_{xA}. In certain embodiments, compounds include those of Formula Id where D₁ is CH, D₂ is CH, D₃ is C—R_{xA}, D₄ is N, and D₅ is CH. In certain embodiments, compounds include those of Formula Id where D₁ is CH, D₂ is N, D₃ is CH, D₄ is C—R_{xA}, and D₅ is CH. In certain embodiments, compounds include those of Formula Id where D₁, D₂, and D₃ are CH, D₄ is C—R_{xA}, and D₅ is N. In certain embodiments, compounds include those of Formula Id where R_{xA} is halo-C₁-C₆ alkyl or C₁-C₆ halo-alkoxy. In certain embodiments, compounds include those of Formula Id where R_{xA} is halo-C₁-C₆ alkyl. In certain embodiments, compounds include those of Formula Id where R_{xA} is selected from the group consisting of trifluoromethyl, difluoromethyl, fluoromethyl, trifluoroethyl, difluoroethyl, and fluoroethyl. In certain embodiments, compounds include those of Formula Id where R_{xA} is trifluoromethyl.

In certain embodiments of the above Embodiment D, compounds of Formula Id, or pharmaceutically acceptable salts thereof, include those where R_{y} is selected from the group consisting of halogen, C₃-C₇ cycloalkyl, and C₁-C₆ alkyl. In certain embodiments, compounds include those where R_{y} is C₃-C₇ cycloalkyl. In certain embodiments, compounds include those where R_{y} is selected from the group consisting of cyclopropyl, cyclobutyl, and cyclopentyl. In certain embodiments, compounds include those where R_{y} is C₁-C₆ alkyl. In certain embodiments, compounds include those where R_{y} is selected from the group consisting of methyl, ethyl, propyl, and butyl. In certain embodiments, compounds include those where R_{y} is propyl. In certain embodiments, compounds include those where R_{y} is isopropyl.

In some embodiments of the above Embodiment D, compounds of Formula Id, or pharmaceutically acceptable salts thereof, include those where p is 0, 1, or 2. In some embodiments, p is 0. In certain embodiments, R_{z} if present, in each instance is independently selected from the group consisting of halogen, hydroxy, C₁-C₃ alkyl, halo-C₁-C₃ alkyl, C₁-C₃ alkoxy, halo-C₁-C₃ alkoxy, —CN, and —NRR′, wherein each R and R′ is independently H, C₁-C₃ alkyl, or halo-C₁-C₃ alkyl. In further embodiments, R_{z}, if present, in each instance is independently halogen, hydroxy, C₁-C₃ alkyl, or halo-C₁-C₃ alkyl.

In certain embodiments of the above Embodiment D, compounds of Formula Id, or pharmaceutically acceptable salts thereof, include those where p is 0.

In certain embodiments of the above Embodiment D, compounds of Formula Id, or pharmaceutically acceptable salts thereof, include those where Ring A is

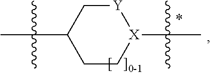

wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In other embodiments of the above Embodiment D, compounds of Formula Id, or pharmaceutically acceptable salts thereof, include those where Ring A is

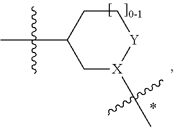

where wherein [symbol] indicates the connection of the ring to the remainder of the molecule, wherein * indicates the connection of Ring A to Ring B; and X is N or CH, Y is O or CH₂, wherein when X is N then Y is CH₂. In certain embodiments, X is N and Y is CH₂. In other embodiments, X is CH and Y is CH₂. In still further embodiments, X is CH and Y is O. In some embodiments, m₁ and m₂ are independently 1 or 2. In some embodiments, one of m₁ and m₂ is 1 and the other is 2. In other embodiments, both of m₁ and m₂ are 1. In still further embodiments, both of m₁ and m₂ are 2. In further embodiments, one of m₁ and m₂ is 3 and the other is 1. In certain embodiments, j₁ and j₂ are independently 1 or 2. In some embodiments, one of j₁ and j₂ is 1 and the other is 2. In other embodiments, both of j₁ and j₂ are 1. In still further embodiments, both of j₁ and j₂ are 2. In still further embodiments, m₁ and m₂ are independently 1 or 2; and j₁ and j₂ are independently 1 or 2. In certain embodiments, one of m₁ and m₂ is 1 and the other is 2; and both of j₁ and j₂ are 1.

In still further embodiments of the above Embodiment D, compounds of Formula Id, or pharmaceutically acceptable salts thereof, include those where m₁ and m₂ are independently 1 or 2; and j₁ and j₂ are independently 1 or 2.

In certain embodiments of the above Embodiment D, compounds of Formula Id, or pharmaceutically acceptable salts thereof, include those where one of m₁ and m₂ is 1 and the other is 2; both of j₁ and j₂ are 1.

In other embodiments of the above Embodiment D, compounds of Formula Id, or pharmaceutically acceptable salts thereof, include those where both of m₁ and m₂ are 2; both of j₁ and j₂ are 1.

In certain embodiments of the above Embodiment D, compounds of Formula Id, or pharmaceutically acceptable salts thereof, include those where Ring A is selected from the group consisting of

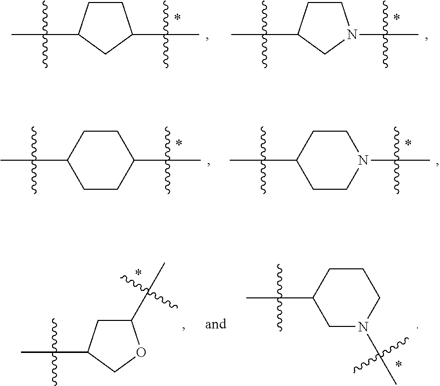 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

In some embodiments of the above Embodiment D, compounds of Formula Id, or pharmaceutically acceptable salts thereof, include those where Ring A is:

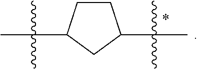 [SMILES: *C1CCC(*)C1]

In certain embodiments, m₁ and m₂ are independently 1 or 2; j₁ and j₂ are independently 1 or 2.

In some embodiments of the above Embodiment D, compounds of Formula Id, or pharmaceutically acceptable salts thereof, include those where Ring A is:

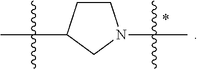 [SMILES: *C1CCN(*)C1]

In certain embodiments, m₁ and m₂ are independently 1 or 2; j₁ and j₂ are independently 1 or 2.

In certain embodiments of the above Embodiment D, compounds of Formula Id, or pharmaceutically acceptable salts thereof, include those where Ring A is:

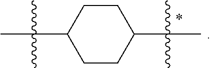 [SMILES: *C1CCC(*)CC1]

In certain embodiments, m₁ and m₂ are independently 1 or 2; j₁ and j₂ are independently 1 or 2.

In some embodiments of the above Embodiment D, compounds of Formula Id, or pharmaceutically acceptable salts thereof, include those where Ring A is:

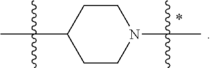 [SMILES: *C1CCN(*)CC1]

In certain embodiments, m₁ and m₂ are independently 1 or 2; j₁ and j₂ are independently 1 or 2.

In some embodiments of the above Embodiment D, compounds of Formula Id, or pharmaceutically acceptable salts thereof, include those where Ring A is:

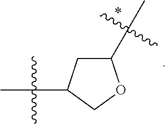 [SMILES: *C1COC(*)C1]

In certain embodiments, m₁ and m₂ are independently 1 or 2; j₁ and j₂ are independently 1 or 2.

In some embodiments of the above Embodiment D, compounds of Formula Id, or pharmaceutically acceptable salts thereof, include those where Ring A is:

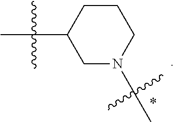 [SMILES: *C1CCCN(*)C1]

In certain embodiments, m₁ and m₂ are independently 1 or 2; j₁ and j₂ are independently 1 or 2.

In certain embodiments of the compounds of Formula I, or pharmaceutically acceptable salts thereof, such as compounds of Formula Ia, Ib, Ic, or Id, or pharmaceutically acceptable salt a of any of the foregoing, Ring A is selected from the group consisting of

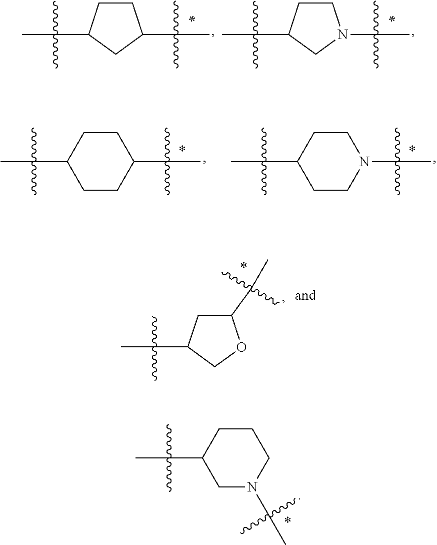 [SMILES: *C1CCC(*)C1 ; *C1CCN(*)C1 ; *C1CCC(*)CC1 ; *C1CCN(*)CC1 ; *C1COC(*)C1 ; *C1CCCN(*)C1]

In certain embodiments of the compounds of Formula I, or pharmaceutically acceptable salts thereof, such as compounds of Formula Ia, Ib, Ic, or Id, or pharmaceutically acceptable salts of any of the foregoing, compounds include those where one of m₁ and m₂ is 1 and the other is 2. In certain embodiments, compounds include those where m₁ and m₂ are each 1. In certain embodiments, compounds include those where one of m₁ and m₂ is 3 and the other is 1. In certain embodiments, compounds include those where m₁ and m₂ are each 2. In certain embodiments, compounds include those where m₁ is 1 and m₂ is 0. In certain embodiments, compounds include those where one of j₁ and j₂ is 1 and the other is 2. In certain embodiments, compounds include those where one of j₁ and j₂ is 3 and the other is 1. In certain embodiments, compounds include those where j₁ and j₂ are each 1. In certain embodiments, compounds include those where j₁ and j₂ are each 2.

In certain embodiments of the compounds of Formula I, or pharmaceutically acceptable salts thereof, such as compounds of Formula Ia, Ib, Ic, or Id, or pharmaceutically acceptable salts of any of the foregoing, compounds include those where j₁, j₂, m₁, and m₂ are as indicated in Table A.

TABLE A

| Spiro Ring | | | | | | | j₁ + j₂ + |
|---|---|---|---|---|---|---|---|
| Size | j₁ | j₂ | j₁ + j₂ | m₁ | m₂ | m₁ + m₂ | m₁ + m₂ |
| 4,5-fused | 1 | 1 | 2 | 2 | 1 | 3 | 5 |
| 4,5-fused | 1 | 1 | 2 | 1 | 2 | 3 | 5 |
| 5,6-fused | 1 | 2 | 3 | 1 | 3 | 4 | 7 |
| 5,5-fused | 1 | 2 | 3 | 1 | 2 | 3 | 6 |
| 5,5-fused | 1 | 2 | 3 | 2 | 1 | 3 | 6 |
| 6,5-fused | 2 | 2 | 4 | 2 | 1 | 3 | 7 |
| 5,6-fused | 2 | 1 | 3 | 3 | 1 | 4 | 7 |
| 4,6-fused | 1 | 1 | 2 | 3 | 1 | 4 | 6 |
| 4,6-fused | 1 | 1 | 2 | 2 | 2 | 4 | 6 |

In certain embodiments of the compounds of Formula I, or pharmaceutically acceptable salts thereof, such as compounds of Formula Ia, Ib, Ic, or Id, or pharmaceutically acceptable salts of any of the foregoing, Ring A is:

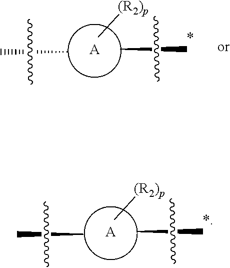

In some embodiments, Ring A is:

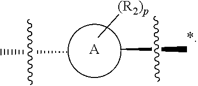

In other embodiments, Ring A is

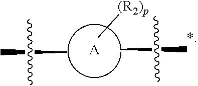

In certain embodiments of the compounds of Formula I, or pharmaceutically acceptable salts thereof, such as compounds of Formula Ia, Ib, Ic, or Id, or pharmaceutically acceptable salts of any of the foregoing, Ring A is:

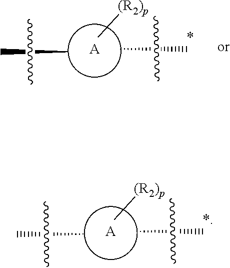

In some embodiments, Ring A is:

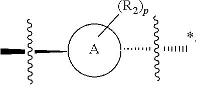

In other embodiments, Ring A is

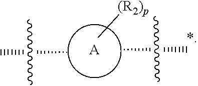

The subject matter described herein includes the following compounds in Table 1, or pharmaceutically acceptable salts thereof. In Table 1, the asterix (*) indicates an isolated isomer or isolated group of isomers, but that the stereochemistry has not been assigned. Individual enantiomers and diastereomers are included in the table below by compound name, and their corresponding structures can be readily determined therefrom. In some instances, the enantiomers or diastereomers of the present disclosure may be identified by their respective properties, for example, retention times by chiral HPLC, NMR peaks, and/or biological activities (e.g., as described further in the Examples), and the absolute stereo configurations of one or more chiral centers are arbitrarily assigned (e.g., stereochemistry of all chiral centers is arbitrarily assigned, or stereochemistry of one chiral center is known and remaining chiral centers arbitrarily assigned, etc.).

TABLE 1

| No. | Structure | Name |
|---|---|---|
| 1A | 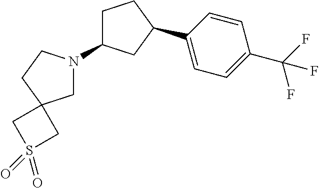 [SMILES: O=S1(=O)CC2(CCN([C@H]3CC[C@@H](c4ccc(C(F)(F)F)cc4)C3)C2)C1] | 6-((1S,3R)-3-(4- (trifluoromethyl)phenyl)cyclopen- tyl)-2-thia-6- azaspiro[3.4]octane 2,2-dioxide |
| 1B | 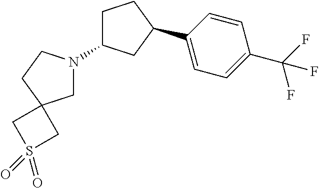 [SMILES: O=S1(=O)CC2(CCN([C@@H]3CC[C@@H](c4ccc(C(F)(F)F)cc4)C3)C2)C1] | 6-((1R,3R)-3-(4- (trifluoromethyl)phenyl)cyclopen- tyl)-2-thia-6- azaspiro[3.4]octane 2,2-dioxide |
| 1C | 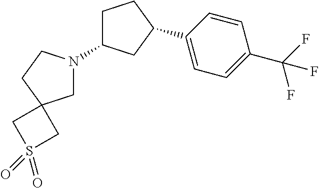 [SMILES: O=S1(=O)CC2(CCN([C@@H]3CC[C@H](c4ccc(C(F)(F)F)cc4)C3)C2)C1] | 6-((1R,3S)-3-(4- (trifluoromethyl)phenyl)cyclopen- tyl)-2-thia-6- azaspiro[3.4]octane 2,2-dioxide |
| 1D | 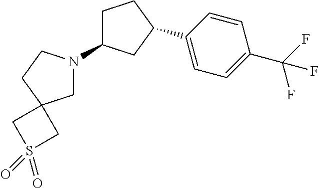 [SMILES: O=S1(=O)CC2(CCN([C@H]3CC[C@H](c4ccc(C(F)(F)F)cc4)C3)C2)C1] | 6-((1S,3S)-3-(4- (trifluoromethyl)phenyl)cyclopen- tyl)-2-thia-6- azaspiro[3.4]octane 2,2-dioxide |
| 2A | 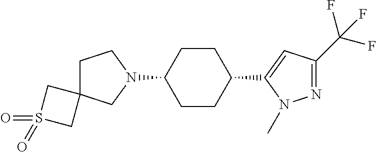 [SMILES: Cn1nc(C(F)(F)F)cc1[C@H]1CC[C@@H](N2CCC3(C2)CS(=O)(=O)C3)CC1] | 6-((1s,4s)-4-(1-methyl-3- (trifluoromethyl)-1H-pyrazol-5- yl)cyclohexyl)-2-thia-6- azaspiro[3.4]octane 2,2-dioxide |
| 2B | 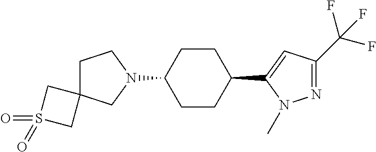 [SMILES: Cn1nc(C(F)(F)F)cc1[C@H]1CC[C@H](N2CCC3(C2)CS(=O)(=O)C3)CC1] | 6-((1r,4r)-4-(1-methyl-3- (trifluoromethyl)-1H-pyrazol-5- yl)cyclohexyl)-2-thia-6- azaspiro[3.4]octane 2,2-dioxide |
| 3A | 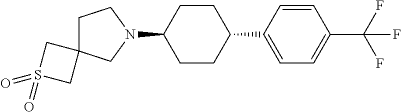 [SMILES: O=S1(=O)CC2(CCN([C@H]3CC[C@H](c4ccc(C(F)(F)F)cc4)CC3)C2)C1] | 6-((1r,4r)-4-(4- (trifluoromethyl)phenyl)cyclo hexyl)-2-thia-6- azaspiro[3.4]octane 2,2-dioxide |
| 3B | 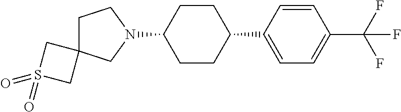 [SMILES: O=S1(=O)CC2(CCN([C@H]3CC[C@@H](c4ccc(C(F)(F)F)cc4)CC3)C2)C1] | 6-((1s,4s)-4-(4- (trifluoromethyl)phenyl)cyclo- hexyl)-2-thia-6- azaspiro[3.4]octane 2,2-dioxide |
| 4 | 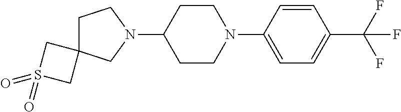 [SMILES: O=S1(=O)CC2(CCN(C3CCN(c4ccc(C(F)(F)F)cc4)CC3)C2)C1] | 6-(1-(4- (trifluoromethyl)phenyl)piperi- din-4-yl)-2-thia-6- azaspiro[3.4]octane 2,2-dioxide |
| 5 | 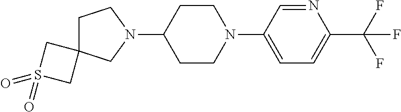 [SMILES: O=S1(=O)CC2(CCN(C3CCN(c4ccc(C(F)(F)F)nc4)CC3)C2)C1] | 6-(1-(6- (trifluoromethyl)pyridin-3- yl)piperidin-4-yl)-2-thia-6- azaspiro[3.4]octane 2,2-dioxide |
| 6 | 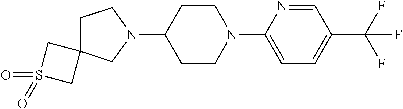 [SMILES: O=S1(=O)CC2(CCN(C3CCN(c4ccc(C(F)(F)F)cn4)CC3)C2)C1] | 6-(1-(5- (trifluoromethyl)pyridin-2- yl)piperidin-4-yl)-2-thia-6- azaspiro[3.4]octane 2,2-dioxide |
| 7 | 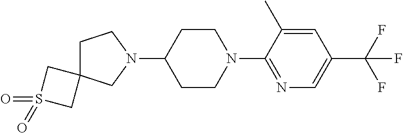 [SMILES: Cc1cc(C(F)(F)F)cnc1N1CCC(N2CCC3(C2)CS(=O)(=O)C3)CC1] | 6-(1-(3-methyl-5- (trifluoromethyl)pyridin-2- yl)piperidin-4-yl)-2-thia-6- azaspiro[3.4]octane 2,2-dioxide |
| 8A | 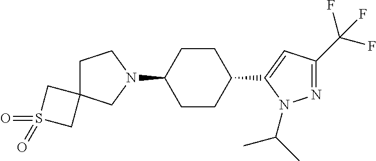 [SMILES: CC(C)n1nc(C(F)(F)F)cc1[C@H]1CC[C@H](N2CCC3(C2)CS(=O)(=O)C3)CC1] | 6-((1r,4r)-4-(1-isopropyl-3- (trifluoromethyl)-1H-pyrazol-5- yl)cyclohexyl)-2-thia-6- azaspiro[3.4]octane 2,2-dioxide |
| 8B | 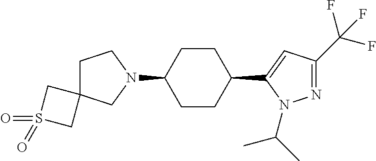 [SMILES: CC(C)n1nc(C(F)(F)F)cc1[C@H]1CC[C@@H](N2CCC3(C2)CS(=O)(=O)C3)CC1] | 6-((1s,4s)-4-(1-isopropyl-3- (trifluoromethyl)-1H-pyrazol-5- yl)cyclohexyl)-2-thia-6- azaspiro[3.4]octane 2,2-dioxide |
| 9A* | 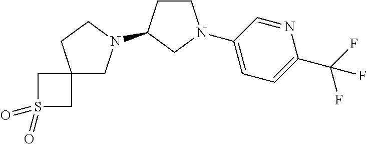 [SMILES: O=S1(=O)CC2(CCN([C@H]3CCN(c4ccc(C(F)(F)F)nc4)C3)C2)C1] | (S)-6-(1-(6- (trifluoromethyl)pyridin-3- yl)pyrrolidin-3-yl)-2-thia-6- azaspiro[3.4]octane 2,2-dioxide |
| 9B* | 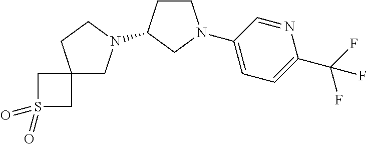 [SMILES: O=S1(=O)CC2(CCN([C@@H]3CCN(c4ccc(C(F)(F)F)nc4)C3)C2)C1] | (R)-6-(1-(6- (trifluoromethyl)pyridin-3- yl)pyrrolidin-3-yl)-2-thia-6- azaspiro[3.4]octane 2,2-dioxide |
| 10A* | 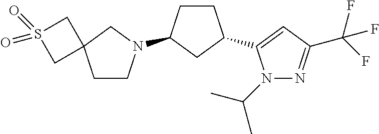 [SMILES: CC(C)n1nc(C(F)(F)F)cc1[C@H]1CC[C@H](N2CCC3(C2)CS(=O)(=O)C3)C1] | 6-((1S,3S)-3-(1-isopropyl-3- (trifluoromethyl)-1H-pyrazol-5- yl)cyclopentyl)-2-thia-6- azaspiro[3.4]octane 2,2-dioxide |
| 10B* | 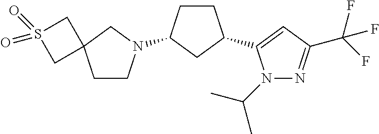 [SMILES: CC(C)n1nc(C(F)(F)F)cc1[C@H]1CC[C@@H](N2CCC3(C2)CS(=O)(=O)C3)C1] | 6-((1R,3S)-3-(1-isopropyl-3- (trifluoromethyl)-1H-pyrazol-5- yl)cyclopentyl)-2-thia-6- azaspiro[3.4]octane 2,2-dioxide |
| 10C* | 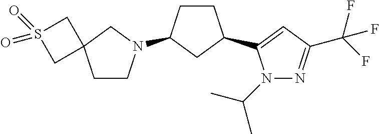 [SMILES: CC(C)n1nc(C(F)(F)F)cc1[C@@H]1CC[C@H](N2CCC3(C2)CS(=O)(=O)C3)C1] | 6-((1S,3R)-3-(1-isopropyl-3- (trifluoromethyl)-1H-pyrazol-5- yl)cyclopentyl)-2-thia-6- azaspiro[3.4]octane 2,2-dioxide |
| 10D* | 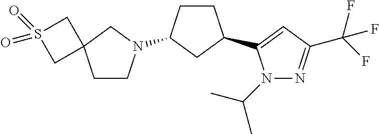 [SMILES: CC(C)n1nc(C(F)(F)F)cc1[C@@H]1CC[C@@H](N2CCC3(C2)CS(=O)(=O)C3)C1] | 6-((1R,3R)-3-(1-isopropyl-3- (trifluoromethyl)-1H-pyrazol-5- yl)cyclopentyl)-2-thia-6- azaspiro[3.4]octane 2,2-dioxide |
| 11A | 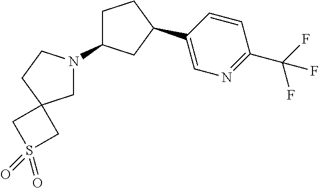 [SMILES: O=S1(=O)CC2(CCN([C@H]3CC[C@@H](c4ccc(C(F)(F)F)nc4)C3)C2)C1] | (cis)-6-(3-(6- (Trifluoromethyl)pyridin-3- yl)cyclopentyl)-2-thia-6- azaspiro[3.4]octane 2,2-dioxide |
| 11B | 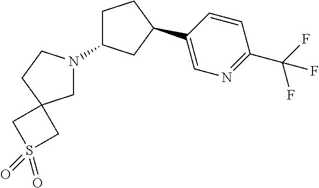 [SMILES: O=S1(=O)CC2(CCN([C@@H]3CC[C@@H](c4ccc(C(F)(F)F)nc4)C3)C2)C1] | (trans)-6-(3-(6- (Trifluoromethyl)pyridin-3- yl)cyclopentyl)-2-thia-6- azaspiro[3.4]octane 2,2-dioxide |
| 11C* | 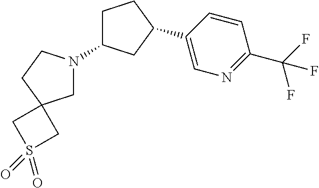 [SMILES: O=S1(=O)CC2(CCN([C@@H]3CC[C@H](c4ccc(C(F)(F)F)nc4)C3)C2)C1] | 6-((1R,3S)-3-(6- (trifluoromethyl)pyridin-3- yl)cyclopentyl)-2-thia-6- azaspiro[3.4]octane 2,2-dioxide |
| 11D* | 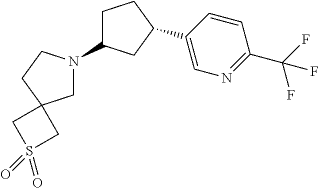 | 6-((1S,3S)-3-(6- (Trifluoromethyl)pyridin-3- yl)cyclopentyl)-2-thia-6- azaspiro[3.4]octane 2,2-dioxide |
| 12A | 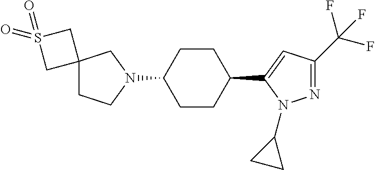 [SMILES: O=S1(=O)CC2(CCN([C@H]3CC[C@H](c4cc(C(F)(F)F)nn4C4CC4)CC3)C2)C1] | 6-((1r,4r)-4-(1-cyclopropyl-3- (trifluoromethyl)-1H-pyrazol-5- yl)cyclohexyl)-2-thia-6- azaspiro[3.4]octane 2,2-dioxide |
| 12B | 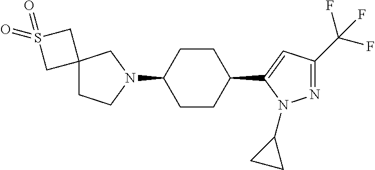 [SMILES: O=S1(=O)CC2(CCN([C@H]3CC[C@@H](c4cc(C(F)(F)F)nn4C4CC4)CC3)C2)C1] | 6-((1s,4s)-4-(1-cyclopropyl-3- (trifluoromethyl)-1H-pyrazol-5- yl)cyclohexyl)-2-thia-6- azaspiro[3.4]octane 2,2-dioxide |
| 13A* | 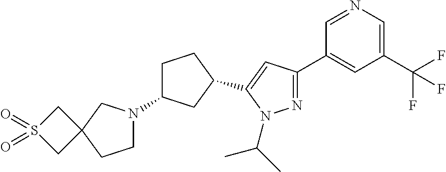 [SMILES: CC(C)n1nc(-c2cncc(C(F)(F)F)c2)cc1[C@H]1CC[C@@H](N2CCC3(C2)CS(=O)(=O)C3)C1] | 6-((1R,3S)-3-(1-isopropyl-3-(5- (trifluoromethyl)pyridin-3-yl)- 1H-pyrazol-5-yl)cyclopentyl)- 2-thia-6-azaspiro[3.4]octane 2,2-dioxide |
| 13B* | 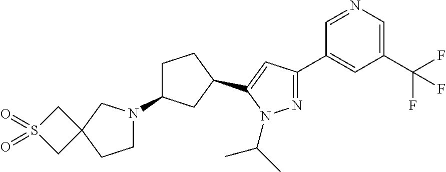 [SMILES: CC(C)n1nc(-c2cncc(C(F)(F)F)c2)cc1[C@@H]1CC[C@H](N2CCC3(C2)CS(=O)(=O)C3)C1] | 6-((1S,3R)-3-(1-isopropyl-3-(5- (trifluoromethyl)pyridin-3-yl)- 1H-pyrazol-5-yl)cyclopentyl)- 2-thia-6-azaspiro[3.4]octane 2,2-dioxide |
| 13C* | 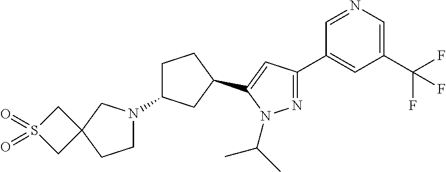 [SMILES: CC(C)n1nc(-c2cncc(C(F)(F)F)c2)cc1[C@@H]1CC[C@@H](N2CCC3(C2)CS(=O)(=O)C3)C1] | 6-((1R,3R)-3-(1-isopropyl-3-(5- (trifluoromethyl)pyridin-3-yl)- 1H-pyrazol-5-yl)cyclopentyl)- 2-thia-6-azaspiro[3.4]octane 2,2-dioxide |
| 13D* | 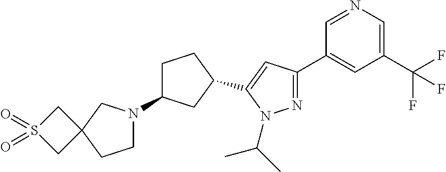 [SMILES: CC(C)n1nc(-c2cncc(C(F)(F)F)c2)cc1[C@H]1CC[C@H](N2CCC3(C2)CS(=O)(=O)C3)C1] | 6-((1S,3S)-3-(1-isopropyl-3-(5- (trifluoromethyl)pyridin-3-yl)- 1H-pyrazol-5-yl)cyclopentyl)- 2-thia-6-azaspiro[3.4]octane 2,2-dioxide |
| 14A | 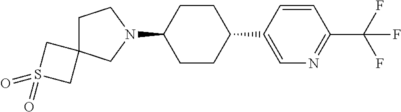 [SMILES: O=S1(=O)CC2(CCN([C@H]3CC[C@H](c4ccc(C(F)(F)F)nc4)CC3)C2)C1] | 6-((1r,4r)-4-(6- (trifluoromethyl)pyridin-3- yl)cyclohexyl)-2-thia-6- azaspiro[3.4]octane 2,2-dioxide |
| 14B | 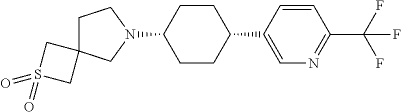 [SMILES: O=S1(=O)CC2(CCN([C@H]3CC[C@@H](c4ccc(C(F)(F)F)nc4)CC3)C2)C1] | 6-((1s,4s)-4-(6- (trifluoromethyl)pyridin-3- yl)cyclohexyl)-2-thia-6- azaspiro[3.4]octane 2,2-dioxide |
| 15 | 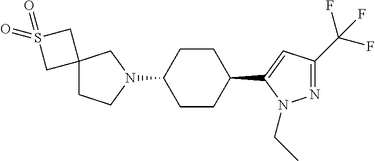 [SMILES: CCn1nc(C(F)(F)F)cc1[C@H]1CC[C@H](N2CCC3(C2)CS(=O)(=O)C3)CC1] | 6-((1r,4r)-4-(1-ethyl-3- (trifluoromethyl)-1H-pyrazol-5- yl)cyclohexyl)-2-thia-6- azaspiro[3.4]octane 2,2-dioxide |
| 16 | 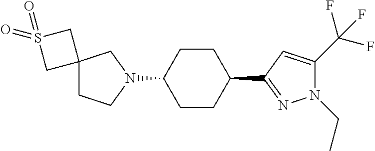 [SMILES: CCn1nc([C@H]2CC[C@H](N3CCC4(C3)CS(=O)(=O)C4)CC2)cc1C(F)(F)F] | 6-((1r,4r)-4-(1-ethyl-5- (trifluoromethyl)-1H-pyrazol-3- yl)cyclohexyl)-2-thia-6- azaspiro[3.4]octane 2,2-dioxide |
| 17A* | 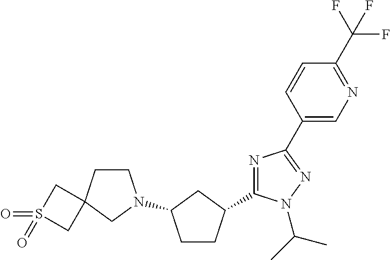 [SMILES: CC(C)n1nc(-c2ccc(C(F)(F)F)nc2)nc1[C@@H]1CC[C@H](N2CCC3(C2)CS(=O)(=O)C3)C1] | 6-((1S,3R)-3-(1-isopropyl-3-(6- (trifluoromethyl)pyridin-3-yl)- 1H-1,2,4-triazol-5- yl)cyclopentyl)-2-thia-6- azaspiro[3.4]octane 2,2-dioxide |
| 17B* | 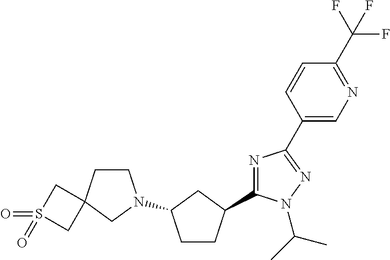 [SMILES: CC(C)n1nc(-c2ccc(C(F)(F)F)nc2)nc1[C@H]1CC[C@H](N2CCC3(C2)CS(=O)(=O)C3)C1] | 6-((1S,3S)-3-(1-isopropyl-3-(6- (trifluoromethyl)pyridin-3-yl)- 1H-1,2,4-triazol-5- yl)cyclopentyl)-2-thia-6- azaspiro[3.4]octane 2,2-dioxide |
| 17C* | 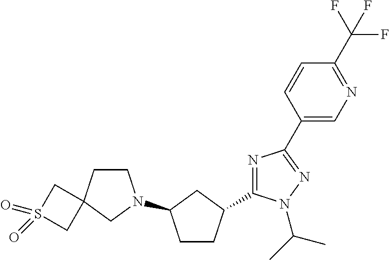 [SMILES: CC(C)n1nc(-c2ccc(C(F)(F)F)nc2)nc1[C@@H]1CC[C@@H](N2CCC3(C2)CS(=O)(=O)C3)C1] | 6-((1R,3R)-3-(1-isopropyl-3-(6- (trifluoromethyl)pyridin-3-yl)- 1H-1,2,4-triazol-5- yl)cyclopentyl)-2-thia-6- azaspiro[3.4]octane 2,2-dioxide |
| 17D* | 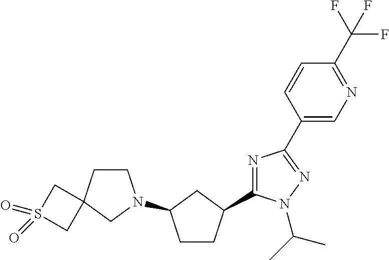 [SMILES: CC(C)n1nc(-c2ccc(C(F)(F)F)nc2)nc1[C@H]1CC[C@@H](N2CCC3(C2)CS(=O)(=O)C3)C1] | 6-((1R,3S)-3-(1-isopropyl-3-(6- (trifluoromethyl)pyridin-3-yl)- 1H-1,2,4-triazol-5- yl)cyclopentyl)-2-thia-6- azaspiro[3.4]octane 2,2-dioxide |
| 18A* | 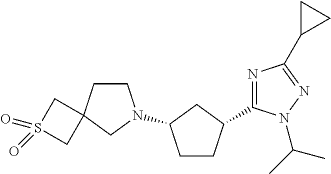 [SMILES: CC(C)n1nc(C2CC2)nc1[C@@H]1CC[C@H](N2CCC3(C2)CS(=O)(=O)C3)C1] | 6-((1S,3R)-3-(3-cyclopropyl-1- isopropyl-1H-1,2,4-triazol-5- yl)cyclopentyl)-2-thia-6- azaspiro[3.4]octane 2,2-dioxide |
| 18B* | 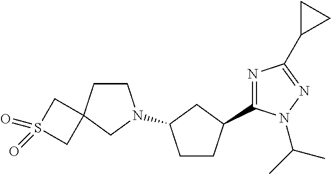 [SMILES: CC(C)n1nc(C2CC2)nc1[C@H]1CC[C@H](N2CCC3(C2)CS(=O)(=O)C3)C1] | 6-((1S,3S)-3-(1-isopropyl-3-(6- (trifluoromethyl)pyridin-3-yl)- 1H-1,2,4-triazol-5- yl)cyclopentyl)-2-thia-6- azaspiro[3.4]octane 2,2-dioxide |
| 18C* | 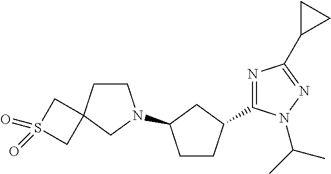 [SMILES: CC(C)n1nc(C2CC2)nc1[C@@H]1CC[C@@H](N2CCC3(C2)CS(=O)(=O)C3)C1] | 6-((1R,3R)-3-(3-cyclopropyl-1- isopropyl-1H-1,2,4-triazol-5- yl)cyclopentyl)-2-thia-6- azaspiro[3.4]octane 2,2-dioxide |
| 18D* | 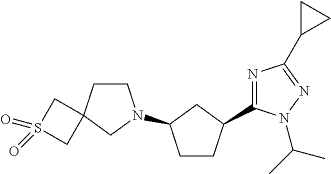 [SMILES: CC(C)n1nc(C2CC2)nc1[C@H]1CC[C@@H](N2CCC3(C2)CS(=O)(=O)C3)C1] | 6-((1R,3S)-3-(1-isopropyl-3-(6- (trifluoromethyl)pyridin-3-yl)- 1H-1,2,4-triazol-5- yl)cyclopentyl)-2-thia-6- azaspiro[3.4]octane 2,2-dioxide |
| 19A* | 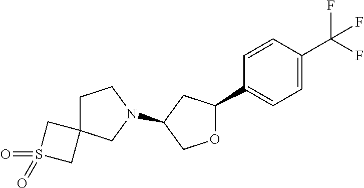 [SMILES: O=S1(=O)CC2(CCN([C@@H]3CO[C@H](c4ccc(C(F)(F)F)cc4)C3)C2)C1] | 6-((3S,5S)-5-(4- (trifluoromethyl)phenyl)tetrahy- drofuran-3-yl)-2-thia-6- azaspiro[3.4]octane 2,2-dioxide |
| 19B* | 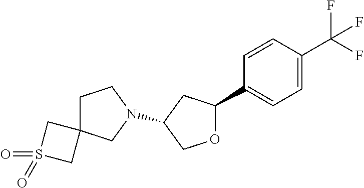 [SMILES: O=S1(=O)CC2(CCN([C@H]3CO[C@H](c4ccc(C(F)(F)F)cc4)C3)C2)C1] | 6-((3R,5S)-5-(4- (trifluoromethyl)phenyl)tetrahy- drofuran-3-y1)-2-thia-6- azaspiro[3.4]octane 2,2-dioxide |
| 19C* | 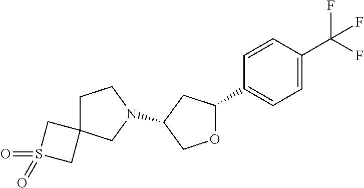 [SMILES: O=S1(=O)CC2(CCN([C@H]3CO[C@@H](c4ccc(C(F)(F)F)cc4)C3)C2)C1] | 6-((3R,5R)-5-(4- (trifluoromethyl)phenyl)tetrahy- drofuran-3-yl)-2-thia-6- azaspiro[3.4]octane 2,2-dioxide |
| 19D* | 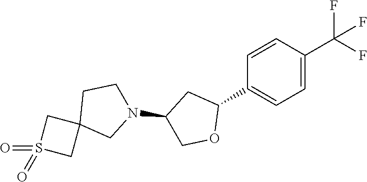 [SMILES: O=S1(=O)CC2(CCN([C@@H]3CO[C@@H](c4ccc(C(F)(F)F)cc4)C3)C2)C1] | 6-((3S,5R)-5-(4- (trifluoromethyl)phenyl)tetrahy- drofuran-3-yl)-2-thia-6- azaspiro[3.4]octane 2,2-dioxide |
| 20A* | 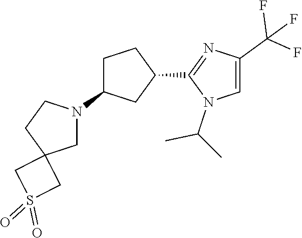 [SMILES: CC(C)n1cc(C(F)(F)F)nc1[C@H]1CC[C@H](N2CCC3(C2)CS(=O)(=O)C3)C1] | 6-((1S,3S)-3-(1-isopropyl-4- (trifluoromethyl)-1H-imidazol- 2-yl)cyclopentyl)-2-thia-6- azaspiro[3.4]octane 2,2-dioxide |
| 20B* | 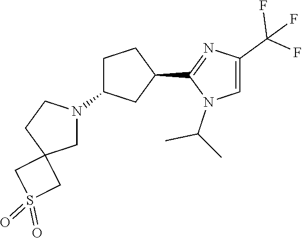 [SMILES: CC(C)n1cc(C(F)(F)F)nc1[C@@H]1CC[C@@H](N2CCC3(C2)CS(=O)(=O)C3)C1] | 6-((1R,3S)-3-(1-isopropyl-4- (trifluoromethyl)-1H-imidazol- 2-yl)cyclopentyl)-2-thia-6- azaspiro[3.4]octane 2,2-dioxide |
| 20C* | 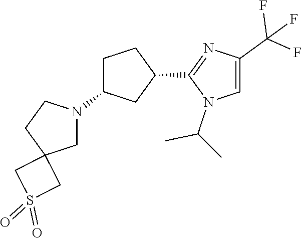 [SMILES: CC(C)n1cc(C(F)(F)F)nc1[C@H]1CC[C@@H](N2CCC3(C2)CS(=O)(=O)C3)C1] | 6-((1S,3R)-3-(1-isopropyl-4- (trifluoromethyl)-1H-imidazol- 2-yl)cyclopentyl)-2-thia-6- azaspiro[3.4]octane 2,2-dioxide |
| 20D* | 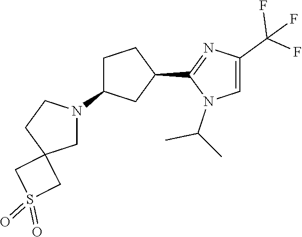 [SMILES: CC(C)n1cc(C(F)(F)F)nc1[C@@H]1CC[C@H](N2CCC3(C2)CS(=O)(=O)C3)C1] | 6-((1R,3R)-3-(1-isopropyl-4- (trifluoromethyl)-1H-imidazol- 2-yl)cyclopentyl)-2-thia-6- azaspiro[3.4]octane 2,2-dioxide |
| 21A | 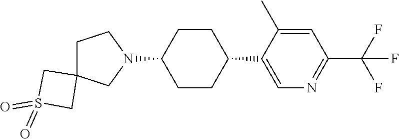 [SMILES: Cc1cc(C(F)(F)F)ncc1[C@H]1CC[C@@H](N2CCC3(C2)CS(=O)(=O)C3)CC1] | 6-((1s,4s)-4-(4-methyl-6- (trifluoromethyl)pyridin-3- yl)cyclohexyl)-2-thia-6- azaspiro[3.4]octane 2,2-dioxide |
| 21B | 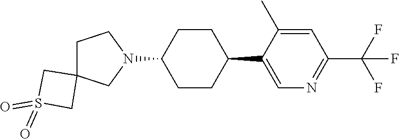 [SMILES: Cc1cc(C(F)(F)F)ncc1[C@H]1CC[C@H](N2CCC3(C2)CS(=O)(=O)C3)CC1] | 6-((1r,4r)-4-(4-methyl-6- (trifluoromethyl)pyridin-3- yl)cyclohexyl)-2-thia-6- azaspiro[3.4]octane 2,2-dioxide |
| 22A* | 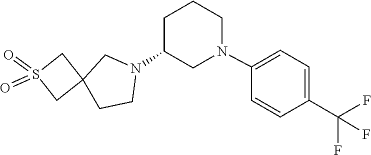 [SMILES: O=S1(=O)CC2(CCN([C@@H]3CCCN(c4ccc(C(F)(F)F)cc4)C3)C2)C1] | (R)-6-(1-(4- (trifluoromethyl)phenyl)piperi- din-3-yl)-2-thia-6- azaspiro[3.4]octane 2,2-dioxide |
| 22B* | 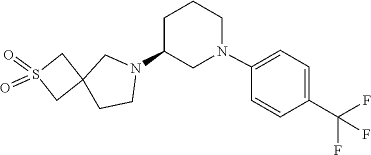 [SMILES: O=S1(=O)CC2(CCN([C@H]3CCCN(c4ccc(C(F)(F)F)cc4)C3)C2)C1] | (S)-6-(1-(4- (trifluoromethyl)phenyl)piperi- din-3-yl)-2-thia-6- azaspiro[3.4]octane 2,2-dioxide |
| 23A | 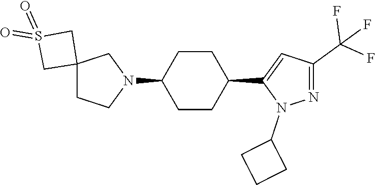 [SMILES: O=S1(=O)CC2(CCN([C@H]3CC[C@@H](c4cc(C(F)(F)F)nn4C4CCC4)CC3)C2)C1] | 6-((1s,4s)-4-(1-cyclobutyl-3- (trifluoromethyl)-1H-pyrazol-5- yl)cyclohexyl)-2-thia-6- azaspiro[3.4]octane 2,2-dioxide |
| 23B | 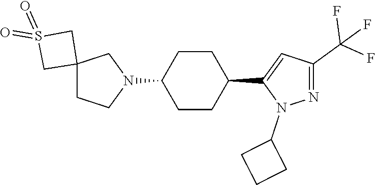 [SMILES: O=S1(=O)CC2(CCN([C@H]3CC[C@H](c4cc(C(F)(F)F)nn4C4CCC4)CC3)C2)C1] | 6-((1r,4r)-4-(1-cyclobutyl-3- (trifluoromethyl)-1H-pyrazol-5- yl)cyclohexyl)-2-thia-6- azaspiro[3.4]octane 2,2-dioxide |
| 24 | 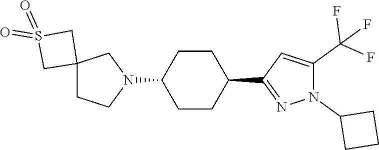 [SMILES: O=S1(=O)CC2(CCN([C@H]3CC[C@H](c4cc(C(F)(F)F)n(C5CCC5)n4)CC3)C2)C1] | 6-((1r,4r)-4-(1-cyclobutyl-5- (trifluoromethyl)-1H-pyrazol-3- yl)cyclohexyl)-2-thia-6- azaspiro[3.4]octane 2,2-dioxide |
| 25A | 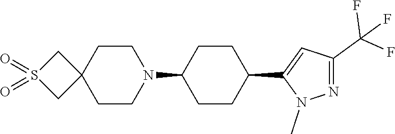 [SMILES: Cn1nc(C(F)(F)F)cc1[C@H]1CC[C@@H](N2CCC3(CC2)CS(=O)(=O)C3)CC1] | 7-((1s,4s)-4-(1-methyl-3- (trifluoromethyl)-1H-pyrazol-5- yl)cyclohexyl)-2-thia-7- azaspiro[3.5]nonane 2,2- dioxide |
| 25B | 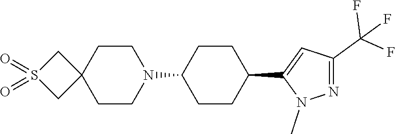 [SMILES: Cn1nc(C(F)(F)F)cc1[C@H]1CC[C@H](N2CCC3(CC2)CS(=O)(=O)C3)CC1] | 7-((1r,4r)-4-(1-methyl-3- (trifluoromethyl)-1H-pyrazol-5- yl)cyclohexyl)-2-thia-7- azaspiro[3.5]nonane 2,2- dioxide |
| 26A* | 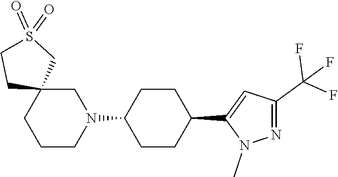 [SMILES: Cn1nc(C(F)(F)F)cc1[C@H]1CC[C@H](N2CCC[C@@]3(CCS(=O)(=O)C3)C2)CC1] | (S)-7-((1r,4S)-4-(1-methyl-3- (trifluoromethyl)-1H-pyrazol-5- yl)cyclohexyl)-2-thia-7- azaspiro[4.5]decane 2,2-dioxide |
| 26B* | 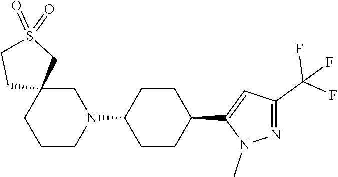 [SMILES: Cn1nc(C(F)(F)F)cc1[C@H]1CC[C@H](N2CCC[C@]3(CCS(=O)(=O)C3)C2)CC1] | (R)-7-((1r,4R)-4-(1-methyl-3- (trifluoromethyl)-1H-pyrazol-5- yl)cyclohexyl)-2-thia-7- azaspiro[4.5]decane 2,2-dioxide |
| 27A* | 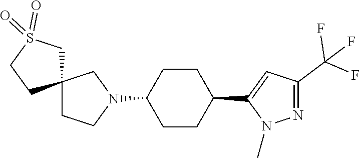 [SMILES: Cn1nc(C(F)(F)F)cc1[C@H]1CC[C@H](N2CC[C@@]3(CCS(=O)(=O)C3)C2)CC1] | (R)-7-((1r,4R)-4-(1-methyl-3- (trifluoromethyl)-1H-pyrazol-5- yl)cyclohexyl)-2-thia-7- azaspiro[4.4]nonane 2,2- dioxide |
| 27B* | 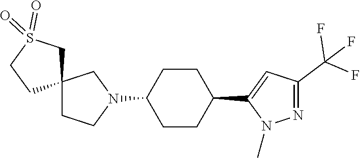 [SMILES: Cn1nc(C(F)(F)F)cc1[C@H]1CC[C@H](N2CC[C@]3(CCS(=O)(=O)C3)C2)CC1] | (S)-7-((1r,4S)-4-(1-methyl-3- (trifluoromethyl)-1H-pyrazol-5- yl)cyclohexyl)-2-thia-7- azaspiro[4.4]nonane 2,2- dioxide |
| 28 | 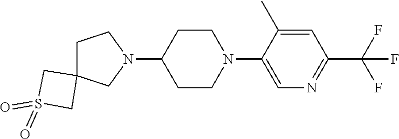 [SMILES: Cc1cc(C(F)(F)F)ncc1N1CCC(N2CCC3(C2)CS(=O)(=O)C3)CC1] | 6-(1-(4-methyl-6- (trifluoromethyl)pyridin-3- yl)piperidin-4-yl)-2-thia-6- azaspiro[3.4]octane 2,2-dioxide |
| 29 | 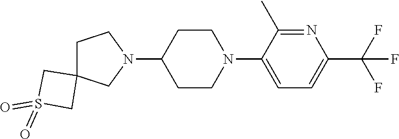 [SMILES: Cc1nc(C(F)(F)F)ccc1N1CCC(N2CCC3(C2)CS(=O)(=O)C3)CC1] | 6-(1-(2-methyl-6- (trifluoromethyl)pyridin-3- yl)piperidin-4-yl)-2-thia-6- azaspiro[3.4]octane 2,2-dioxide |
| 30 | 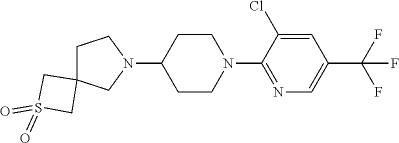 [SMILES: O=S1(=O)CC2(CCN(C3CCN(c4ncc(C(F)(F)F)cc4Cl)CC3)C2)C1] | 6-(1-(3-chloro-5- (trifluoromethyl)pyridin-2- yl)piperidin-4-yl)-2-thia-6- azaspiro[3.4]octane 2,2-dioxide |
| 31 | 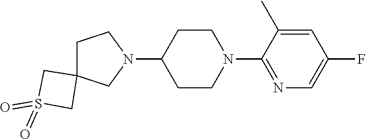 [SMILES: Cc1cc(F)cnc1N1CCC(N2CCC3(C2)CS(=O)(=O)C3)CC1] | 6-(1-(5-fluoro-3-methylpyridin- 2-yl)piperidin-4-yl)-2-thia-6- azaspiro[3.4]octane 2,2-dioxide |
| 32 | 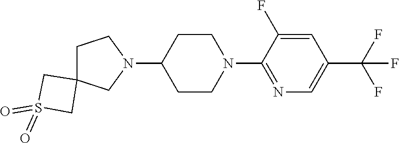 [SMILES: O=S1(=O)CC2(CCN(C3CCN(c4ncc(C(F)(F)F)cc4F)CC3)C2)C1] | 6-(1-(3-fluoro-5- (trifluoromethyl)pyridin-2- yl)piperidin-4-yl)-2-thia-6- azaspiro[3.4]octane 2,2-dioxide |
| 33A | 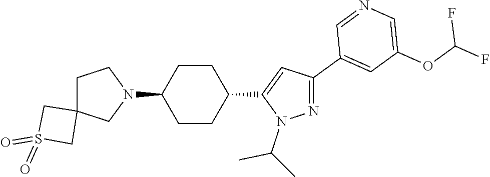 [SMILES: CC(C)n1nc(-c2cncc(OC(F)F)c2)cc1[C@H]1CC[C@H](N2CCC3(C2)CS(=O)(=O)C3)CC1] | (trans)-6-(4-(3-(5- (difluoromethoxy)pyridin-3-yl)- 1-isopropyl-1H-pyrazol-5- yl)cyclohexyl)-2-thia-6- azaspiro[3.4]octane 2,2-dioxide |
| 33B | 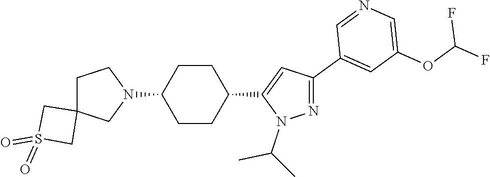 [SMILES: CC(C)n1nc(-c2cncc(OC(F)F)c2)cc1[C@H]1CC[C@@H](N2CCC3(C2)CS(=O)(=O)C3)CC1] | (cis)-6-(4-(3-(5- (difluoromethoxy)pyridin-3-yl)- 1-isopropyl-1H-pyrazol-5- yl)cyclohexyl)-2-thia-6- azaspiro[3.4]octane 2,2-dioxide |
| 35 | 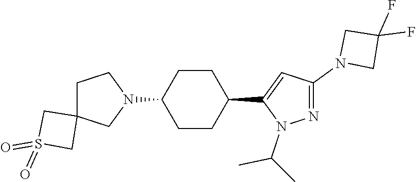 [SMILES: CC(C)n1nc(N2CC(F)(F)C2)cc1[C@H]1CC[C@H](N2CCC3(C2)CS(=O)(=O)C3)CC1] | 6-((1r,4r)-4-(3-(3,3- difluoroazetidin-1-yl)-1- isopropyl-1H-pyrazol-5- yl)cyclohexyl)-2-thia-6- azaspiro[3.4]octane 2,2-dioxide |
| 36A* | 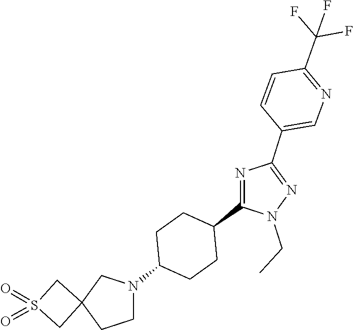 [SMILES: CCn1nc(-c2ccc(C(F)(F)F)nc2)nc1[C@H]1CC[C@H](N2CCC3(C2)CS(=O)(=O)C3)CC1] | 6-((1r,4r)-4-(1-ethyl-3-(6- (trifluoromethyl)pyridin-3-yl)- 1H-1,2,4-triazol-5- yl)cyclohexyl)-2-thia-6- azaspiro[3.4]octane 2,2-dioxide |
| 36B* | 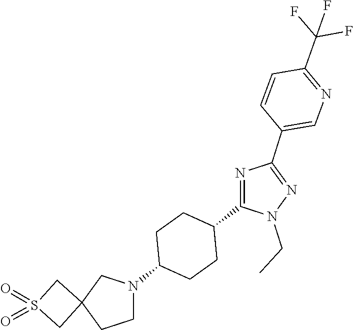 [SMILES: CCn1nc(-c2ccc(C(F)(F)F)nc2)nc1[C@H]1CC[C@@H](N2CCC3(C2)CS(=O)(=O)C3)CC1] | 6-((1s,4s)-4-(1-ethyl-3-(6- (trifluoromethyl)pyridin-3-yl)- 1H-1,2,4-triazol-5- yl)cyclohexyl)-2-thia-6- azaspiro[3.4]octane 2,2-dioxide |
| 38A* | 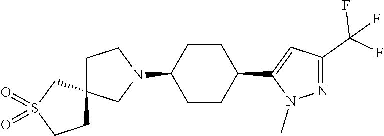 [SMILES: Cn1nc(C(F)(F)F)cc1[C@H]1CC[C@@H](N2CC[C@]3(CCS(=O)(=O)C3)C2)CC1] | (S)-7-((1s,4R)-4-(1-methyl-3- (trifluoromethyl)-1H-pyrazol-5- yl)cyclohexyl)-2-thia-7- azaspiro[4.4]nonane 2,2- dioxide |
| 38B* | 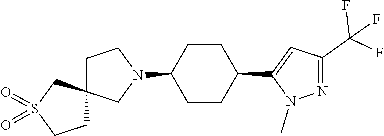 [SMILES: Cn1nc(C(F)(F)F)cc1[C@H]1CC[C@@H](N2CC[C@@]3(CCS(=O)(=O)C3)C2)CC1] | (R)-7-((1s,4S)-4-(1- methyl-3-(trifluoromethyl)-1H- pyrazol-5-yl)cyclohexyl)-2- thia-7-azaspiro[4.4]nonane 2,2- dioxide |
| 38C* | 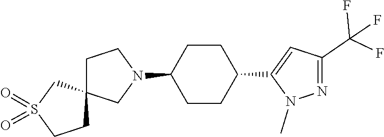 [SMILES: Cn1nc(C(F)(F)F)cc1[C@H]1CC[C@H](N2CC[C@]3(CCS(=O)(=O)C3)C2)CC1] | (S)-7-((1r,4S)-4-(1- methyl-3-(trifluoromethyl)-1H- pyrazol-5-yl)cyclohexyl)-2- thia-7-azaspiro[4.4]nonane 2,2- dioxide |
| 38D* | 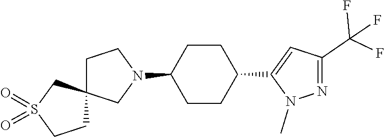 [SMILES: Cn1nc(C(F)(F)F)cc1[C@H]1CC[C@H](N2CC[C@@]3(CCS(=O)(=O)C3)C2)CC1] | (R)-7-((1r,4R)-4-(1-methyl-3- (trifluoromethyl)-1H-pyrazol-5- yl)cyclohexyl)-2-thia-7- azaspiro[4.4]nonane 2,2- dioxide |
| 40A | 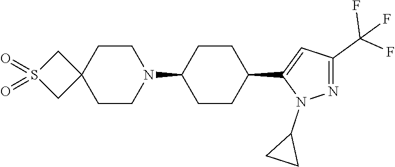 [SMILES: O=S1(=O)CC2(CCN([C@H]3CC[C@@H](c4cc(C(F)(F)F)nn4C4CC4)CC3)CC2)C1] | 7-((1r,4r)-4-(1-cyclopropyl-3- (trifluoromethyl)-1H-pyrazol-5- yl)cyclohexyl)-2-thia-7- azaspiro[3.5]nonane 2,2- dioxide |
| 40B | 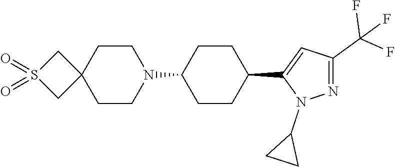 [SMILES: O=S1(=O)CC2(CCN([C@H]3CC[C@H](c4cc(C(F)(F)F)nn4C4CC4)CC3)CC2)C1] | 7-((1s,4s)-4-(1-cyclopropyl-3- (trifluoromethyl)-1H-pyrazol-5- yl)cyclohexyl)-2-thia-7- azaspiro[3.5]nonane 2,2- dioxide |
| 42A* | 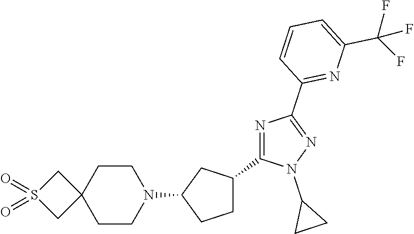 [SMILES: O=S1(=O)CC2(CCN([C@H]3CC[C@@H](c4nc(-c5cccc(C(F)(F)F)n5)nn4C4CC4)C3)CC2)C1] | 7-((1S,3R)-3-(1-cyclopropyl-3- (6-(trifluoromethyl)pyridin-2- yl)-1H-1,2,4-triazol-5- yl)cyclopentyl)-2-thia-7- azaspiro[3.5]nonane 2,2- dioxide |
| 42B* | 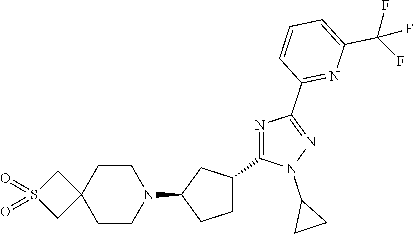 [SMILES: O=S1(=O)CC2(CCN([C@@H]3CC[C@@H](c4nc(-c5cccc(C(F)(F)F)n5)nn4C4CC4)C3)CC2)C1] | 7-((1R,3R)-3-(1-cyclopropyl-3- (6-(trifluoromethyl)pyridin-2- yl)-1H-1,2,4-triazol-5- yl)cyclopentyl)-2-thia-7- azaspiro[3.5]nonane 2,2- dioxide |
| 42C* | 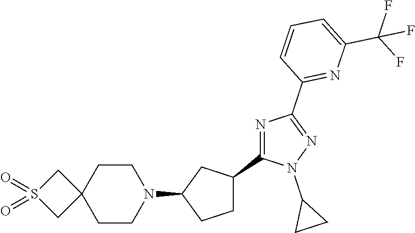 [SMILES: O=S1(=O)CC2(CCN([C@@H]3CC[C@H](c4nc(-c5cccc(C(F)(F)F)n5)nn4C4CC4)C3)CC2)C1] | 7-((1S,3S)-3-(1-cyclopropyl-3- (6-(trifluoromethyl)pyridin-2- yl)-1H-1,2,4-triazol-5- yl)cyclopentyl)-2-thia-7- azaspiro[3.5]nonane 2,2- dioxide |
| 42D* | 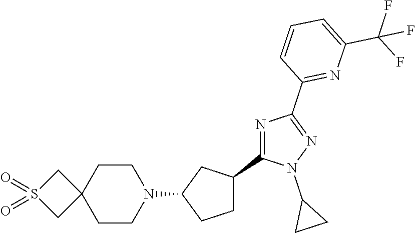 [SMILES: O=S1(=O)CC2(CCN([C@H]3CC[C@H](c4nc(-c5cccc(C(F)(F)F)n5)nn4C4CC4)C3)CC2)C1] | 7-((1R,3S)-3-(1-cyclopropyl-3- (6-(trifluoromethyl)pyridin-2- yl)-1H-1,2,4-triazol-5- yl)cyclopentyl)-2-thia-7- azaspiro[3.5]nonane 2,2- dioxide |
| 43A* | 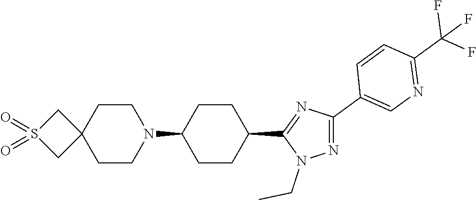 [SMILES: CCn1nc(-c2ccc(C(F)(F)F)nc2)nc1[C@H]1CC[C@@H](N2CCC3(CC2)CS(=O)(=O)C3)CC1] | (trans)-7-(-4-(1-ethyl-3-(6- (trifluoromethyl)pyridin-3-yl)- 1H-1,2,4-triazol-5- yl)cyclohexyl)-2-thia-7- azaspiro[3.5]nonane 2,2- dioxide |
| 43B* | 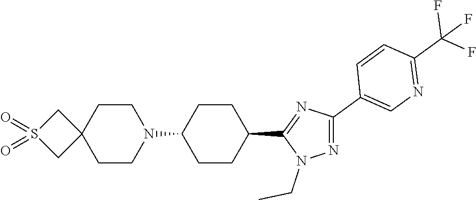 [SMILES: CCn1nc(-c2ccc(C(F)(F)F)nc2)nc1[C@H]1CC[C@H](N2CCC3(CC2)CS(=O)(=O)C3)CC1] | (cis)-7-(-4-(1-ethyl-3-(6- (trifluoromethyl)pyridin-3-yl)- 1H-1,2,4-triazol-5- yl)cyclohexyl)-2-thia-7- azaspiro[3.5]nonane 2,2- dioxide |
| 44 | 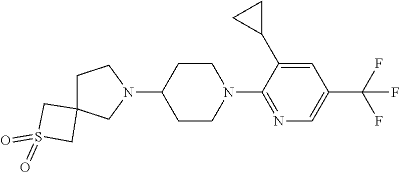 [SMILES: O=S1(=O)CC2(CCN(C3CCN(c4ncc(C(F)(F)F)cc4C4CC4)CC3)C2)C1] | 6-(1-(3-cyclopropyl-5- (trifluoromethyl)pyridin-2- yl)piperidin-4-yl)-2-thia-6- azaspiro[3.4]octane 2,2-dioxide |
| 45 | 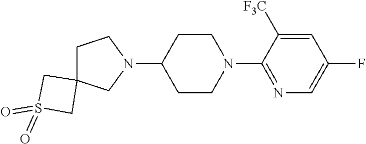 [SMILES: O=S1(=O)CC2(CCN(C3CCN(c4ncc(F)cc4C(F)(F)F)CC3)C2)C1] | 6-(1-(5-fluoro-3- (trifluoromethyl)pyridin-2- yl)piperidin-4-yl)-2-thia-6- azaspiro[3.4]octane 2,2-dioxide |
| 46A* | 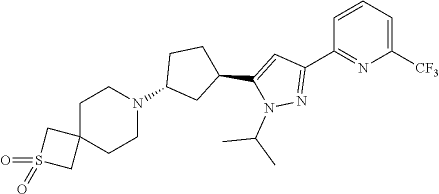 [SMILES: CC(C)n1nc(-c2cccc(C(F)(F)F)n2)cc1[C@@H]1CC[C@@H](N2CCC3(CC2)CS(=O)(=O)C3)C1] | 7-((1R,3R)-3-(1-isopropyl-3-(6- (trifluoromethyl)pyridin-2-yl)- 1H-pyrazol-5-yl)cyclopentyl)- 2-thia-7-azaspiro[3.5 ]nonane 2,2-dioxide |
| 46B* | 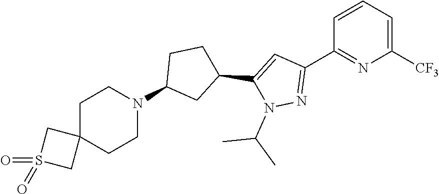 [SMILES: CC(C)n1nc(-c2cccc(C(F)(F)F)n2)cc1[C@@H]1CC[C@H](N2CCC3(CC2)CS(=O)(=O)C3)C1] | 7-((1S,3R)-3-(1-isopropyl-3-(6- (trifluoromethyl)pyridin-2-yl)- 1H-pyrazol-5-yl)cyclopentyl)- 2-thia-7-azaspiro[3.5]nonane 2,2-dioxide |
| 46C* | 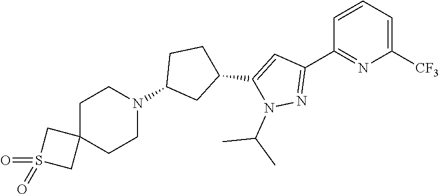 [SMILES: CC(C)n1nc(-c2cccc(C(F)(F)F)n2)cc1[C@H]1CC[C@@H](N2CCC3(CC2)CS(=O)(=O)C3)C1] | 7-((1R,3S)-3-(1-isopropyl-3-(6- (trifluoromethyl)pyridin-2-yl)- 1H-pyrazol-5-yl)cyclopentyl)- 2-thia-7-azaspiro[3.5]nonane 2,2-dioxide |
| 46D* | 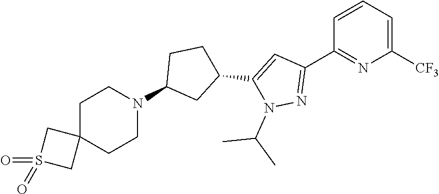 [SMILES: CC(C)n1nc(-c2cccc(C(F)(F)F)n2)cc1[C@H]1CC[C@H](N2CCC3(CC2)CS(=O)(=O)C3)C1] | 7-((1S,3S)-3-(1-isopropyl-3-(6- (trifluoromethyl)pyridin-2-yl)- 1H-pyrazol-5-yl)cyclopentyl)- 2-thia-7-azaspiro[3.5]nonane 2,2-dioxide |
| 47A | 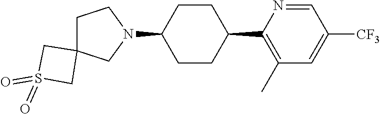 [SMILES: Cc1cc(C(F)(F)F)cnc1[C@H]1CC[C@@H](N2CCC3(C2)CS(=O)(=O)C3)CC1] | 6-((1s,4s)-4-(3-methyl-5- (trifluoromethyl)pyridin-2- yl)cyclohexyl)-2-thia-6- azaspiro[3.4]octane 2,2-dioxide |
| 47B | 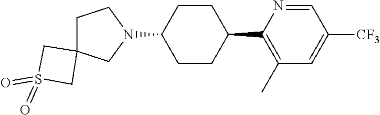 [SMILES: Cc1cc(C(F)(F)F)cnc1[C@H]1CC[C@H](N2CCC3(C2)CS(=O)(=O)C3)CC1] | 6-((1r,4r)-4-(3-methyl-5- (trifluoromethyl)pyridin-2- yl)cyclohexyl)-2-thia-6- azaspiro[3.4]octane 2,2-dioxide |
| 48A | 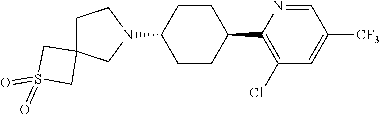 [SMILES: O=S1(=O)CC2(CCN([C@H]3CC[C@H](c4ncc(C(F)(F)F)cc4Cl)CC3)C2)C1] | 6-((1r,4r)-4-(3-chloro-5- (trifluoromethyl)pyridin-2- yl)cyclohexyl)-2-thia-6- azaspiro[3.4]octane 2,2-dioxide |
| 48B | 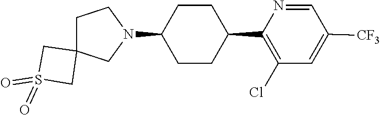 [SMILES: O=S1(=O)CC2(CCN([C@H]3CC[C@@H](c4ncc(C(F)(F)F)cc4Cl)CC3)C2)C1] | 6-((1s,4s)-4-(3-chloro-5- (trifluoromethyl)pyridin-2- yl)cyclohexyl)-2-thia-6- azaspiro[3.4]octane 2,2-dioxide |
| 49 | 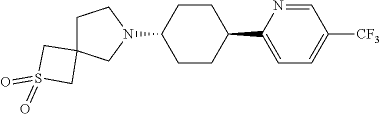 [SMILES: O=S1(=O)CC2(CCN([C@H]3CC[C@H](c4ccc(C(F)(F)F)cn4)CC3)C2)C1] | 6-((1r,4r)-4-(5- (trifluoromethyl)pyridin-2- yl)cyclohexyl)-2-thia-6- azaspiro[3.4]octane 2,2-dioxide |
| 50A | 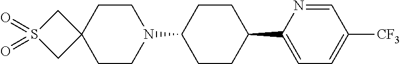 [SMILES: O=S1(=O)CC2(CCN([C@H]3CC[C@H](c4ccc(C(F)(F)F)cn4)CC3)CC2)C1] | 7-((1r,4r)-4-(5- (trifluoromethyl)pyridin-2- yl)cyclohexyl)-2-thia-7- azaspiro[3.5]nonane 2,2- |
| | | dioxide |
| 50B | 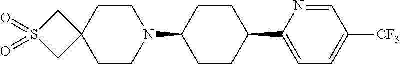 [SMILES: O=S1(=O)CC2(CCN([C@H]3CC[C@@H](c4ccc(C(F)(F)F)cn4)CC3)CC2)C1] | 7-((1s,4s)-4-(5- (trifluoromethyl)pyridin-2- yl)cyclohexyl)-2-thia-7- azaspiro[3.5]nonane 2,2- dioxide |
| 51A | 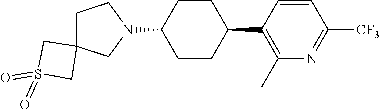 [SMILES: Cc1nc(C(F)(F)F)ccc1[C@H]1CC[C@H](N2CCC3(C2)CS(=O)(=O)C3)CC1] | 6-((1r,4r)-4-(2-methyl-6- (trifluoromethyl)pyridin-3- yl)cyclohexyl)-2-thia-6- azaspiro[3.4]octane 2,2-dioxide |
| 51B | 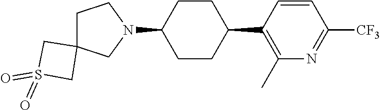 [SMILES: Cc1nc(C(F)(F)F)ccc1[C@H]1CC[C@@H](N2CCC3(C2)CS(=O)(=O)C3)CC1] | 6-((1s,4s)-4-(2-methyl-6- (trifluoromethyl)pyridin-3- yl)cyclohexyl)-2-thia-6- azaspiro[3.4]octane 2,2-dioxide |
| 52 | 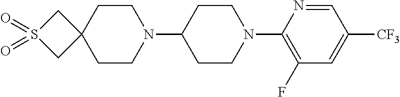 [SMILES: O=S1(=O)CC2(CCN(C3CCN(c4ncc(C(F)(F)F)cc4F)CC3)CC2)C1] | 7-(1-(3-fluoro-5- (trifluoromethyl)pyridin-2- yl)piperidin-4-yl)-2-thia-7- azaspiro[3.5]nonane 2,2- dioxide |
| 53A* | 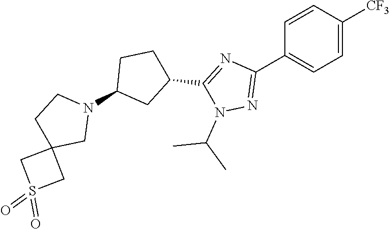 [SMILES: CC(C)n1nc(-c2ccc(C(F)(F)F)cc2)nc1[C@H]1CC[C@H](N2CCC3(C2)CS(=O)(=O)C3)C1] | 6-((1S,3S)-3-(1-isopropyl-3-(4- (trifluoromethyl)phenyl)-1H- 1,2,4-triazol-5-yl)cyclopentyl)- 2-thia-6-azaspiro[3.4]octane 2,2-dioxide |
| 53B* | 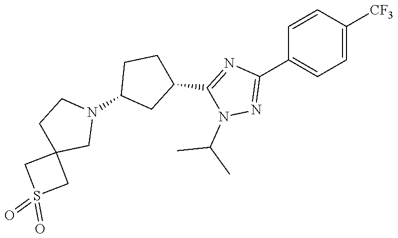 [SMILES: CC(C)n1nc(-c2ccc(C(F)(F)F)cc2)nc1[C@H]1CC[C@@H](N2CCC3(C2)CS(=O)(=O)C3)C1] | 6-((1R,3S)-3-(1-isopropyl-3-(4- (trifluoromethyl)phenyl)-1H- 1,2,4-triazol-5-yl)cyclopentyl)- 2-thia-6-azaspiro[3.4]octane 2,2-dioxide |
| 53C* | 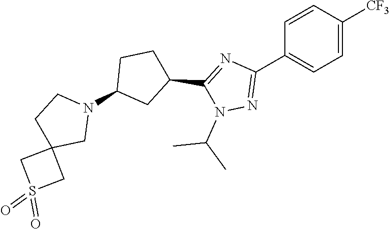 [SMILES: CC(C)n1nc(-c2ccc(C(F)(F)F)cc2)nc1[C@@H]1CC[C@H](N2CCC3(C2)CS(=O)(=O)C3)C1] | 6-((1S,3R)-3-(1-isopropyl-3-(4- (trifluoromethyl)phenyl)-1H- 1,2,4-triazol-5-yl)cyclopentyl)- 2-thia-6-azaspiro[3.4]octane 2,2-dioxide |
| 53D* | 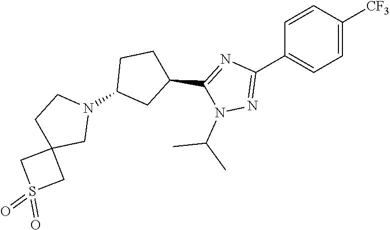 [SMILES: CC(C)n1nc(-c2ccc(C(F)(F)F)cc2)nc1[C@@H]1CC[C@@H](N2CCC3(C2)CS(=O)(=O)C3)C1] | 6-((1R,3R)-3-(1-isopropyl-3-(4- (trifluoromethyl)phenyl)-1H- 1,2,4-triazol-5-yl)cyclopentyl)- 2-thia-6-azaspiro[3.4]octane 2,2-dioxide |
| 54A* | 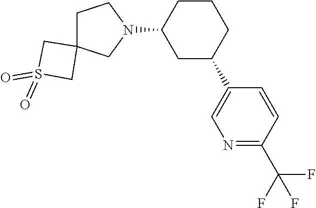 [SMILES: O=S1(=O)CC2(CCN([C@@H]3CCC[C@H](c4ccc(C(F)(F)F)nc4)C3)C2)C1] | 6-((1R,3S)-3-(6- (trifluoromethyl)pyridin-3- yl)cyclohexyl)-2-thia-6- azaspiro[3.4]octane 2,2-dioxide |
| 54B* | 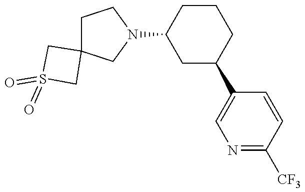 [SMILES: O=S1(=O)CC2(CCN([C@@H]3CCC[C@@H](c4ccc(C(F)(F)F)nc4)C3)C2)C1] | 6-((1R,3R)-3-(6- (trifluoromethyl)pyridin-3- yl)cyclohexyl)-2-thia-6- azaspiro[3.4]octane 2,2-dioxide |
| 54C* | 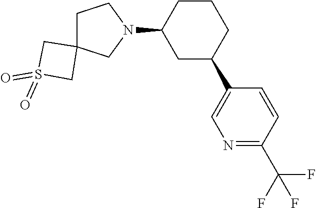 [SMILES: O=S1(=O)CC2(CCN([C@H]3CCC[C@@H](c4ccc(C(F)(F)F)nc4)C3)C2)C1] | 6-((1S,3R)-3-(6- (trifluoromethyl)pyridin-3- yl)cyclohexyl)-2-thia-6- azaspiro[3.4]octane 2,2-dioxide |
| 54D* | 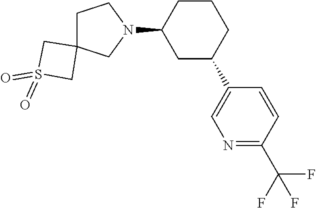 [SMILES: O=S1(=O)CC2(CCN([C@H]3CCC[C@H](c4ccc(C(F)(F)F)nc4)C3)C2)C1] | 6-((1S,3S)-3-(6- (trifluoromethyl)pyridin-3- yl)cyclohexyl)-2-thia-6- azaspiro[3.4]octane 2,2-dioxide |
| 55A* | 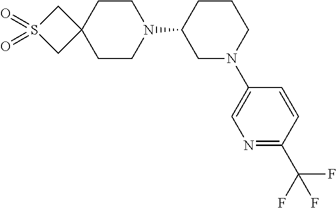 [SMILES: O=S1(=O)CC2(CCN([C@@H]3CCCN(c4ccc(C(F)(F)F)nc4)C3)CC2)C1] | (R)-7-(1-(6- (trifluoromethyl)pyridin-3- yl)piperidin-3-yl)-2-thia-7- azaspiro[3.5]nonane 2,2- dioxide |
| 55B* | 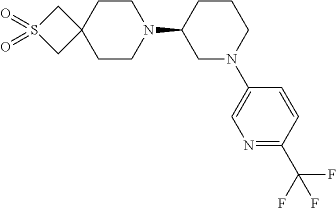 [SMILES: O=S1(=O)CC2(CCN([C@H]3CCCN(c4ccc(C(F)(F)F)nc4)C3)CC2)C1] | (S)-7-(1-(6- (trifluoromethyl)pyridin-3- yl)piperidin-3-yl)-2-thia-7- azaspiro[3.5]nonane 2,2- dioxide |
| 56A* | 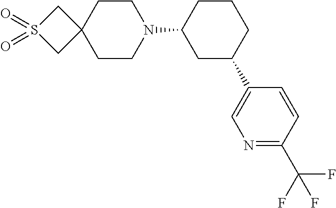 [SMILES: O=S1(=O)CC2(CCN([C@@H]3CCC[C@H](c4ccc(C(F)(F)F)nc4)C3)CC2)C1] | 7-((1R,3S)-3-(6- (trifluoromethyl)pyridin-3- yl)cyclohexyl)-2-thia-7- azaspiro[3.5]nonane 2,2- dioxide |
| 56B* | 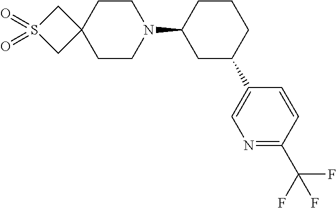 [SMILES: O=S1(=O)CC2(CCN([C@H]3CCC[C@H](c4ccc(C(F)(F)F)nc4)C3)CC2)C1] | 7-((1S,3S)-3-(6- (trifluoromethyl)pyridin-3- yl)cyclohexyl)-2-thia-7- azaspiro[3.5]nonane 2,2- dioxide |
| 56C* | 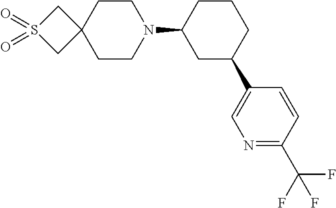 [SMILES: O=S1(=O)CC2(CCN([C@H]3CCC[C@@H](c4ccc(C(F)(F)F)nc4)C3)CC2)C1] | 7-((1S,3R)-3-(6- (trifluoromethyl)pyridin-3- yl)cyclohexyl)-2-thia-7- azaspiro[3.5]nonane 2,2- dioxide |
| 56D* | 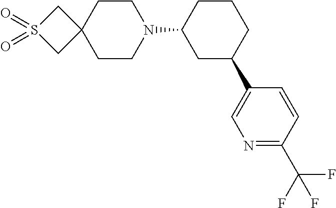 [SMILES: O=S1(=O)CC2(CCN([C@@H]3CCC[C@@H](c4ccc(C(F)(F)F)nc4)C3)CC2)C1] | 7-((1R,3R)-3-(6- (trifluoromethyl)pyridin-3- yl)cyclohexyl)-2-thia-7- azaspiro[3.5]nonane 2,2- dioxide |
| 57A* | 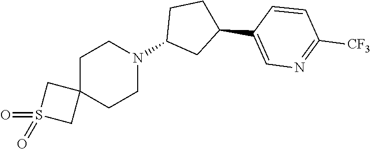 [SMILES: O=S1(=O)CC2(CCN([C@@H]3CC[C@@H](c4ccc(C(F)(F)F)nc4)C3)CC2)C1] | 7-((1R,3R)-3-(6- (trifluoromethyl)pyridin-3- yl)cyclopentyl)-2-thia-7- azaspiro[3.5]nonane 2,2- dioxide |
| 57B* | 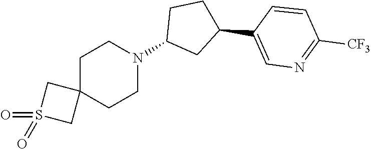 [SMILES: O=S1(=O)CC2(CCN([C@@H]3CC[C@@H](c4ccc(C(F)(F)F)nc4)C3)CC2)C1] | 7-((1S,3R)-3-(6- (trifluoromethyl)pyridin-3- yl)cyclopentyl)-2-thia-7- azaspiro[3.5]nonane 2,2- dioxide |
| 57C* | 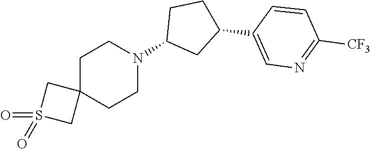 [SMILES: O=S1(=O)CC2(CCN([C@@H]3CC[C@H](c4ccc(C(F)(F)F)nc4)C3)CC2)C1] | 7-((1R,3S)-3-(6- (trifluoromethyl)pyridin-3- yl)cyclopentyl)-2-thia-7- azaspiro[3.5]nonane 2,2- dioxide |
| 57D* | 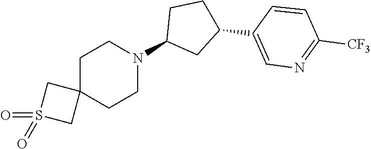 [SMILES: O=S1(=O)CC2(CCN([C@H]3CC[C@H](c4ccc(C(F)(F)F)nc4)C3)CC2)C1] | 7-((1S,3S)-3-(6- (trifluoromethyl)pyridin-3- yl)cyclopentyl)-2-thia-7- azaspiro[3.5]nonane 2,2- dioxide |
| 58A | 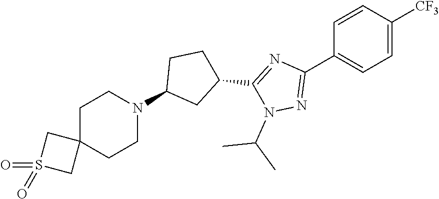 [SMILES: CC(C)n1nc(-c2ccc(C(F)(F)F)cc2)nc1[C@H]1CC[C@H](N2CCC3(CC2)CS(=O)(=O)C3)C1] | 7-((1S,3S)-3-(1-isopropyl-3-(4- (trifluoromethyl)phenyl)-1H- 1,2,4-triazol-5-yl)cyclopentyl)- 2-thia-7-azaspiro[3.5]nonane 2,2-dioxide |
| 58B | 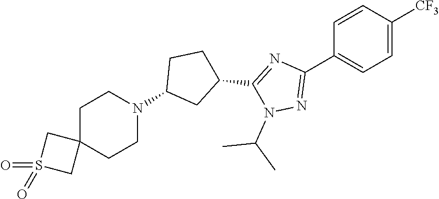 [SMILES: CC(C)n1nc(-c2ccc(C(F)(F)F)cc2)nc1[C@H]1CC[C@@H](N2CCC3(CC2)CS(=O)(=O)C3)C1] | 7-((1R,3S)-3-(1-isopropyl-3-(4- (trifluoromethyl)phenyl)-1H- 1,2,4-triazol-5-yl)cyclopentyl)- 2-thia-7-azaspiro[3.5]nonane 2,2-dioxide |
| 59 | 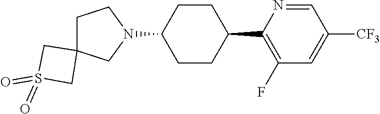 [SMILES: O=S1(=O)CC2(CCN([C@H]3CC[C@H](c4ncc(C(F)(F)F)cc4F)CC3)C2)C1] | 6-((1r,4r)-4-(3-fluoro-5- (trifluoromethyl)pyridin-2- yl)cyclohexyl)-2-thia-6- azaspiro[3.4]octane 2,2-dioxide |
| 60A | 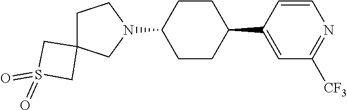 [SMILES: O=S1(=O)CC2(CCN([C@H]3CC[C@H](c4ccnc(C(F)(F)F)c4)CC3)C2)C1] | 6-((1r,4r)-4-(2- (trifluoromethyl)pyridin-4- yl)cyclohexyl)-2-thia-6- azaspiro[3.4]octane 2,2-dioxide |
| 60B | 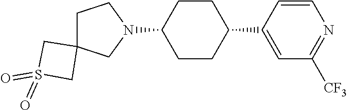 [SMILES: O=S1(=O)CC2(CCN([C@H]3CC[C@@H](c4ccnc(C(F)(F)F)c4)CC3)C2)C1] | 6-((1s,4s)-4-(2- (trifluoromethyl)pyridin-4- yl)cyclohexyl)-2-thia-6- azaspiro[3.4]octane 2,2-dioxide |
| 61A* | 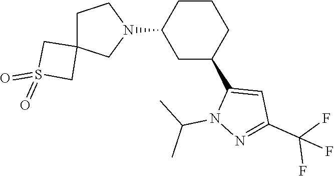 [SMILES: CC(C)n1nc(C(F)(F)F)cc1[C@@H]1CCC[C@@H](N2CCC3(C2)CS(=O)(=O)C3)C1] | 6-((1R,3R)-3-(1-isopropyl-3- (trifluoromethyl)-1H-pyrazol-5- yl)cyclohexyl)-2-thia-6- azaspiro[3.4]octane 2,2-dioxide |
| 61B* | 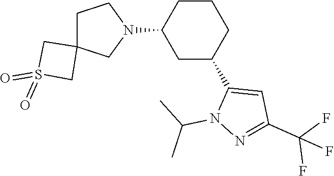 [SMILES: CC(C)n1nc(C(F)(F)F)cc1[C@H]1CCC[C@@H](N2CCC3(C2)CS(=O)(=O)C3)C1] | 6-((1R,3S)-3-(1-isopropyl-3- (trifluoromethyl)-1H-pyrazol-5- yl)cyclohexyl)-2-thia-6- azaspiro[3.4]octane 2,2-dioxide |
| 61C* | 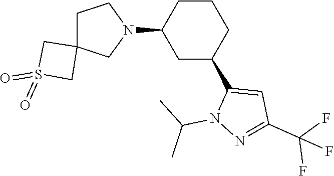 [SMILES: CC(C)n1nc(C(F)(F)F)cc1[C@@H]1CCC[C@H](N2CCC3(C2)CS(=O)(=O)C3)C1] | 6-((1S,3R)-3-(1-isopropyl-3- (trifluoromethyl)-1H-pyrazol-5- yl)cyclohexyl)-2-thia-6- azaspiro[3.4]octane 2,2-dioxide |
| 61D* | 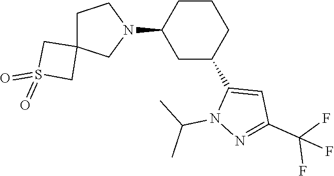 [SMILES: CC(C)n1nc(C(F)(F)F)cc1[C@H]1CCC[C@H](N2CCC3(C2)CS(=O)(=O)C3)C1] | 6-((1S,3S)-3-(1-isopropyl-3- (trifluoromethyl)-1H-pyrazol-5- yl)cyclohexyl)-2-thia-6- azaspiro[3.4]octane 2,2-dioxide |
| 62A | 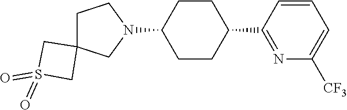 [SMILES: O=S1(=O)CC2(CCN([C@H]3CC[C@@H](c4cccc(C(F)(F)F)n4)CC3)C2)C1] | 6-((1s,4s)-4-(6- (trifluoromethyl)pyridin-2- yl)cyclohexyl)-2-thia-6- azaspiro[3.4]octane 2,2-dioxide |
| 62B | 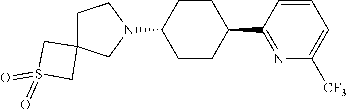 [SMILES: O=S1(=O)CC2(CCN([C@H]3CC[C@H](c4cccc(C(F)(F)F)n4)CC3)C2)C1] | 6-((1r,4r)-4-(6- (trifluoromethyl)pyridin-2- yl)cyclohexyl)-2-thia-6- azaspiro[3.4]octane 2,2-dioxide |
| 63A | 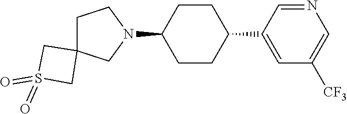 [SMILES: O=S1(=O)CC2(CCN([C@H]3CC[C@H](c4cncc(C(F)(F)F)c4)CC3)C2)C1] | 6-((1r,4r)-4-(5- (trifluoromethyl)pyridin-3- yl)cyclohexyl)-2-thia-6- azaspiro[3.4]octane 2,2-dioxide |
| 63B | 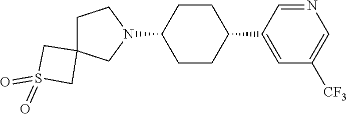 [SMILES: O=S1(=O)CC2(CCN([C@H]3CC[C@@H](c4cncc(C(F)(F)F)c4)CC3)C2)C1] | 6-((1s,4s)-4-(5- (trifluoromethyl)pyridin-3- yl)cyclohexyl)-2-thia-6- azaspiro[3.4]octane 2,2-dioxide |
| 64A | 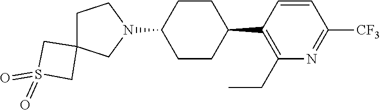 [SMILES: CCc1nc(C(F)(F)F)ccc1[C@H]1CC[C@H](N2CCC3(C2)CS(=O)(=O)C3)CC1] | 6-((1r,4r)-4-(2-ethyl-6- (trifluoromethyl)pyridin-3- yl)cyclohexyl)-2-thia-6- azaspiro[3.4]octane 2,2-dioxide |
| 64B | 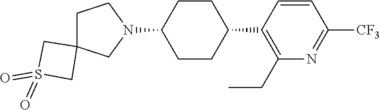 [SMILES: CCc1nc(C(F)(F)F)ccc1[C@H]1CC[C@@H](N2CCC3(C2)CS(=O)(=O)C3)CC1] | 6-((1s,4s)-4-(2-ethyl-6- (trifluoromethyl)pyridin-3- yl)cyclohexyl)-2-thia-6- azaspiro[3.4]octane 2,2-dioxide |
| 65A | 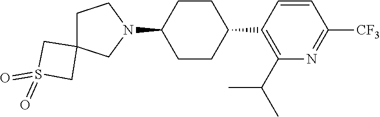 [SMILES: CC(C)c1nc(C(F)(F)F)ccc1[C@H]1CC[C@H](N2CCC3(C2)CS(=O)(=O)C3)CC1] | 6-((1r,4r)-4-(2-isopropyl-6- (trifluoromethyl)pyridin-3- yl)cyclohexyl)-2-thia-6- azaspiro[3.4]octane 2,2-dioxide |
| 65B | 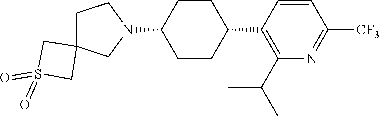 [SMILES: CC(C)c1nc(C(F)(F)F)ccc1[C@H]1CC[C@@H](N2CCC3(C2)CS(=O)(=O)C3)CC1] | 6-((1s,4s)-4-(2-isopropyl-6- (trifluoromethyl)pyridin-3- yl)cyclohexyl)-2-thia-6- azaspiro[3.4]octane 2,2-dioxide |
| 66A* | 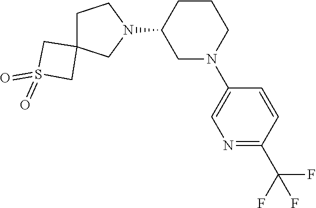 [SMILES: O=S1(=O)CC2(CCN([C@@H]3CCCN(c4ccc(C(F)(F)F)nc4)C3)C2)C1] | (R)-6-(1-(6- (trifluoromethyl)pyridin-3- yl)piperidin-3-yl)-2-thia-6- azaspiro[3.4]octane 2,2-dioxide |
| 66B* | 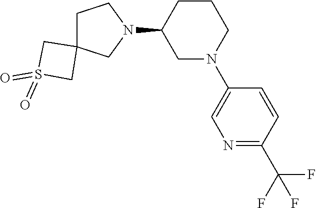 [SMILES: O=S1(=O)CC2(CCN([C@H]3CCCN(c4ccc(C(F)(F)F)nc4)C3)C2)C1] | (S)-6-(1-(6- (trifluoromethyl)pyridin-3- yl)piperidin-3-yl)-2-thia-6- azaspiro[3.4]octane 2,2-dioxide |
| 67 | 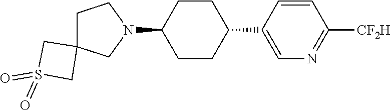 [SMILES: O=S1(=O)CC2(CCN([C@H]3CC[C@H](c4ccc(C(F)F)nc4)CC3)C2)C1] | 6-((1r,4r)-4-(6- (difluoromethyl)pyridin-3- yl)cyclohexyl)-2-thia-6- azaspiro[3.4]octane 2,2-dioxide |
| 68A* | 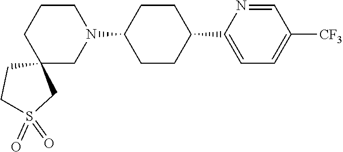 [SMILES: O=S1(=O)CC[C@@]2(CCCN([C@H]3CC[C@@H](c4ccc(C(F)(F)F)cn4)CC3)C2)C1] | (R)-7-((1s,4S)-4-(5- (trifluoromethyl)pyridin-2- yl)cyclohexyl)-2-thia-7- azaspiro[4.5]decane 2,2-dioxide |
| 68B* | 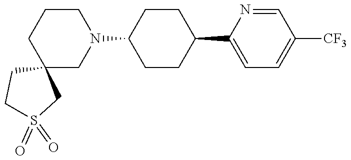 [SMILES: O=S1(=O)CC[C@@]2(CCCN([C@H]3CC[C@H](c4ccc(C(F)(F)F)cn4)CC3)C2)C1] | (R)-7-((1r,4R)-4-(5- (trifluoromethyl)pyridin-2- yl)cyclohexyl)-2-thia-7- azaspiro[4.5]decane 2,2-dioxide |
| 68C* | 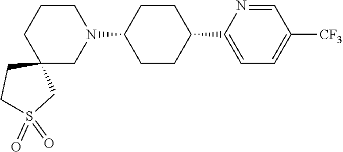 [SMILES: O=S1(=O)CC[C@]2(CCCN([C@H]3CC[C@@H](c4ccc(C(F)(F)F)cn4)CC3)C2)C1] | (S)-7-((1s,4R)-4-(5- (trifluoromethyl)pyridin-2- yl)cyclohexyl)-2-thia-7- azaspiro[4.5]decane 2,2-dioxide |
| 68D* | 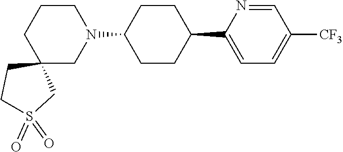 [SMILES: O=S1(=O)CC[C@]2(CCCN([C@H]3CC[C@H](c4ccc(C(F)(F)F)cn4)CC3)C2)C1] | (S)-7-((1r,4S)-4-(5- (trifluoromethyl)pyridin-2- yl)cyclohexyl)-2-thia-7- azaspiro[4.5]decane 2,2-dioxide |
| 69A* | 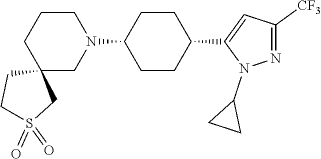 [SMILES: O=S1(=O)CC[C@@]2(CCCN([C@H]3CC[C@@H](c4cc(C(F)(F)F)nn4C4CC4)CC3)C2)C1] | (R)-7-((1s,4S)-4-(1- cyclopropyl-3- (trifluoromethyl)-1H-pyrazol-5- yl)cyclohexyl)-2-thia-7- azaspiro[4.5]decane 2,2-dioxide |
| 69B* | 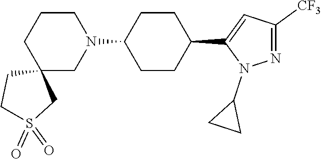 [SMILES: O=S1(=O)CC[C@@]2(CCCN([C@H]3CC[C@H](c4cc(C(F)(F)F)nn4C4CC4)CC3)C2)C1] | (R)-7-((1r,4R)-4-(1- cyclopropyl-3- (trifluoromethyl)-1H-pyrazol-5- yl)cyclohexyl)-2-thia-7- azaspiro[4.5]decane 2,2-dioxide |
| 69C* | 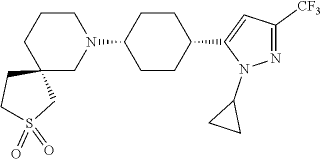 [SMILES: O=S1(=O)CC[C@]2(CCCN([C@H]3CC[C@@H](c4cc(C(F)(F)F)nn4C4CC4)CC3)C2)C1] | (S)-7-((1s,4R)-4-(1- cyclopropyl-3- (trifluoromethyl)-1H-pyrazol-5- yl)cyclohexyl)-2-thia-7- azaspiro[4.5]decane 2,2-dioxide |
| 69D* | 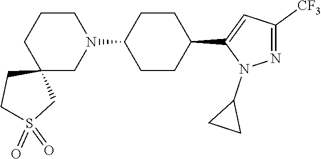 [SMILES: O=S1(=O)CC[C@]2(CCCN([C@H]3CC[C@H](c4cc(C(F)(F)F)nn4C4CC4)CC3)C2)C1] | (S)-7-((1r,4S)-4-(1-cyclopropyl- 3-(trifluoromethyl)-1H-pyrazol- 5-yl)cyclohexyl)-2-thia-7- azaspiro[4.5]decane 2,2-dioxide |
| 70A | 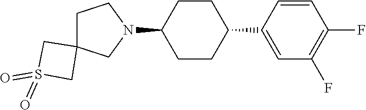 [SMILES: O=S1(=O)CC2(CCN([C@H]3CC[C@H](c4ccc(F)c(F)c4)CC3)C2)C1] | 6-((1r,4r)-4-(3,4- difluorophenyl)cyclohexyl)-2- thia-6-azaspiro[3.4]octane 2,2- dioxide |
| 70B | 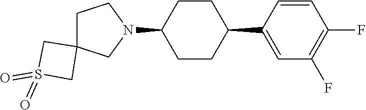 [SMILES: O=S1(=O)CC2(CCN([C@H]3CC[C@@H](c4ccc(F)c(F)c4)CC3)C2)C1] | 6-((1s,4s)-4-(3,4- difluorophenyl)cyclohexyl)-2- thia-6-azaspiro[3.4]octane 2,2- dioxide |
| 71A* | 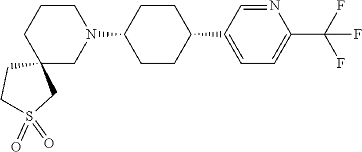 [SMILES: O=S1(=O)CC[C@@]2(CCCN([C@H]3CC[C@@H](c4ccc(C(F)(F)F)nc4)CC3)C2)C1] | (R)-7-((1s,4S)-4-(6- (trifluoromethyl)pyridin-3- yl)cyclohexyl)-2-thia-7- azaspiro[4.5]decane 2,2-dioxide |
| 71B* | 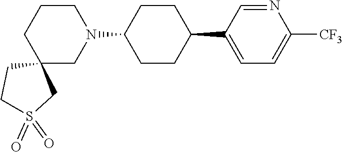 [SMILES: O=S1(=O)CC[C@@]2(CCCN([C@H]3CC[C@H](c4ccc(C(F)(F)F)nc4)CC3)C2)C1] | (R)-7-((1r,4R)-4-(6- (trifluoromethyl)pyridin-3- yl)cyclohexyl)-2-thia-7- azaspiro[4.5]decane 2,2-dioxide |
| 71C* | 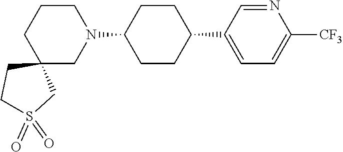 [SMILES: O=S1(=O)CC[C@]2(CCCN([C@H]3CC[C@@H](c4ccc(C(F)(F)F)nc4)CC3)C2)C1] | (S)-7-((1s,4R)-4-(6- (trifluoromethyl)pyridin-3- yl)cyclohexyl)-2-thia-7- azaspiro[4.5]decane 2,2-dioxide |
| 71D* | 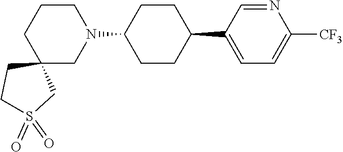 [SMILES: O=S1(=O)CC[C@]2(CCCN([C@H]3CC[C@H](c4ccc(C(F)(F)F)nc4)CC3)C2)C1] | (S)-7-((1r,4S)-4-(6- (trifluoromethyl)pyridin-3- yl)cyclohexyl)-2-thia-7- azaspiro[4.5]decane 2,2-dioxide |
| 72A | 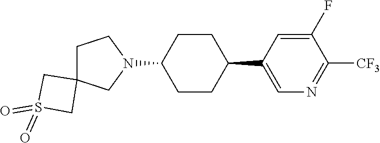 [SMILES: O=S1(=O)CC2(CCN([C@H]3CC[C@H](c4cnc(C(F)(F)F)c(F)c4)CC3)C2)C1] | 6-((1r,4r)-4-(5-fluoro-6- (trifluoromethyl)pyridin-3- yl)cyclohexyl)-2-thia-6- azaspiro[3.4]octane 2,2-dioxide |
| 72B | 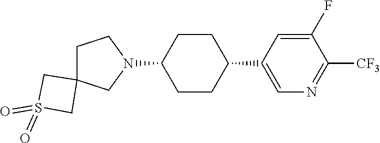 [SMILES: O=S1(=O)CC2(CCN([C@H]3CC[C@@H](c4cnc(C(F)(F)F)c(F)c4)CC3)C2)C1] | 6-((1s,4s)-4-(5-fluoro-6- (trifluoromethyl)pyridin-3- yl)cyclohexyl)-2-thia-6- azaspiro[3.4]octane 2,2-dioxide |
| 73 | 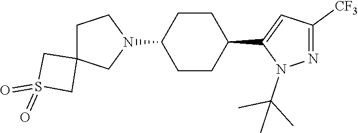 [SMILES: CC(C)(C)n1nc(C(F)(F)F)cc1[C@H]1CC[C@H](N2CCC3(C2)CS(=O)(=O)C3)CC1] | 6-((1r,4r)-4-(1-(tert-butyl)-3- (trifluoromethyl)-1H-pyrazol-5- yl)cyclohexyl)-2-thia-6- azaspiro[3.4]octane 2,2-dioxide |
| 74A | 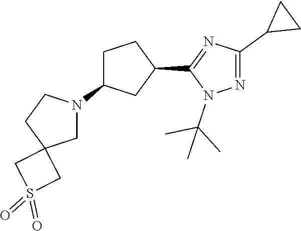 [SMILES: CC(C)(C)n1nc(C2CC2)nc1[C@@H]1CC[C@H](N2CCC3(C2)CS(=O)(=O)C3)C1] | 6-((1S,3R)-3-(1-(tert-butyl)-3- cyclopropyl-1H-1,2,4-triazol-5- yl)cyclopentyl)-2-thia-6- azaspiro[3.4]octane 2,2-dioxide |
| 74B | 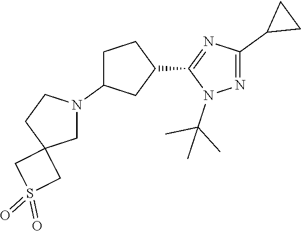 | 6-((1R,3S)-3-(1-(tert-butyl)-3- cyclopropyl-1H-1,2,4-triazol-5- yl)cyclopentyl)-2-thia-6- azaspiro[3.4]octane 2,2-dioxide |
| 75 | 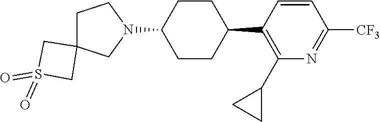 [SMILES: O=S1(=O)CC2(CCN([C@H]3CC[C@H](c4ccc(C(F)(F)F)nc4C4CC4)CC3)C2)C1] | 6-((1r,4r)-4-(2-cyclopropyl-6- (trifluoromethyl)pyridin-3- yl)cyclohexyl)-2-thia-6- azaspiro[3.4]octane 2,2-dioxide |
| 77A | 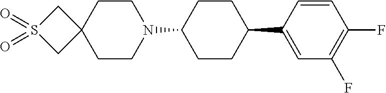 [SMILES: O=S1(=O)CC2(CCN([C@H]3CC[C@H](c4ccc(F)c(F)c4)CC3)CC2)C1] | 7-((1r,4r)-4-(3,4- difluorophenyl)cyclohexyl)-2- thia-7-azaspiro[3.5]nonane 2,2- dioxide |
| 77B | 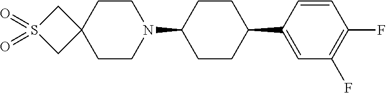 [SMILES: O=S1(=O)CC2(CCN([C@H]3CC[C@@H](c4ccc(F)c(F)c4)CC3)CC2)C1] | 7-((1s,4s)-4-(3,4- difluorophenyl)cyclohexyl)-2- thia-7-azaspiro[3.5]nonane 2,2- dioxide |
| 78A* | 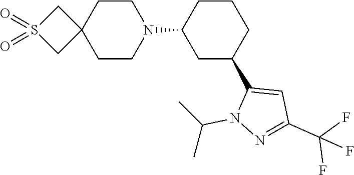 [SMILES: CC(C)n1nc(C(F)(F)F)cc1[C@@H]1CCC[C@@H](N2CCC3(CC2)CS(=O)(=O)C3)C1] | 7-((1R,3R)-3-(1-isopropyl-3- (trifluoromethyl)-1H-pyrazol-5- yl)cyclohexyl)-2-thia-7- azaspiro[3.5]nonane 2,2- dioxide |
| 78B* | 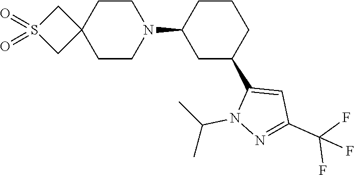 [SMILES: CC(C)n1nc(C(F)(F)F)cc1[C@@H]1CCC[C@H](N2CCC3(CC2)CS(=O)(=O)C3)C1] | 7-((1S,3R)-3-(1-isopropyl-3- (trifluoromethyl)-1H-pyrazol-5- yl)cyclohexyl)-2-thia-7- azaspiro[3.5]nonane 2,2- dioxide |
| 78C* | 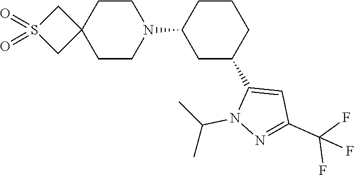 [SMILES: CC(C)n1nc(C(F)(F)F)cc1[C@H]1CCC[C@@H](N2CCC3(CC2)CS(=O)(=O)C3)C1] | 7-((1R,3S)-3-(1-isopropyl-3- (trifluoromethyl)-1H-pyrazol-5- yl)cyclohexyl)-2-thia-7- azaspiro[3.5]nonane 2,2- dioxide |
| 78D* | 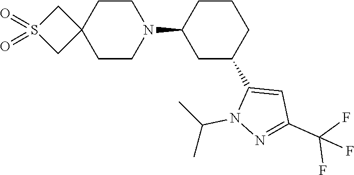 [SMILES: CC(C)n1nc(C(F)(F)F)cc1[C@H]1CCC[C@H](N2CCC3(CC2)CS(=O)(=O)C3)C1] | 7-((1S,3S)-3-(1-isopropyl-3- (trifluoromethyl)-1H-pyrazol-5- yl)cyclohexyl)-2-thia-7- azaspiro[3.5]nonane 2,2- dioxide |
| 79 | 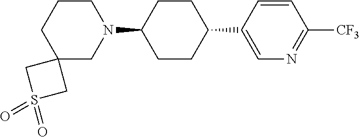 [SMILES: O=S1(=O)CC2(CCCN([C@H]3CC[C@H](c4ccc(C(F)(F)F)nc4)CC3)C2)C1] | 6-((1r,4r)-4-(6- (trifluoromethyl)pyridin-3- yl)cyclohexyl)-2-thia-6- azaspiro[3.5]nonane 2,2- dioxide |
| 80A | 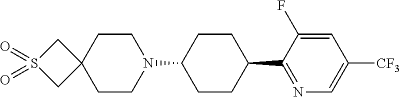 [SMILES: O=S1(=O)CC2(CCN([C@H]3CC[C@H](c4ncc(C(F)(F)F)cc4F)CC3)CC2)C1] | 7-((1r,4r)-4-(3-fluoro-5- (trifluoromethyl)pyridin-2- yl)cyclohexyl)-2-thia-7- azaspiro[3.5]nonane 2,2- dioxide |
| 80B | 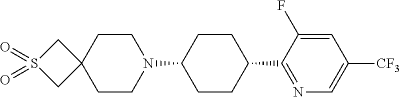 [SMILES: O=S1(=O)CC2(CCN([C@H]3CC[C@@H](c4ncc(C(F)(F)F)cc4F)CC3)CC2)C1] | 7-((1s,4s)-4-(3-fluoro-5- (trifluoromethyl)pyridin-2- yl)cyclohexyl)-2-thia-7- azaspiro[3.5]nonane 2,2- dioxide |
| 81A* | 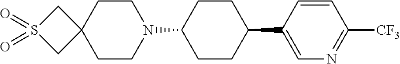 [SMILES: O=S1(=O)CC2(CCN([C@H]3CC[C@H](c4ccc(C(F)(F)F)nc4)CC3)CC2)C1] | 7-((1r,4r)-4-(6- (trifluoromethyl)pyridin-3- yl)cyclohexyl)-2-thia-7- azaspiro[3.5]nonane 2,2- |
| | | dioxide |
| 81B* | 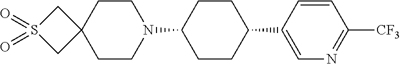 [SMILES: O=S1(=O)CC2(CCN([C@H]3CC[C@@H](c4ccc(C(F)(F)F)nc4)CC3)CC2)C1] | 7-((1s,4s)-4-(6- (trifluoromethyl)pyridin-3- yl)cyclohexyl)-2-thia-7- azaspiro[3.5]nonane 2,2- dioxide |
| 82A* | 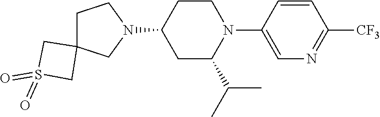 [SMILES: CC(C)[C@@H]1C[C@H](N2CCC3(C2)CS(=O)(=O)C3)CCN1c1ccc(C(F)(F)F)nc1] | 6-((2S,4R)-2-isopropyl-1-(6- (trifluoromethyl)pyridin-3- yl)piperidin-4-yl)-2-thia-6- azaspiro[3.4]octane 2,2-dioxide |
| 82B* | 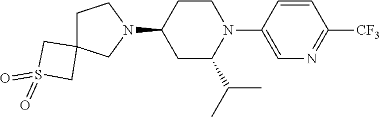 [SMILES: CC(C)[C@@H]1C[C@@H](N2CCC3(C2)CS(=O)(=O)C3)CCN1c1ccc(C(F)(F)F)nc1] | 6-((2S,4S)-2-isopropyl-1-(6- (trifluoromethyl)pyridin-3- yl)piperidin-4-yl)-2-thia-6- azaspiro[3.4]octane 2,2-dioxide |
| 82C* | 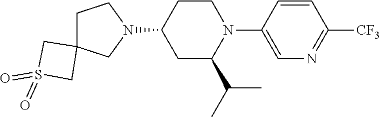 [SMILES: CC(C)[C@H]1C[C@H](N2CCC3(C2)CS(=O)(=O)C3)CCN1c1ccc(C(F)(F)F)nc1] | 6-((2R,4R)-2-isopropyl-1-(6- (trifluoromethyl)pyridin-3- yl)piperidin-4-yl)-2-thia-6- azaspiro[3.4]octane 2,2-dioxide |
| 82D* | 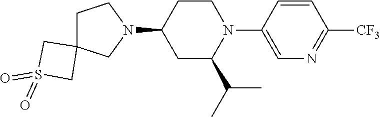 [SMILES: CC(C)[C@H]1C[C@@H](N2CCC3(C2)CS(=O)(=O)C3)CCN1c1ccc(C(F)(F)F)nc1] | 6-((2R,4S)-2-isopropyl-1-(6- (trifluoromethyl)pyridin-3- yl)piperidin-4-yl)-2-thia-6- azaspiro[3.4]octane 2,2-dioxide |
| 83 | 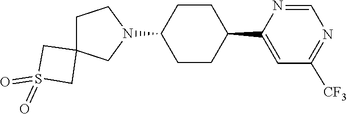 [SMILES: O=S1(=O)CC2(CCN([C@H]3CC[C@H](c4cc(C(F)(F)F)ncn4)CC3)C2)C1] | 6-((1r,4r)-4-(6- (trifluoromethyl)pyrimidin-4- yl)cyclohexyl)-2-thia-6- azaspiro[3.4]octane 2,2-dioxide |
| 84A* | 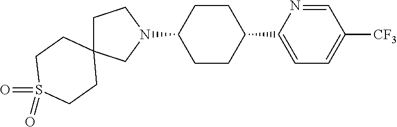 [SMILES: O=S1(=O)CCC2(CCN([C@H]3CC[C@@H](c4ccc(C(F)(F)F)cn4)CC3)C2)CC1] | 2-((1s,4s)-4-(5- (trifluoromethyl)pyridin-2- yl)cyclohexyl)-8-thia-2- azaspiro[4.5]decane 8,8-dioxide |
| 84B* | 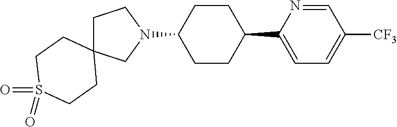 [SMILES: O=S1(=O)CCC2(CCN([C@H]3CC[C@H](c4ccc(C(F)(F)F)cn4)CC3)C2)CC1] | 2-((1r,4r)-4-(5- (trifluoromethyl)pyridin-2- yl)cyclohexyl)-8-thia-2- azaspiro[4.5]decane 8,8-dioxide |
| 85A | 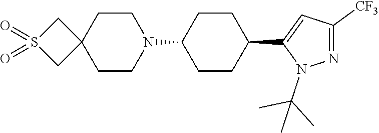 [SMILES: CC(C)(C)n1nc(C(F)(F)F)cc1[C@H]1CC[C@H](N2CCC3(CC2)CS(=O)(=O)C3)CC1] | 7-((1r,4r)-4-(1-(tert-butyl)-3- (trifluoromethyl)-1H-pyrazol-5- yl)cyclohexyl)-2-thia-7- azaspiro[3.5]nonane 2,2- dioxide |
| 85B | 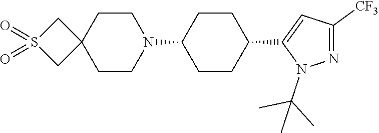 [SMILES: CC(C)(C)n1nc(C(F)(F)F)cc1[C@H]1CC[C@@H](N2CCC3(CC2)CS(=O)(=O)C3)CC1] | 7-((1s,4s)-4-(1-(tert-butyl)-3- (trifluoromethyl)-1H-pyrazol-5- yl)cyclohexyl)-2-thia-7- azaspiro[3.5]nonane 2,2- dioxide |
| 87 | 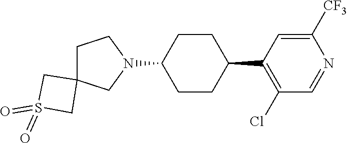 [SMILES: O=S1(=O)CC2(CCN([C@H]3CC[C@H](c4cc(C(F)(F)F)ncc4Cl)CC3)C2)C1] | 6-((1r,4r)-4-(5-chloro-2- (trifluoromethyl)pyridin-4- yl)cyclohexyl)-2-thia-6- azaspiro[3.4]octane 2,2-dioxide |
| 88 | 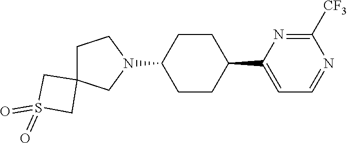 [SMILES: O=S1(=O)CC2(CCN([C@H]3CC[C@H](c4ccnc(C(F)(F)F)n4)CC3)C2)C1] | 6-((1r,4r)-4-(2- (trifluoromethyl)pyrimidin-4- yl)cyclohexyl)-2-thia-6- azaspiro[3.4]octane 2,2-dioxide |
| 90A | 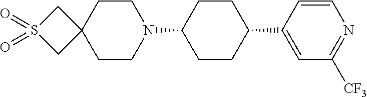 [SMILES: O=S1(=O)CC2(CCN([C@H]3CC[C@@H](c4ccnc(C(F)(F)F)c4)CC3)CC2)C1] | 7-((1s,4s)-4-(2- (trifluoromethyl)pyridin-4- yl)cyclohexyl)-2-thia-7- azaspiro[3.5]nonane 2,2- dioxide |
| 90B | 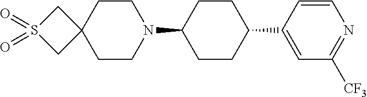 [SMILES: O=S1(=O)CC2(CCN([C@H]3CC[C@H](c4ccnc(C(F)(F)F)c4)CC3)CC2)C1] | 7-((1r,4r)-4-(2- (trifluoromethyl)pyridin-4- yl)cyclohexyl)-2-thia-7- azaspiro[3.5]nonane 2,2- dioxide |
| 91A* | 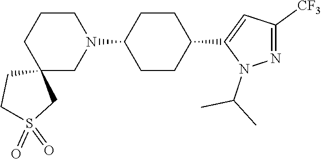 [SMILES: CC(C)n1nc(C(F)(F)F)cc1[C@H]1CC[C@@H](N2CCC[C@@]3(CCS(=O)(=O)C3)C2)CC1] | (R)-7-((1s,4S)-4-(1-isopropyl-3- (trifluoromethyl)-1H-pyrazol-5- yl)cyclohexyl)-2-thia-7- azaspiro[4.5]decane 2,2-dioxide |
| 91B* | 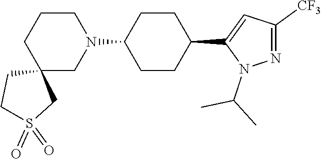 [SMILES: CC(C)n1nc(C(F)(F)F)cc1[C@H]1CC[C@H](N2CCC[C@@]3(CCS(=O)(=O)C3)C2)CC1] | (R)-7-((1r,4R)-4-(1-isopropyl- 3-(trifluoromethyl)-1H-pyrazol- 5-yl)cyclohexyl)-2-thia-7- azaspiro[4.5]decane 2,2-dioxide |
| 91C* | 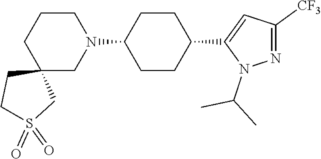 [SMILES: CC(C)n1nc(C(F)(F)F)cc1[C@H]1CC[C@@H](N2CCC[C@]3(CCS(=O)(=O)C3)C2)CC1] | (S)-7-((1s,4S)-4-(1-isopropyl-3- (trifluoromethyl)-1H-pyrazol-5- yl)cyclohexyl)-2-thia-7- azaspiro[4.5]decane 2,2-dioxide |
| 91D* | 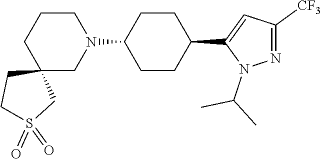 [SMILES: CC(C)n1nc(C(F)(F)F)cc1[C@H]1CC[C@H](N2CCC[C@]3(CCS(=O)(=O)C3)C2)CC1] | (S)-7-((1r,4R)-4-(1-isopropyl-3- (trifluoromethyl)-1H-pyrazol-5- yl)cyclohexyl)-2-thia-7- azaspiro[4.5]decane 2,2-dioxide |
| 92A | 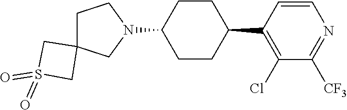 [SMILES: O=S1(=O)CC2(CCN([C@H]3CC[C@H](c4ccnc(C(F)(F)F)c4Cl)CC3)C2)C1] | 6-((1r,4r)-4-(3-chloro-2- (trifluoromethyl)pyridin-4- yl)cyclohexyl)-2-thia-6- azaspiro[3.4]octane 2,2-dioxide |
| 92B | 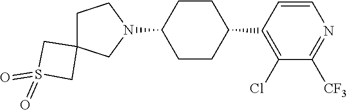 [SMILES: O=S1(=O)CC2(CCN([C@H]3CC[C@@H](c4ccnc(C(F)(F)F)c4Cl)CC3)C2)C1] | 6-((1s,4s)-4-(3-chloro-2- (trifluoromethyl)pyridin-4- yl)cyclohexyl)-2-thia-6- azaspiro[3.4]octane 2,2-dioxide |
| 93 | 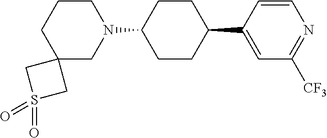 [SMILES: O=S1(=O)CC2(CCCN([C@H]3CC[C@H](c4ccnc(C(F)(F)F)c4)CC3)C2)C1] | 6-((1r,4r)-4-(2- (trifluoromethyl)pyridin-4- yl)cyclohexyl)-2-thia-6- azaspiro[3.5]nonane 2,2- dioxide |
| 94A | 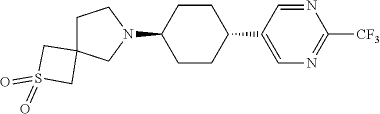 [SMILES: O=S1(=O)CC2(CCN([C@H]3CC[C@H](c4cnc(C(F)(F)F)nc4)CC3)C2)C1] | 6-((1r,4r)-4-(2- (trifluoromethyl)pyrimidin-5- yl)cyclohexyl)-2-thia-6- azaspiro[3.4]octane 2,2-dioxide |
| 94B | 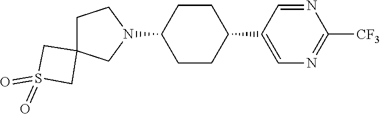 [SMILES: O=S1(=O)CC2(CCN([C@H]3CC[C@@H](c4cnc(C(F)(F)F)nc4)CC3)C2)C1] | 6-((1s,4s)-4-(2- (trifluoromethyl)pyrimidin-5- yl)cyclohexyl)-2-thia-6- azaspiro[3.4]octane 2,2-dioxide |
| 95A | 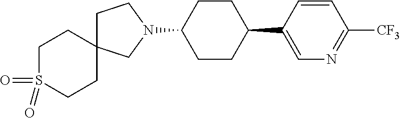 [SMILES: O=S1(=O)CCC2(CCN([C@H]3CC[C@H](c4ccc(C(F)(F)F)nc4)CC3)C2)CC1] | 2-((1r,4r)-4-(6- (trifluoromethyl)pyridin-3- yl)cyclohexyl)-8-thia-2- azaspiro[4.5]decane 8,8-dioxide |
| 95B | 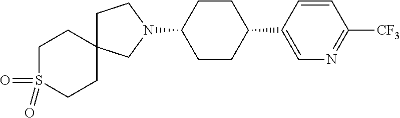 [SMILES: O=S1(=O)CCC2(CCN([C@H]3CC[C@@H](c4ccc(C(F)(F)F)nc4)CC3)C2)CC1] | 2-((1s,4s)-4-(6- (trifluoromethyl)pyridin-3- yl)cyclohexyl)-8-thia-2- azaspiro[4.5]decane 8,8-dioxide |
| 96 | 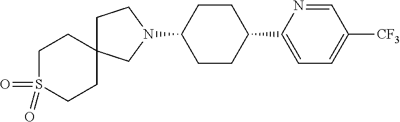 [SMILES: O=S1(=O)CCC2(CCN([C@H]3CC[C@@H](c4ccc(C(F)(F)F)cn4)CC3)C2)CC1] | 6-((1s,4s)-4-(5- (trifluoromethyl)pyridin-2- yl)cyclohexyl)-2-thia-6- azaspiro[3.4]octane 2,2-dioxide |

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable excipients. Suitable pharmaceutically acceptable excipients may include, for example, inert solid diluents and fillers, liquid diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, the pharmaceutical composition comprises a compound of Formula I, or a pharmaceutically acceptable salt thereof, and a pharmaceutically acceptable excipient. In some embodiments, the pharmaceutical composition comprises a compound of Formula Ia, or a pharmaceutically acceptable salt thereof, and a pharmaceutically acceptable excipient. In some embodiments, the pharmaceutical composition comprises a compound of Formula Ib, or a pharmaceutically acceptable salt thereof, and a pharmaceutically acceptable excipient. In some embodiments, the pharmaceutical composition comprises a compound of Formula Ic, or a pharmaceutically acceptable salt thereof, and a pharmaceutically acceptable excipient. In some embodiments, the pharmaceutical composition comprises a compound of Formula Id, or a pharmaceutically acceptable salt thereof, and a pharmaceutically acceptable excipient. In some embodiments, the pharmaceutical composition comprises a compound of Table 1, or a pharmaceutically acceptable salt thereof, and a pharmaceutically acceptable excipient.

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or tablet, such as enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained, or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

Described herein are methods for promoting myelination of central nervous system neurons in a subject suffering from a myelin-related disorder, the method comprising administering to the subject a therapeutically effective amount of a compound of Formula I, or a pharmaceutical composition comprising the same. In certain embodiments, the subject matter disclosed herein is directed to a compound of Formula I or a pharmaceutically acceptable salt thereof for promoting myelination of central nervous system neurons in a subject suffering from a myelin-related disorder. In another embodiment, the subject matter described herein is directed to the use of a compound of Formula I or a pharmaceutically acceptable salt thereof for the manufacture of a medicament for promoting myelination of central nervous system neurons in a subject suffering from a myelin-related disorder.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, inhibits enzyme mediated synthesis of one or more sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, promotes accumulation of Δ8,9-unsaturated sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, inhibits one or more of CYP51, sterol-14-reductase, or EBP enzyme mediated synthesis of sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, induces, promotes, and/or modulates oligodendrocyte precursor cell (OPC) differentiation, proliferation and/or maturation. In certain embodiments, the induction of OPC differentiation is characterized by an increase in myelin basic protein (MBP) expression.

In certain embodiments, the subject matter described herein is directed to a method of treating a disorder in a subject in need thereof, the method comprising administering to the subject in need thereof a therapeutically effective amount of a compound of Formula I or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

In certain embodiments, the subject matter disclosed herein is directed to a compound of Formula I or a pharmaceutically acceptable salt thereof, for use in treating a disorder in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

In certain embodiments, the subject matter disclosed herein is directed to the use of a compound of Formula I, or a pharmaceutically acceptable salt thereof, in the manufacture of a medicament for treating a disorder in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

In certain embodiments, the subject matter disclosed herein is directed to a method of promoting myelination in a subject in need thereof, comprising administering to the subject a therapeutically effective amount of a compound of Formula I, or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

In certain embodiments, the subject matter disclosed herein is directed to a compound of Formula I, or a pharmaceutically acceptable salt thereof, or a pharmaceutical composition comprising the compound, for use in promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

In certain embodiments, the subject matter disclosed herein is directed to use of a compound of Formula I, or a pharmaceutically acceptable salt thereof, or a pharmaceutical composition comprising the compound, in the manufacture of a medicament for promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

In certain embodiments, the subject matter disclosed herein is directed to a method of inducing endogenous oligodendrocyte precursor cell (OPC) differentiation in a subject in need thereof, the method comprising administering to the subject a therapeutically effective amount of a compound of Formula I, or a pharmaceutically acceptable salt thereof, or a pharmaceutical composition comprising the same. In certain embodiments, the subject is suffering from a myelin-related disorder. In certain embodiments, the myelin-related disorder is multiple sclerosis.

Such myelin-related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, and radiation-induced demyelination.

The compound of Formula I or a pharmaceutically acceptable salt thereof can be administered alone or in combination with another agent to a subject suffering from a myelin-related disorder to promote myelination of neurons (e.g., neuronal axons). A myelin-related disorder can include any disease, condition (e.g., those occurring from traumatic spinal cord injury and cerebral infarction), or disorder resulting in abnormalities of the myelin sheath. Abnormalities can be caused by loss of myelin referred to as demyelination, dysfunctional myelin referred to as dysmyelination, or failure to form enough myelin referred to as hypomyelination. A myelin related disorder as described herein can arise from a genetic disorder or from one or more of a variety of neurotoxic insults. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In other embodiments, the compound of Formula I is a compound of Formula Id, or a pharmaceutically acceptable salt thereof. In some embodiments, the compound of Formula I is a compound of Table 1, or a pharmaceutically acceptable salt thereof.

“Demyelination” as used herein, refers to the act of demyelinating, or the damage or loss of part or all of the myelin sheath insulating the nerves, and is the hallmark of myelin-related disorders. In certain embodiments, demyelination refers to the damage or loss of part or all of the myelin sheath insulating a subset of nerves in an individual, such as, for example, one or more nerves localized in a particular area of the body (e.g., neurons in the brain or spinal cord, or both brain and spinal cord; or the optic nerve).

Myelination of neurons requires oligodendrocytes. The term “myelination”, as used herein, refers to the generation of the nerve's myelin sheath by replacing myelin producing cells or restoring their function. The neurons that undergo remyelination may be in the brian, spinal cord, or both the brain and spinal cord. Restoring the function of a myelin producing cell may include, for example, increasing the rate of myelin production in a cell (or cells) with a less-than-average production level. Such increase may encompass raising the rate of myelin production up to or exceeding average production level; but also may encompass raising the rate of myelin production to a level that is still less than average, but higher than the previous level.

“Promoting Myelination” as used herein refers to increasing the rate of myelin production rather than a mere net increase in the amount of myelin as compared to a baseline level of myelin production rate in a subject. An increase in the rate of myelin production can be determined using imaging techniques or functional measurements. In some embodiments, myelination is promoted by increasing the differentiation of OPCs, increasing the accumulation of 8,9-unsaturated sterol intermediates in the biosynthetic pathway, increasing the formation of OPCs, or any combinations thereof. Such activities may be evaluated, for example, using one or more in vitro assays, such as those described herein or known to one of skill in the art.

A “baseline level of myelin production rate” as used herein, refers to the rate of myelin production in subject being treated before the onset of treatment.

### V. Methods of Preparing Compounds of Formula I and Pharmaceutically Acceptable Salts Thereof

Compounds can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I, or pharmaceutically acceptable salts thereof, may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

### EXAMPLES

The Examples provide exemplary methods for preparing compounds. Those skilled in the art will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art. The asterix (*) indicates an isolated isomer or isolated group of isomers, but that the stereochemistry has not been assigned.

### Example A: 6-((1S,3R)-3-(4-(Trifluoromethyl)phenyl)cyclopentyl)-2-thia-6-azaspiro[3.4]octane 2,2-dioxide (Compound 1A) and 6-((1R,3R)-3-(4-(Trifluoromethyl)phenyl)cyclopentyl)-2-thia-6-azaspiro[3.4]octane 2,2-dioxide (Compound 1B)

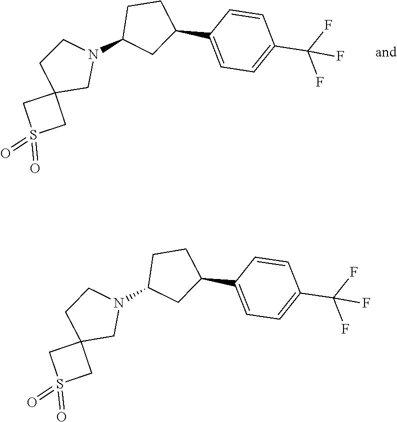 [SMILES: O=S1(=O)CC2(CCN([C@H]3CC[C@@H](c4ccc(C(F)(F)F)cc4)C3)C2)C1 ; O=S1(=O)CC2(CCN([C@@H]3CC[C@@H](c4ccc(C(F)(F)F)cc4)C3)C2)C1]

### Step 1: (R)-3-(4-(Trifluoromethyl)phenyl)cyclopentanone

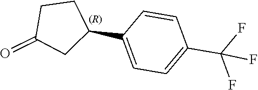

To a solution of (4-(trifluoromethyl)phenyl)boronic acid (500 mg, 2.63 mmol), cyclopent-2-enone (240 mg, 2.92 mmol) and H₂O (48 mg, 2.66 mmol) in 1,4-dioxane (5 mL) was added Rh(acac)(C₂H₄)₂ (8 mg, 0.03 mmol) and (R)-BINAP (23 mg, 0.04 mmol). The reaction mixture was stirred at 100° C. for 5 h. The reaction mixture was concentrated under reduced pressure and the resulting residue was purified by silica column chromatography (0˜10% ethyl acetate in petroleum ether) to provide the title compound (181 mg, 30% yield). LCMS (ESI) [M+H]⁺=229.1.

### Step 2: 6-((3R)-3-(4-(Trifluoromethyl)phenyl)cyclopentyl)-2-thia-6-azaspiro[3.4]octane 2,2-dioxide

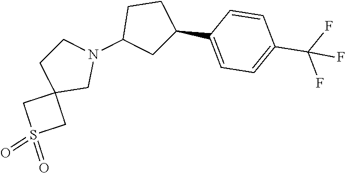 [SMILES: O=S1(=O)CC2(CCN(C3CC[C@@H](c4ccc(C(F)(F)F)cc4)C3)C2)C1]

To a solution of (R)-3-(4-(trifluoromethyl)phenyl)cyclopentanone (68 mg, 0.30 mmol) and 2-thia-6-azaspiro[3.4]octane 2,2-dioxide hydrochloride (50 mg, 0.25 mmol) in anhydrous methanol (3.0 mL) was added two drops of acetic acid. The reaction mixture was stirred at 25° C. for 5 minutes. NaBH₃CN (80 mg, 1.27 mmol) was added and the mixture stirred at 25° C. for 16 h. The reaction mixture was quenched with a saturated aq. NaHCO₃ solution (50 mL) and extracted with dichloromethane (50 mL×3). The combined organic layers were concentrated under reduced pressure and the resulting residue was purified by prep-TLC (10% methanol in dichloromethane) to provide the title compound (60 mg, 63% yield). LCMS (ESI) [M+H]⁺=374.0.

### Step 3A: 6-((1S,3R)-3-(4-(Trifluoromethyl)phenyl)cyclopentyl)-2-thia-6-azaspiro[3.4]octane 2,2-dioxide (Compound 1A)

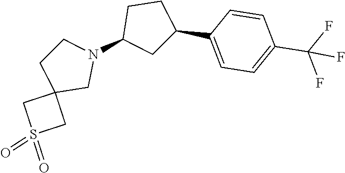 [SMILES: O=S1(=O)CC2(CCN([C@H]3CC[C@@H](c4ccc(C(F)(F)F)cc4)C3)C2)C1]

6-((3R)-3-(4-(Trifluoromethyl)phenyl)cyclopentyl)-2-thia-6-azaspiro[3.4]octane 2,2-dioxide (90 mg, 0.24 mmol) was separated by chiral SFC (Daicel Chiralpak AD, 0.1% NH₃ in water/EtOH 20:80) to provide the first eluting peak as a pure single undefined enantiomer of the title compound (40 mg, 42% yield). LCMS (ESI) [M+H]⁺=374.1. ¹H NMR (400 MHz, CDCl₃) δ 7.55 (d, J=8.0 Hz, 2H), 7.36 (d, J=8.0 Hz, 2H), 4.09 (s, 4H), 3.20-3.09 (m, 1H), 2.93-2.80 (m, 2H), 2.79-2.71 (m, 3H), 2.29-2.09 (m, 4H), 2.02-1.91 (m, 1H), 1.86-1.70 (m, 2H), 1.69-1.60 (m, 1H). The relative cis stereochemistry was assigned based on ¹H NMR analysis.

### Step 3B: 6-((1R,3R)-3-(4-(Trifluoromethyl)phenyl)cyclopentyl)-2-thia-6-azaspiro[3.4]octane 2,2-dioxide (Compound 1B)

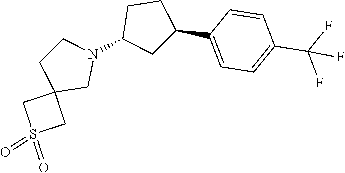 [SMILES: O=S1(=O)CC2(CCN([C@@H]3CC[C@@H](c4ccc(C(F)(F)F)cc4)C3)C2)C1]

6-((3R)-3-(4-(Trifluoromethyl)phenyl)cyclopentyl)-2-thia-6-azaspiro[3.4]octane 2,2-dioxide (90 mg, 0.24 mmol) was separated by chiral SFC (Daicel Chiralpak AD, 0.1% NH₃ in water/EtOH 20:80) to provide the second eluting peak as a pure single undefined enantiomer of the title compound (45 mg, 46% yield). LCMS (ESI) [M+H]⁺=374.1. ¹H NMR (400 MHz, DMSO-d6) δ 7.64 (d, J=8.0 Hz, 2H), 7.48 (d, J=7.6 Hz, 2H), 4.15 (s, 4H), 3.39-3.21 (m, 1H), 3.32-3.22 (m, 1H), 2.95-2.56 (m, 5H), 2.19-2.08 (m, 5H), 1.81-1.72 (m, 1H), 1.67-1.62 (m, 1H). The relative trans stereochemistry was assigned based on ¹H NMR analysis.

### Example B: 6-((1R,3S)-3-(4-(Trifluoromethyl)phenyl)cyclopentyl)-2-thia-6-azaspiro[3.4]octane 2,2-dioxide (Compound 1C) and 6-((1S,3S)-3-(4-(Trifluoromethyl)phenyl)cyclopentyl)-2-thia-6-azaspiro[3.4]octane 2,2-dioxide (Compound 1D)

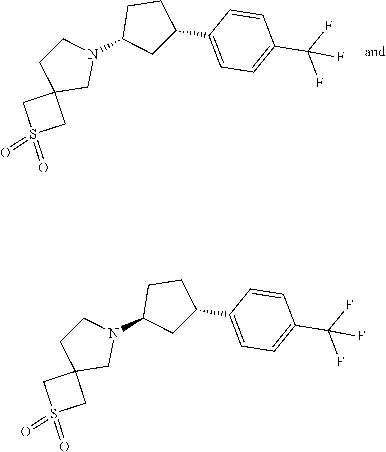 [SMILES: O=S1(=O)CC2(CCN([C@@H]3CC[C@H](c4ccc(C(F)(F)F)cc4)C3)C2)C1 ; O=S1(=O)CC2(CCN([C@H]3CC[C@H](c4ccc(C(F)(F)F)cc4)C3)C2)C1]

### Step 1: (S)-3-(4-(Trifluoromethyl)phenyl)cyclopentanone

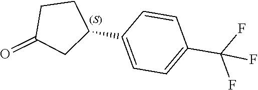

To a solution of (4-(trifluoromethyl)phenyl)boronic acid (500 mg, 2.63 mmol), cyclopent-2-enone (240 mg, 2.92 mmol) and H₂O (48 mg, 2.66 mmol) in 1,4-dioxane (5 mL) was added Rh(acac)(C₂H₄)₂ (8 mg, 0.03 mmol) and (S)-BINAP (23 mg, 0.04 mmol). The reaction mixture was stirred at 100° C. for 5 h and concentrated under reduced pressure. The residue was purified by silica column chromatography (0˜10% ethyl acetate in petroleum ether) to provide the title compound (150 mg, 25% yield). LCMS (ESI) [M+H]⁺=229.1

### Step 2: 6-((3S)-3-(4-(Trifluoromethyl)phenyl)cyclopentyl)-2-thia-6-azaspiro[3.4]octane 2,2-dioxide

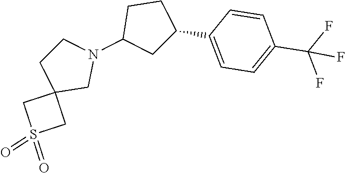 [SMILES: O=S1(=O)CC2(CCN(C3CC[C@H](c4ccc(C(F)(F)F)cc4)C3)C2)C1]

To a solution of (S)-3-(4-(trifluoromethyl)phenyl)cyclopentanone (67 mg, 0.29 mmol) and 2-thia-6-azaspiro[3.4]octane 2,2-dioxide hydrochloride (50 mg, 0.25 mmol) in anhydrous methanol (3.0 mL) was added two drops of acetic acid and the mixture was stirred at 25° C. for 5 minutes. NaBH₃CN (80 mg, 1.27 mmol) was added and the mixture was stirred at 25° C. for 16 h. The reaction mixture was quenched with a saturated aq. NaHCO₃ solution (50 mL) and extracted with dichloromethane (50 mL×3). The combined organic layer was concentrated under reduced pressure and the resulting residue was purified by prep-TLC (10% methanol in dichloromethane) to provide the title compound (75 mg, 72% yield). LCMS (ESI) [M+H]⁺=374.0.

### Step 3A: 6-((1R,3S)-3-(4-(Trifluoromethyl)phenyl)cyclopentyl)-2-thia-6-azaspiro[3.4]octane 2,2-dioxide (Compound 1C)

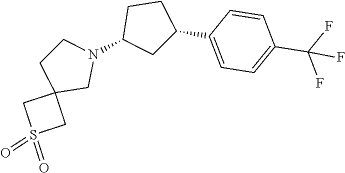 [SMILES: O=S1(=O)CC2(CCN([C@@H]3CC[C@H](c4ccc(C(F)(F)F)cc4)C3)C2)C1]

6-((3S)-3-(4-(Trifluoromethyl)phenyl)cyclopentyl)-2-thia-6-azaspiro[3.4]octane 2,2-dioxide (75 mg, 0.201 mmol) was separated by chiral SFC (Daicel Chiralpak AD-H, 0.1% NH₃ in water/MeOH 25:75) to provide the first eluting peak as a pure single undefined enantiomer of the title compound (30 mg, 39% yield). LCMS (ESI): [M+H]⁺=374.0. ¹H NMR (400 MHz, DMSO-d₆) δ 7.64 (d, J=8.4 Hz, 2H), 7.50 (d, J=8.0 Hz, 2H), 4.14 (s, 4H), 3.22-3.09 (m, 1H), 2.78-2.56 (m, 5H), 2.21-2.17 (m, 1H), 2.13-2.02 (m, 3H), 1.89-1.82 (m, 1H), 1.72-1.67 (m, J=5.6 Hz, 2H), 1.59-1.47 (m, 1H). The relative cis stereochemistry was assigned based on ¹H NMR analysis.

### Step 3B: 6-((1S,3S)-3-(4-(Trifluoromethyl)phenyl)cyclopentyl)-2-thia-6-azaspiro[3.4]octane 2,2-dioxide (Compound 1D)

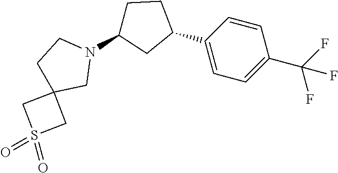 [SMILES: O=S1(=O)CC2(CCN([C@H]3CC[C@H](c4ccc(C(F)(F)F)cc4)C3)C2)C1]

6-((3S)-3-(4-(Trifluoromethyl)phenyl)cyclopentyl)-2-thia-6-azaspiro[3.4]octane 2,2-dioxide (75 mg, 0.201 mmol) from Compound 1C step 2 was separated by chiral SFC (Daicel Chiralpak AD-H, 0.1% NH₃ in water/MeOH 25:75) to provide the second eluting peak as a pure single undefined enantiomer of the title compound (23 mg, 29% yield). LCMS (ESI): [M+H]⁺=374.0. ¹H NMR (400 MHz, DMSO-d₆) δ 7.66 (d, J=8.0 Hz, 2H), 7.49 (d, J=8.0 Hz, 2H), 4.21 (s, 4H), 3.52-3.42 (m, 2H), 2.76-2.57 (m, 3H), 2.39-2.03 (m, 6H), 1.89-1.45 (m, 3H). The relative trans stereochemistry was assigned based on ¹H NMR analysis.

### Example C: 6-((1s,4s)-4-(1-Methyl-3-(trifluoromethyl)-1H-pyrazol-5-yl)cyclohexyl)-2-thia-6-azaspiro[3.4]octane 2,2-dioxide (Compound 2A) and 6-((1s,4r)-4-(1-Methyl-3-(trifluoromethyl)-1H-pyrazol-5-yl)cyclohexyl)-2-thia-6-azaspiro[3.4]octane 2,2-dioxide (Compound 2B)

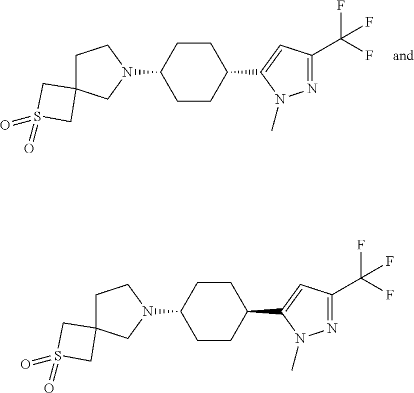 [SMILES: Cn1nc(C(F)(F)F)cc1[C@H]1CC[C@@H](N2CCC3(C2)CS(=O)(=O)C3)CC1 ; "and" ; Cn1nc(C(F)(F)F)cc1[C@H]1CC[C@H](N2CCC3(C2)CS(=O)(=O)C3)CC1]

### Step 1: 1-Methyl-5-(1,4-dioxaspiro[4.5]dec-7-en-8-yl)-3-(trifluoromethyl)-1H-pyrazole

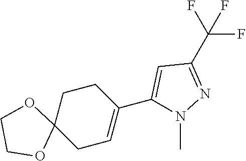 [SMILES: Cn1nc(C(F)(F)F)cc1C1=CCC2(CC1)OCCO2]

A solution of 5-bromo-1-methyl-3-(trifluoromethyl)-1H-pyrazole (15.0 g, 65.5 mmol), potassium phosphate (27.8 g, 131 mmol), SPhos Pd G₃ (2.56 g, 3.28 mmol) and 4,4,5,5-tetramethyl-2-(1,4-dioxaspiro[4.5]dec-7-en-8-yl)-1,3,2-dioxaborolane (17.4 g, 65.5 mmol) in 1,4-dioxane (300 mL) and water (75.0 mL) was stirred at 70° C. for 18 h. The reaction mixture was extracted with EtOAc (150 mL, 120 mL, 100 mL). The combined organic layer was washed with brine, dried over Na₂SO₄, filtered and concentrated under reduced pressure to give the crude title compound (20.0 g, 95.3% yield). LCMS (ESI) [M+H]⁺=289.0.

### Step 2: 1-Methyl-5-(1,4-dioxaspiro[4.5]decan-8-yl)-3-(trifluoromethyl)-1H-pyrazole

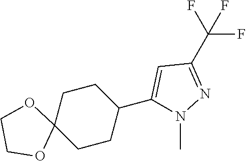 [SMILES: Cn1nc(C(F)(F)F)cc1C1CCC2(CC1)OCCO2]

A solution of 1-methyl-5-(1,4-dioxaspiro[4.5]dec-7-en-8-yl)-3-(trifluoromethyl)-1H-pyrazole (20.0 g, 69.3 mmol) and Pd(OH)₂/C (4.00 g, 69.3 mmol, 10% purity) in MeOH (300 mL) was stirred at 50° C. under hydrogen (30 psi) for 18 h. The reaction mixture was filtered through a pad of celite and concentrated to give the crude title compound (20.0 g, 99.3% yield). LCMS (ESI) [M+H]⁺=291.2.

### Step 3: 4-(1-Methyl-3-(trifluoromethyl)-1H-pyrazol-5-yl)cyclohexan-1-one

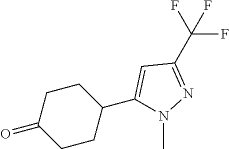 [SMILES: Cn1nc(C(F)(F)F)cc1C1CCC(=O)CC1]

A solution of 1-methyl-5-(1,4-dioxaspiro[4.5]decan-8-yl)-3-(trifluoromethyl)-1H-pyrazole (20.0 g, 68.9 mmol) in AcOH (105 mL) and H₂O (35.0 mL) was stirred at 50° C. for 8 h. The pH of the solution was adjusted to 7 with 1N aq. NaOH. The reaction mixture was extracted with EtOAc (150 mL, 120 mL, 100 mL). The combined organic layer was washed with brine, dried over Na₂SO₄, filtered and concentrated under reduced pressure. The crude product was triturated with isopropyl ether at 25° C. for 20 mins to give the title compound (10.0 g, 58.4% yield). LCMS (ESI) [M+H]⁺=247.1. ¹H NMR (400 MHz, DMSO-d₆) δ 6.57 (s, 1H), 3.85-3.94 (m, 3H), 3.26-3.36 (m, 1H), 2.52-2.66 (m, 2H), 2.23-2.48 (m, 2H), 2.10-2.22 (m, 2H), 1.80 (qd, J=12.0, 4.00 Hz, 2H).

### Step 4A: 6-((1s,4s)-4-(1-Methyl-3-(trifluoromethyl)-1H-pyrazol-5-yl)cyclohexyl)-2-thia-6-azaspiro[3.4]octane 2,2-dioxide (Compound 2A)

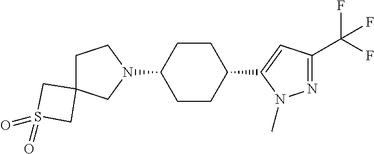 [SMILES: Cn1nc(C(F)(F)F)cc1[C@H]1CC[C@@H](N2CCC3(C2)CS(=O)(=O)C3)CC1]

A solution of 4-(1-methyl-3-(trifluoromethyl)-1H-pyrazol-5-yl)cyclohexan-1-one (59 mg, 0.24 mmol), 2-thia-6-azaspiro[3.4]octane 2,2-dioxide hydrochloride (95 mg, 0.48 mmol), acetic acid (0.028 mL, 0.48 mmol) in 1 2-dichloroethane (1.2 mL) was stirred at 25° C. for 1 h. Sodium triacetoxyborohydride (102 mg, 0.48 mmol) was added and the reaction mixture was stirred at 60° C. for 18 h. The mixture was neutralized with 1N aq. NH₄Cl (0.5 mL), diluted with DMSO, filtered and purified by prep-HPLC (acetonitrile/water gradient with 0.1% TFA) to give the first eluting peak as a pure single stereoisomer of the above titled compound (3.8 mg, 4% yield). LCMS (ESI) [M+H]⁺=392.2. ¹H NMR (400 MHz, DMSO-d₆) δ 6.52 (s, 1H), 4.22-4.08 (m, 4H), 3.84 (s, 3H), 2.87-2.76 (m, 1H), 2.74 (s, 2H), 2.61 (t, J=7.3 Hz, 2H), 2.37-2.30 (m, 1H), 2.13-2.05 (m, 2H), 1.88-1.80 (m, 2H), 1.79-1.65 (m, 2H), 1.63-1.49 (m, 4H). The relative cis stereochemistry was assigned based on ¹H NMR analysis.

### Step 4B: 6-((1s,4r)-4-(1-Methyl-3-(trifluoromethyl)-1H-pyrazol-5-yl)cyclohexyl)-2-thia-6-azaspiro[3.4]octane 2,2-dioxide (Compound 2B)

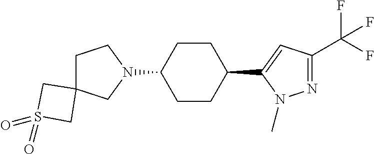 [SMILES: Cn1nc(C(F)(F)F)cc1[C@H]1CC[C@H](N2CCC3(C2)CS(=O)(=O)C3)CC1]

The crude reaction mixture from Step 4A was purified by prep-HPLC (acetonitrile/water gradient with 0.1% TFA) to give the second eluting peak as a pure single stereoisomer of the above titled compound (6.7 mg, 6% yield). LCMS (ESI) [M+H]⁺=392.2. ¹H NMR (400 MHz, DMSO-d₆) δ 6.49 (s, 1H), 4.18-4.04 (m, 4H), 3.84 (s, 3H), 2.80 (s, 2H), 2.75-2.62 (m, 3H), 2.21-2.11 (m, 1H), 2.07 (t, J=7.2 Hz, 2H), 2.00-1.86 (m, 4H), 1.41-1.22 (m, 4H). The relative trans stereochemistry was assigned based on ¹H NMR analysis.

### Example D: 6-((1r,4r)-4-(4-(Trifluoromethyl)phenyl)cyclohexyl)-2-thia-6-azaspiro[3.4]octane 2,2-dioxide (Compound 3A) and 6-((1s,4s)-4-(4-(Trifluoromethyl)phenyl)cyclohexyl)-2-thia-6-azaspiro[3.4]octane 2,2-dioxide (Compound 3B)

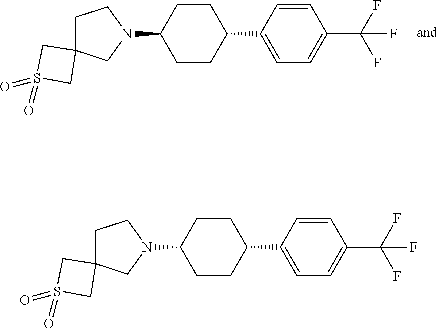 [SMILES: O=S1(=O)CC2(CCN([C@H]3CC[C@H](c4ccc(C(F)(F)F)cc4)CC3)C2)C1 ; O=S1(=O)CC2(CCN([C@H]3CC[C@@H](c4ccc(C(F)(F)F)cc4)CC3)C2)C1]

### Step 1: 8-[4-(Trifluoromethyl)phenyl]-1,4-dioxaspiro[4.5]dec-7-ene

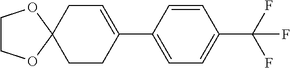 [SMILES: FC(F)(F)c1ccc(C2=CCC3(CC2)OCCO3)cc1]

To a suspension of 4-bromobenzotrifluoride (1000 mg, 4.44 mmol), 1,4-dioxa-spiro[4,5]dec-7-en-8-boronic acid pinacol ester (1182.8 mg, 4.44 mmol) and K₂CO₃ (1843 mg, 13.33 mmol) in 1,4-dioxane (10 mL) and water (2 mL) was added 1,1′-bis(diphenylphosphino)ferrocene palladium dichloride (325 mg, 0.44 mmol). The reaction mixture was stirred at 80° C. under a N2 atmosphere for 2 h. The mixture was concentrated under reduced pressure. The residue was purified by silica column chromatography (0˜20% ethyl acetate in petroleum ether) to provide the title compound (1200 mg, 95% yield). LCMS (ESI) [M+H]⁺=285.1.

### Step 2: 8-[4-(Trifluoromethyl)phenyl]-1,4-dioxaspiro[4.5]decane

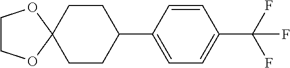 [SMILES: FC(F)(F)c1ccc(C2CCC3(CC2)OCCO3)cc1]

To a solution of 8-[4-(trifluoromethyl)phenyl]-1,4-dioxaspiro[4.5]dec-7-ene (1200 mg, 4.22 mmol) in ethanol (10 mL) was added Platinum(IV)oxide (191.7 mg, 0.84 mmol) and the mixture was stirred under H₂ (15 psi) at 25° C. for 1 h. The mixture was filtered and concentrated to provide the title compound (1200 mg, 99.3% yield). ¹H NMR (400 MHz, CD₃OD) δ 7.54 (d, J=8.0 Hz, 2H), 7.39 (d, J=8.0 Hz, 2H), 3.97-3.92 (m, 4H), 2.69-2.66 (m, 1H), 1.85-1.81 (m, 6H), 1.77-1.68 (m, 2H).

### Step 3: 4-[4-(Trifluoromethyl)phenyl]cyclohexanone

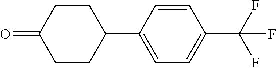 [SMILES: O=C1CCC(c2ccc(C(F)(F)F)cc2)CC1]

A suspension of 8-[4-(trifluoromethyl)phenyl]-1,4-dioxaspiro[4.5]decane (1200 mg, 4.19 mmol) and hydrochloric acid (4 mL, 24 mmol, 6 M) in water (10 mL) was stirred at 25° C. for 2 h. The mixture was diluted with water (10 mL) and extracted with ethyl acetate (50 mL×3). The combined organic layer was washed with brine (25 mL×3), dried over Na₂SO₄, filtered and concentrated under reduced pressure. The residue was purified by silica column chromatography (0˜10% ethyl acetate in petroleum ether) to provide the title compound (940 mg, 93% yield). LCMS (ESI) [M+H]⁺=243.1.

### Step 4A: 6-((1r,4r)-4-(4-(Trifluoromethyl)phenyl)cyclohexyl)-2-thia-6-azaspiro[3.4]octane 2,2-dioxide (Compound 3A)

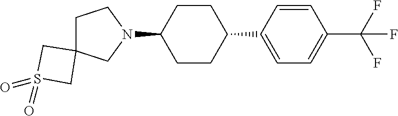 [SMILES: O=S1(=O)CC2(CCN([C@H]3CC[C@H](c4ccc(C(F)(F)F)cc4)CC3)C2)C1]

To a solution of 2-thia-6-azaspiro[3.4]octane 2,2-dioxide hydrochloride (50.0 mg, 0.25 mmol) in anhydrous dichloromethane (2 mL) was added triethylamine (0.04 mL, 0.25 mmol). The mixture was stirred at 25° C. for 30 minutes and 4-[4-(trifluoromethyl)phenyl]cyclohexanone (61.2 mg, 0.25 mmol) and acetic acid (0.05 mL) were added and the mixture was stirred at 25° C. for 4 h. NaBH(OAc)₃ (161 mg, 0.76 mmol) was added and the mixture was stirred at 25° C. for 1.5 h. The reaction mixture was concentrated and the residue was purified by silica column chromatography (0˜50% ethyl acetate in petroleum ether) to provide the cis and trans mixture of isomers (60 mg, 61.2% yield). LCMS (ESI) [M+H]⁺=388.1. The cis and trans mixture of isomers was then separated by chiral SFC (Daicel Chiral, 0.1% NH₃ in water/EtOH 45:65) to afford the first eluting peak as a pure single stereoisomer of the title compound (7.3 mg, 12% yield). LCMS (ESI) [M+H]⁺=388.1. ¹H NMR (400 MHz, CD₃OD) δ 7.57 (d, J=8.4 Hz, 2H), 7.42 (d, J=8.4 Hz, 2H), 4.18-4.09 (m, 4H), 3.01 (s, 2H), 2.85 (t, J=7.2 Hz, 2H), 2.68-2.56 (m, 1H), 2.30 (t, J=11.2 Hz, 1H), 2.21 (t, J=7.2 Hz, 2H), 2.15 (d, J=11.2 Hz, 2H), 1.95 (d, J=13.2 Hz, 2H), 1.64-1.53 (m, 2H), 1.47-1.38 (m, 2H). The relative trans stereochemistry was assigned based on ¹H NMR analysis.

### Step 4B: 6-((1s,4s)-4-(4-(Trifluoromethyl)phenyl)cyclohexyl)-2-thia-6-azaspiro[3.4]octane 2,2-dioxide (Compound 3B)

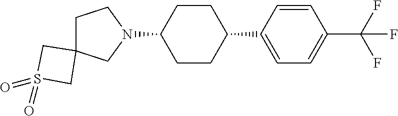 [SMILES: O=S1(=O)CC2(CCN([C@H]3CC[C@@H](c4ccc(C(F)(F)F)cc4)CC3)C2)C1]

The cis and trans mixture of isomers from step 4A were separated by chiral SFC (Daicel Chiral, 0.1% NH₃ in water/EtOH 45:65) to afford the second eluting peak as a pure single stereoisomer of the title compound (38.5 mg, 62.9% yield). LCMS (ESI) [M+H]⁺=388.1. ¹H NMR (400 MHz, CD₃OD) δ 7.57 (d, J=8.4 Hz, 2H), 7.44 (d, J=8.4 Hz, 2H), 4.13 (s, 4H), 2.94 (s, 2H), 2.82 (t, J=6.8 Hz, 2H), 2.75 (s, 1H), 2.49 (s, 1H), 2.23-2.19 (m, 2H), 2.08-2.00 (m, 2H), 1.95 (s, 2H), 1.75-1.67 (m, 2H), 1.63 (s, 2H). The relative cis stereochemistry was assigned based on ¹H NMR analysis.

### Example E: 6-(1-(4-(Trifluoromethyl)phenyl)piperidin-4-yl)-2-thia-6-azaspiro[3.4]octane 2,2-dioxide (Compound 4)

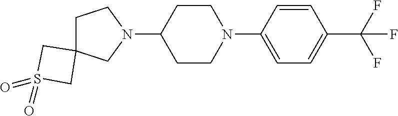 [SMILES: O=S1(=O)CC2(CCN(C3CCN(c4ccc(C(F)(F)F)cc4)CC3)C2)C1]

### Step 1: 8-(4-(Trifluoromethyl)phenyl)-1,4-dioxa-8-azaspiro[4.5]decane

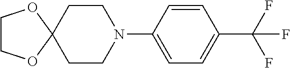 [SMILES: FC(F)(F)c1ccc(N2CCC3(CC2)OCCO3)cc1]

To a solution of 4-fluorobenzotrifluoride (1.146 g, 6.98 mmol) and 1,4-dioxa-8-azaspiro[4.5]decane (1.0 g, 6.98 mmol) in N,N-dimethylacetamide (10 mL) was added K₂CO₃ (2.41 g, 17.46 mmol) at 0° C. The mixture was stirred at 120° C. for 19 h. The reaction mixture was concentrated under reduced pressure and the crude was purified by silica column chromatography (1-10% ethyl acetate in petroleum ether) to provide the title compound (200 mg, 10% yield). LCMS (ESI) [M+H]⁺=288.0.

### Step 2: 1-[4-(Trifluoromethyl)phenyl]piperidin-4-one

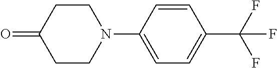 [SMILES: O=C1CCN(c2ccc(C(F)(F)F)cc2)CC1]

To a suspension of 8-[4-(trifluoromethyl)phenyl]-1,4-dioxa-8-azaspiro[4.5]decane (200 mg, 0.69 mmol) in water (2 mL) was added hydrochloric acid (0.58 mL, 3.48 mmol, 6 M) at 20° C. The reaction mixture was stirred for 2 h. The pH of the reaction mixture was adjusted to pH 7 with a saturated aq. NaHCO₃ solution (10 mL) at 0° C. and the mixture extracted with ethyl acetate (20 mL×3). The combined organic layer was washed with brine (10 mL), dried over Na₂SO₄, filtered and concentrated. The residue was purified by silica column chromatography (0-30% ethyl acetate in petroleum ether) to provide the title compound (150 mg, 76.2% yield). LCMS (ESI) [M+H]⁺=244.1.

### Step 3: 6-(1-(4-(Trifluoromethyl)phenyl)piperidin-4-yl)-2-thia-6-azaspiro[3.4]octane 2,2-dioxide

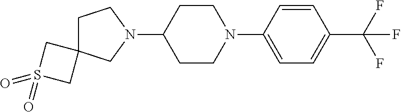 [SMILES: O=S1(=O)CC2(CCN(C3CCN(c4ccc(C(F)(F)F)cc4)CC3)C2)C1]

To a solution of 1-[4-(trifluoromethyl)phenyl]piperidin-4-one (50 mg, 0.21 mmol) and 2-thia-6-azaspiro[3.4]octane 2,2-dioxide hydrochloride (41 mg, 0.21 mmol) in methanol (2 mL) was added NaBH₃CN (65 mg, 1.03 mmol) and acetic acid (0.01 mL) at 0° C. The mixture was stirred at 80° C. for 16 h. The reaction mixture was purified by reverse phase chromatography (gradient of acetonitrile and 0.05% NH₄OH in water) to provide the title compound (24.5 mg, 29.1% yield). LCMS (ESI) [M+H]⁺=389.0. ¹H NMR (400 MHz, CD₃OD) δ 7.45 (d, J=8.8 Hz, 2H), 7.07-7.01 (m, 1H), 7.04 (d, J=8.8 Hz, 1H), 4.16-4.07 (m, 4H), 3.85 (d, J=13.2 Hz, 2H), 2.96 (s, 2H), 2.93-2.85 (m, 2H), 2.84-2.78 (m, 1H), 2.82 (t, J=7.2 Hz, 2H), 2.44-2.32 (m, 1H), 2.20 (t, J=7.2 Hz, 2H), 2.04-1.97 (m, 2H), 1.65-1.54 (m, 2H).

### Example F: 6-(1-(6-(trifluoromethyl)pyridin-3-yl)piperidin-4-yl)-2-thia-6-azaspiro[3.4]octane 2,2-dioxide (Compound 5)

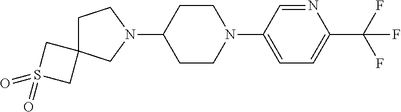 [SMILES: O=S1(=O)CC2(CCN(C3CCN(c4ccc(C(F)(F)F)nc4)CC3)C2)C1]

The title compound was synthesized generally following the procedure for Compound 4, using 5-fluoro-2-(trifluoromethyl)pyridine in the place of 4-fluorobenzotrifluoride in Step 1. LCMS (ESI) [M+H]⁺=390.2. 1H NMR (400 MHz, CD₃OD) δ 8.33 (s, 1H), 7.74-7.67 (m, 1H), 6.91 (d, J=9.2 Hz, 1H), 4.48-4.44 (m, 2H), 4.20-4.12 (m, 4H), 3.20 (s, 2H), 3.08-2.98 (m, 4H), 2.81-2.79 (m, 1H), 2.28 (t, J=7.2 Hz, 2H), 2.07-2.04 (m, 2H), 1.54-1.51 (m, 2H).

### Example G: 6-(1-(5-(trifluoromethyl)pyridin-2-yl)piperidin-4-yl)-2-thia-6-azaspiro[3.4]octane 2,2-dioxide (Compound 6)

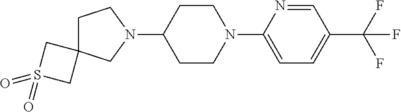 [SMILES: O=S1(=O)CC2(CCN(C3CCN(c4ccc(C(F)(F)F)cn4)CC3)C2)C1]

The title compound was synthesized generally following the procedure Compound 4, using 2-fluoro-5-(trifluoromethyl)pyridine in place of 4-fluorobenzotrifluoride in Step 1. LCMS (ESI) [M+H]⁺=390.1. ¹H NMR (400 MHz, CD₃OD) δ 8.34 (s, 1H), 7.73 (dd, J=9.2, 2.4, Hz, 1H), 6.93 (d, J=9.2 Hz, 1H), 4.55-4.52 (m, 2H), 4.27-4.15 (m, 4H), 3.43 (s, 2H), 3.26 (t, J=7.2 Hz, 2H), 3.12-2.94 (m, 3H), 2.36 (t, J=7.2 Hz, 2H), 2.14-2.11 (m, 2H), 1.60-1.56 (m, 2H).

### Example H: 6-(1-(3-Methyl-5-(trifluoromethyl)pyridin-2-yl)piperidin-4-yl)-2-thia-6-azaspiro[3.4]octane 2,2-dioxide (Compound 7)

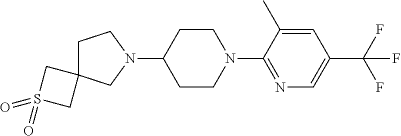 [SMILES: Cc1cc(C(F)(F)F)cnc1N1CCC(N2CCC3(C2)CS(=O)(=O)C3)CC1]

### Step 1: 1-(3-Methyl-5-(trifluoromethyl)pyridin-2-yl)piperidin-4-ol

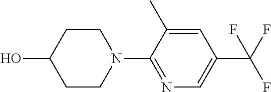 [SMILES: Cc1cc(C(F)(F)F)cnc1N1CCC(O)CC1]

A solution of 2-chloro-3-methyl-5-(trifluoromethyl)pyridine (196 mg, 1.0 mmol), cesium fluoride (456 mg, 3.0 mmol), N,N-diisopropylethylamine (0.52 mL) and piperidin-4-ol (131 mg, 1.30 mmol) in dimethyl sulfoxide (5.0 mL) was stirred at 100° C. for 18 h. The mixture was diluted with DMSO, filtered and purified by preparative reverse phase HPLC (acetonitrile/water gradient with 0.1% TFA) to give the title compound (254 mg, 98% yield). LCMS (ESI) [M+H]⁺=261.2. ¹H NMR (400 MHz, DMSO-d₆) δ 8.39 (s, 1H), 7.78 (s, 1H), 4.70 (d, J=4.2 Hz, 1H), 3.74-3.62 (m, 1H), 3.56-3.45 (m, 2H), 2.99-2.88 (m, 2H), 2.28 (s, 3H), 1.89-1.78 (m, 2H), 1.57-1.44 (m, 2H).

### Step 2: 1-(3-Methyl-5-(trifluoromethyl)pyridin-2-yl)piperidin-4-one

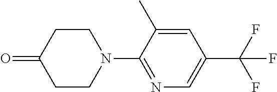 [SMILES: Cc1cc(C(F)(F)F)cnc1N1CCC(=O)CC1]

To a solution of 1-(3-methyl-5-(trifluoromethyl)pyridin-2-yl)piperidin-4-ol (250 mg, 0.96 mmol) in dichloromethane (4.8 mL) was slowly added Dess-Martin periodinane (815 mg, 1.92 mmol). The reaction mixture was stirred at 25° C. for 4 h. The reaction mixture was diluted with 1N aq. NaHCO₃ (2 mL), 1N aq. Na₂S₂O₃ (2 mL), and dicholoromethane (5 mL). The aqueous layer was extracted with dicholoromethane (2×5 mL). The combined organic layer was concentrated under reduced pressure. The mixture was diluted with DMSO, filtered and purified by preparative reverse phase HPLC (acetonitrile/water gradient with 0.1% TFA) to give the title compound (94 mg, 38% yield). LCMS (ESI) [M+H]⁺=259.2.

### Step 3: 6-(1-(3-Methyl-5-(trifluoromethyl)pyridin-2-yl)piperidin-4-yl)-2-thia-6-azaspiro[3.4]octane 2,2-dioxide

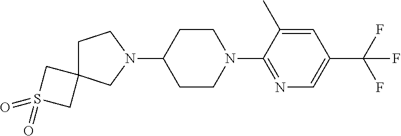 [SMILES: Cc1cc(C(F)(F)F)cnc1N1CCC(N2CCC3(C2)CS(=O)(=O)C3)CC1]

A solution of 1-(3-Methyl-5-(trifluoromethyl)pyridin-2-yl)piperidin-4-one (47 mg, 0.18 mmol), 2-thia-6-azaspiro[3.4]octane 2,2-dioxide hydrogen chloride (71 mg, 0.36 mmol), 4 A mol. sieves (15 mg) in 1 2-dichloroethane (0.9 mL) was stirred at 25° C. for 4 h. Acetic acid (0.021 mL, 0.36 mmol) and sodium triacetoxyborohydride (76 mg, 0.36 mmol) were added and the reaction mixture was stirred at 25° C. for 18 h. The mixture was neutralized with 1N aq. NH₄Cl (0.5 mL), diluted with DMSO, filtered and purified by prep-HPLC (acetonitrile/water gradient with 0.1% TFA) to give the title compound (8.2 mg, 11% yield). LCMS (ESI) [M+H]⁺=404.3. ¹H NMR (400 MHz, CD₃OD) δ 8.30 (s, 1H), 7.70 (s, 1H), 4.18-4.06 (m, 4H), 3.67 (d, J=13.0 Hz, 2H), 2.98 (s, 2H), 2.94-2.79 (m, 4H), 2.43-2.31 (m, 4H), 2.21 (t, J=7.2 Hz, 2H), 2.06-1.97 (m, 2H), 1.72-1.58 (m, 2H).

### Example I: 6-((1r,4r)-4-(1-Isopropyl-3-(trifluoromethyl)-1H-pyrazol-5-yl)cyclohexyl)-2-thia-6-azaspiro[3.4]octane 2,2-dioxide (Compound 8A) and 6-((1s,4s)-4-(1-Isopropyl-3-(trifluoromethyl)-1H-pyrazol-5-yl)cyclohexyl)-2-thia-6-azaspiro[3.4]octane 2,2-dioxide (Compound 8B)

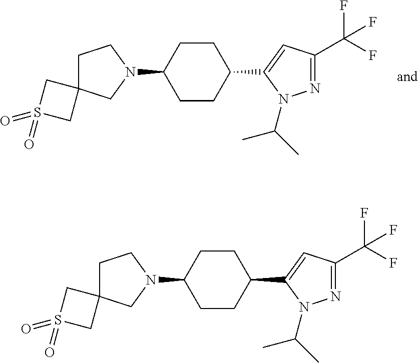 [SMILES: CC(C)n1nc(C(F)(F)F)cc1[C@H]1CC[C@H](N2CCC3(C2)CS(=O)(=O)C3)CC1 ; CC(C)n1nc(C(F)(F)F)cc1[C@H]1CC[C@@H](N2CCC3(C2)CS(=O)(=O)C3)CC1]

### Step 1: 1-Isopropyl-5-(1,4-dioxaspiro[4.5]dec-7-en-8-yl)-3-(trifluoromethyl)-1H-pyrazole

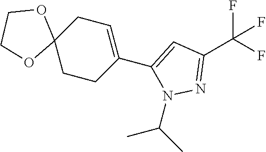 [SMILES: CC(C)n1nc(C(F)(F)F)cc1C1=CCC2(CC1)OCCO2]

To a suspension of 5-bromo-1-isopropyl-3-(trifluoromethyl)pyrazole (2 g, 7.78 mmol), 1,4-dioxa-spiro[4,5]dec-7-en-8-boronic acid pinacol ester (1035 mg, 3.89 mmol) and Cs₂CO₃ (7605 mg, 23.34 mmol) in 1,4-dioxane (20 mL) and water (5 mL) was added bis(di-tert-butyl(4-dimethylaminophenyl)phosphine)dichloropalladium(II) (551 mg, 0.78 mmol). The mixture was stirred at 80° C. under a N2 atmosphere for 2 h. The mixture was concentrated under reduced pressure and the resulting residue was purified by silica column chromatography (0-30% ethyl acetate in petroleum ether) to afford the title compound (700 mg, 28% yield). LCMS (ESI) [M+H]⁺=317.2.

### Step 2: 1-Isopropyl-5-(1,4-dioxaspiro[4.5]decan-8-yl)-3-(trifluoromethyl)-1H-pyrazole

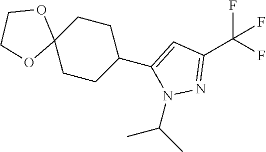 [SMILES: CC(C)n1nc(C(F)(F)F)cc1C1CCC2(CC1)OCCO2]

To a solution of 1-isopropyl-5-(1,4-dioxaspiro[4.5]dec-7-en-8-yl)-3-(trifluoromethyl)-1H-pyrazole (700 mg, 2.21 mmol) in methanol (5 mL) was added and 10% palladium on carbon (471 mg, 0.44 mmol). The suspension was degassed under vacuum and purged with H₂ (15 psi) three times. The mixture was stirred under H₂ (15 psi) at 25° C. for 16 h. The resulting mixture was filtered and the filtrate was concentrated under vacuum to afford the title compound (700 mg, 99% yield). LCMS (ESI) [M+H]⁺=319.2.

### Step 3: 4-(1-Isopropyl-3-(trifluoromethyl)-1H-pyrazol-5-yl)cyclohexanone

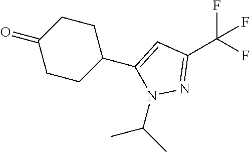 [SMILES: CC(C)n1nc(C(F)(F)F)cc1C1CCC(=O)CC1]

To a solution of 5-(1,4-dioxaspiro[4.5]decan-8-yl)-1-isopropyl-3-(trifluoromethyl)pyrazole (700 mg, 2.2 mmol) in tetrahydrofuran (10 mL) was added hydrochloric acid (5 mL, 30 mmol, 6M). The mixture was stirred at 25° C. for 30 minutes. The mixture was diluted with water (5 mL) and the pH was adjusted to pH ˜9 with a saturated aq. NaHCO₃ solution. The resulting mixture was extracted with ethyl acetate (50 mL×2). The combined organic layer was washed with water (10 mL×2), dried over anhydrous sodium sulfate, filtered and concentrated under vacuum. The residue was purified by silica column chromatography (0˜70% ethyl acetate in petroleum ether) to afford the title compound (450 mg, 75% yield). LCMS (ESI) [M+H]⁺=275.2.

### Step 4A: 6-((1r,4r)-4-(1-Isopropyl-3-(trifluoromethyl)-1H-pyrazol-5-yl)cyclohexyl)-2-thia-6-azaspiro[3.4]octane 2,2-dioxide (Compound 8A)

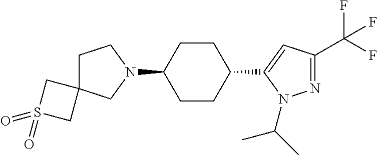 [SMILES: CC(C)n1nc(C(F)(F)F)cc1[C@H]1CC[C@H](N2CCC3(C2)CS(=O)(=O)C3)CC1]

To a solution of 4-[2-isopropyl-5-(trifluoromethyl)pyrazol-3-yl]cyclohexanone (140 mg, 0.51 mmol) in methanol (5 mL) was added 2-thia-6-azaspiro[3.4]octane 2,2-dioxide hydrochloride (115 mg, 0.58 mmol), acetic acid (0.1 mL) and NaBH₃CN (160 mg, 2.55 mmol). The mixture was stirred at 70° C. for 3 h. The reaction mixture was diluted with water (5 mL) and the pH was adjusted to pH ˜9 with a saturated aq. NaHCO₃ solution. The resulting solution was extracted with ethyl acetate (50 mL×2). The combined organic layers were washed with water (10 mL×2), dried over anhydrous sodium sulfate, filtered and concentrated under vacuum. The residue was purified by reverse phase chromatography (acetonitrile/water gradient with 0.05% NH₃ and 10 mM NH₄HCO₃) to give the second eluting peak as a pure single stereoisomer of the above titled compound (49.5 mg, 23% yield). LCMS (ESI) [M+H]⁺=420.2. ¹H NMR (400 MHz, CD₃OD) δ 6.33 (s, 1H), 4.69-4.62 (m, 1H), 4.17-4.08 (m, 4H), 2.97 (s, 2H), 2.83 (t, J=7.2 Hz, 2H), 2.79-2.71 (m, 1H), 2.31-2.23 (m, 1H), 2.20 (t, J=7.2 Hz, 2H), 2.14-2.11 (m, 2H), 2.01-1.97 (m, 2H), 1.58-1.48 (m, 2H), 1.46 (d, J=6.4 Hz, 6H), 1.45-1.36 (m, 2H). The relative trans stereochemistry was assigned based on ¹H NMR analysis.

### Step 4B: 6-((1s,4s)-4-(1-Isopropyl-3-(trifluoromethyl)-1H-pyrazol-5-yl)cyclohexyl)-2-thia-6-azaspiro[3.4]octane 2,2-dioxide (Compound 8B)

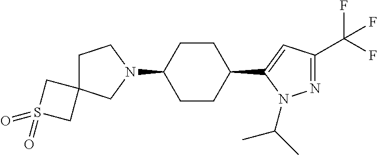 [SMILES: CC(C)n1nc(C(F)(F)F)cc1[C@H]1CC[C@@H](N2CCC3(C2)CS(=O)(=O)C3)CC1]

The crude reaction mixture from step 4A was purified by reverse phase chromatography (acetonitrile/water gradient with 0.05% NH₃ and 10 mM NH₄HCO₃) to give the first eluting peak as a pure single stereoisomer of the above titled compound (45.8 mg, 21% yield). LCMS (ESI) [M+H]⁺=420.3. ¹H NMR (400 MHz, CD₃OD) δ 6.35 (s, 1H), 4.69-4.62 (m, 1H), 4.14-4.08 (m, 4H), 2.93-2.84 (m, 3H), 2.75 (t, J=7.2 Hz, 2H), 2.42-2.37 (m, 1H), 2.19 (t, J=7.2 Hz, 2H), 1.99-1.91 (m, 2H), 1.90-1.81 (m, 2H), 1.72-1.63 (m, 4H), 1.46 (d, J=6.4 Hz, 6H). The relative cis stereochemistry was assigned based on ¹H NMR analysis.

### Example J: (S)-6-(1-(6-(Trifluoromethyl)pyridin-3-yl)pyrrolidin-3-yl)-2-thia-6-azaspiro[3.4]octane 2,2-dioxide and (R)-6-(1-(6-(trifluoromethyl)pyridin-3-yl)pyrrolidin-3-yl)-2-thia-6-azaspiro[3.4]octane 2,2-dioxide (Compounds 9A* and 9B*)

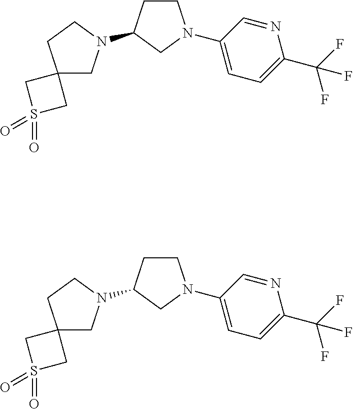 [SMILES: O=S1(=O)CC2(CCN([C@H]3CCN(c4ccc(C(F)(F)F)nc4)C3)C2)C1 ; O=S1(=O)CC2(CCN([C@@H]3CCN(c4ccc(C(F)(F)F)nc4)C3)C2)C1]

Title compounds were synthesized following a procedure similar to synthesis of Compound 4, using 1,4-dioxa-7-azaspiro[4.4]nonane and 5-fluoro-2-(trifluoromethyl)pyridine in step 1. The racemic mixture (130 mg, 0.35 mmol) was separated by chiral SFC (Daicel Chiralcel OJ-H, 0.1% NH₃ in water/EtOH 70:30) to provide Compound 9A* as the second eluting peak as a pure single undefined enantiomer of the title compound (41.8 mg, 32% yield). LCMS (ESI) [M+H]⁺=376.1. ¹H NMR (400 MHz, CDCl₃) δ 8.00 (d, J=2.8 Hz, 1H), 7.49 (d, J=8.8 Hz, 1H), 6.81 (dd, J=8.8, 2.8 Hz, 1H), 4.10 (s, 4H), 3.55-3.50 (m, 2H), 3.43-3.38 (m, 1H), 3.26-3.22 (m, 1H), 3.11-3.05 (m, 1H), 2.98-2.94 (m, 1H), 2.89-2.86 (m, 1H), 2.83-2.78 (m, 2H), 2.25-2.19 (m, 3H), 2.08-2.00 (m, 1H).

Compound 9B* was obtained as a pure single undefined enantiomer of the title compound (first eluting peak 44.3 mg, 0.1121 mmol, 32.4% yield). LCMS (ESI) [M+H]⁺=376.1. 1H NMR (400 MHz, CDCl₃) δ 8.00 (d, J=2.8 Hz, 1H), 7.49 (d, J=8.8 Hz, 1H), 6.81 (dd, J=8.8, 2.8 Hz, 1H), 4.10 (s, 4H), 3.55-3.50 (m, 2H), 3.43-3.37 (m, 1H), 3.26-3.22 (m, 1H), 3.11-3.05 (m, 1H), 2.98-2.94 (m, 1H), 2.89-2.86 (m, 1H), 2.85-2.79 (m, 2H), 2.26-2.19 (m, 3H), 2.09-1.97 (m, 1H).

### Example K (Compounds 10A*, 10B*, 10C*, and 10D*): 6-((1S,3S)-3-(1-Isopropyl-3-(trifluoromethyl)-1H-pyrazol-5-yl)cyclopentyl)-2-thia-6-azaspiro[3.4]octane 2,2-dioxide; 6-((1R,3S)-3-(1-isopropyl-3-(trifluoromethyl)-1H-pyrazol-5-yl)cyclopentyl)-2-thia-6-azaspiro[3.4]octane 2,2-dioxide; 6-((1S,3R)-3-(1-isopropyl-3-(trifluoromethyl)-1H-pyrazol-5-yl)cyclopentyl)-2-thia-6-azaspiro[3.4]octane 2,2-dioxide; 6-((1R,3R)-3-(1-isopropyl-3-(trifluoromethyl)-1H-pyrazol-5-yl)cyclopentyl)-2-thia-6-azaspiro[3.4]octane 2,2-dioxide

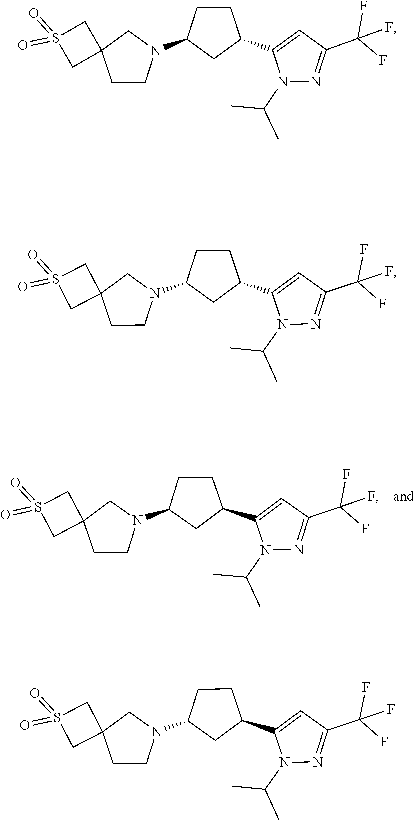 [SMILES: CC(C)n1nc(C(F)(F)F)cc1[C@H]1CC[C@H](N2CCC3(C2)CS(=O)(=O)C3)C1 ; CC(C)n1nc(C(F)(F)F)cc1[C@H]1CC[C@@H](N2CCC3(C2)CS(=O)(=O)C3)C1 ; CC(C)n1nc(C(F)(F)F)cc1[C@@H]1CC[C@H](N2CCC3(C2)CS(=O)(=O)C3)C1 ; CC(C)n1nc(C(F)(F)F)cc1[C@@H]1CC[C@@H](N2CCC3(C2)CS(=O)(=O)C3)C1]

### Step 1: 1-Isopropyl-3-(trifluoromethyl)-1H-pyrazole

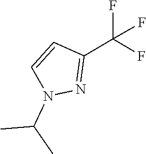

To a stirred solution of 3-(trifluoromethyl)pyrazole (7.5 g, 55.11 mmol) in N,N-dimethylformamide (80 mL) was added 2-iodopropane (28 g, 165.34 mmol) and Cs₂CO₃ (90 g, 275.57 mmol) at 25° C. The reaction mixture was stirred at 25° C. for 16 h. The reaction mixture was filtered and the filtrate was diluted with water (100 mL) and extracted with dichloromethane (100 mL×4). The combined organic layer was washed with brine (50 mL×5), dried over Na₂SO₄, filtered, and concentrated under reduced pressure to provide the title compound (6.57 g, 67% yield). LCMS (ESI) [M+H]⁺=178.9. ¹H NMR (400 MHz, CDCl₃) δ 7.46 (d, J=1.2 Hz, 1H), 6.50 (d, J=1.2 Hz, 1H), 4.60-4.53 (m, 1H), 1.54 (d, J=6.8 Hz, 6H).

### Step 2: 5-Bromo-1-isopropyl-3-(trifluoromethyl)-1H-pyrazole

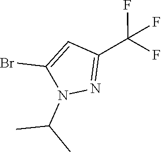

To a cooled to −78° C. solution of 1-isopropyl-3-(trifluoromethyl)-1H-pyrazole (2.0 g, 11.23 mmol) in tetrahydrofuran (40 mL) was added n-butyllithium (9 mL, 22.45 mmol) dropwise. The reaction mixture was stirred at −78° C. for 15 minutes. Bromine (2 mL, 33.68 mmol) was added dropwise over 10 minutes. The reaction mixture was stirred at −30° C. for 1.5 h then quenched with a saturated aq. NaHCO₃ solution (10 mL). The resulting mixture was extracted with tert-butyl methyl ether (50 mL×3). The combined organic layer was dried with anhydrous Na₂SO₄, filtered and concentrated under vacuum to provide the title compound (1.6 g, 55% yield). LCMS (ESI) [M+H+2]⁺=259.0. ¹H NMR (400 MHz, CDCl₃) δ 6.54 (s, 1H), 4.78-4.74 (m, 1H), 1.51 (d, J=6.4 Hz, 6H).

### Step 3: 3-(1-Isopropyl-3-(trifluoromethyl)-1H-pyrazol-5-yl)cyclopent-2-en-1-one

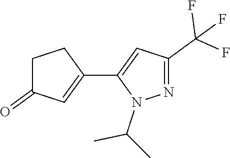 [SMILES: CC(C)n1nc(C(F)(F)F)cc1C1=CC(=O)CC1]

To a solution of 5-bromo-1-isopropyl-3-(trifluoromethyl)-1H-pyrazole (1 g, 3.89 mmol), 3-(4,4,5,5-tetramethyl-1,3,2-dioxaborolan-2-yl)cyclopent-2-en-1-one (729 mg, 3.5 mmol) in 1,4-dioxane (12 mL) and water (3 mL) was added K₂CO₃ (1613 mg, 11.67 mmol) and bis(di-tert-butyl(4-dimethylaminophenyl)phosphine)dichloropalladium(II) (276 mg, 0.39 mmol). The mixture was stirred at 80° C. under a N2 atmosphere for 2 h. The reaction mixture was concentrated under reduced pressure and the residue was purified by silica column chromatography (0˜30% ethyl acetate in petroleum ether) to afford the title compound (400 mg, 40% yield). LCMS (ESI) [M+H]⁺=259.1. ¹H NMR (400 MHz, CDCl₃) δ 6.78 (s, 1H), 6.39 (t, J=1.6 Hz, 1H), 4.80-4.73 (m, 1H), 3.05-3.02 (m, 2H), 2.61-2.54 (m, 2H), 1.56-1.56 (m, 6H).

### Step 4: 3-(1-Isopropyl-3-(trifluoromethyl)-1H-pyrazol-5-yl)cyclopentanone

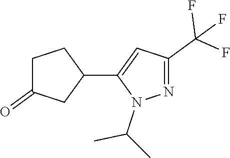 [SMILES: CC(C)n1nc(C(F)(F)F)cc1C1CCC(=O)C1]

To a solution of 3-(1-isopropyl-3-(trifluoromethyl)-1H-pyrazol-5-yl)cyclopent-2-en-1-one (400 mg, 1.55 mmol) in methanol (5 mL) was added 10% palladium on carbon (248 mg, 0.46 mmol). The reaction mixture was stirred at 25° C. under H₂ (15 psi) for 2 h. The resulting mixture was filtered and the filtrate was concentrated under reduced pressure. The residue was purified by silica column chromatography (0˜15% ethyl acetate in petroleum ether) to afford the title compound (320 mg, 80% yield). LCMS (ESI) [M+H]⁺=261.2.

### Step 5: 6-(3-(1-Isopropyl-3-(trifluoromethyl)-1H-pyrazol-5-yl)cyclopentyl)-2-thia-6-azaspiro[3.4]octane 2,2-dioxide

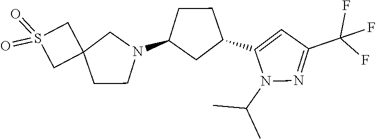 [SMILES: CC(C)n1nc(C(F)(F)F)cc1[C@H]1CC[C@H](N2CCC3(C2)CS(=O)(=O)C3)C1]

Racemic 3-(1-isopropyl-3-(trifluoromethyl)-1H-pyrazol-5-yl) (650 mg, 2.5 mmol) was separated by chiral SFC (Phenomenex-Cellulose-2, 0.1% NH₃ in water/IPA 20:80) to provide the first eluting peak as a pure single undefined enantiomer of 3-(1-isopropyl-3-(trifluoromethyl)-1H-pyrazol-5-yl)cyclopentanone (230 mg, 35% yield). To a solution of the material (110 mg, 0.42 mmol) in methanol (5 mL) was added 2-thia-6-azaspiro[3.4]octane 2,2-dioxide hydrochloride (105 mg, 0.53 mmol), acetic acid (0.02 mL) and NaBH₃CN (133 mg, 2.11 mmol). The mixture was stirred at 70° C. for 3 h. The reaction mixture was diluted with water (5 mL) and the pH was adjusted to pH ˜9 with a saturated aq. NaHCO₃ solution (1 mL). The resulting solution was extracted with dichloromethane (20 mL×2). The combined organic layer was washed with water (10 mL×2), dried over anhydrous sodium sulfate, filtered and concentrated under reduced pressure.

Compound 10A*: The residue was purified by prep-TLC (10% methanol in dichloromethane) to provide the second eluting peak as a pure single undefined enantiomer of the title compound (39.8 mg, 22% yield). LCMS (ESI) [M+H]⁺=406.2. ¹H NMR (400 MHz, DMSO-d₆) δ 6.54 (s, 1H), 4.68-4.61 (m, 1H), 4.17-4.10 (m, 4H), 3.28-3.24 (m, 1H), 2.76-2.52 (m, 5H), 2.22-2.18 (m, 1H), 2.12-2.03 (m, 3H), 1.88-1.79 (m, 1H), 1.69-1.61 (m, 2H), 1.59-1.49 (m, 1H), 1.37 (dd, J=6.4, 2.8 Hz, 6H).

Compound 10B*: The residue from the preparation of compound 10A above was purified by prep-TLC (10% methanol in dichloromethane) to provide the first eluting peak as a pure single undefined enantiomer of the title compound (43.1 mg, 24% yield). LCMS (ESI) [M+H]⁺=406.2. ¹H NMR (400 MHz, DMSO-d₆) δ 6.50 (s, 1H), 4.68-4.59 (m, 1H), 4.15-4.10 (m, 4H), 3.34-3.33 (m, 1H), 2.88-2.72 (m, 3H), 2.66-2.54 (m, 2H), 2.15-2.00 (m, 4H), 1.94-1.91 (m, 1H), 1.79-1.69 (m, 1H), 1.62-1.51 (m, 2H), 1.37 (dd, J=6.4, 5.2 Hz, 6H).

Compound 10C*: Racemic 3-(1-isopropyl-3-(trifluoromethyl)-1H-pyrazol-5-yl)cyclopentanone from the preparation of compound 10A (650 mg, 2.5 mmol) was separated by chiral SFC (Phenomenex-Cellulose-2, 0.1% NH₃ in water/IPA 20:80) to provide the second eluting peak as a pure single undefined enantiomer of 3-(1-isopropyl-3-(trifluoromethyl)-1H-pyrazol-5-yl)cyclopentanone (230 mg, 35% yield). Reductive alkylation of the above material (110 mg, 0.42 mmol), using the conditions described for the preparation of compound 10A step 5, followed by prep-TLC (10% methanol in dichloromethane) provided the second eluting peak as a pure single undefined enantiomer of the title compound (50 mg, 27% yield). LCMS (ESI) [M+H]⁺=406.2. ¹H NMR (400 MHz, DMSO-d₆) δ 6.54 (s, 1H), 4.68-4.62 (m, 1H), 4.14 (s, 4H), 3.28-3.23 (m, 1H), 2.75-2.55 (m, 5H), 2.25-2.16 (m, 1H), 2.12-2.04 (m, 3H), 1.84 (m, 1H), 1.65 (m, 2H), 1.59-1.50 (m, 1H), 1.37 (dd, J=6.4, 2.8 Hz, 6H).

Compound 10D*: The residue from the preparation of compound 10C was purified by prep-TLC (10% methanol in dichloromethane) to provide the first eluting peak as a pure single undefined enantiomer of the title compound (33.8 mg, 19% yield). LCMS (ESI) [M+H]⁺=406.2. ¹H NMR (400 MHz, DMSO-d₆) δ 6.51 (s, 1H), 4.67-4.60 (m, 1H), 4.15 (brs, 4H), 3.30-3.20 (m, 1H), 2.76 (m, 2H), 2.61 (m, 2H), 2.18-1.98 (m, 5H), 1.92 (m, 1H), 1.79-1.71 (m, 1H), 1.61-1.53 (m, 2H), 1.39-1.36 (m, 6H).

### Example L: (Cis)-6-(3-(6-(Trifluoromethyl)pyridin-3-yl)cyclopentyl)-2-thia-6-azaspiro[3.4]octane 2,2-dioxide (Compound 11A) and (Trans)-6-(3-(6-(Trifluoromethyl)pyridin-3-yl)cyclopentyl)-2-thia-6-azaspiro[3.4]octane 2,2-dioxide (Compound 11B)

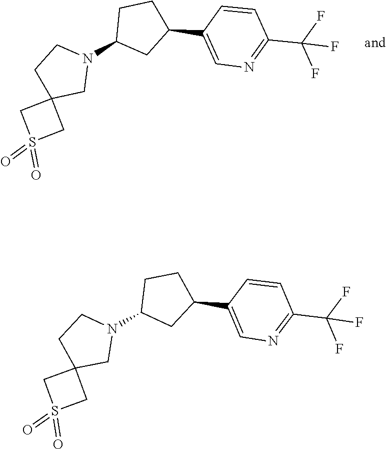 [SMILES: O=S1(=O)CC2(CCN([C@H]3CC[C@@H](c4ccc(C(F)(F)F)nc4)C3)C2)C1 ; O=S1(=O)CC2(CCN([C@@H]3CC[C@@H](c4ccc(C(F)(F)F)nc4)C3)C2)C1]

The title compounds were synthesized following a procedure similar to that used in the synthesis of Compounds 1A and 1B, using (6-(trifluoromethyl)pyridin-3-yl)boronic acid in step 1.

(Cis)-6-(3-(6-(Trifluoromethyl)pyridin-3-yl)cyclopentyl)-2-thia-6-azaspiro[3.4]octane 2,2-dioxide (Compound 11A): The racemic mixture was separated by chiral SFC (Daicel Chiralpak AD-H, 0.1% NH₃ in water/EtOH 30:70) to provide the first eluting peak as a pure single undefined enantiomer of the title compound (30.7 mg, 37% yield). LCMS (ESI) [M+H]⁺=375.2. ¹H NMR (400 MHz, DMSO-d₆) δ 8.68 (s, 1H), 7.99 (d, J=8.0 Hz, 1H), 7.82 (d, J=8.4 Hz, 1H), 4.19-4.12 (m, 4H), 3.30-3.14 (m, 2H), 2.80-2.72 (m, 2H), 2.69-2.69 (m, 2H), 2.28-2.18 (m, 1H), 2.16-2.04 (m, 3H), 1.94-1.81 (m, 1H), 2.16-2.04 (m, 2H), 1.59 (d, J=10.0 Hz, 1H). The relative stereochemistry as a cis isomer was assigned based on ¹H NMR analysis, absolute stereochemistry was not assigned.

(Trans)-6-(3-(6-(Trifluoromethyl)pyridin-3-yl)cyclopentyl)-2-thia-6-azaspiro[3.4]octane 2,2-dioxide (Compound 11B): The racemic mixture was separated by chiral SFC (Daicel Chiralpak AD-H, 0.1% NH₃ in water/EtOH 30:70) to provide the second eluting peak as a pure single undefined enantiomer of the title compound (36.8 mg, 44% yield). LCMS (ESI) [M+H]⁺=375.2. ¹H NMR (400 MHz, DMSO-d₆) δ 8.67 (s, 1H), 7.96 (d, J=8.0 Hz, 1H), 7.82 (d, J=8.4 Hz, 1H), 4.19-4.12 (m, 4H), 3.03-2.70 (m, 4H), 2.66-2.57 (m, 2H), 2.20-2.05 (m, 4H), 2.03-1.93 (m, 1H), 1.84-1.70 (m, 1H), 1.65-1.56 (m, 2H). The relative stereochemistry as a trans isomer was assigned based on ¹H NMR analysis, absolute stereochemistry was not assigned.

### Example M: (Compounds 11C* and 11D*): 6-((1R,3S)-3-(6-(Trifluoromethyl)pyridin-3-yl)cyclopentyl)-2-thia-6-azaspiro[3.4]octane 2,2-dioxide and 6-((1S,3S)-3-(6-(Trifluoromethyl)pyridin-3-yl)cyclopentyl)-2-thia-6-azaspiro[3.4]octane 2,2-dioxide

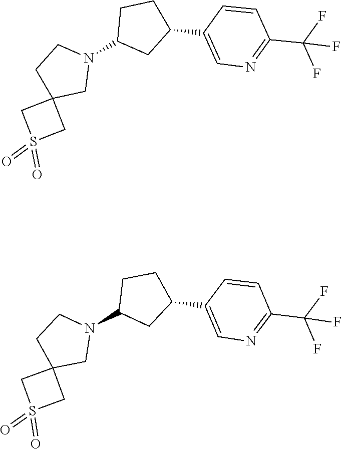

The title compound was synthesized following a procedure similar to the procedure for the synthesis of Compound 1C and 1D, using (6-(trifluoromethyl)pyridin-3-yl)boronic acid in step 1.

Compound 11C*: The racemic mixture was separated by chiral SFC (Daicel Chiralpak IG, 0.1% NH₃ in water/MeOH 45:55) to provide the first eluting peak as a pure single undefined enantiomer of the title compound (34 mg, 28% yield). LCMS (ESI) [M+H]⁺=375.2. ¹H NMR (400 MHz, CDCl₃) δ 8.61 (s, 1H), 7.75 (d, J=7.2 Hz, 1H), 7.63 (d, J=8.4 Hz, 1H), 4.10 (s, 4H), 3.24-3.15 (m, 1H), 2.94-2.69 (m, 5H), 2.34-2.26 (m, 1H), 2.24-2.13 (m, 3H), 2.09-1.92 (m, 1H), 1.86-1.57 (m, 2H), 1.68-1.53 (m, 1H). The stereochemistry of the pyridine ring attachment was assigned, the stereochemistry of the other stereocenter was arbitrarily assigned.

Compound 11D*: The racemic mixture was separated by chiral SFC (Daicel Chiralpak IG, 0.1% NH₃ in water/MeOH 45:55) to provide the second eluting peak as a pure single undefined enantiomer of the title compound (30 mg, 25% yield). LCMS (ESI) [M+H]⁺=375.2. ¹H NMR (400 MHz, CDCl₃) δ 8.60 (d, J=1.6 Hz, 1H), 7.72-7.68 (m, 1H), 7.64-7.60 (m, 1H), 4.09 (s, 4H), 3.41-3.29 (m, 1H), 2.91-2.80 (m, 3H), 2.76-2.71 (m, 2H), 2.27-2.05 (m, 5H), 1.88-1.78 (m, 1H), 1.72-1.63 (m, 2H). The stereochemistry of the pyridine ring attachment was assigned, the stereochemistry of the other stereocenter was arbitrarily assigned.

### Example N: 6-((1r,4r)-4-(1-Cyclopropyl-3-(trifluoromethyl)-1H-pyrazol-5-yl)cyclohexyl)-2-thia-6-azaspiro[3.4]octane 2,2-dioxide (Compound 12A) and 6-((1s,4s)-4-(1-Cyclopropyl-3-(trifluoromethyl)-1H-pyrazol-5-yl)cyclohexyl)-2-thia-6-azaspiro[3.4]octane 2,2-dioxide (Compound 12B)

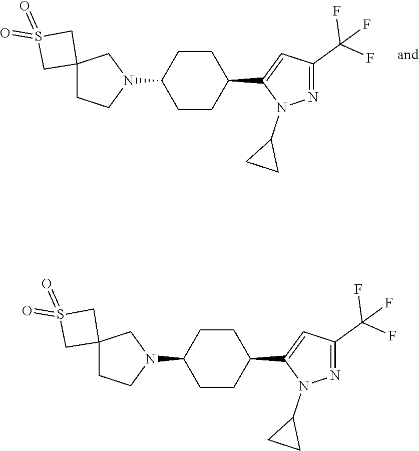 [SMILES: O=S1(=O)CC2(CCN([C@H]3CC[C@H](c4cc(C(F)(F)F)nn4C4CC4)CC3)C2)C1 ; O=S1(=O)CC2(CCN([C@H]3CC[C@@H](c4cc(C(F)(F)F)nn4C4CC4)CC3)C2)C1]

### Step 1: 4,4,4-Trifluoro-1-(1,4-dioxaspiro[4.5]decan-8-yl)butane-1,3-dione

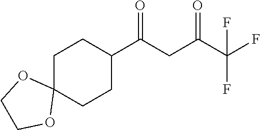 [SMILES: O=C(CC(=O)C(F)(F)F)C1CCC2(CC1)OCCO2]

A cooled to 0° C. solution of 1-(1,4-dioxaspiro[4.5]decan-8-yl)ethan-1-one (1.0 g, 5.43 mmol), ethyl 2,2,2-trifluoroacetate (1.93 g, 13.6 mmol) and sodium methoxide (6.20 mL, 27.1 mmol, 25 mass % in methanol) in 1,4-dioxane (36.2 mL) was stirred at 0-25° C. for 18 h. The reaction mixture was diluted with EtOAc (50 mL) and 1N aq. NH₄Cl (30 mL) and neutralized to pH ˜6-7 with 2N aq. HCl. The aqueous layer was extracted with EtOAc (2×25 mL). The combined organic layer was washed with brine (25 mL), dried (Na₂SO₄) and concentrated under reduced pressure to give the crude title compound (2.16 g, 100% yield). LCMS (ESI) [M+H]⁺=281.3.

### Step 2: 4-(1-Cyclopropyl-3-(trifluoromethyl)-1H-pyrazol-5-yl)cyclohexan-1-one

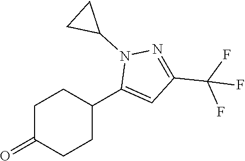 [SMILES: O=C1CCC(c2cc(C(F)(F)F)nn2C2CC2)CC1]

A solution of 4,4,4-trifluoro-1-(1,4-dioxaspiro[4.5]decan-8-yl)butane-1,3-dione (638 mg, 2.23 mmol), cyclopropyl hydrazine hydrochloride (297 mg, 2.73 mmol) and triethylamine (0.38 mL, 2.73 mmol) in hexafluoro-2-propanol (3.8 mL) was stirred at 25° C. for 18 h. AcOH (0.66 mL, 11.5 mmol) and water (2 mL) was added and the reaction mixture was stirred at 50° C. for 18 h. The reaction mixture was diluted with dichloromethane (20 mL) and 1N aq. NaHCO₃ (14 mL). The aqueous layer was extracted with dichloromethane (2×20 mL). The combined organic layer was dried (Na₂SO₄) and concentrated under reduced pressure to give the crude title compound (0.72 g, 100% yield). LCMS (ESI) [M+H]⁺=273.0.

### Step 3A: 6-((1r,4r)-4-(1-Cyclopropyl-3-(trifluoromethyl)-1H-pyrazol-5-yl)cyclohexyl)-2-thia-6-azaspiro[3.4]octane 2,2-dioxide (Compound 12A)

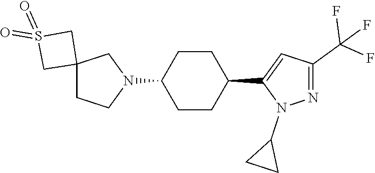 [SMILES: O=S1(=O)CC2(CCN([C@H]3CC[C@H](c4cc(C(F)(F)F)nn4C4CC4)CC3)C2)C1]

A solution of 4-(1-cyclopropyl-3-(trifluoromethyl)-1H-pyrazol-5-yl)cyclohexan-1-one (190 mg, 0.70 mmol), 2-thia-6-azaspiro[3.4]octane 2,2-dioxide trifluoroacetic acid (384 mg, 1.40 mmol) and N,N-diisopropylethylamine (1.22 mL, 6.98 mmol) in dichloromethane (6.98 mL) was stirred at 30° C. for 4 h. Sodium triacetoxyborohydride (444 mg, 2.09 mmol) was added and the reaction mixture was stirred at 25° C. for 18 h. The mixture was neutralized with 1N aq. NH₄Cl (0.5 mL), diluted with DMSO, filtered and purified by prep-HPLC (acetonitrile/water gradient with 0.1% TFA) to give the second eluting peak as a pure single stereoisomer of the above titled compound (32 mg, 11% yield). LCMS (ESI) [M+H]⁺=418.2. ¹H NMR (400 MHz, DMSO-d₆) δ 6.50 (s, 1H), 4.17-4.06 (m, 4H), 3.76-3.67 (m, 1H), 2.96-2.86 (m, 1H), 2.81 (s, 2H), 2.67 (t, J=7.2 Hz, 2H), 2.22-2.13 (m, 1H), 2.07 (t, J=7.2 Hz, 2H), 2.02-1.94 (m, 4H), 1.49-1.23 (m, 4H), 1.11-0.99 (m, 4H). The relative trans stereochemistry was assigned based on ¹H NMR analysis.

### Step 3B: 6-((1s,4s)-4-(1-Cyclopropyl-3-(trifluoromethyl)-1H-pyrazol-5-yl)cyclohexyl)-2-thia-6-azaspiro[3.4]octane 2,2-dioxide (Compound 12B)

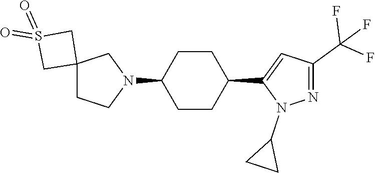 [SMILES: O=S1(=O)CC2(CCN([C@H]3CC[C@@H](c4cc(C(F)(F)F)nn4C4CC4)CC3)C2)C1]

Purification of the crude mixture of Step 3A by prep-HPLC (acetonitrile/water gradient with 0.1% TFA) afforded the first eluting peak as a pure single stereoisomer of the above titled compound (55 mg, 19% yield). LCMS (ESI) [M+H]⁺=418.2. ¹H NMR (400 MHz, DMSO-d₆) δ 6.53 (s, 1H), 4.21-4.10 (m, 4H), 3.76-3.66 (m, 1H), 3.07-2.97 (m, 1H), 2.74 (s, 2H), 2.61 (t, J=7.3 Hz, 2H), 2.36-2.32 (m, 1H), 2.09 (t, J=7.4 Hz, 2H), 1.90-1.79 (m, 2H), 1.79-1.62 (m, 4H), 1.62-1.51 (m, 2H), 1.14-0.99 (m, 4H). The relative cis stereochemistry was assigned based on ¹H NMR analysis.

### Example O: (Compounds 13A*, 13B*, 13C*, and 13D*): 6-((1R,3S)-3-(1-Isopropyl-3-(5-(trifluoromethyl)pyridin-3-yl)-1H-pyrazol-5-yl)cyclopentyl)-2-thia-6-azaspiro[3.4]octane 2,2-dioxide; 6-((1S,3R)-3-(1-isopropyl-3-(5-(trifluoromethyl)pyridin-3-yl)-1H-pyrazol-5-yl)cyclopentyl)-2-thia-6-azaspiro[3.4]octane 2,2-dioxide; 6-((1R,3R)-3-(1-isopropyl-3-(5-(trifluoromethyl)pyridin-3-yl)-1H-pyrazol-5-yl)cyclopentyl)-2-thia-6-azaspiro[3.4]octane 2,2-dioxide; 6-((1S,3S)-3-(1-isopropyl-3-(5-(trifluoromethyl)pyridin-3-yl)-1H-pyrazol-5-yl)cyclopentyl)-2-thia-6-azaspiro[3.4]octane 2,2-dioxide

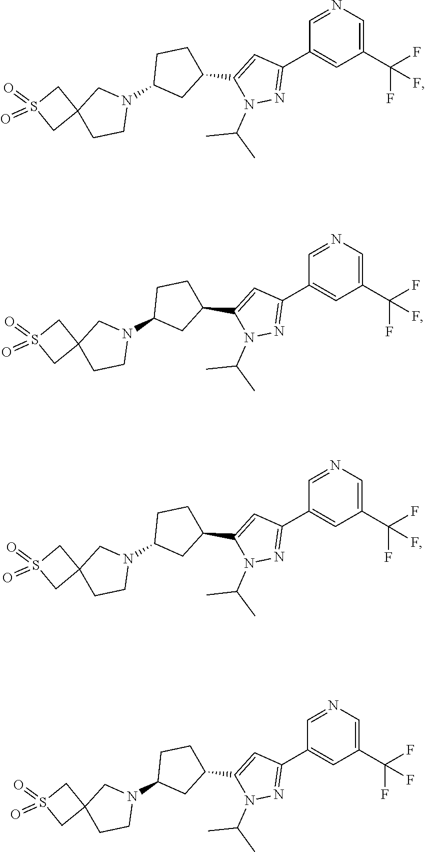 [SMILES: CC(C)n1nc(-c2cncc(C(F)(F)F)c2)cc1[C@H]1CC[C@@H](N2CCC3(C2)CS(=O)(=O)C3)C1 ; CC(C)n1nc(-c2cncc(C(F)(F)F)c2)cc1[C@@H]1CC[C@H](N2CCC3(C2)CS(=O)(=O)C3)C1 ; CC(C)n1nc(-c2cncc(C(F)(F)F)c2)cc1[C@@H]1CC[C@@H](N2CCC3(C2)CS(=O)(=O)C3)C1 ; CC(C)n1nc(-c2cncc(C(F)(F)F)c2)cc1[C@H]1CC[C@H](N2CCC3(C2)CS(=O)(=O)C3)C1]

### Step 1: 3-Bromocyclopent-2-enone

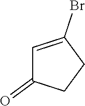

To a suspension of Ph₃PBr₂ (4.73 g, 11.2 mmol) in dichloromethane (10 mL) was added cyclopent-4-ene-1,3-dione (1.0 g, 10.2 mmol) and triethylamine (1.56 mL, 11.2 mmol) at 25° C. The reaction mixture was stirred at 25° C. for 18 h. A yellow suspension was formed. The reaction mixture was concentrated under vacuum and 2-methoxy-2-methylpropane (50 mL) was added. The reaction mixture was stirred and filtered. The organic layer was concentrated under reduced pressure and purified by silica column chromatography (10%˜20% ethyl acetate in petroleum ether) to provide the title compound (1.10 g, 67% yield). ¹H NMR (400 MHz, DMSO-d₆) δ 6.66-6.62 (m, 1H), 3.01-2.94 (m, 2H), 2.50-2.47 (m, 2H).

### Step 2: 3-(4,4,5,5-Tetramethyl-1,3,2-dioxaborolan-2-yl)cyclopent-2-enone

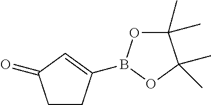

A mixture of 4-bromocyclopent-2-enone (1.10 g, 6.83 mmol), 4,4,4′,4′,5,5,5′,5′-octamethyl-2,2′-bi(1,3,2-dioxaborolane) (1.91 g, 7.52 mmol), bis(di-tert-butyl(4-dimethylaminophenyl)phosphine)dichloropalladium(II) (500 mg, 0.68 mmol) and KOAc (2.01 g, 20.5 mmol) in 1,4-dioxane (10 mL) was degassed and purged with N2 three times. The reaction mixture was stirred at 100° C. under N2 for 12 h. A brown suspension was formed. The reaction mixture was quenched with water (30 mL) and extracted with ethyl acetate (50 mL×3). The combined organic layer was washed with brine (50 mL), dried over anhydrous sodium sulfate, filtered and concentrated. The residue was purified by silica column chromatography (2%˜3% methanol in dichloromethane) to provide the title compound (1.30 g, 91% yield). ¹H NMR (400 MHz, DMSO-d₆) δ 6.45-6.40 (m, 1H), 2.68-2.63 (m, 2H), 2.28-2.23 (m, 2H), 1.27 (s, 12H).

### Step 3: 3-(3-Bromo-1-isopropyl-1H-pyrazol-5-yl)cyclopent-2-enone

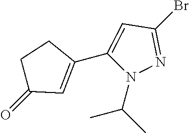

A suspension of 3-(4,4,5,5-tetramethyl-1,3,2-dioxaborolan-2-yl)cyclopent-2-enone (1.30 g, 6.25 mmol), 3,5-dibromo-1-isopropyl-1H-pyrazole (1.67 g, 6.25 mmol), Cs₂CO₃ (6.11 g, 18.7 mmol) and bis(di-tert-butyl(4-dimethylaminophenyl)phosphine)dichloropalladium(II) (457 mg, 0.62 mmol) in 1,4-dioxane (30 mL) and water (6 mL) was degassed and purged with N2 three times. The reaction mixture was stirred at 100° C. under N2 for 1.5 h. The reaction mixture was filtered and the filtrate was concentrated. The residue was purified by silica column chromatography (40%-50% ethyl acetate in petroleum ether) to provide the title compound (1.0 g, 59% yield). ¹H NMR (400 MHz, CDCl₃) δ 6.85 (s, 1H), 6.64 (d, J=1.6 Hz, 1H), 5.04-4.94 (m, 1H), 3.31-3.28 (m, 2H), 2.90-2.84 (m, 2H), 1.85 (d, J=6.4 Hz, 6H). LCMS (ESI) [M+H]⁺=269.2.

### Step 4: 3-(1-Isopropyl-3-(5-(trifluoromethyl)pyridin-3-yl)-1H-pyrazol-5-yl)cyclopent-2-enone

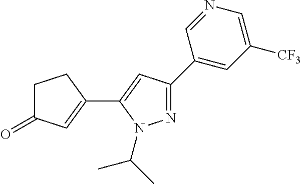 [SMILES: CC(C)n1nc(-c2cncc(C(F)(F)F)c2)cc1C1=CC(=O)CC1]

A suspension of 3-(3-bromo-1-isopropyl-1H-pyrazol-5-yl)cyclopent-2-enone (1.0 g, 3.7 mmol), 3-(4,4,5,5-tetramethyl-1,3,2-dioxaborolan-2-yl)-5-(trifluoromethyl)pyridine (1.01 g, 3.7 mmol), bis(di-tert-butyl(4-dimethylaminophenyl)phosphine)dichloropalladium(II) (270 mg, 0.4 mmol) and Cs₂CO₃ (3630 mg, 11.1 mmol) in 1,4-dioxane (20 mL) and water (4 mL) was degassed and purged with N2 three times. The reaction mixture was stirred at 100° C. under N2 for 2 h. The reaction mixture was filtered and the filtrate was concentrated. The residue was purified by silica column chromatography (30%-50% ethyl acetate in petroleum ether) to provide the title compound (1000 mg, 80% yield). LCMS (ESI) [M+H]⁺=336.1.

### Step 5: 3-(1-Isopropyl-3-(5-(trifluoromethyl)pyridin-3-yl)-1H-pyrazol-5-yl)cyclopentanone

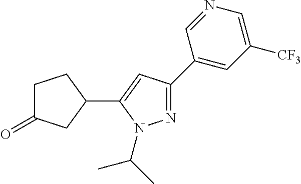 [SMILES: CC(C)n1nc(-c2cncc(C(F)(F)F)c2)cc1C1CCC(=O)C1]

To a solution of 3-(1-isopropyl-3-(5-(trifluoromethyl)pyridin-3-yl)-1H-pyrazol-5-yl)cyclopent-2-enone (1.0 g, 2.98 mmol) in methanol (20 mL) was added 10% palladium on carbon (0.32 g, 0.30 mmol) at 25° C. The reaction mixture was stirred at 25° C. under H₂ (15 psi) for 20 h. The reaction mixture was filtered and the solid was washed with methanol (10 mL×2). The combined organic layer was concentrated under vacuum. The residue was purified by silica column chromatography (30%˜50% ethyl acetate in petroleum ether) to provide the title compound (700 mg, 69.6% yield). ¹H NMR (400 MHz, CDCl₃) δ 9.47 (s, 1H), 9.09 (d, J=1.2 Hz, 1H), 8.64 (s, 1H), 6.72 (s, 1H), 4.88-4.75 (m, 1H), 3.82-3.75 (m, 1H), 3.10-3.00 (m, 1H), 2.88-2.78 (m, 2H), 2.73-2.61 (m, 2H), 2.47-2.39 (m, 1H), 1.93-1.86 (m, 6H). LCMS (ESI) [M+H]⁺=338.1. 800 mg of the above racemic mixture of 3-(1-isopropyl-3-(5-(trifluoromethyl)pyridin-3-yl)-1H-pyrazol-5-yl)cyclopentanone was separated by chiral SFC (Chiral Pak AD-3, 0.05% DEA in ethanol/CO₂ 5:95 to 40:60) to provide the second eluting peak as a pure single undefined enantiomer of 3-(1-isopropyl-3-(5-(trifluoromethyl)pyridin-3-yl)-1H-pyrazol-5-yl)cyclopentanone (370 mg, 46.2% yield).

### Step 6A: 6-((1R,3S)-3-(1-Isopropyl-3-(5-(trifluoromethyl)pyridin-3-yl)-1H-pyrazol-5-yl)cyclopentyl)-2-thia-6-azaspiro[3.4]octane 2,2-dioxide (Compound 13A*)

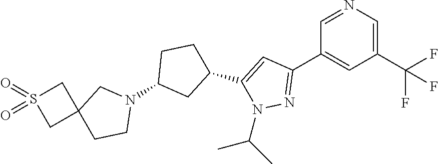 [SMILES: CC(C)n1nc(-c2cncc(C(F)(F)F)c2)cc1[C@H]1CC[C@@H](N2CCC3(C2)CS(=O)(=O)C3)C1]

To a solution of 3-(1-isopropyl-3-(5-(trifluoromethyl)pyridin-3-yl)-1H-pyrazol-5-yl)cyclopentanone (105 mg, 0.31 mmol, single undefined enantiomer from step 5), and 2-thia-6-azaspiro[3.4]octane 2,2-dioxide hydrochloride (95 mg, 0.48 mmol) in anhydrous methanol (5 mL) was added acetic acid (18.6 mg, 031 mmol) and NaBH₃CN (100 mg, 1.59 mmol). The reaction mixture was quenched with a saturated aq. NaHCO₃ solution (5 mL). The mixture was extracted with dichloromethane (20 mL×3). The combined organic layer was washed with brine (25 mL×3), dried over sodium sulfate, filtered and concentrated under reduced pressure. The cis and trans mixture of the title product was separated by reverse phase chromatography (acetonitrile/water gradient with 0.05% NH₃ and 10 mM NH₄HCO₃). Isolated as the first eluting peak as a pure single undefined enantiomer of the title compound (40 mg, 31% yield). LCMS (ESI) [M+H]⁺=483.2. ¹H NMR (400 MHz, CDCl₃) δ 9.16 (s, 1H), 8.76 (s, 1H), 8.32 (s, 1H), 6.36 (s, 1H), 4.55-4.44 (m, 1H), 4.10 (s, 4H), 3.37-3.32 (m, 1H), 2.90-2.83 (m, 3H), 2.78-2.71 (m, 2H), 2.30-2.22 (m, 1H), 2.21-2.17 (m, 2H), 2.14-2.04 (m, 2H), 1.93-1.85 (m, 1H), 1.79-1.65 (m, 2H), 1.54 (d, J=6.4 Hz, 6H). The relative cis stereochemistry was assigned based on ¹H NMR analysis.

### Step 6B: 6-((1S,3R)-3-(1-Isopropyl-3-(5-(trifluoromethyl)pyridin-3-yl)-1H-pyrazol-5-yl)cyclopentyl)-2-thia-6-azaspiro[3.4]octane 2,2-dioxide (Compound 13B*)

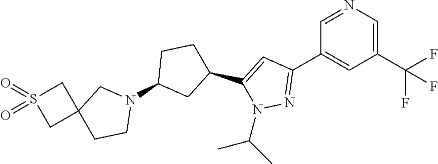 [SMILES: CC(C)n1nc(-c2cncc(C(F)(F)F)c2)cc1[C@@H]1CC[C@H](N2CCC3(C2)CS(=O)(=O)C3)C1]

Isolated as the second eluting peak of Step 6A as a pure single undefined enantiomer of the title compound (13 mg, 10% yield). LCMS (ESI) [M+H]⁺=483.2. ¹H NMR (400 MHz, CDCl₃) δ 9.16 (s, 1H), 8.77 (s, 1H), 8.32 (s, 1H), 6.42 (s, 1H), 4.57-4.42 (m, 1H), 4.10 (s, 4H), 3.25-3.17 (m, 1H), 2.92-2.86 (m, 2H), 2.79-2.74 (m, 3H), 2.34-2.28 (m, 1H), 2.21-2.18 (m, 3H), 2.06-1.90 (m, 1H), 1.84-1.76 (m, 2H), 1.72-1.62 (m, 1H), 1.54 (d, J=6.4 Hz, 6H). The relative cis stereochemistry was assigned based on ¹H NMR analysis.

### Step 6C: 6-((1R,3R)-3-(1-Isopropyl-3-(5-(trifluoromethyl)pyridin-3-yl)-1H-pyrazol-5-yl)cyclopentyl)-2-thia-6-azaspiro[3.4]octane 2,2-dioxide (Compound 13C*)

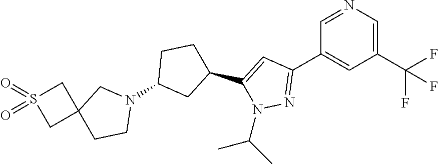 [SMILES: CC(C)n1nc(-c2cncc(C(F)(F)F)c2)cc1[C@@H]1CC[C@@H](N2CCC3(C2)CS(=O)(=O)C3)C1]

The title compound was synthesized following a procedure similar to compound 13A. The racemic mixture of 6-(3-(1-Isopropyl-3-(5-(trifluoromethyl)pyridin-3-yl)-1H-pyrazol-5-yl)cyclopentyl)-2-thia-6-azaspiro[3.4]octane 2,2-dioxide (160.0 mg, 0.33 mmol) was separated by chiral SFC (Daicel Chiralpak IG, 0.1% NH₃ in water/EtOH 60:40). Isolated as the first eluting peak as a pure single undefined enantiomer of the titled compound (31.6 mg, 18.6% yield). LCMS (ESI) [M+H]⁺=483.2. ¹H NMR (400 MHz, CDCl₃) δ 9.16 (s, 1H), 8.76 (s, 1H), 8.32 (s, 1H), 6.36 (s, 1H), 4.53-4.47 (m, 1H), 4.10 (s, 4H), 3.35-3.31 (m, 1H), 2.87-2.83 (m, 3H), 2.75-2.72 (m, 2H), 2.25-2.18 (m, 3H), 2.13-2.02 (m, 2H), 1.93-1.86 (m, 1H), 1.79-1.69 (m, 2H), 1.54 (t, J=7.2 Hz, 6H). The relative trans stereochemistry was assigned based on ¹H NMR analysis.

### Step 6D: 6-((1S,3S)-3-(1-Isopropyl-3-(5-(trifluoromethyl)pyridin-3-yl)-1H-pyrazol-5-yl)cyclopentyl)-2-thia-6-azaspiro[3.4]octane 2,2-dioxide (Compound 13D*)

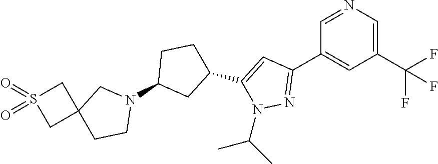 [SMILES: CC(C)n1nc(-c2cncc(C(F)(F)F)c2)cc1[C@H]1CC[C@H](N2CCC3(C2)CS(=O)(=O)C3)C1]

The title compound was synthesized following a procedure similar to compound 13A. Isolated as the second eluting peak as a pure single undefined enantiomer of the titled compound (55.9 mg, 34.2% yield). LCMS (ESI) [M+H]⁺=483.2. ¹H NMR (400 MHz, CDCl₃) δ 9.16 (d, J=1.6 Hz, 1H), 8.76 (d, J=1.2 Hz, 1H), 8.32 (s, 1H), 6.42 (s, 1H), 4.53-4.43 (m, 1H), 4.10 (s, 4H), 3.21-3.12 (m, 1H), 2.90-2.84 (m, 2H), 2.77-2.73 (m, 3H), 2.33-2.27 (m, 1H), 2.21-2.14 (m, 3H), 2.02-1.96 (m, 1H), 1.84-1.69 (m, 3H), 1.54 (d, J=6.4 Hz, 6H). The relative trans stereochemistry was assigned based on ¹H NMR analysis.

### Example P: 6-((1r,4r)-4-(6-(Trifluoromethyl)pyridin-3-yl)cyclohexyl)-2-thia-6-azaspiro[3.4]octane 2,2-dioxide (Compound 14A) and 6-((1s,4s)-4-(6-(Trifluoromethyl)pyridin-3-yl)cyclohexyl)-2-thia-6-azaspiro[3.4]octane 2,2-dioxide (Compound 14B)

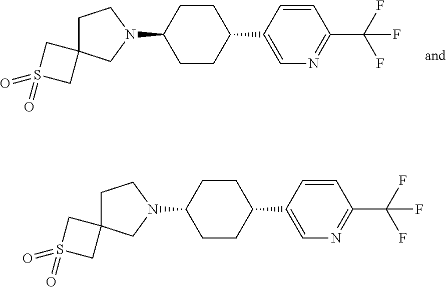 [SMILES: O=S1(=O)CC2(CCN([C@H]3CC[C@H](c4ccc(C(F)(F)F)nc4)CC3)C2)C1 ; O=S1(=O)CC2(CCN([C@H]3CC[C@@H](c4ccc(C(F)(F)F)nc4)CC3)C2)C1]

The title compounds were synthesized following a procedure similar to the procedure used in the synthesis of Compound 3A and 3B, using 5-bromo-2-(trifluoromethyl)pyridine in step 1. The residue was purified by reverse phase chromatography (acetonitrile/water gradient with 0.05% NH₃ and 10 mM NH₄HCO₃).

6-((1r,4r)-4-(6-(Trifluoromethyl)pyridin-3-yl)cyclohexyl)-2-thia-6-azaspiro[3.4]octane 2,2-dioxide (Compound 14A).

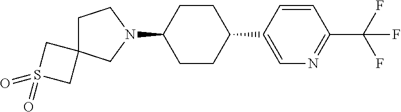 [SMILES: O=S1(=O)CC2(CCN([C@H]3CC[C@H](c4ccc(C(F)(F)F)nc4)CC3)C2)C1]

Provided as the first eluting peak as a pure single stereoisomer of the title compound (77.8 mg, 32.2% yield). LCMS (ESI) [M+H]⁺=389.2. ¹H NMR (400 MHz, CD₃OD) δ 8.60 (d, J=1.8 Hz, 1H), 7.92 (dd, J=8.4, 2.0 Hz, 1H), 7.74 (d, J=8.0 Hz, 1H), 4.23-4.03 (m, 4H), 2.99 (s, 2H), 2.84 (t, J=7.2 Hz, 2H), 2.74-2.72 (m, 1H), 2.30-2.29 (m, 1H), 2.24-2.13 (m, 4H), 1.98 (d, J=12.4 Hz, 2H), 1.66-1.62 (m, 2H), 1.51-1.38 (m, 2H). The relative trans stereochemistry was assigned based on ¹H NMR analysis.

6-((1s,4s)-4-(6-(Trifluoromethyl)pyridin-3-yl)cyclohexyl)-2-thia-6-azaspiro[3.4]octane 2,2-dioxide (Compound 14B).

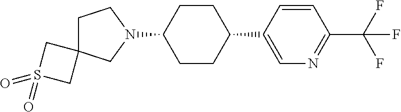 [SMILES: O=S1(=O)CC2(CCN([C@H]3CC[C@@H](c4ccc(C(F)(F)F)nc4)CC3)C2)C1]

Provide as the second eluting peak as a pure single stereoisomer of the title compound (33.8 mg, 14% yield). LCMS (ESI) [M+H]⁺=389.2. ¹H NMR (400 MHz, CD₃OD) δ 8.56 (d, J=1.6 Hz, 1H), 7.89 (dd, J=8.0, 1.6 Hz, 1H), 7.71 (d, J=8.4 Hz, 1H), 4.09 (s, 4H), 2.86 (s, 2H), 2.83-2.70 (m, 3H), 2.42 (d, J=3.2 Hz, 1H), 2.16 (t, J=7.2 Hz, 2H), 1.99-1.89 (m, 4H), 1.70-1.57 (m, 4H). The relative cis stereochemistry was assigned based on ¹H NMR analysis.

### Example O: 6-((1r,4r)-4-(1-Ethyl-3-(trifluoromethyl)-1H-pyrazol-5-yl)cyclohexyl)-2-thia-6-azaspiro[3.4]octane 2,2-dioxide (Compound 15) and 6-((1r,4r)-4-(1-Ethyl-5-(trifluoromethyl)-1H-pyrazol-3-yl)cyclohexyl)-2-thia-6-azaspiro[3.4]octane 2,2-dioxide (Compound 16)

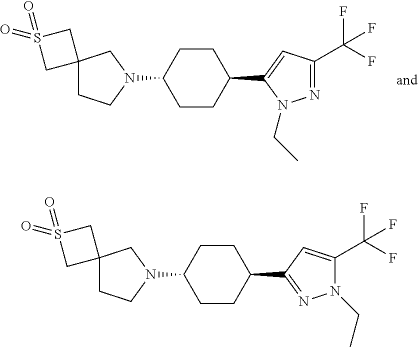 [SMILES: CCn1nc(C(F)(F)F)cc1[C@H]1CC[C@H](N2CCC3(C2)CS(=O)(=O)C3)CC1 ; CCn1nc([C@H]2CC[C@H](N3CCC4(C3)CS(=O)(=O)C4)CC2)cc1C(F)(F)F]

### Step 1: 4-(1-Ethyl-3-(trifluoromethyl)-1H-pyrazol-5-yl)cyclohexan-1-one and 4-(1-ethyl-5-(trifluoromethyl)-1H-pyrazol-3-yl)cyclohexan-1-one

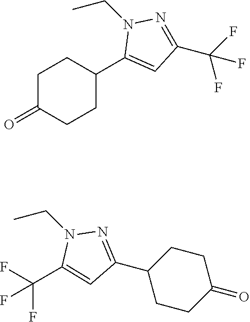 [SMILES: CCn1nc(C(F)(F)F)cc1C1CCC(=O)CC1 ; CCn1nc(C2CCC(=O)CC2)cc1C(F)(F)F]

A solution of 4,4,4-trifluoro-1-(1,4-dioxaspiro[4.5]decan-8-yl)butane-1,3-dione (500 mg, 1.78 mmol), ethyl hydrazine hydrochloride (207 mg, 2.14 mmol), triethylamine (0.298 mL, 2.14 mmol) and trifluoroacetic acid (0.324 mL, 4.28 mmol) in isopropanol (8.9 mL) was stirred at 60° C. for 18 h. AcOH (1.02 mL, 17.8 mmol) and water (4.5 mL) was added and the reaction mixture was stirred at 50° C. for 5 h. The reaction mixture was diluted with dichloromethane (20 mL) and 1N aq. NaHCO₃ (20 mL). The aqueous layer was extracted with dichloromethane (2×20 mL). The combined organic layer was dried (Na₂SO₄) and concentrated under reduced pressure to give the crude mixture of the title compounds (420 mg, 90% yield). LCMS (ESI) [M+H]⁺=261.2.

### Step 2: 6-((1r,4r)-4-(1-Ethyl-3-(trifluoromethyl)-1H-pyrazol-5-yl)cyclohexyl)-2-thia-6-azaspiro[3.4]octane 2,2-dioxide (Compound 15)

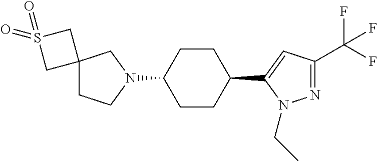 [SMILES: CCn1nc(C(F)(F)F)cc1[C@H]1CC[C@H](N2CCC3(C2)CS(=O)(=O)C3)CC1]

A solution of crude 4-(1-ethyl-3-(trifluoromethyl)-1H-pyrazol-5-yl)cyclohexan-1-one and 4-(1-ethyl-5-(trifluoromethyl)-1H-pyrazol-3-yl)cyclohexan-1-one (416 mg, 2.40 mmol), 2-thia-6-azaspiro[3.4]octane 2,2-dioxide hydrochloride (474 mg, 2.40 mmol) and 4 A molecular sieves (60 mg) in 1,2-dichloroethane (8.0 mL) was stirred at 40° C. for 18 h. Acetic acid (0.183 mL, 3.20 mmol) and sodium triacetoxyborohydride (678 mg, 3.20 mmol) were added and the reaction mixture was stirred at 25° C. for 18 h. The reaction mixture was diluted with 1N aq. NH₄Cl (1 mL), 1N aq. NaHCO₃ (10 mL) and 10% methanol in dichloromethane (20 mL). The aqueous layer was extracted with 10% methanol in dichloromethane (2×10 mL). The combined organic layer was concentrated under reduced pressure. The residue was diluted with DMSO, filtered and purified by prep-HPLC (acetonitrile/water gradient with 0.1% TFA) to provide the first eluting peak as a pure single stereoisomer of the title compound (33 mg, 5% yield). LCMS (ESI) [M+H]⁺=406.2. ¹H NMR (400 MHz, DMSO-d₆) δ 6.48 (s, 1H), 4.20-4.03 (m, 6H), 2.80 (s, 2H), 2.77-2.62 (m, 3H), 2.23-2.11 (m, 1H), 2.07 (t, J=7.2 Hz, 2H), 2.00-1.92 (m, 2H), 1.92-1.83 (m, 2H), 1.49-1.21 (m, 8H). The relative trans stereochemistry was assigned based on ¹H NMR analysis.

### 6-((1r,4r)-4-(1-Ethyl-5-(trifluoromethyl)-1H-pyrazol-3-yl)cyclohexyl)-2-thia-6-azaspiro[3.4]octane 2,2-dioxide (Compound 16)

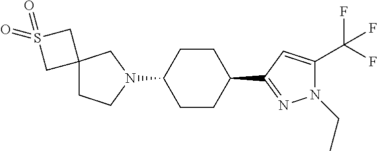 [SMILES: CCn1nc([C@H]2CC[C@H](N3CCC4(C3)CS(=O)(=O)C4)CC2)cc1C(F)(F)F]

The title compound was synthesized following a procedure similar to Compound 15. The residue was purified by prep-HPLC (acetonitrile/water gradient with 0.1% TFA) to provide the second eluting peak as a pure single stereoisomer of the title compound (4.9 mg, 1% yield). LCMS (ESI) [M+H]⁺=406.2. ¹H NMR (400 MHz, DMSO) δ 6.67 (s, 1H), 4.21-4.03 (m, 6H), 2.79 (s, 2H), 2.65 (t, J=7.2 Hz, 2H), 2.59-2.52 (m, 1H), 2.17-2.02 (m, 3H), 1.99-1.90 (m, 4H), 1.42-1.18 (m, 7H). The relative trans stereochemistry was assigned based on ¹H NMR analysis.

### Example R: (Compounds 17A*, 17B*, 17C*, and 17D*): 6-((1S,3R)-3-(1-Isopropyl-3-(6-(trifluoromethyl)pyridin-3-yl)-1H-1,2,4-triazol-5-yl)cyclopentyl)-2-thia-6-azaspiro[3.4]octane 2,2-dioxide; 6-((1S,3S)-3-(1-isopropyl-3-(6-(trifluoromethyl)pyridin-3-yl)-1H-1,2,4-triazol-5-yl)cyclopentyl)-2-thia-6-azaspiro[3.4]octane 2,2-dioxide; 6-((1R,3R)-3-(1-isopropyl-3-(6-(trifluoromethyl)pyridin-3-yl)-1H-1,2,4-triazol-5-yl)cyclopentyl)-2-thia-6-azaspiro[3.4]octane 2,2-dioxide; 6-((1R,3S)-3-(1-isopropyl-3-(6-(trifluoromethyl)pyridin-3-yl)-1H-1,2,4-triazol-5-yl)cyclopentyl)-2-thia-6-azaspiro[3.4]octane 2,2-dioxide

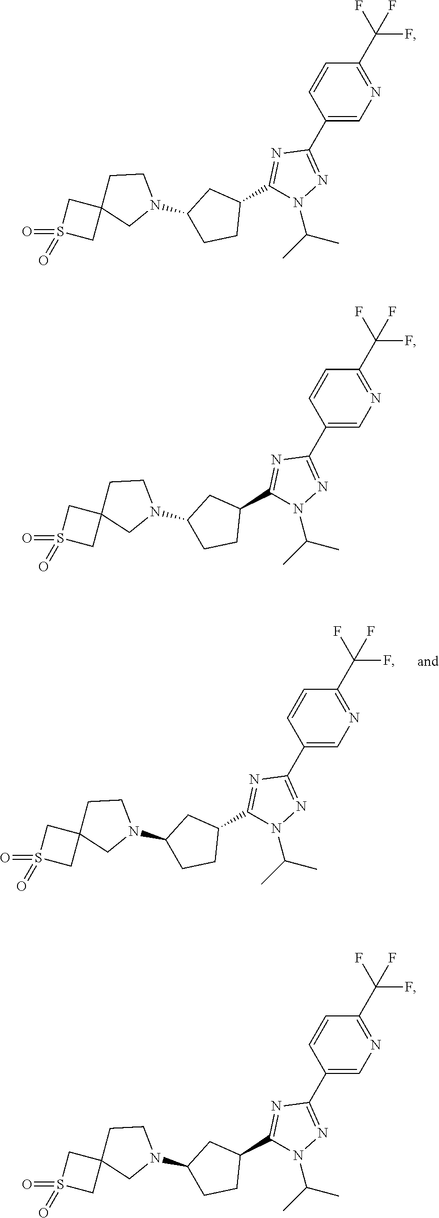 [SMILES: CC(C)n1nc(-c2ccc(C(F)(F)F)nc2)nc1[C@@H]1CC[C@H](N2CCC3(C2)CS(=O)(=O)C3)C1 ; CC(C)n1nc(-c2ccc(C(F)(F)F)nc2)nc1[C@H]1CC[C@H](N2CCC3(C2)CS(=O)(=O)C3)C1 ; CC(C)n1nc(-c2ccc(C(F)(F)F)nc2)nc1[C@@H]1CC[C@@H](N2CCC3(C2)CS(=O)(=O)C3)C1 ; CC(C)n1nc(-c2ccc(C(F)(F)F)nc2)nc1[C@H]1CC[C@@H](N2CCC3(C2)CS(=O)(=O)C3)C1]

6-(3-(1-Isopropyl-3-(6-(trifluoromethyl)pyridin-3-yl)-1H-1,2,4-triazol-5-yl)cyclopentyl)-2-thia-6-azaspiro[3.4]octane 2,2-dioxide was synthesized following a procedure similar to that followed for Compounds 36A-36D but instead using 3-oxocyclopentanecarboxylic acid and isoproylhydrazine HCl in the triazole formation reaction (J. Org. Chem. 2011, 76, 1177). The diastereomeric mixture was separated by chiral SFC (Phenomenex Amylose-1, 0.1% ammonium hydroxide in methanol/CO₂ 15:85) to provide two peaks constituting cis/trans mixtures of 6-(3-(1-Isopropyl-3-(6-(trifluoromethyl)pyridin-3-yl)-1H-1,2,4-triazol-5-yl)cyclopentyl)-2-thia-6-azaspiro[3.4]octane 2,2-dioxide. Peak 1 was then further separated by chiral SFC (Chiralcel OX, 0.1% ammonium hydroxide in methanol/CO₂ 25:75) to provide the first eluting peak as a pure single undefined enantiomer of the title compound (Compound 17A*; 3.8 mg, 1.4% yield). LCMS (ESI) [M+H]⁺=484.2.

The diastereomeric mixture was separated by chiral SFC (Phenomenex Amylose-1, 0.1% ammonium hydroxide in methanol/CO₂ 15:85) to provide two peaks each constituting cis/trans mixtures of 6-(3-(1-Isopropyl-3-(6-(trifluoromethyl)pyridin-3-yl)-1H-1,2,4-triazol-5-yl)cyclopentyl)-2-thia-6-azaspiro[3.4]octane 2,2-dioxide. Peak 2 was separated by chiral SFC (Chiralcel OX, 0.1% ammonium hydroxide in methanol/CO₂ 25:75) to provide the first eluting peak as a pure single undefined enantiomer of the title compound (Compound 17B*; 3.5 mg, 1.3% yield). LCMS (ESI) [M+H]⁺=484.2.

The diastereomeric mixture was separated by chiral SFC (Phenomenex Amylose-1, 0.1% ammonium hydroxide in methanol/CO₂ 15:85) to provide two peaks constituting cis/trans mixtures of 6-(3-(1-Isopropyl-3-(6-(trifluoromethyl)pyridin-3-yl)-1H-1,2,4-triazol-5-yl)cyclopentyl)-2-thia-6-azaspiro[3.4]octane 2,2-dioxide. Peak 1 was then further separated by chiral SFC (Chiralcel OX, 0.1% ammonium hydroxide in methanol/CO₂ 25:75) to provide the second eluting peak as a pure single undefined enantiomer of the title compound (Compound 17C*) (13 mg, 5% yield). LCMS (ESI) [M+H]⁺=484.2. ¹H NMR (400 MHz, DMSO-d₆) δ 9.34-9.20 (m, 1H), 8.59-8.47 (m, 1H), 8.01-7.94 (m, 1H), 4.76 (hept, J=6.6 Hz, 1H), 4.18-4.09 (m, 4H), 3.53-3.42 (m, 1H), 2.81-2.58 (m, 5H), 2.29-2.19 (m, 1H), 2.16-2.02 (m, 3H), 2.01-1.64 (m, 4H), 1.45 (dd, J=6.5, 2.9 Hz, 6H).

The diastereomeric mixture was separated by chiral SFC (Phenomenex Amylose-1, 0.1% ammonium hydroxide in methanol/CO₂ 15:85) to provide two peaks constituting cis/trans mixtures of 6-(3-(1-Isopropyl-3-(6-(trifluoromethyl)pyridin-3-yl)-1H-1,2,4-triazol-5-yl)cyclopentyl)-2-thia-6-azaspiro[3.4]octane 2,2-dioxide. Peak 2 was then further separated by chiral SFC (Chiralcel OX, 0.1% ammonium hydroxide in methanol/CO₂ 25:75) to provide the second eluting peak as a pure single undefined enantiomer of the title compound (Compound 17D*) (13 mg, 5% yield). LCMS (ESI) [M+H]+=484.2. ¹H NMR δ 9.30-9.24 (m, 1H), 8.58-8.51 (m, 1H), 8.00-7.95 (m, 1H), 4.76 (hept, J=6.5 Hz, 1H), 4.19-4.07 (m, 4H), 3.54-3.42 (m, 1H), 2.83-2.58 (m, 5H), 2.30-2.20 (m, 1H), 2.15-2.02 (m, 3H), 2.01-1.65 (m, 4H), 1.45 (dd, J=6.5, 2.9 Hz, 6H).

### Example S: (Compounds 18A*, 18B*, 18C*, and 18D*): 6-((1S,3R)-3-(3-Cyclopropyl-1-isopropyl-1H-1,2,4-triazol-5-yl)cyclopentyl)-2-thia-6-azaspiro[3.4]octane 2,2-dioxide; 6-((1S,3S)-3-(1-isopropyl-3-(6-(trifluoromethyl)pyridin-3-yl)-1H-1,2,4-triazol-5-yl)cyclopentyl)-2-thia-6-azaspiro[3.4]octane 2,2-dioxide; 6-((1R,3R)-3-(3-cyclopropyl-1-isopropyl-1H-1,2,4-triazol-5-yl)cyclopentyl)-2-thia-6-azaspiro[3.4]octane 2,2-dioxide; 6-((1R,3S)-3-(1-isopropyl-3-(6-(trifluoromethyl)pyridin-3-yl)-1H-1,2,4-triazol-5-yl)cyclopentyl)-2-thia-6-azaspiro[3.4]octane 2,2-dioxide

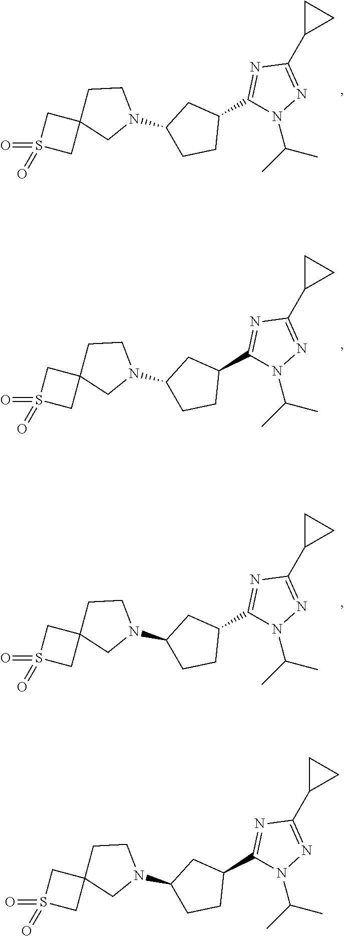 [SMILES: CC(C)n1nc(C2CC2)nc1[C@@H]1CC[C@H](N2CCC3(C2)CS(=O)(=O)C3)C1 ; CC(C)n1nc(C2CC2)nc1[C@H]1CC[C@H](N2CCC3(C2)CS(=O)(=O)C3)C1 ; CC(C)n1nc(C2CC2)nc1[C@@H]1CC[C@@H](N2CCC3(C2)CS(=O)(=O)C3)C1 ; CC(C)n1nc(C2CC2)nc1[C@H]1CC[C@@H](N2CCC3(C2)CS(=O)(=O)C3)C1]

6-(3-(3-cyclopropyl-1-isopropyl-1H-1,2,4-triazol-5-yl)cyclopentyl)-2-thia-6-azaspiro[3.4]octane 2,2-dioxide was synthesized following a procedure similar to Compound 36A but instead using 3-oxocyclopentanecarboxylic acid, cyclopropylcarbamidine HCl and isoproylhydrazine HCl in the triazole formation reaction (J. Org. Chem. 2011, 76, 1177).

The diastereomeric mixture was separated by chiral SFC (Chiralpak IC, 0.1% ammonium hydroxide in methanol/CO₂ 30:70) to provide the first eluting peak as a racemic mixture of 6-(3-(3-cyclopropyl-1-isopropyl-1H-1,2,4-triazol-5-yl)cyclopentyl)-2-thia-6-azaspiro[3.4]octane 2,2-dioxide. Peak 1 was then separated by chiral SFC (Chiralpak ID, 0.1% ammonium hydroxide in methanol/CO₂ 25:75) to provide the first eluting peak as a pure single undefined enantiomer of title compound (Compound 18A*) (12 mg, 3% yield). LCMS (ESI) [M+H]⁺=379.2. ¹H NMR (400 MHz, DMSO-d₆) δ 4.51 (hept, J=6.6 Hz, 1H), 4.19-4.08 (m, 4H), 3.47-3.34 (m, 1H), 2.82-2.69 (m, 3H), 2.64-2.56 (m, 2H), 2.12-1.98 (m, 3H), 1.94-1.83 (m, 4H), 1.75-1.45 (m, 2H), 1.32 (dd, J=6.5, 3.8 Hz, 6H), 0.85-0.67 (m, 4H).

The diastereomeric mixture was separated by chiral SFC (Chiralpak IC, 0.1% ammonium hydroxide in methanol/CO₂ 30:70) to provide the first eluting peak as a racemic mixture of 6-(3-(3-cyclopropyl-1-isopropyl-1H-1,2,4-triazol-5-yl)cyclopentyl)-2-thia-6-azaspiro[3.4]octane 2,2-dioxide. Peak 1 was then separated by chiral SFC (Chiralpak ID, 0.1% ammonium hydroxide in methanol/CO₂ 25:75) to provide the second eluting peak as a pure single undefined enantiomer of title compound (Compound 18B*) (12.2 mg, 3.1% yield). LCMS (ESI) [M+H]⁺=379.20. ¹H NMR (400 MHz, DMSO-d6) δ 4.51 (hept, J=6.6 Hz, 1H), 4.19-4.08 (m, 4H), 3.47-3.34 (m, 1H), 2.82-2.69 (m, 3H), 2.64-2.56 (m, 2H), 2.12-1.98 (m, 3H), 1.94-1.83 (m, 4H), 1.75-1.45 (m, 2H), 1.32 (dd, J=6.5, 3.8 Hz, 6H), 0.85-0.67 (m, 4H).

The diastereomeric mixture was separated by chiral SFC (Chiralpak IC, 0.1% ammonium hydroxide in methanol/CO₂ 30:70) to provide the second eluting peak as a pure single undefined enantiomer of 6-(3-(3-cyclopropyl-1-isopropyl-1H-1,2,4-triazol-5-yl)cyclopentyl)-2-thia-6-azaspiro[3.4]octane 2,2-dioxide (Compound 18C*) (6 mg, 1.7% yield). LCMS (ESI) [M+H]⁺=379.20. ¹H NMR (400 MHz, DMSO-d₆) δ 4.52 (hept, J=6.7 Hz, 1H), 4.18-4.07 (m, 4H), 3.42-3.20 (m, 2H), 2.77-2.54 (m, 5H), 2.14-1.57 (m, 8H), 1.32 (dd, J=6.5, 2.5 Hz, 6H), 0.85-0.69 (m, 4H).

The diastereomeric mixture was separated by chiral SFC (Chiralpak IC, 0.1% ammonium hydroxide in methanol/CO₂ 30:70) to provide the third eluting peak as a pure single undefined enantiomer of 6-(3-(3-cyclopropyl-1-isopropyl-1H-1,2,4-triazol-5-yl)cyclopentyl)-2-thia-6-azaspiro[3.4]octane 2,2-dioxide (Compound 18D*) (21 mg, 5.6% yield). LCMS (ESI) [M+H]⁺=379.20. ¹H NMR (400 MHz, DMSO-d6) δ 4.52 (hept, J=6.7 Hz, 1H), 4.18-4.07 (m, 4H), 3.42-3.20 (m, 2H), 2.77-2.54 (m, 5H), 2.14-1.57 (m, 8H), 1.32 (dd, J=6.5, 2.5 Hz, 6H), 0.85-0.69 (m, 4H).

### Example T: (Compounds 19A*, 19B*, 19C*, and 19D*): 6-((3S,5S)-5-(4-(trifluoromethyl)phenyl)tetrahydrofuran-3-yl)-2-thia-6-azaspiro[3.4]octane 2,2-dioxide, 6-((3R,5S)-5-(4-(trifluoromethyl)phenyl)tetrahydrofuran-3-yl)-2-thia-6-azaspiro[3.4]octane 2,2-dioxide, 6-((3R,5R)-5-(4-(trifluoromethyl)phenyl)tetrahydrofuran-3-yl)-2-thia-6-azaspiro[3.4]octane 2,2-dioxide, 6-((3S,5R)-5-(4-(trifluoromethyl)phenyl)tetrahydrofuran-3-yl)-2-thia-6-azaspiro[3.4]octane 2,2-dioxide

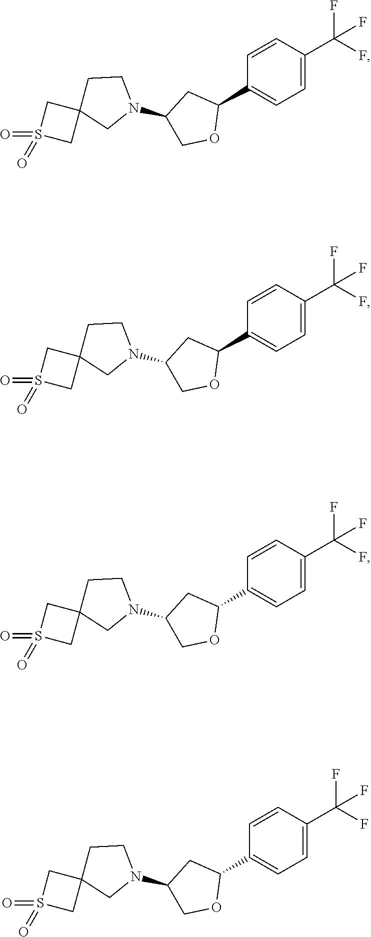 [SMILES: O=S1(=O)CC2(CCN([C@@H]3CO[C@H](c4ccc(C(F)(F)F)cc4)C3)C2)C1 ; O=S1(=O)CC2(CCN([C@H]3CO[C@H](c4ccc(C(F)(F)F)cc4)C3)C2)C1 ; O=S1(=O)CC2(CCN([C@H]3CO[C@@H](c4ccc(C(F)(F)F)cc4)C3)C2)C1 ; O=S1(=O)CC2(CCN([C@@H]3CO[C@@H](c4ccc(C(F)(F)F)cc4)C3)C2)C1]

### Step 1: 1-(4-(Trifluoromethyl)phenyl)but-3-en-1-ol

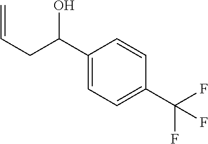

To a stirred solution of 4-(trifluoromethyl)benzaldehyde (2 g, 11.49 mmol) in tetrahydrofuran (20 mL) was added allylmagnesium bromide (13.8 mL, 13.8 mmol, 1M) dropwise at 0° C. The reaction mixture was stirred at 0° C. for 2 h. The reaction mixture was quenched with a saturated NH₄Cl aq. solution (20 mL) and extracted with ethyl acetate (100 mL×2). The combined organic layer was dried over sodium sulfate, filtered and concentrated under reduced pressure. The resulting residue was purified by silica column chromatography (0˜50% ethyl acetate in petroleum ether) to provide the title compound (1500 mg, 60% yield). ¹H NMR (400 MHz, CDCl₃) δ 7.63 (d, J=8.0 Hz, 2H), 7.50 (d, J=8.4 Hz, 2H), 5.90-5.75 (m, 1H), 5.26-5.15 (m, 2H), 4.89-4.73 (m, 1H), 2.62-2.43 (m, 2H), 2.26-2.13 (m, 1H).

### Step 2: 3, 4-Dibromo-1-(4-(trifluoromethyl)phenyl)butan-1-ol

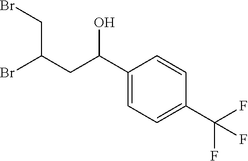 [SMILES: OC(CC(Br)CBr)c1ccc(C(F)(F)F)cc1]

To a solution of 1-(4-(trifluoromethyl)phenyl)but-3-en-1-ol (2 g, 9.25 mmol) in dichloromethane (10 mL) was added Br₂ (0.5 mL, 9.76 mmol) in dichloromethane (10 mL) at −30° C. The reaction mixture was stirred at −30° C. for 2 h. The reaction mixture was concentrated under reduced pressure and the resulting residue was used directly for the next step.

### Step 3: 4-Bromo-2-(4-(trifluoromethyl)phenyl)tetrahydrofuran

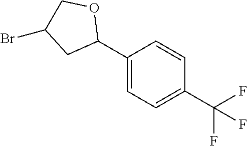 [SMILES: FC(F)(F)c1ccc(C2CC(Br)CO2)cc1]

To a solution of 3, 4-dibromo-1-(4-(trifluoromethyl)phenyl)butan-1-ol (3.48 g, 9.25 mmol) in anhydrous methanol (20 mL) was added K₂CO₃ (5.11 g, 37 mmol) at 20° C. The reaction mixture was stirred at 20° C. for 16 h, quenched with a saturated NH₄Cl aq. solution (20 mL) and extracted with ethyl acetate (100 mL×2). The combined organic layer was dried over sodium sulfate, filtered and concentrated under reduced pressure. The resulting residue was purified by silica column chromatography (0˜10% ethyl acetate in petroleum ether) to provide the title compound (1600 mg, 58% yield). ¹H NMR (400 MHz, CDCl₃) δ 7.62 (d, J=8.0 Hz, 2H), 7.47 (d, J=8.0 Hz, 2H), 5.42-5.31 (m, 1H), 4.63-4.54 (m, 2H), 4.33-4.28 (m, 1H), 2.76-2.68 (m, 1H), 2.33-2.23 (m, 1H).

### Step 4: 5-(4-(Trifluoromethyl)phenyl)tetrahydrofuran-3-yl acetate

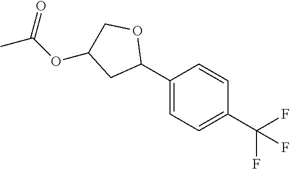 [SMILES: CC(=O)OC1COC(c2ccc(C(F)(F)F)cc2)C1]

To a solution of 4-bromo-2-(4-(trifluoromethyl)phenyl)tetrahydrofuran (1600 mg, 5.42 mmol) in dimethyl sulfoxide (60 mL) was added potassium acetate (1600 mg, 16.30 mmol). The reaction mixture was stirred at 20° C. for 16 h, quenched with a saturated NH₄Cl aq. solution (50 mL) and extracted with ethyl acetate (100 mL×2). The combined organic layer was dried over anhydrous Na₂SO₄, filtered and concentrated under reduced pressure. The residue was used directly for next step.

### Step 5: 5-(4-(Trifluoromethyl)phenyl)tetrahydrofuran-3-ol

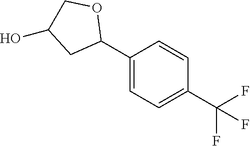 [SMILES: OC1COC(c2ccc(C(F)(F)F)cc2)C1]

To a solution of 5-(4-(Trifluoromethyl)phenyl)tetrahydrofuran-3-yl acetate (1486 mg, 5.42 mmol) in anhydrous methanol (60 mL) was added K₂CO₃ (2.26 g, 16.36 mmol). The reaction mixture was stirred at 20° C. for 16 h, quenched with a saturated NH₄Cl aq. solution (60 mL) and extracted with dichloromethane (50 mL×2). The combined organic layer was dried over anhydrous Na₂SO₄, filtered and concentrated under reduced pressure. The residue was used directly for next step. ¹H NMR (400 MHz, CDCl₃) δ 7.63-7.59 (m, 2H), 7.55-7.45 (m, 2H), 5.27-4.93 (m, 1H), 4.70-4.57 (m, 1H), 4.30-4.11 (m, 1H), 3.97-3.91 (m, 1H), 2.76-2.37 (m, 1H), 1.95-1.87 (m, 1H), 1.82-1.73 (m, 1H).

### Step 6: 5-(4-(Trifluoromethyl)phenyl)dihydrofuran-3(2H)-one

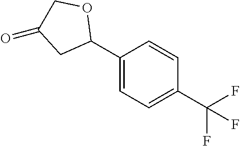 [SMILES: O=C1COC(c2ccc(C(F)(F)F)cc2)C1]

To a solution of 5-(4-(trifluoromethyl)phenyl)tetrahydrofuran-3-ol (800 mg, 3.45 mmol) in anhydrous dichloromethane (48 mL) was added Dess-Martin periodinane (3.02 g, 7.13 mmol). The reaction mixture was stirred at 25° C. for 5 h, quenched with a saturated Na₂SO₃ aq. solution (50 mL) and extracted with ethyl acetate (100 mL×2). The combined organic layer was dried over sodium sulfate, filtered and concentrated under reduced pressure. The residue was purified by silica column chromatography (0˜20% ethyl acetate in petroleum ether) to provide a racemic mixture of the title compound (400 mg, 50% yield). The racemic mixture (429 mg, 1.86 mmol) was separated by chiral SFC (Daicel Chiralpak AD-H, 0.1% NH₃ in water/EtOH 90:10) to provide the first eluting peak as a pure single undefined enantiomer of the title compound (300 mg, 58% yield).

### Step 7: 6-((3R,5R)-5-(4-(Trifluoromethyl)phenyl)tetrahydrofuran-3-yl)-2-thia-6-azaspiro[3.4]octane 2,2-dioxide, 6-((3S,5R)-5-(4-(trifluoromethyl)phenyl) tetrahydrofuran-3-yl)-2-thia-6-azaspiro[3.4]octane 2,2-dioxide

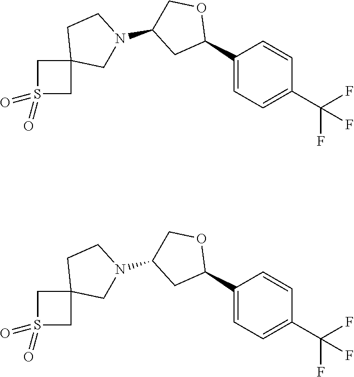 [SMILES: O=S1(=O)CC2(CCN([C@H]3CO[C@@H](c4ccc(C(F)(F)F)cc4)C3)C2)C1 ; O=S1(=O)CC2(CCN([C@@H]3CO[C@@H](c4ccc(C(F)(F)F)cc4)C3)C2)C1]

The mixture of the diastereomers (98.0 mg, 0.26 mmol) was separated by chiral SFC (Daicel Chiralpak AD-H (250 mm*30 mm, 5 μm); 0.1% NH₃H₂O/MeOH=65:65; 60 mL/min) to provide the title Compound 19A* (the first peak on SFC, 42 mg, 0.11 mmol, 42% yield) and the title Compound 19B* (the second peak on SFC, 31 mg, 0.08 mmol, 32% yield). LCMS (ESI): [M+H]⁺=376.2.

Compound 19A*: ¹H NMR (400 MHz, CDCl₃) δ 7.61 (d, J=8.4 Hz, 2H), 7.47 (d, J=8.4 Hz, 2H), 5.00 (dd, J=6.8, 9.6 Hz, 1H), 4.07-3.94 (m, 6H), 3.27-3.18 (m, 1H), 2.88-2.70 (m, 4H), 2.54-5.49 (m, 1H), 2.19-2.11 (m, 2H), 1.81-1.78 (m, 1H).

Compound 19B*: ¹H NMR (400 MHz, CDCl₃) δ 7.61 (d, J=8.0 Hz, 2H), 7.43 (d, J=8.0 Hz, 2H), 5.13 (dd, J=7.2, 10.4 Hz, 1H), 4.19 (dd, J=6.0, 8.8 Hz, 1H), 4.09 (s, 4H), 3.83 (dd, J=6.4, 8.4 Hz, 1H), 3.12-3.06 (m, 1H), 2.94-2.81 (m, 2H), 2.79-2.72 (m, 2H), 2.47-2.33 (m, 1H), 2.19 (t, J=7.2 Hz, 2H), 1.97-1.93 (m, 1H).

### Step 8: 6-((3S,5S)-5-(4-(Trifluoromethyl)phenyl)tetrahydrofuran-3-yl)-2-thia-6-azaspiro[3.4]octane 2,2-dioxide, 6-((3R,5S)-5-(4-(trifluoromethyl)phenyl)tetrahydrofuran-3-yl)-2-thia-6-azaspiro[3.4]octane 2,2-dioxide

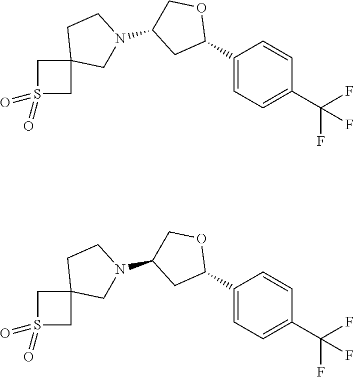 [SMILES: O=S1(=O)CC2(CCN([C@@H]3CO[C@H](c4ccc(C(F)(F)F)cc4)C3)C2)C1 ; O=S1(=O)CC2(CCN([C@H]3CO[C@H](c4ccc(C(F)(F)F)cc4)C3)C2)C1]

To a solution of (S)-5-(4-(trifluoromethyl)phenyl)dihydrofuran-3(2H)-one (Peak 2, 50 mg, 0.22 mmol) and 2-thia-6-azaspiro[3.4]octane 2,2-dioxide hydrochloride (66 mg, 0.34 mmol) in anhydrous methanol (4 mL) was added two drops of acetic acid, and the reaction mixture was stirred at 25° C. for 5 minutes. NaBH₃CN (70 mg, 1.12 mmol) was then added and stirred at 70° C. for 2 h. The reaction mixture was quenched by saturated NaHCO₃ solution (20 mL) and extracted with dichloromethane (50 mL×2) and ethyl acetate (50 mL×2). The combined organic layers were concentrated under reduced pressure and the resulting residue was purified by prep-TLC (10% methanol in dichloromethane) to provide the mixture of diastereoisomers (51 mg, 0.13 mmol, 60% yield). LCMS (ESI) [M+H]⁺=376.2.

The mixture of the diastereomers (51 mg, 0.13 mmol) was separated by chiral SFC (Daicel Chiralpak AD (250 mm*30 mm, 10 μm); 0.1% NH₃ in H₂O/MeOH=65:65, 70 mL/min) to provide the title Compound 19C* (the first peak on SFC, 17 mg, 0.04 mmol, 33% yield) and the title Compound 19D* (the second peak on SFC, 21 mg, 0.05 mmol, 41% yield). LCMS (ESI): [M+H]⁺=376.2.

Compound 19C*: ¹H NMR (400 MHz, CDCl₃) δ 7.60 (d, J=8.0 Hz, 2H), 7.47 (d, J=8.0 Hz, 2H), 5.13-4.85 (m, 1H), 4.11-3.93 (m, 6H), 3.28-3.17 (m, 1H), 2.93-2.61 (m, 4H), 2.56-2.48 (m, 1H), 2.19-2.12 (m, 2H), 1.91-1.73 (m, 1H).

Compound 19D*: ¹H NMR (400 MHz, CDCl₃) δ 7.61 (d, J=8.0 Hz, 2H), 7.43 (d, J=8.0 Hz, 2H), 5.13 (dd, J=7.2, 10.4 Hz, 1H), 4.19 (dd, J=6.0, 8.8 Hz, 1H), 4.09 (s, 4H), 3.83 (dd, J=6.4, 8.4 Hz, 1H), 3.12-3.06 (m, 1H), 2.94-2.81 (m, 2H), 2.79-2.72 (m, 2H), 2.47-2.33 (m, 1H), 2.19 (t, J=7.2 Hz, 2H), 1.97-1.93 (m, 1H).

### Example U: (Compounds 20A*, 20B*, 20C*, and 20D*): 6-((1S,3S)-3-(1-Isopropyl-4-(trifluoromethyl)-1H-imidazol-2-yl)cyclopentyl)-2-thia-6-azaspiro[3.4]octane 2,2-dioxide; 6-((1R,3S)-3-(1-isopropyl-4-(trifluoromethyl)-1H-imidazol-2-yl)cyclopentyl)-2-thia-6-azaspiro[3.4]octane 2,2-dioxide; 6-((1S,3R)-3-(1-isopropyl-4-(trifluoromethyl)-1H-imidazol-2-yl)cyclopentyl)-2-thia-6-azaspiro[3.4]octane 2,2-dioxide; 6-((1R,3R)-3-(1-isopropyl-4-(trifluoromethyl)-1H-imidazol-2-yl)cyclopentyl)-2-thia-6-azaspiro[3.4]octane 2,2-dioxide

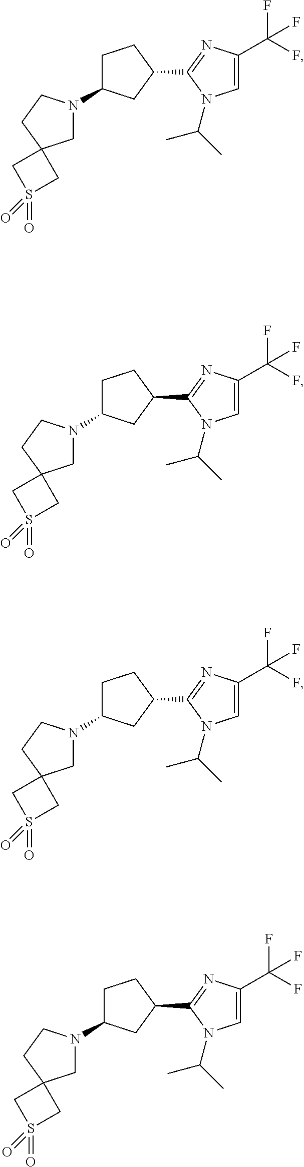 [SMILES: CC(C)n1cc(C(F)(F)F)nc1[C@H]1CC[C@H](N2CCC3(C2)CS(=O)(=O)C3)C1 ; CC(C)n1cc(C(F)(F)F)nc1[C@@H]1CC[C@@H](N2CCC3(C2)CS(=O)(=O)C3)C1 ; CC(C)n1cc(C(F)(F)F)nc1[C@H]1CC[C@@H](N2CCC3(C2)CS(=O)(=O)C3)C1 ; CC(C)n1cc(C(F)(F)F)nc1[C@@H]1CC[C@H](N2CCC3(C2)CS(=O)(=O)C3)C1]

### Step 1: 1-Isopropyl-4-(trifluoromethyl)imidazole

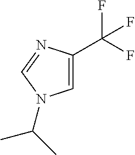

To a stirred solution of 2-iodopropane (3.75 g, 22.05 mmol) and 4-(trifluoromethyl)-1H-imidazole (1.0 g, 7.35 mmol) in N,N-dimethylformamide (10 mL) was added Cs₂CO₃ (11.97 g, 36.74 mmol)) at 25° C. The reaction mixture was stirred at 25° C. for 16 h. The mixture was quenched with water (50 mL) and extracted with ethyl acetate (60 mL×3). The combined organic layer was washed with brine (50 mL×3), dried over sodium sulfate, filtered and concentrated. The crude residue was purified by silica column chromatography (0-50% ethyl acetate in petroleum ether) to provide the title compound (1 g, 76.4% yield). ¹H NMR (400 MHz, CD₃OD) δ 7.86 (s, 1H), 7.74 (s, 1H), 4.60-4.47 (m, 1H), 1.51 (d, J=6.4 Hz, 6H).

### Step 2: 2-Bromo-1-isopropyl-4-(trifluoromethyl)imidazole

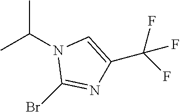

To a stirred solution of 1-isopropyl-4-(trifluoromethyl)imidazole (5000.0 mg, 28.07 mmol) in anhydrous tetrahydrofuran (50 mL) was added n-butyllithium (13.47 mL, 33.68 mmol) at −75° C. under a nitrogen atmosphere. The reaction was stirred at −76° C. for 30 minutes. Then NBS (5495 mg, 30.87 mmol) in anhydrous tetrahydrofuran (10 mL) was added and the reaction was stirred for 3 h. The reaction was quenched with aq. saturated NH₄Cl (30 mL) and extracted with ethyl acetate (200 mL×2). The combined organic layer was washed with brine (50 mL×2), dried over MgSO₄, filtered and concentrated under reduced pressure. The residue was purified by silica column chromatography (100% petroleum ether) to provide the title compound (4500 mg, 62.4% yield).

### Step 3: 3-(1-Isopropyl-4-(trifluoromethyl)-1H-imidazol-2-yl)cyclopent-2-en-1-one

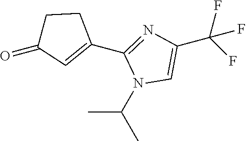 [SMILES: CC(C)n1cc(C(F)(F)F)nc1C1=CC(=O)CC1]

To a stirred solution of 2-bromo-1-isopropyl-4-(trifluoromethyl)imidazole (2.0 g, 7.8 mmol), 3-(4,4,5,5-tetramethyl-1,3,2-dioxaborolan-2-yl)cyclopent-2-en-1-one (2.43 g, 11.7 mmol) and 1,1′-bis(diphenylphosphino)ferrocene palladium dichloride (569 mg, 0.8 mmol) in 1,4-dioxane (20 mL) and water (5 mL) was added Cs₂CO₃ (6.34 g, 19.5 mmol). The mixture was stirred at 80° C. under N2 for 6 h. The reaction mixture was diluted with water (50 mL) and extracted with ethyl acetate (50 mL×3). The combined organic layer was washed with brine (50 mL×2), dried over Na₂SO₄, filtered and concentrated under reduced pressure. The residue was purified by silica column chromatography (100% petroleum ether) to provide the title compound (1.0 g, 39.8% yield). LCMS (ESI) [M+H]⁺=259.0.

### Step 4: 3-(1-Isopropyl-4-(trifluoromethyl)-1H-imidazol-2-yl)cyclopentan-1-one

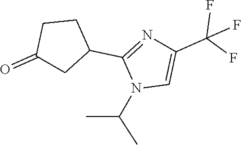 [SMILES: CC(C)n1cc(C(F)(F)F)nc1C1CCC(=O)C1]

To a solution of 3-(1-isopropyl-4-(trifluoromethyl)-1H-imidazol-2-yl)cyclopent-2-en-1-one (900.0 mg, 3.49 mmol) in ethanol (10 mL) was added 10% palladium on carbon (742 mg, 0.70 mmol) under N2. The suspension was degassed and purged with H₂ (15 psi) three times. The mixture was stirred under H₂ (15 psi) at 25° C. for 16 h. The resulting mixture was filtered and the filtrate was concentrated under reduced pressure to provide the title compound (900 mg, 99% yield). LCMS (ESI) [M+H]⁺=261.1.

Step 5: 6-((1S,3S)-3-(1-Isopropyl-4-(trifluoromethyl)-1H-imidazol-2-yl)cyclopentyl)-2-thia-6-azaspiro[3.4]octane 2,2-dioxide (Compound 20A*): 3-(1-Isopropyl-4-(trifluoromethyl)-1H-imidazol-2-yl)cyclopentan-1-one (900.0 mg, 3.46 mmol) was purified by chiral SFC (Daicel Chiralcel OD, 0.1% NH₃ in water/EtOH 55:45) to provide the first eluting peak as a pure single undefined enantiomer of 3-(1-isopropyl-4-(trifluoromethyl)-1H-imidazol-2-yl)cyclopentan-1-one (400 mg, 44.4% yield). To a solution of the above material (50.0 mg, 0.19 mmol), 2-thia-6-azaspiro[3.4]octane 2,2-dioxide hydrochloride (42.0 mg, 0.21 mmol) in anhydrous methanol (3 mL) was added NaBH₃CN (40 mg, 0.63 mmol). The reaction was stirred at 50° C. for 1.5 h. The reaction was concentrated and the resulting residue was purified by reverse phase chromatography (acetonitrile/water gradient with 0.05% NH₄OH) to provide the racemic mixture of the title compound. The racemic mixture was separated by chiral SFC (Daicel Chiralcel OD, 0.1% NH₃ in water/ethanol 55:45) to provide the first eluting peak as a pure single undefined enantiomer of the title compound (13.6 mg, 17.3% yield; Compound 20A*). LCMS (ESI) [M+H]⁺=406.2. ¹H NMR (400 MHz, CD₃OD) δ 7.63 (s, 1H), 4.62-4.54 (m, 1H), 4.20-4.06 (m, 4H), 3.57-3.43 (m, 1H), 3.04-2.88 (m, 3H), 2.79 (t, J=7.2 Hz, 2H), 2.22 (t, J=7.2 Hz, 2H), 2.17-1.96 (m, 4H), 1.93-1.85 (m, 1H), 1.68-1.60 (m, 1H), 1.46 (dd, J=6.8, 2.0 Hz, 6H).

Compound 20B* was obtained as the second eluting peak as a pure single undefined enantiomer of the title compound (42.8 mg, 54.4% yield). LCMS (ESI) [M+H]⁺=406.2. ¹H NMR (400 MHz, CD₃OD) δ 7.59 (s, 1H), 4.58-4.47 (m, 1H), 4.16-4.01 (m, 4H), 3.29-3.25 (m, 1H), 2.90 (s, 2H), 2.86-2.68 (m, 3H), 2.27-2.15 (m, 3H), 2.11-2.01 (m, 1H), 1.98-1.86 (m, 3H), 1.83-1.75 (m, 1H), 1.41 (dd, J=6.8, 1.6 Hz, 6H). The relative cis stereochemistry was assigned based on ¹H NMR analysis.

Racemic 3-(1-isopropyl-4-(trifluoromethyl)-1H-imidazol-2-yl)cyclopentan-1-one (900.0 mg, 3.46 mmol) prepared as described above was purified by chiral SFC (Daicel Chiralcel OD, 0.1% NH₃ in water/EtOH 55:45) to provide the second eluting peak as a pure single undefined enantiomer of 3-(1-isopropyl-4-(trifluoromethyl)-1H-imidazol-2-yl)cyclopentan-1-one (300 mg, 33.3% yield). LCMS (ESI) [M+H]⁺=261.1. Following a procedure similar to example 44, the above material (50.0 mg, 0.19 mmol) was converted to the racemic mixture of the title compound. The racemic mixture was separated by chiral SFC (Daicel Chiralcel OD, 0.1% NH₃ in water/ethanol 55:45) to provide the first eluting peak as a pure single undefined enantiomer of the title compound (59 mg, 37.9% yield; Compound 20C*). LCMS (ESI) [M+H]⁺=406.2. ¹H NMR (400 MHz, CD₃OD) δ 7.65 (s, 1H), 4.59-4.52 (m, 1H), 4.23-4.08 (m, 4H), 3.58-3.46 (m, 1H), 3.05-2.89 (m, 3H), 2.80 (t, J=7.2 Hz, 2H), 2.24 (t, J=7.2 Hz, 2H), 2.20-1.95 (m, 4H), 1.94-1.85 (m, 1H), 1.66-1.62 (m, 1H), 1.47 (dd, J=6.8, 2.0 Hz, 6H).

The racemic mixture was separated by chiral SFC (Daicel Chiralcel OD, 0.1% NH₃ in water/ethanol 55:45) to provide the second eluting peak as a pure single undefined enantiomer of the title compound (35.7 mg, 22.7% yield; Compound 20D*). LCMS (ESI) [M+H]⁺=406.2. ¹H NMR (400 MHz, CD₃OD) δ 7.63 (s, 1H), 4.56-4.50 (m, 1H), 4.20-4.04 (m, 4H), 3.33-3.29 (m, 1H), 2.92 (s, 2H), 2.88-2.66 (m, 3H), 2.31-2.16 (m, 3H), 2.15-2.04 (m, 1H), 2.03-1.86 (m, 3H), 1.84-1.73 (m, 1H), 1.45 (dd, J=6.8, 2.0 Hz, 6H).

### Example V: 6-((1s,4s)-4-(4-Methyl-6-(trifluoromethyl)pyridin-3-yl)cyclohexyl)-2-thia-6-azaspiro[3.4]octane 2,2-dioxide (Compound 21A) and 6-((1r,4r)-4-(4-Methyl-6-(trifluoromethyl)pyridin-3-yl)cyclohexyl)-2-thia-6-azaspiro[3.4]octane 2,2-dioxide (Compound 21B)

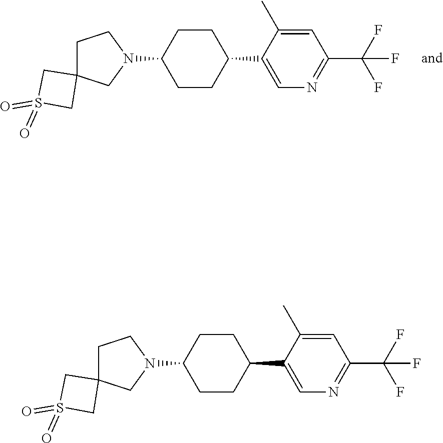 [SMILES: Cc1cc(C(F)(F)F)ncc1[C@H]1CC[C@@H](N2CCC3(C2)CS(=O)(=O)C3)CC1 ; Cc1cc(C(F)(F)F)ncc1[C@H]1CC[C@H](N2CCC3(C2)CS(=O)(=O)C3)CC1]

The title compounds were synthesized following a procedure similar to Compound 3A using 5-bromo-4-methyl-2-(trifluoromethyl)pyridine in step 1. The mixture of the stereoisomers was purified by prep-TLC (10% ethyl acetate in methanol).

### 6-((1s,4s)-4-(4-Methyl-6-(trifluoromethyl)pyridin-3-yl)cyclohexyl)-2-thia-6-azaspiro[3.4]octane 2,2-dioxide (Compound 21A)

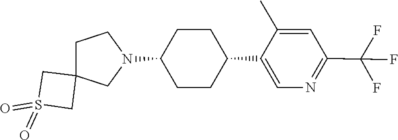 [SMILES: Cc1cc(C(F)(F)F)ncc1[C@H]1CC[C@@H](N2CCC3(C2)CS(=O)(=O)C3)CC1]

Provided as the first eluting peak as a pure single stereoisomer of the above titled compound (30 mg, 9.7% yield). LCMS (ESI) [M+H]⁺=403.2. ¹H NMR (400 MHz, CDCl₃) δ 8.50 (s, 1H), 7.45 (s, 1H), 4.08 (s, 4H), 2.93 (s, 2H), 2.82-2.71 (m, 3H), 2.41 (s, 3H), 2.31-2.24 (m, 1H), 2.19-2.11 (m, 4H), 1.92-1.89 (m, 2H), 1.65-1.54 (m, 2H), 1.44-1.25 (m, 2H). The relative cis stereochemistry was assigned based on ¹H NMR analysis.

### 6-((1r,4r)-4-(4-Methyl-6-(trifluoromethyl)pyridin-3-yl)cyclohexyl)-2-thia-6-azaspiro[3.4]octane 2,2-dioxide (Compound 21B)

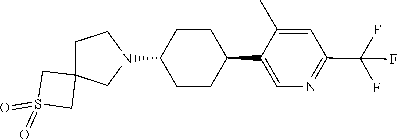 [SMILES: Cc1cc(C(F)(F)F)ncc1[C@H]1CC[C@H](N2CCC3(C2)CS(=O)(=O)C3)CC1]

Provided as the second eluting peak as a pure single stereoisomer of the above titled compound (61 mg, 21.5% yield). LCMS (ESI) [M+H]⁺=403.2. ¹H NMR (400 MHz, CDCl₃) δ 8.50 (s, 1H), 7.45 (s, 1H), 4.09 (s, 4H), 2.93 (s, 2H), 2.83-2.73 (m, 3H), 2.42 (s, 3H), 2.19-2.12 (m, 4H), 1.93-1.90 (m, 2H), 1.65-1.55 (m, 3H), 1.41-1.26 (m, 2H). The relative trans stereochemistry was assigned based on ¹H NMR analysis.

### Example W: (Compounds 22A* and 22B*): (R)-6-(1-(4-(Trifluoromethyl)phenyl)piperidin-3-yl)-2-thia-6-azaspiro[3.4]octane 2,2-dioxide and (S)-6-(1-(4-(trifluoromethyl)phenyl)piperidin-3-yl)-2-thia-6-azaspiro[3.4]octane 2,2-dioxide

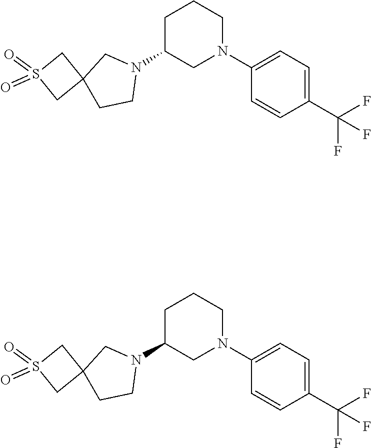 [SMILES: O=S1(=O)CC2(CCN([C@@H]3CCCN(c4ccc(C(F)(F)F)cc4)C3)C2)C1 ; O=S1(=O)CC2(CCN([C@H]3CCCN(c4ccc(C(F)(F)F)cc4)C3)C2)C1]

### 6-(1-(4-(Trifluoromethyl)phenyl)azepan-4-yl)-2-thia-6-azaspiro[3.4]octane 2,2-dioxide

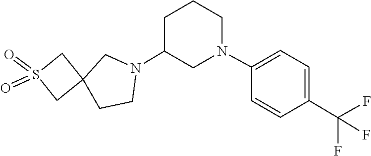 [SMILES: O=S1(=O)CC2(CCN(C3CCCN(c4ccc(C(F)(F)F)cc4)C3)C2)C1]

The title compound was synthesized following a procedure similar to Compound 4 using 1,4-dioxa-7-azaspiro[4.5]decane in step 1. The racemic mixture was separated by chiral SFC (Daicel Chiralpak AD-H, 0.1% NH₃ in water/IPA 70:30) to provide the first eluting peak as a pure single undefined enantiomer of the title compound (15.6 mg, 19% yield; Compound 22A*). LCMS (ESI) [M+H]⁺=389.0. ¹H NMR (400 MHz, CD₃OD) δ 7.50-7.42 (m, 2H), 7.02 (d, J=8.8 Hz, 2H), 4.15-4.05 (m, 4H), 3.90-3.98 (m, 1H), 3.80-3.70 (m, 1H), 3.00-2.95 (m, 2H), 2.92-2.75 (m, 4H), 2.47-2.37 (m, 1H), 2.19 (t, J=7.2 Hz, 2H), 2.12-2.04 (m, 1H), 1.89-1.80 (m, 1H), 1.72-1.57 (m, 1H), 1.52-1.39 (m, 1H).

The same purification conditions provided was the second eluting peak as a pure single undefined enantiomer of the title compound (20.0 mg, 24% yield; Compound 22B*). LCMS (ESI) [M+H]⁺=389.0. ¹H NMR (400 MHz, CD₃OD) δ7.45 (d, J=8.8 Hz, 2H), 7.02 (d, J=8.8 Hz, 2H), 4.18-4.04 (m, 4H), 3.95-3.90 (m, 1H), 3.73 (d, J=12.8 Hz, 1H), 3.00-2.95 (m, 2H), 2.92-2.74 (m, 4H), 2.47-2.37 (m, 1H), 2.19 (t, J=7.2 Hz, 2H), 2.11-2.03 (m, 1H), 1.89-1.79 (m, 1H), 1.71-1.58 (m, 1H), 1.53-1.39 (m, 1H).

### Example X: 6-((1s,4s)-4-(1-Cyclobutyl-3-(trifluoromethyl)-1H-pyrazol-5-yl)cyclohexyl)-2-thia-6-azaspiro[3.4]octane 2,2-dioxide (Compound 23A), 6-((1r,4r)-4-(1-Cyclobutyl-3-(trifluoromethyl)-1H-pyrazol-5-yl)cyclohexyl)-2-thia-6-azaspiro[3.4]octane 2,2-dioxide (Compound 23B), and 6-((1r,4r)-4-(1-cyclobutyl-5-(trifluoromethyl)-1H-pyrazol-3-yl)cyclohexyl)-2-thia-6-azaspiro[3.4]octane 2,2-dioxide (Compound 24)

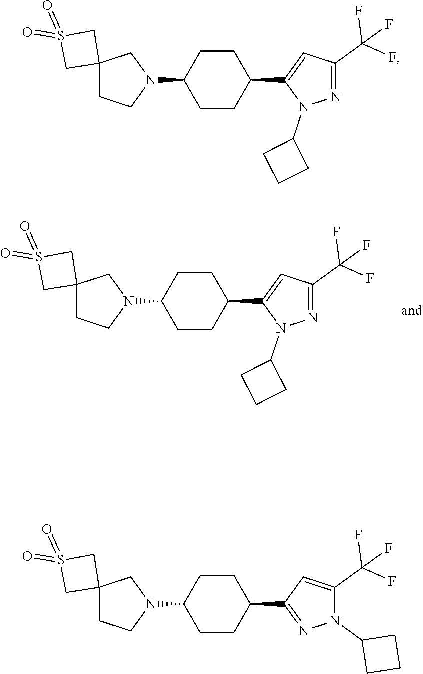 [SMILES: O=S1(=O)CC2(CCN([C@H]3CC[C@@H](c4cc(C(F)(F)F)nn4C4CCC4)CC3)C2)C1 ; O=S1(=O)CC2(CCN([C@H]3CC[C@H](c4cc(C(F)(F)F)nn4C4CCC4)CC3)C2)C1 ; O=S1(=O)CC2(CCN([C@H]3CC[C@H](c4cc(C(F)(F)F)n(C5CCC5)n4)CC3)C2)C1]

The title compounds were synthesized following a procedure similar to Compound 12A using cyclobutyl hydrazine hydrochloride in step 2. Separation of the isomers by reverse phase HPLC provided the purified products, Compounds 23A, 23B, and 24.

### 6-((1s,4s)-4-(1-Cyclobutyl-3-(trifluoromethyl)-1H-pyrazol-5-yl)cyclohexyl)-2-thia-6-azaspiro[3.4]octane 2,2-dioxide (Compound 23A)

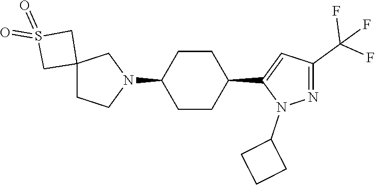 [SMILES: O=S1(=O)CC2(CCN([C@H]3CC[C@@H](c4cc(C(F)(F)F)nn4C4CCC4)CC3)C2)C1]

LCMS (ESI) [M+H]⁺=432.2. ¹H NMR (400 MHz, DMSO-d₆) δ 6.50 (s, 1H), 4.97 (m, 1H), 4.21-4.07 (m, 4H), 2.89-2.78 (m, 1H), 2.73 (s, 2H), 2.60 (t, J=7.2 Hz, 2H), 2.55-2.51 (m, 2H), 2.41-2.29 (m, 3H), 2.09 (t, J=7.3 Hz, 2H), 1.88-1.77 (m, 4H), 1.76-1.64 (m, 2H), 1.61-1.48 (m, 4H). Cis stereochemistry was assigned based on ¹H NMR analysis.

### 6-((1r,4r)-4-(1-Cyclobutyl-3-(trifluoromethyl)-1H-pyrazol-5-yl)cyclohexyl)-2-thia-6-azaspiro[3.4]octane 2,2-dioxide (Compound 23B)

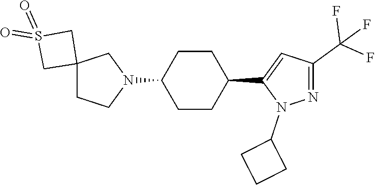 [SMILES: O=S1(=O)CC2(CCN([C@H]3CC[C@H](c4cc(C(F)(F)F)nn4C4CCC4)CC3)C2)C1]

LCMS (ESI) [M+H]⁺=432.2. ¹H NMR (400 MHz, DMSO-d₆) δ 6.48 (s, 1H), 5.03-4.91 (m, 1H), 4.22-4.01 (m, 4H), 2.85-2.60 (m, 5H), 2.59-2.51 (m, 2H), 2.41-2.32 (m, 2H), 2.22-2.02 (m, 3H), 2.01-1.91 (m, 2H), 1.88-1.74 (m, 4H), 1.48-1.22 (m, 4H). Trans stereochemistry was assigned based on ¹H NMR analysis.

### 6-((1r,4r)-4-(1-cyclobutyl-5-(trifluoromethyl)-1H-pyrazol-3-yl)cyclohexyl)-2-thia-6-azaspiro[3.4]octane 2,2-dioxide (Compound 24)

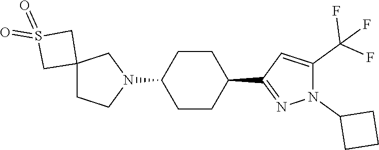 [SMILES: O=S1(=O)CC2(CCN([C@H]3CC[C@H](c4cc(C(F)(F)F)n(C5CCC5)n4)CC3)C2)C1]

LCMS (ESI) [M+H]⁺=432.2. ¹H NMR (400 MHz, DMSO-d₆) δ 6.50 (s, 1H), 4.97 (m, 1H), 4.21-4.07 (m, 4H), 2.89-2.78 (m, 1H), 2.73 (s, 2H), 2.60 (t, J=7.2 Hz, 2H), 2.55-2.51 (m, 2H), 2.41-2.29 (m, 3H), 2.09 (t, J=7.3 Hz, 2H), 1.88-1.77 (m, 4H), 1.76-1.64 (m, 2H), 1.61-1.48 (m, 4H). Trans stereochemistry was assigned based on ¹H NMR analysis.

### Example Y: 7-((1s,4s)-4-(1-Methyl-3-(trifluoromethyl)-1H-pyrazol-5-yl)cyclohexyl)-2-thia-7-azaspiro[3.5]nonane 2,2-dioxide (Compound 25A) and 7-((1r,4r)-4-(1-methyl-3-(trifluoromethyl)-1H-pyrazol-5-yl)cyclohexyl)-2-thia-7-azaspiro[3.5]nonane 2,2-dioxide (Compound 25B)

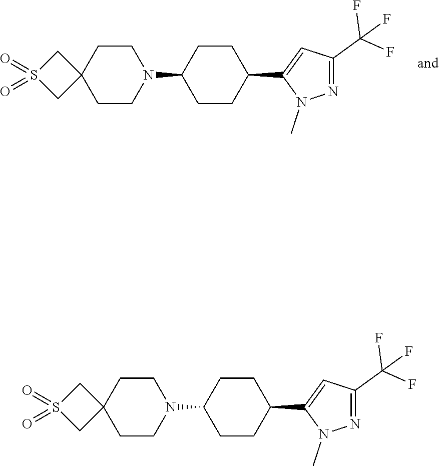 [SMILES: Cn1nc(C(F)(F)F)cc1[C@H]1CC[C@@H](N2CCC3(CC2)CS(=O)(=O)C3)CC1 ; Cn1nc(C(F)(F)F)cc1[C@H]1CC[C@H](N2CCC3(CC2)CS(=O)(=O)C3)CC1]

The title compounds were synthesized following a procedure similar to Compound 15 using methyl hydrazine hydrochloride in step 1 and 2-thia-7-azaspiro[3.5]nonane 2,2-dioxide hydrochloride in step 2. Purification of the crude mixture by reverse phase HPLC afforded the title compounds.

Compound 25A: 7-((1s,4s)-4-(1-Methyl-3-(trifluoromethyl)-1H-pyrazol-5-yl)cyclohexyl)-2-thia-7-azaspiro[3.5]nonane 2,2-dioxide. LCMS (ESI) [M+H]⁺=406.2. ¹H NMR (400 MHz, DMSO-d₆) δ 6.47 (s, 1H), 3.91 (s, 4H), 3.84 (s, 3H), 2.72-2.63 (m, 1H), 2.49-2.42 (m, 4H), 2.39-2.29 (m, 1H), 1.96-1.89 (m, 2H), 1.83-1.70 (m, 6H), 1.47-1.30 (m, 4H). Cis stereochemistry was assigned based on ¹H NMR analysis.

Compound 25B: 7-((1r,4r)-4-(1-methyl-3-(trifluoromethyl)-1H-pyrazol-5-yl)cyclohexyl)-2-thia-7-azaspiro[3.5]nonane 2,2-dioxide. LCMS (ESI) [M+H]⁺=406.2. ¹H NMR (400 MHz, DMSO-d₆) δ 6.49 (s, 1H), 3.91 (s, 4H), 3.84 (s, 3H), 2.97-2.86 (m, 1H), 2.47-2.24 (m, 4H), 2.23-2.17 (m, 1H), 1.93-1.82 (m, 2H), 1.79 (t, J=5.4 Hz, 4H), 1.76-1.67 (m, 2H), 1.66-1.56 (m, 2H), 1.56-1.45 (m, 2H). Trans stereochemistry was assigned based on ¹H NMR analysis.

### Example Z: (Compounds 26A* and 26B*): (S)-7-((1r,4S)-4-(1-Methyl-3-(trifluoromethyl)-1H-pyrazol-5-yl)cyclohexyl)-2-thia-7-azaspiro[4.5]decane 2,2-dioxide and (R)-7-((1r,4R)-4-(1-methyl-3-(trifluoromethyl)-1H-pyrazol-5-yl)cyclohexyl)-2-thia-7-azaspiro[4.5]decane 2,2-dioxide

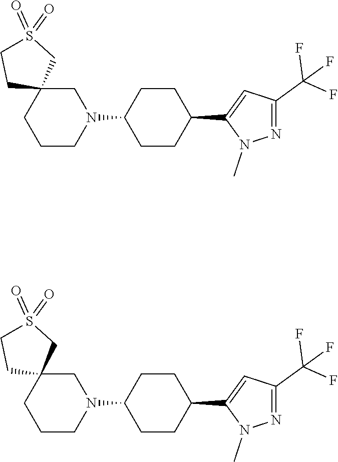 [SMILES: Cn1nc(C(F)(F)F)cc1[C@H]1CC[C@H](N2CCC[C@@]3(CCS(=O)(=O)C3)C2)CC1 ; Cn1nc(C(F)(F)F)cc1[C@H]1CC[C@H](N2CCC[C@]3(CCS(=O)(=O)C3)C2)CC1]

### 7-((1r,4r)-4-(1-methyl-3-(trifluoromethyl)-1H-pyrazol-5-yl)cyclohexyl)-2-thia-7-azaspiro[4.5]decane 2,2-dioxide

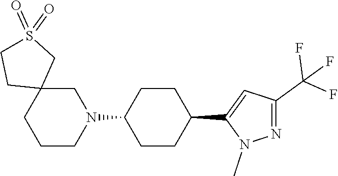 [SMILES: Cn1nc(C(F)(F)F)cc1[C@H]1CC[C@H](N2CCCC3(CCS(=O)(=O)C3)C2)CC1]

The racemic mixture of 7-((1r,4r)-4-(1-methyl-3-(trifluoromethyl)-1H-pyrazol-5-yl)cyclohexyl)-2-thia-7-azaspiro[4.5]decane 2,2-dioxide was synthesized following a procedure similar to Compound 15 using methyl hydrazine hydrochloride in step 1 and 2-thia-7-azaspiro[4.5]decane 2,2-dioxide hydrochloride in step 2.

The mixture was separated by chiral SFC (Chiralpak IC, 0.1% ammonium hydroxide in methanol/CO₂ 25:75) to provide the first eluting peak as a pure single undefined enantiomer of the title compound (4.3 mg, 4% yield; Compound 26A*). LCMS (ESI) [M+H]⁺=420.2. ¹H NMR (400 MHz, DMSO) δ 6.48 (s, 1H), 3.84 (s, 3H), 3.20-3.12 (m, 3H), 2.88 (d, J=13.6 Hz, 1H), 2.73-2.57 (m, 3H), 2.44-2.33 (m, 2H), 2.26-2.19 (m, 1H), 2.09-1.98 (m, 1H), 1.96-1.72 (m, 5H), 1.64-1.49 (m, 2H), 1.49-1.31 (m, 6H). The stereochemistry of chiral center of spirocycle was arbitrarily assigned.

Also provided was the second eluting peak as a pure single undefined enantiomer of the title compound (4.0 mg, 4% yield; Compound 26B*). LCMS (ESI) [M+H]⁺=420.2. ¹H NMR (400 MHz, DMSO-d₆) δ 6.48 (s, 1H), 3.84 (s, 3H), 3.20-3.12 (m, 3H), 2.88 (d, J=13.6 Hz, 1H), 2.72-2.55 (m, 3H), 2.44-2.32 (m, 2H), 2.26-2.19 (m, 1H), 2.10-1.98 (m, 1H), 1.97-1.73 (m, 5H), 1.64-1.50 (m, 2H), 1.49-1.30 (m, 6H). The stereochemistry of chiral center of spirocycle was arbitrarily assigned.

### Example AA: (Compounds 27A* and 27B*): (R)-7-((1r,4R)-4-(1-Methyl-3-(trifluoromethyl)-1H-pyrazol-5-yl)cyclohexyl)-2-thia-7-azaspiro[4.4]nonane 2,2-dioxide and (S)-7-((1r,4S)-4-(1-methyl-3-(trifluoromethyl)-1H-pyrazol-5-yl)cyclohexyl)-2-thia-7-azaspiro[4.4]nonane 2,2-dioxide

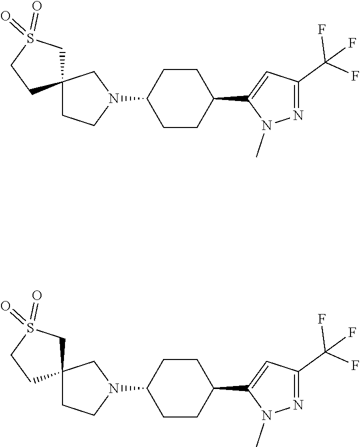 [SMILES: Cn1nc(C(F)(F)F)cc1[C@H]1CC[C@H](N2CC[C@@]3(CCS(=O)(=O)C3)C2)CC1 ; Cn1nc(C(F)(F)F)cc1[C@H]1CC[C@H](N2CC[C@]3(CCS(=O)(=O)C3)C2)CC1]

### 7-((1r,4r)-4-(1-methyl-3-(trifluoromethyl)-1H-pyrazol-5-yl)cyclohexyl)-2-thia-7-azaspiro[4.4]nonane 2,2-dioxide

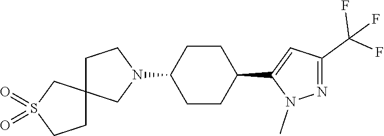 [SMILES: Cn1nc(C(F)(F)F)cc1[C@H]1CC[C@H](N2CCC3(CCS(=O)(=O)C3)C2)CC1]

The racemic mixture of 7-((1r,4r)-4-(1-methyl-3-(trifluoromethyl)-1H-pyrazol-5-yl)cyclohexyl)-2-thia-7-azaspiro[4.4]nonane 2,2-dioxide was synthesized following a procedure similar to Compound 15 using methyl hydrazine hydrochloride in step 1 and 2-thia-7-azaspiro[4.4]nonane 2,2-dioxide hydrochloride in step 2.

The mixture was separated by chiral SFC (Chiralpak IA, 0.1% ammonium hydroxide in methanol/CO₂ 15:85) to provide the first eluting peak as a pure single undefined enantiomer of the title compound (18.5 mg, 18% yield). Compound 27A*: LCMS (ESI) [M+H]⁺=406.2. ¹H NMR (400 MHz, DMSO-d₆) δ 6.49 (s, 1H), 3.84 (s, 3H), 3.20-3.13 (m, 2H), 3.12-3.04 (m, 2H), 2.76-2.65 (m, 3H), 2.64-2.56 (m, 1H), 2.55-2.51 (m, 1H), 2.19-2.06 (m, 3H), 2.00-1.74 (m, 6H), 1.45-1.22 (m, 4H).

Also provided was the second eluting peak as a pure single undefined enantiomer of the title compound (18.3 mg, 18% yield). Compound 27B*: LCMS (ESI) [M+H]⁺=406.2. ¹H NMR (400 MHz, DMSO-d₆) δ 6.49 (s, 1H), 3.84 (s, 3H), 3.20-3.13 (m, 2H), 3.13-3.04 (m, 2H), 2.76-2.65 (m, 3H), 2.64-2.52 (m, 2H), 2.21-2.04 (m, 3H), 2.01-1.82 (m, 5H), 1.82-1.72 (m, 1H), 1.45-1.22 (m, 4H). Stereochemistry of chiral center in spirocycle was arbitrarily assigned.

### Example BB: 6-(1-(4-Methyl-6-(trifluoromethyl)pyridin-3-yl)piperidin-4-yl)-2-thia-6-azaspiro[3.4]octane 2,2-dioxide (Compound 28)

### Step 1: 8-(4-Methyl-6-(trifluoromethyl)pyridin-3-yl)-1,4-dioxa-8-azaspiro[4.5]decane

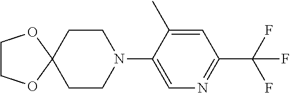 [SMILES: Cc1cc(C(F)(F)F)ncc1N1CCC2(CC1)OCCO2]

To a solution of 5-bromo-4-methyl-2-(trifluoromethyl)pyridine (250 mg, 1.04 mmol) and 1,4-dioxa-8-azaspiro[4.5]decane (225 mg, 1.58 mmol) in tetrahydrofuran (5 mL) was added sodium t-butoxide (300 mg, 3.13 mmol) and Pd-PEPPSI-IHept^{Cl}-Py (113 mg, 0.11 mmol) in a glovebox. The reaction mixture was stirred at 60° C. under a N2 atmosphere for 16 h. The reaction mixture was quenched with water (20 mL) and extracted with ethyl acetate (60 mL×3). The combined organic layer was washed with brine (20 mL), dried over Na₂SO₄, filtered and concentrated. The residue was purified by silica column chromatography (0-30% ethyl acetate in petroleum ether) to provide the title compound (280 mg, 89% yield). LCMS (ESI) [M+H]⁺=303.1.

### Step 2: 1-(4-Methyl-6-(trifluoromethyl)pyridin-3-yl)piperidin-4-one

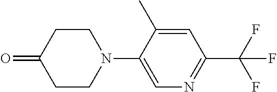 [SMILES: Cc1cc(C(F)(F)F)ncc1N1CCC(=O)CC1]

8-(4-methyl-6-(trifluoromethyl)pyridin-3-yl)-1,4-dioxa-8-azaspiro[4.5]decane (280 mg, 0.93 mmol) and hydrochloric acid (12 mL, 72 mmol, 6M) were stirred at 25° C. for 16 h. The pH of the reaction mixture was adjusted to pH=9 with a saturated aq. NaHCO₃ solution (30 mL) at 0° C. The mixture was extracted with ethyl acetate (50 mL×3). The combined organic layer was washed with brine (20 mL), dried over Na₂SO₄, filtered and concentrated. The residue was purified by silica column chromatography (0-30% ethyl acetate in petroleum ether) to provide the title compound (200 mg, 79.4% yield). LCMS (ESI) [M+H]⁺=259.1.

### Step 3: 6-(1-(4-Methyl-6-(trifluoromethyl)pyridin-3-yl)piperidin-4-yl)-2-thia-6-azaspiro[3.4]octane 2,2-dioxide, Compound 28

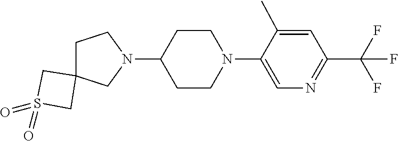 [SMILES: Cc1cc(C(F)(F)F)ncc1N1CCC(N2CCC3(C2)CS(=O)(=O)C3)CC1]

To a solution of 1-(4-methyl-6-(trifluoromethyl)pyridin-3-yl)piperidin-4-one (80 mg, 0.31 mmol) and 2-thia-6-azaspiro[3.4]octane 2,2-dioxide (100 mg, 0.62 mmol) in anhydrous methanol (4 mL) was added NaBH₃CN (100 mg, 1.59 mmol) and acetic acid (0.08 mL) at 20° C. The reaction mixture was stirred at 50° C. for 15 h. The reaction was quenched with a saturated aq. NaHCO₃ solution (6 mL) and extracted with dichloromethane (50 mL×3). The combined organic layers were washed with brine (20 mL), dried over Na₂SO₄, filtered and concentrated under reduced pressure. The resulting residue was purified by prep-TLC (10% methanol in dichloromethane) to provide the title compound (33.4 mg, 26.2% yield). LCMS (ESI) [M+H]⁺=404.2. ¹H NMR (400 MHz, CDCl₃) δ 8.30 (s, 1H), 7.46 (s, 1H), 4.09 (s, 4H), 3.24 (d, J=12.0 Hz, 2H), 2.92 (s, 2H), 2.87-2.71 (m, 4H), 2.36 (s, 3H), 2.35-2.27 (m, 1H), 2.19 (t, J=7.2 Hz, 2H), 1.99 (d, J=12.8 Hz, 2H), 1.77-1.64 (m, 2H).

### Example CC: 6-(1-(2-methyl-6-(trifluoromethyl)pyridin-3-yl)piperidin-4-yl)-2-thia-6-azaspiro[3.4]octane 2,2-dioxide (Compound 29)

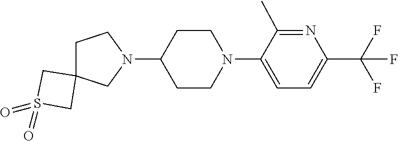 [SMILES: Cc1nc(C(F)(F)F)ccc1N1CCC(N2CCC3(C2)CS(=O)(=O)C3)CC1]

The title compound was synthesized following a procedure similar to Compound 28 using 3-bromo-2-methyl-6-(trifluoromethyl)pyridine in step 1. Purification of the crude mixture by reverse phase HPLC afforded the title compound.

LCMS (ESI) [M+H]⁺=404.2. ¹H NMR (400 MHz, CDCl₃) δ 7.46 (d, J=8.4 Hz, 1H), 7.31 (d, J=8.4 Hz, 1H), 4.09 (s, 4H), 3.21 (d, J=12.0 Hz, 2H), 2.93 (s, 2H), 2.80 (t, J=7.2 Hz, 2H), 2.71 (t, J=10.8 Hz, 2H), 2.58 (s, 3H), 2.37-2.27 (m, 1H), 2.19 (t, J=7.2 Hz, 2H), 1.99 (d, J=11.6 Hz, 2H), 1.80-1.65 (m, 2H).

### Example DD: 6-(1-(3-chloro-5-(trifluoromethyl)pyridin-2-yl)piperidin-4-yl)-2-thia-6-azaspiro[3.4]octane 2,2-dioxide (Compound 30)

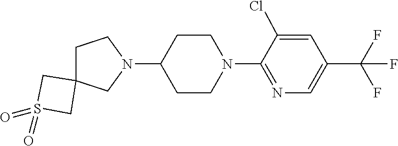 [SMILES: O=S1(=O)CC2(CCN(C3CCN(c4ncc(C(F)(F)F)cc4Cl)CC3)C2)C1]

The title compound was synthesized following a procedure similar to Compound 28 using 2-bromo-3-chloro-5-(trifluoromethyl)pyridine in step 1. Purification of the crude mixture by reverse phase HPLC afforded the title compound. LCMS (ESI) [M+H]⁺=424.0. ¹H NMR (400 MHz, DMSO-d₆) δ 8.52 (s, 1H), 8.16 (s, 1H), 4.17-4.09 (m, 4H), 3.93 (d, J=12.8 Hz, 2H), 2.99 (t, J=11.6 Hz, 2H), 2.90-2.80 (m, 2H), 2.75-2.65 (m, 2H), 2.43-2.32 (m, 1H), 2.09 (s, 2H), 1.91 (d, J=11.2 Hz, 2H), 1.60-1.45 (m, 2H).

### Example EE: 6-(1-(5-fluoro-3-methylpyridin-2-yl)piperidin-4-yl)-2-thia-6-azaspiro[3.4]octane 2,2-dioxide (Compound 31)

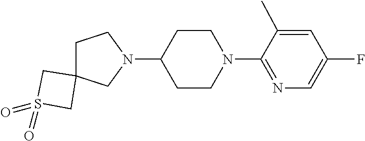 [SMILES: Cc1cc(F)cnc1N1CCC(N2CCC3(C2)CS(=O)(=O)C3)CC1]

The title compound was synthesized following a procedure similar to Compound 28 using 2-bromo-5-fluoro-3-methylpyridine in step 1. Purification of the crude mixture by reverse phase HPLC afforded the title compound. LCMS (ESI) [M+H]⁺=353.9. ¹H NMR (400 MHz, CD₃OD) δ 7.96 (d, J=2.8 Hz, 1H), 7.39 (dd, J=8.8, 2.4 Hz, 1H), 4.28-4.15 (m, 4H), 3.43-3.35 (m, 4H), 3.21 (t, J=7.2 Hz, 2H), 2.88-2.77 (m, 3H), 2.40-2.25 (m, 5H), 2.15-2.05 (m, 2H), 1.95-1.80 (m, 2H).

### Example FF: 6-(1-(3-fluoro-5-(trifluoromethyl)pyridin-2-yl)piperidin-4-yl)-2-thia-6-azaspiro[3.4]octane 2,2-dioxide (Compound 32)

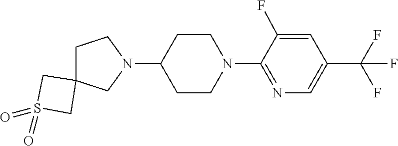 [SMILES: O=S1(=O)CC2(CCN(C3CCN(c4ncc(C(F)(F)F)cc4F)CC3)C2)C1]

The title compound was synthesized following a procedure similar to Compound 4 using 2,3-difluoro-5-(trifluoromethyl)pyridine in step 1. Purification of the crude mixture by reverse phase HPLC afforded the title compound. LCMS (ESI) [M+H]⁺=408.2. ¹H NMR (400 MHz, DMSO-d₆) δ 8.32 (s, 1H), 7.92-7.88 (m, 1H), 4.16-4.08 (m, 6H), 3.14-3.08 (m, 2H), 2.79 (s, 2H), 2.67-2.64 (m, 2H), 2.50-2.39 (s, 1H), 2.09-2.06 (m, 2H), 1.89-1.87 (m, 2H), 1.48-1.42 (m, 2H).

### Example GG: (Trans)-6-(4-(3-(5-(Difluoromethoxy)pyridin-3-yl)-1-isopropyl-1H-pyrazol-5-yl)cyclohexyl)-2-thia-6-azaspiro[3.4]octane 2,2-dioxide (Compound 33A) and (Cis)-6-(4-(3-(5-(Difluoromethoxy)pyridin-3-yl)-1-isopropyl-1H-pyrazol-5-yl)cyclohexyl)-2-thia-6-azaspiro[3.4]octane 2,2-dioxide (Compound 33B)

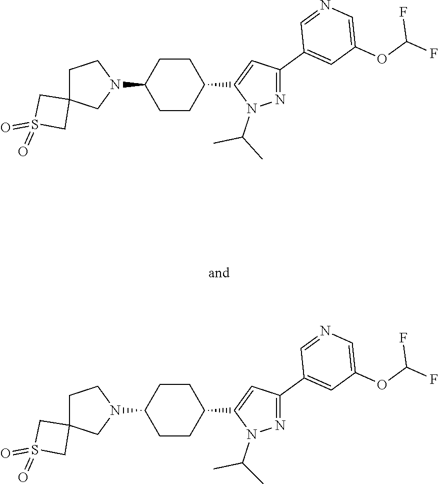 [SMILES: CC(C)n1nc(-c2cncc(OC(F)F)c2)cc1[C@H]1CC[C@H](N2CCC3(C2)CS(=O)(=O)C3)CC1 ; CC(C)n1nc(-c2cncc(OC(F)F)c2)cc1[C@H]1CC[C@@H](N2CCC3(C2)CS(=O)(=O)C3)CC1]

### Step 1: [5-(Difluoromethoxy)-3-pyridyl]boronic acid

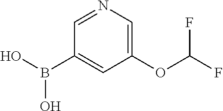 [SMILES: OB(O)c1cncc(OC(F)F)c1]

To a solution of 3-bromo-5-(difluoromethoxy)pyridine (1 g, 4.5 mmol) and 4,4,4′,4′,5,5,5′,5′-octamethyl-2,2′-bi(1,3,2-dioxaborolane) (2 g, 9 mmol) in 1,4-dioxane (15 mL) was added potassium acetate (1.3 g, 13.4 mmol) and 1,1′-bis(diphenylphosphino)ferrocene palladium dichloride (326 mg, 0.5 mmol). The mixture was stirred at 80° C. under N2 for 16 h. The crude product was used for the next step without further purification. LCMS (ESI) [M+H]⁺=190.0.

### Step 2: 4-(3-(5-(Difluoromethoxy)pyridin-3-yl)-1-isopropyl-1H-pyrazol-5-yl)cyclohexan-1-one

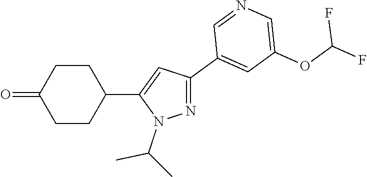 [SMILES: CC(C)n1nc(-c2cncc(OC(F)F)c2)cc1C1CCC(=O)CC1]

To a solution of [5-(difluoromethoxy)-3-pyridyl]boronic acid (662 mg, 3.51 mmol) and 4-(5-bromo-2-isopropyl-pyrazol-3-yl)cyclohexanone (500 mg, 1.75 mmol) and K₂CO₃ (727 mg, 5.3 mmol) in 1,4-dioxane (10 mL) and water (2 mL) was added 1,1′-bis(diphenylphosphino)ferrocene palladium dichloride (128 mg, 0.18 mmol). The mixture was stirred at 100° C. under N2 for 16 h. The mixture was diluted with water (25 mL) and extracted with ethyl acetate (30 mL×3). The combined organic layer was washed with brine (25 mL×3), dried over anhydrous sodium sulfate, filtered and concentrated. The residue was purified by silica chromatography to provide the title compound (400 mg, 65% yield). LCMS (ESI) [M+H]⁺=350.0.

### Step 3A: 6-((1r,4r)-4-(3-(5-(Difluoromethoxy)pyridin-3-yl)-1-isopropyl-1H-pyrazol-5-yl)cyclohexyl)-2-thia-6-azaspiro[3.4]octane 2,2-dioxide (Compound 33A)

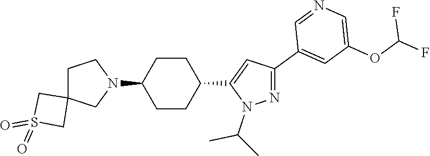 [SMILES: CC(C)n1nc(-c2cncc(OC(F)F)c2)cc1[C@H]1CC[C@H](N2CCC3(C2)CS(=O)(=O)C3)CC1]

To a solution of 4-(3-(5-(difluoromethoxy)pyridin-3-yl)-1-isopropyl-1H-pyrazol-5-yl)cyclohexan-1-one (100 mg, 0.29 mmol) in methanol (5 mL) was added 2-thia-6-azaspiro[3.4]octane 2,2-dioxide hydrochloride (68 mg, 0.34 mmol), acetic acid (0.02 mL) and NaBH₃CN (90 mg, 1.43 mmol). The mixture was stirred at 50° C. for 3 h. The reaction mixture was diluted with water (10 mL) and the pH was adjusted to pH ˜9 with a saturated aq. NaHCO₃ solution (10 mL). The mixture was extracted with dichloromethane (20 mL×3). The combined organic layer was washed with brine (25 mL×3), dried over sodium sulfate, filtered and concentrated under reduced pressure. The residue was purified by prep-HPLC (acetonitrile/water gradient with 0.05% NH₃ and 10 mM NH₄HCO₃) to give the first eluting peak as a pure single stereoisomer of the above titled compound (58 mg, 40% yield). LCMS (ESI) [M+H]⁺=495.0. ¹H NMR (400 MHz, CDCl₃) δ 8.84 (d, J=1.6 Hz, 1H), 8.37 (d, J=2.4 Hz, 1H), 7.89 (s, 1H), 6.81-6.21 (m, 2H), 4.53-4.34 (m, 1H), 4.08 (s, 4H), 2.95-2.76 (m, 4H), 2.60 (t, J=11.2 Hz, 1H), 2.23 (d, J=11.2 Hz, 1H), 2.17 (t, J=7.2 Hz, 2H), 2.13-2.01 (m, 4H), 1.59 (s, 2H), 1.54 (d, J=6.4 Hz, 6H), 1.49-1.29 (m, 2H). The trans relative stereochemistry was assigned based on ¹H NMR analysis.

### Step 3B: (Cis)-6-(4-(3-(5-(Difluoromethoxy)pyridin-3-yl)-1-isopropyl-1H-pyrazol-5-yl)cyclohexyl)-2-thia-6-azaspiro[3.4]octane 2,2-dioxide (Compound 33B)

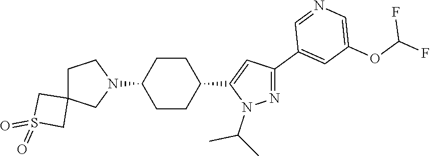 [SMILES: CC(C)n1nc(-c2cncc(OC(F)F)c2)cc1[C@H]1CC[C@@H](N2CCC3(C2)CS(=O)(=O)C3)CC1]

The title compound was synthesized following a procedure similar to Compound 33A. The crude mixture was purified by prep-HPLC (acetonitrile/water gradient with 0.05% NH₃ and 10 mM NH₄HCO₃) to give the second eluting peak as a pure single stereoisomer of the above titled compound (32.6 mg, 22% yield). LCMS (ESI) [M+H]⁺=495.0. ¹H NMR (400 MHz, CDCl₃) (400 MHz, CDCl₃) δ 8.87 (d, J=1.6 Hz, 1H), 8.37 (d, J=2.4 Hz, 1H), 7.89 (s, 1H), 6.96-6.23 (m, 2H), 4.47 (td, J=6.4, 12.8 Hz, 1H), 4.25-3.88 (m, 4H), 2.85 (s, 2H), 2.77-2.72 (m, 2H), 2.72-2.65 (m, 1H), 2.44 (s, 1H), 2.20-2.16 (m, 2H), 1.99-1.80 (m, 4H), 1.72-1.60 (m, 4H), 1.54 (d, J=6.4 Hz, 6H). The relative cis stereochemistry was assigned based on ¹H NMR analysis.

### Example HH: 6-((1r,4r)-4-(3-(3,3-Difluoroazetidin-1-yl)-1-isopropyl-1H-pyrazol-5-yl)cyclohexyl)-2-thia-6-azaspiro[3.4]octane 2,2-dioxide (Compound 35)

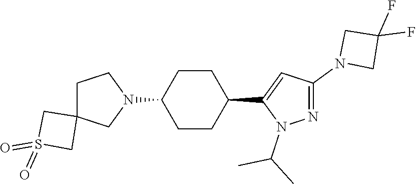 [SMILES: CC(C)n1nc(N2CC(F)(F)C2)cc1[C@H]1CC[C@H](N2CCC3(C2)CS(=O)(=O)C3)CC1]

### Step 1: 6-((1r,4r)-4-(3-Bromo-1-isopropyl-1H-pyrazol-5-yl)cyclohexyl)-2-thia-6-azaspiro[3.4]octane 2,2-dioxide

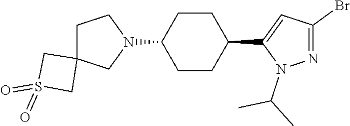 [SMILES: CC(C)n1nc(Br)cc1[C@H]1CC[C@H](N2CCC3(C2)CS(=O)(=O)C3)CC1]

To a solution of 2-thia-6-azaspiro[3.4]octane 2,2-dioxide (735 mg, 4.56 mmol) and 4-(3-bromo-1-isopropyl-1H-pyrazol-5-yl)cyclohexanone (1000.0 mg, 3.51 mmol) in methanol (20 mL) was added acetic acid (631 mg, 10.52 mmol) and NaBH₃CN (661 mg, 10.52 mmol). The mixture was stirred at 50° C. for 16 h. The reaction mixture was diluted with aq. NaHCO₃ (50 mL) and extracted with dichloromethane (50 mL×3). The combined organic layer was dried over Na₂SO₄, filtered and concentrated under reduced pressure. The residue was purified by silica column chromatography (0-2% methanol in dichloromethane) to give the second eluting peak as a pure single stereoisomer of the above titled compound (1000 mg, 66% yield). ¹H NMR (400 MHz, CDCl₃) δ 5.95 (s, 1H), 4.46-4.31 (m, 1H), 4.08 (s, 4H), 2.92 (s, 2H), 2.80 (t, J=6.8 Hz, 2H), 2.54 (t, J=11.6 Hz, 1H), 2.32-2.16 (m, 3H), 2.11-1.89 (m, 4H), 1.48 (d, J=6.4 Hz, 6H), 1.45-1.21 (m, 4H).

### Step 2: 2-((1s,4s)-4-(3-(5-(Difluoromethoxy)pyridin-3-yl)-1-isopropyl-1H-pyrazol-5-yl)cyclohexyl)-7-oxa-2-azaspiro[3.5]nonane

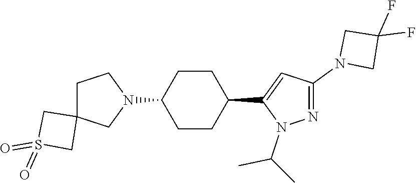 [SMILES: CC(C)n1nc(N2CC(F)(F)C2)cc1[C@H]1CC[C@H](N2CCC3(C2)CS(=O)(=O)C3)CC1]

To a solution of 6-((1r,4r)-4-(3-bromo-1-isopropyl-1H-pyrazol-5-yl)cyclohexyl)-2-thia-6-azaspiro[3.4]octane 2,2-dioxide (80 mg, 0.19 mmol) and 3,3-difluoroazetidine hydrochloride (36 mg, 0.28 mmol) in tetrahydrofuran (3 mL) was added sodium tert-butoxide (54 mg, 0.56 mmol) and Pd-PEPPSI-IHeptCl-Py (21 mg, 0.02 mmol) in a glovebox. The resulting mixture was stirred at 60° C. under a N2 atmosphere for 16 h. The reaction mixture was filtered and concentrated under reduced pressure. The resulting residue was purified by reverse phase chromatography (acetonitrile/water gradient with 0.05% NH₃ and 10 mM NH₄HCO₃) to provide the title compound (32.6 mg, 38% yield) as a pure single stereoisomer. LCMS (ESI) [M+H]⁺=443.1. ¹H NMR (400 MHz, CD₃OD) δ 5.42 (s, 1H), 4.48-4.41 (m, 1H), 4.19-4.16 (m, 3H), 4.13-4.08 (m, 5H), 2.97 (s, 2H), 2.82 (t, J=7.2 Hz, 2H), 2.64-2.58 (m, 1H), 2.27-2.18 (m, 3H), 2.11-2.08 (m, 2H), 1.97-1.94 (m, 2H), 1.53-1.43 (m, 3H), 1.39 (d, J=6.4 Hz, 6H), 1.38-1.28 (m, 1H). The relative trans stereochemistry was assigned based on ¹H NMR analysis.

### Example II: (Compounds 36A* and 36B*): 6-((1r,4r)-4-(1-Ethyl-3-(6-(trifluoromethyl)pyridin-3-yl)-1H-1,2,4-triazol-5-yl)cyclohexyl)-2-thia-6-azaspiro[3.4]octane 2,2-dioxide and 6-((1s,4s)-4-(1-ethyl-3-(6-(trifluoromethyl)pyridin-3-yl)-1H-1,2,4-triazol-5-yl)cyclohexyl)-2-thia-6-azaspiro[3.4]octane 2,2-dioxide

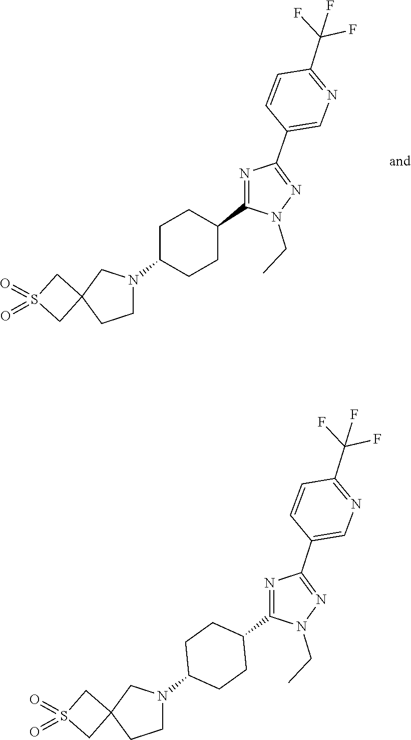 [SMILES: CCn1nc(-c2ccc(C(F)(F)F)nc2)nc1[C@H]1CC[C@H](N2CCC3(C2)CS(=O)(=O)C3)CC1 ; CCn1nc(-c2ccc(C(F)(F)F)nc2)nc1[C@H]1CC[C@@H](N2CCC3(C2)CS(=O)(=O)C3)CC1]

### Step 1: 4-(1-Ethyl-3-(6-(trifluoromethyl)pyridin-3-yl)-1H-1,2,4-triazol-5-yl)cyclohexan-1-one

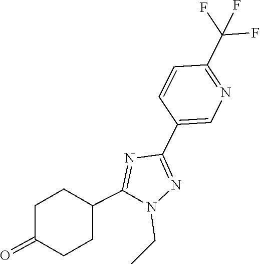 [SMILES: CCn1nc(-c2ccc(C(F)(F)F)nc2)nc1C1CCC(=O)CC1]

Following the general procedure in J. Org. Chem. 2011, 76, 1177, the title compound (130 mg, 54% yield) was obtained using silica flash column chromatography (heptanes/isopropylaceate). LCMS (ESI) [M+H]⁺=353.2.

### Step 2: 6-(4-(1-Ethyl-3-(6-(trifluoromethyl)pyridin-3-yl)-1H-1,2,4-triazol-5-yl)cyclohexyl)-2-thia-6-azaspiro[3.4]octane 2,2-dioxide

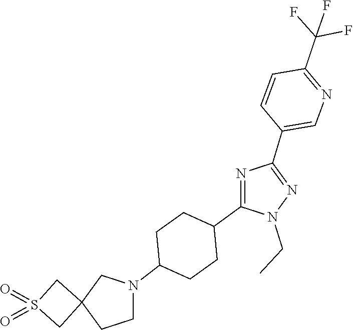 [SMILES: CCn1nc(-c2ccc(C(F)(F)F)nc2)nc1C1CCC(N2CCC3(C2)CS(=O)(=O)C3)CC1]

To a mixture of 4-(1-ethyl-3-(6-(trifluoromethyl)pyridin-3-yl)-1H-1,2,4-triazol-5-yl)cyclohexan-1-one (160 mg, 0.47 mmol) and 2-thia-6-azaspiro[3.4]octane 2,2-dioxide hydrochloride (197 mg, 0.95 mmol) in dichloroethane (2.4 mL) was added 4 angstrom molecular sieves (200 mg). The reaction was stirred at 40° C. for 18 h whereupon acetic acid (27 μL, 0.47 mmol) and sodium triacetoxyborohydride (150 mg, 0.71 mmol) were added. The reaction was stirred for an additional 18 h at 25° C. then diluted with DCM, washed with an aq. saturated sodium bicarbonate solution, dried over Mg₂SO₄, filtered and concentrated in vacuo.

The mixture of cis and trans isomers was separated by reverse phase HPLC (acetonitrile/water gradient with 0.1% TFA) to afford the first eluting peak as a pure single stereoisomer of the title compound (41 mg, 14.5%), Compound 36A*. LCMS (ESI) [M+H]⁺=484.2. ¹H NMR (400 MHz, DMSO-d₆) δ 9.33 (d, J=2.0 Hz, 1H), 8.60-8.51 (m, 1H), 8.06-7.98 (m, 1H), 4.45-4.32 (m, 3H), 4.29-4.21 (m, 3H), 4.00-3.52 (m, 3H), 3.39-3.25 (m, 2H), 2.49-2.40 (m, 1H), 2.35-2.25 (m, 1H), 2.14-1.75 (m, 9H), 1.43 (t, J=7.2 Hz, 3H).

The second eluting peak was a pure single stereoisomer of the title compound (38 mg, 13.4%) Compound 36B*. LCMS (ESI) [M+H]⁺=484.2. ¹H NMR (400 MHz, DMSO-d₆) δ 9.26 (d, J=2.1 Hz, 1H), 8.56-8.48 (m, 1H), 8.02-7.97 (m, 1H), 4.45-4.33 (m, 3H), 4.32-4.24 (m, 3H), 3.86 (dd, J=12.0, 5.9 Hz, 1H), 3.68-3.53 (m, 2H), 3.42-3.28 (m, 1H), 3.08-2.98 (m, 1H), 2.49-2.37 (m, 2H), 2.35-2.25 (m, 1H), 2.15 (dt, J=22.8, 11.5 Hz, 2H), 2.07-2.01 (m, 2H), 1.78-1.49 (m, 4H), 1.43 (t, J=7.2 Hz, 3H).

### Example JJ: (Compounds 38A*, 38B*, 38C* and 38D*) (S)-7-((1s,4R)-4-(1-Methyl-3-(trifluoromethyl)-1H-pyrazol-5-yl)cyclohexyl)-2-thia-7-azaspiro[4.4]nonane 2,2-dioxide; (R)-7-((1s,4S)-4-(1-methyl-3-(trifluoromethyl)-1H-pyrazol-5-yl)cyclohexyl)-2-thia-7-azaspiro[4.4]nonane 2,2-dioxide; (S)-7-((1r,4S)-4-(1-methyl-3-(trifluoromethyl)-1H-pyrazol-5-yl)cyclohexyl)-2-thia-7-azaspiro[4.4]nonane 2,2-dioxide and (R)-7-((1r,4R)-4-(1-methyl-3-(trifluoromethyl)-1H-pyrazol-5-yl)cyclohexyl)-2-thia-7-azaspiro[4.4]nonane 2,2-dioxide

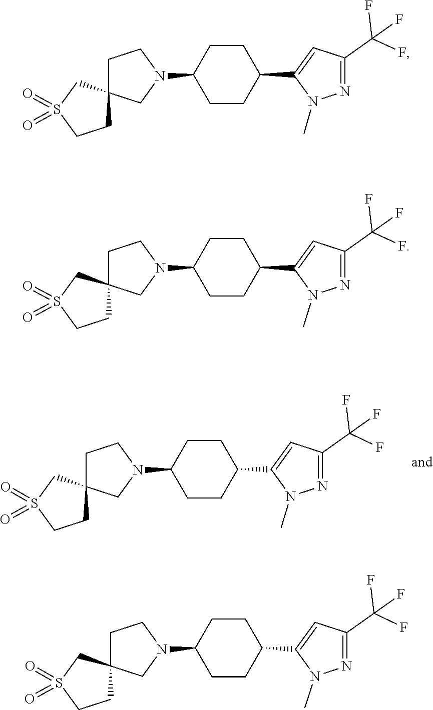 [SMILES: Cn1nc(C(F)(F)F)cc1[C@H]1CC[C@@H](N2CC[C@]3(CCS(=O)(=O)C3)C2)CC1 ; Cn1nc(C(F)(F)F)cc1[C@H]1CC[C@@H](N2CC[C@@]3(CCS(=O)(=O)C3)C2)CC1 ; Cn1nc(C(F)(F)F)cc1[C@H]1CC[C@H](N2CC[C@]3(CCS(=O)(=O)C3)C2)CC1 ; Cn1nc(C(F)(F)F)cc1[C@H]1CC[C@H](N2CC[C@@]3(CCS(=O)(=O)C3)C2)CC1]

The title compounds were synthesized generally following the procedure of Compound 15, using methyl hydrazine in step 1. The mixture of isomers was separated by chiral SFC (Chiralpak IA, 0.1% ammonium hydroxide in methanol/CO₂ 15:85) to provide 4 isomers as pure single undefined enantiomers.

Compound 38A*: LCMS (ESI) [M+H]⁺=406.2. ¹H NMR (400 MHz, DMSO-d₆) δ 6.47 (s, 1H), 3.84 (s, 3H), 3.23-3.07 (m, 4H), 2.84-2.76 (m, 1H), 2.71-2.61 (m, 2H), 2.59-2.52 (m, 1H), 2.43 (d, J=9.1 Hz, 1H), 2.31-2.27 (m, 1H), 2.24-2.07 (m, 2H), 1.96-1.64 (m, 6H), 1.61-1.48 (m, 4H).

Compound 38B*: LCMS (ESI) [M+H]⁺=406.2.

Compound 38C*: LCMS (ESI) [M+H]⁺=406.2. ¹H NMR (400 MHz, DMSO-d₆) δ 6.49 (s, 1H), 3.84 (s, 3H), 3.20-3.13 (m, 2H), 3.12-3.04 (m, 2H), 2.76-2.65 (m, 3H), 2.64-2.56 (m, 1H), 2.55-2.51 (m, 1H), 2.19-2.06 (m, 3H), 2.00-1.74 (m, 6H), 1.45-1.22 (m, 4H)

Compound 38D*: LCMS (ESI) [M+H]⁺=406.2. ¹H NMR (400 MHz, DMSO-d₆) (400 MHz, DMSO-d₆) δ 6.49 (s, 1H), 3.84 (s, 3H), 3.20-3.13 (m, 2H), 3.13-3.04 (m, 2H), 2.76-2.65 (m, 3H), 2.64-2.52 (m, 2H), 2.21-2.04 (m, 3H), 2.01-1.82 (m, 5H), 1.82-1.72 (m, 1H), 1.45-1.22 (m, 4H).

### Example KK: 7-((1r,4r)-4-(1-Cyclopropyl-3-(trifluoromethyl)-1H-pyrazol-5-yl)cyclohexyl)-2-thia-7-azaspiro[3.5]nonane 2,2-dioxide (Compound 40A) and 7-((1s,4s)-4-(1-cyclopropyl-3-(trifluoromethyl)-1H-pyrazol-5-yl)cyclohexyl)-2-thia-7-azaspiro[3.5]nonane 2,2-dioxide (Compound 40B)

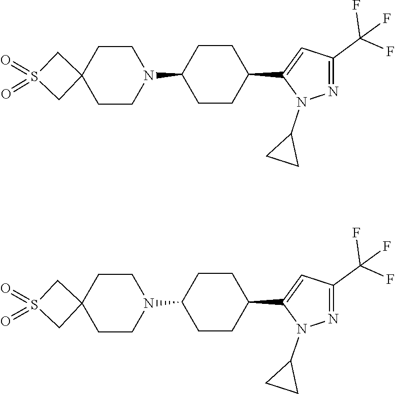 [SMILES: O=S1(=O)CC2(CCN([C@H]3CC[C@@H](c4cc(C(F)(F)F)nn4C4CC4)CC3)CC2)C1 ; O=S1(=O)CC2(CCN([C@H]3CC[C@H](c4cc(C(F)(F)F)nn4C4CC4)CC3)CC2)C1]

The title compounds were synthesized generally following the procedure of Compound 15, using cyclopropyl hydrazine in step 1 and 2-thia-7-azaspiro[3.5]nonane 2,2-dioxide hydrochloride in step 2. Purification of the crude mixture by reverse phase HPLC afforded the title compounds.

7-((1r,4r)-4-(1-cyclopropyl-3-(trifluoromethyl)-1H-pyrazol-5-yl)cyclohexyl)-2-thia-7-azaspiro[3.5]nonane 2,2-dioxide (Compound 40A). LCMS (ESI) [M+H]⁺=432.2. ¹H NMR (400 MHz, DMSO-d₆) δ 6.49 (s, 1H), 3.91 (s, 4H), 3.75-3.65 (m, 1H), 3.18-3.07 (m, 1H), 2.46-2.27 (m, 4H), 2.26-2.18 (m, 1H), 1.92-1.84 (m, 2H), 1.83-1.73 (m, 6H), 1.73-1.62 (m, 2H), 1.58-1.46 (m, 2H), 1.14-0.98 (m, 4H). The relative stereochemistry was assigned by ¹H NMR analysis.

7-((1s,4s)-4-(1-cyclopropyl-3-(trifluoromethyl)-1H-pyrazol-5-yl)cyclohexyl)-2-thia-7-azaspiro[3.5]nonane 2,2-dioxide (Compound 40B). LCMS (ESI) [M+H]⁺=432.2. ¹H NMR (400 MHz, DMSO-d₆) δ 6.49 (s, 1H), 3.91 (s, 4H), 3.75-3.65 (m, 1H), 2.93-2.82 (m, 1H), 2.48-2.41 (m, 4H), 2.40-2.30 (m, 1H), 2.05-1.96 (m, 2H), 1.86-1.79 (m, 2H), 1.76 (t, J=5.3 Hz, 4H), 1.47-1.32 (m, 4H), 1.14-0.98 (m, 4H). The relative stereochemistry was assigned by ¹H NMR analysis.

### Example LL: (Compounds 42A*, 42B*, 42C*, and 42D*): 7-((1S,3R)-3-(1-Cyclopropyl-3-(6-(trifluoromethyl)pyridin-2-yl)-1H-1,2,4-triazol-5-yl)cyclopentyl)-2-thia-7-azaspiro[3.5]nonane 2,2-dioxide, 7-((1R,3R)-3-(1-cyclopropyl-3-(6-(trifluoromethyl)pyridin-2-yl)-1H-1,2,4-triazol-5-yl)cyclopentyl)-2-thia-7-azaspiro[3.5]nonane 2,2-dioxide, 7-((1S,3S)-3-(1-Cyclopropyl-3-(6-(trifluoromethyl)pyridin-2-yl)-1H-1,2,4-triazol-5-yl)cyclopentyl)-2-thia-7-azaspiro[3.5]nonane 2,2-dioxide, and 7-((1R,3S)-3-(1-Cyclopropyl-3-(6-(trifluoromethyl)pyridin-2-yl)-1H-1,2,4-triazol-5-yl)cyclopentyl)-2-thia-7-azaspiro[3.5]nonane 2,2-dioxide

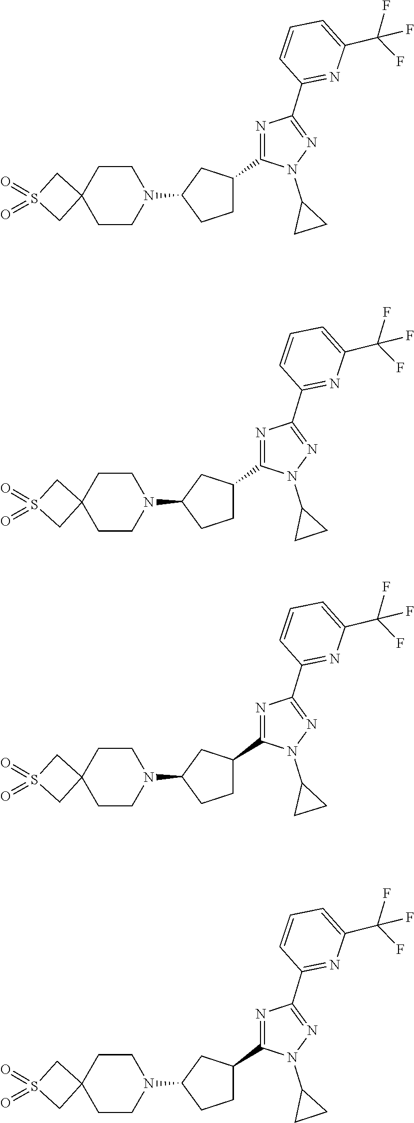 [SMILES: O=S1(=O)CC2(CCN([C@H]3CC[C@@H](c4nc(-c5cccc(C(F)(F)F)n5)nn4C4CC4)C3)CC2)C1 ; O=S1(=O)CC2(CCN([C@@H]3CC[C@@H](c4nc(-c5cccc(C(F)(F)F)n5)nn4C4CC4)C3)CC2)C1 ; O=S1(=O)CC2(CCN([C@@H]3CC[C@H](c4nc(-c5cccc(C(F)(F)F)n5)nn4C4CC4)C3)CC2)C1 ; O=S1(=O)CC2(CCN([C@H]3CC[C@H](c4nc(-c5cccc(C(F)(F)F)n5)nn4C4CC4)C3)CC2)C1]

The title compound was synthesized following a procedure similar to that of Compound 36A. The cis/trans mixture was separated by chiral SFC (Chiralpak IH, 0.1% ammonium hydroxide in methanol/CO₂ 15:85) to provide the second eluting peak as a pure single undefined stereoisomer of the title compound (14 mg, 5% yield; Compound 42A*). LCMS (ESI) [M+H]⁺=496.1. ¹H NMR (400 MHz, DMSO-d₆) δ 8.28 (d, J=7.9 Hz, 1H), 8.21-8.12 (m, 1H), 7.94-7.88 (m, 1H), 4.00-3.85 (m, 4H), 3.78-3.68 (m, 1H), 3.61-3.48 (m, 1H), 3.30-3.23 (m, 1H), 2.77-2.66 (m, 1H), 2.47-2.39 (m, 2H), 2.38-2.27 (m, 2H), 2.17-2.07 (m, 1H), 1.97-1.85 (m, 2H), 1.84-1.72 (m, 5H), 1.72-1.61 (m, 1H), 1.24-1.06 (m, 4H).

Also provided was the first eluting peak as a pure single undefined stereoisomer of title compound (44 mg, 17% yield; Compound 42B*). LCMS (ESI) [M+H]⁺=496.1. ¹H NMR (400 MHz, DMSO-d₆) δ 8.27 (d, J=7.9 Hz, 1H), 8.21-8.12 (m, 1H), 7.94-7.88 (m, 1H), 3.92 (s, 4H), 3.79-3.69 (m, 1H), 3.69-3.56 (m, 1H), 2.82 (p, J=7.6 Hz, 1H), 2.49-2.28 (m, 4H), 2.25-2.14 (m, 1H), 2.14-1.96 (m, 3H), 1.94-1.75 (m, 5H), 1.60-1.48 (m, 1H), 1.24-1.07 (m, 4H).

The title compound was synthesized following a procedure similar to that of Compound 36A. The cis/trans mixture was separated by chiral SFC (Chiralpak IA, 0.1% ammonium hydroxide in methanol/CO₂ 25:75) to provide the second eluting peak as a pure single undefined stereoisomer of the title compound (47 mg, 25% yield; Compound 42C*). LCMS (ESI) [M+H]⁺=496.1. ¹H NMR (400 MHz, DMSO-d₆) δ 8.27 (d, J=7.9 Hz, 1H), 8.23-8.16 (m, 1H), 7.97-7.90 (m, 1H), 4.19-4.07 (m, 4H), 3.80-3.55 (m, 5H), 3.06-2.92 (m, 2H), 2.62-2.54 (m, 1H), 2.28-2.14 (m, 5H), 2.08-1.92 (m, 4H), 1.24-1.12 (m, 4H).

Also provided was the first eluting peak as a pure single undefined stereoisomer (cis or trans) of the title compound (9.5 mg, 5% yield; Compound 42D*). LCMS (ESI) [M+H]⁺=496.1. ¹H NMR (400 MHz, DMSO-d₆) δδ 8.27 (d, J=7.9 Hz, 1H), 8.22-8.12 (m, 1H), 7.95-7.87 (m, 1H), 3.92 (s, 4H), 3.79-3.69 (m, 1H), 3.69-3.56 (m, 1H), 2.88-2.75 (m, 1H), 2.42-2.36 (m, 4H), 2.25-2.08 (m, 1H), 2.14-2.02 (m, 1H), 2.06-1.95 (m, 2H), 1.95-1.79 (m, 1H), 1.79 (t, J=5.4 Hz, 4H), 1.61-1.46 (m, 1H), 1.26-1.00 (m, 4H).

### Example MM: (Compounds 43A* and 43B*): (trans)-7-(-4-(1-Ethyl-3-(6-(trifluoromethyl)pyridin-3-yl)-1H-1,2,4-triazol-5-yl)cyclohexyl)-2-thia-7-azaspiro[3.5]nonane 2,2-dioxide and (cis)-7-(-4-(1-ethyl-3-(6-(trifluoromethyl)pyridin-3-yl)-1H-1,2,4-triazol-5-yl)cyclohexyl)-2-thia-7-azaspiro[3.5]nonane 2,2-dioxide

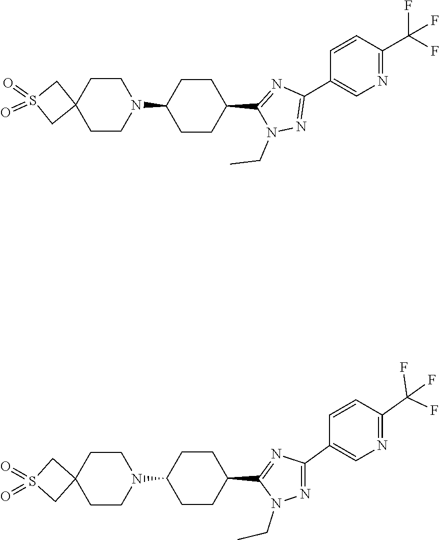 [SMILES: CCn1nc(-c2ccc(C(F)(F)F)nc2)nc1[C@H]1CC[C@@H](N2CCC3(CC2)CS(=O)(=O)C3)CC1 ; CCn1nc(-c2ccc(C(F)(F)F)nc2)nc1[C@H]1CC[C@H](N2CCC3(CC2)CS(=O)(=O)C3)CC1]

### 7-(4-(1-Ethyl-3-(6-(trifluoromethyl)pyridin-3-yl)-1H-1,2,4-triazol-5-yl)cyclohexyl)-2-thia-7-azaspiro[3.5]nonane 2,2-dioxide

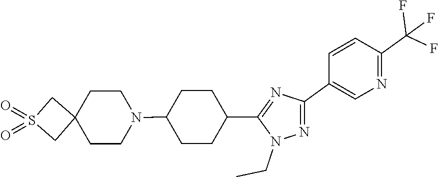 [SMILES: CCn1nc(-c2ccc(C(F)(F)F)nc2)nc1C1CCC(N2CCC3(CC2)CS(=O)(=O)C3)CC1]

The title compounds were synthesized following a procedure similar to Compound 36A. The cis/trans mixture was purified by reverse phase HPLC affording the title compounds.

Compound 43A: LCMS (ESI) [M+H]⁺=498.2 ¹H NMR (400 MHz, DMSO-d₆) δ 9.30-9.25 (m, 1H), 8.57-8.50 (m, 1H), 8.03-7.96 (m, 1H), 4.24 (q, J=7.2 Hz, 2H), 3.94-3.89 (m, 4H), 3.22-3.15 (m, 1H), 2.49-2.35 (m, 4H), 2.31-2.25 (m, 1H), 2.08-1.88 (m, 4H), 1.84-1.76 (m, 4H), 1.70-1.60 (m, 2H), 1.60-1.50 (m, 2H), 1.41 (t, J=7.2 Hz, 3H). The relative stereochemistry was assigned by ¹H NMR analysis.

Compound 43B: LCMS (ESI) [M+H]⁺=498.2 ¹H NMR (400 MHz, DMSO-d₆) δ 9.26 (d, J=2.0 Hz, 1H), 8.56-8.49 (m, 1H), 8.01-7.95 (m, 1H), 4.25 (q, J=7.2 Hz, 2H), 3.94-3.89 (m, 4H), 2.98-2.86 (m, 1H), 2.49-2.37 (m, 5H), 1.98-1.91 (m, 2H), 1.88-1.73 (m, 6H), 1.72-1.58 (m, 2H), 1.50-1.37 (m, 5H). The relative stereochemistry was assigned by ¹H NMR analysis.

### Example NN: 6-(1-(3-Cyclopropyl-5-(trifluoromethyl)pyridin-2-yl)piperidin-4-yl)-2-thia-6-azaspiro[3.4]octane 2,2-dioxide, Compound 44

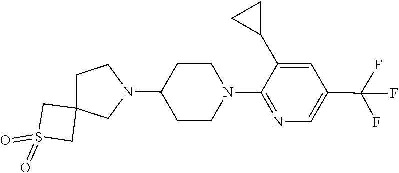 [SMILES: O=S1(=O)CC2(CCN(C3CCN(c4ncc(C(F)(F)F)cc4C4CC4)CC3)C2)C1]

### Step 1: 8-(3-Chloro-5-(trifluoromethyl)pyridin-2-yl)-1,4-dioxa-8-azaspiro[4.5]decane

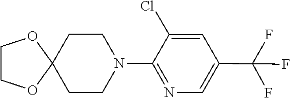 [SMILES: FC(F)(F)c1cnc(N2CCC3(CC2)OCCO3)c(Cl)c1]

To a solution of 1,4-dioxa-8-azaspiro[4.5]decane (0.42 mL, 5.01 mmol) and 3-chloro-2-fluoro-5-(trifluoromethyl)pyridine (1000 mg, 5.01 mmol) in dimethyl sulfoxide (10 mL) was added K₂CO₃ (2078 mg, 15.04 mmol) at 25° C. The reaction mixture was stirred at 100° C. for 16 h. The mixture was diluted with ethyl acetate (200 mL) and washed with water (50 mL) and brine (20 mL×3). The organic layer was dried over sodium sulfate, filtered and concentrated. The residue was purified by silica column chromatography (0-30% ethyl acetate in petroleum ether) to provide the title compound (1500 mg, 92.7% yield). LCMS (ESI) [M+H]⁺=323.1.

### Step 2: 1-[3-Chloro-5-(trifluoromethyl)-2-pyridyl]piperidin-4-one

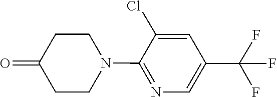 [SMILES: O=C1CCN(c2ncc(C(F)(F)F)cc2Cl)CC1]

To a suspension of 8-[3-chloro-5-(trifluoromethyl)-2-pyridyl]-1,4-dioxa-8-azaspiro[4.5]decane (1500.0 mg, 4.64 mmol) in water (20 mL) was added HCl (1.94 mL, 11.84 mmol, 6M) at 20° C. The reaction mixture was stirred at 20° C. for 2 h then cooled to 0° C. The pH was adjusted to pH ˜7 with a saturated aq. NaHCO₃ solution (10 mL). The mixture was extracted with ethyl acetate (40 mL×2). The combined extracts were washed with brine (10 mL), dried over Na₂SO₄, filtered and concentrated. The residue was purified by silica column chromatography (0-30% ethyl acetate in petroleum ether) to provide the title compound (1000 mg, 56.4% yield). LCMS (ESI) [M+H]⁺=279.1.

### Step 3:1-[3-Cyclopropyl-5-(trifluoromethyl)-2-pyridyl]piperidin-4-one

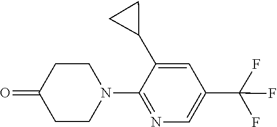 [SMILES: O=C1CCN(c2ncc(C(F)(F)F)cc2C2CC2)CC1]

To a suspension of 1-[3-chloro-5-(trifluoromethyl)-2-pyridyl]piperidin-4-one (200.0 mg, 0.72 mmol), potassium (cyclopropylmethyl)trifluoroborate (581 mg, 3.59 mmol) and Cs₂CO₃ (701 mg, 2.15 mmol) in dioxane (5 mL) and water (1 mL) was added [2-(2-aminophenyl)phenyl]-chloro-palladium; bis(1-adamantyl)-butyl-phosphane (48 mg, 0.07 mmol). The reaction mixture was stirred under N2 at 90° C. for 16 h. The reaction mixture was diluted with water (25 mL) and extracted with ethyl acetate (50 mL×2). The combined organic layer was dried over Na₂SO₄, filtered and concentrated under reduced pressure. The residue was purified by silica column chromatography (0-30% ethyl acetate in petroleum ether) to provide the title compound (200 mg, 98% yield). LCMS (ESI) [M+H]⁺=285.1.

### Step 4: 6-(1-(3-Cyclopropyl-5-(trifluoromethyl)psyridin-2-yl)piperidin-4-yl)-2-thia-6-azaspiro[3.4]octane 2,2-dioxide, Compound 44

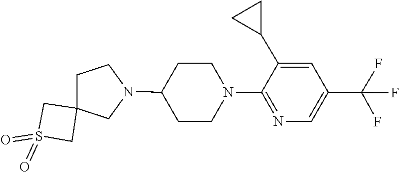 [SMILES: O=S1(=O)CC2(CCN(C3CCN(c4ncc(C(F)(F)F)cc4C4CC4)CC3)C2)C1]

To a solution of 1-[3-cyclopropyl-5-(trifluoromethyl)-2-pyridyl]piperidin-4-one (50.0 mg, 0.18 mmol), 2-thia-6-azaspiro[3.4]octane 2,2-dioxide (34 mg, 0.21 mmol) and acetic acid (0.1 mL) in methanol (2.5 mL) was added NaBH₃CN (55 mg, 0.88 mmol). The reaction mixture was stirred at 60° C. for 2 h. The reaction mixture was diluted with water (10 mL) and extracted with ethyl acetate (25 mL×3). The organic layer was washed with brine (25 mL×3), dried over Na₂SO₄, filtered and concentrated under reduced pressure. The residue was purified by silica column chromatography (0-50% ethyl acetate in petroleum ether) to provide the title compound (36.9 mg, 46.4% yield). LCMS (ESI) [M+H]⁺=429.7. ¹H NMR (400 MHz, CD₃OD) δ 8.26 (s, 1H), 7.40 (d, J=2.4 Hz, 1H), 4.14-4.12 (m, 4H), 3.98-3.95 (m, 2H), 3.00 (s, 2H), 2.99-2.91 (m, 2H), 2.86-2.82 (m, 2H), 2.46-2.38 (m, 1H), 2.21 (t, J=7.2 Hz, 2H), 2.10-1.98 (m, 3H), 1.76-1.63 (m, 2H), 1.14-1.10 (m, 2H), 0.79-0.77 (m, 2H).

### Example 00: 6-(1-(5-Fluoro-3-(trifluoromethyl)pyridin-2-yl)piperidin-4-yl)-2-thia-6-azaspiro[3.4]octane 2,2-dioxide (Compound 45)

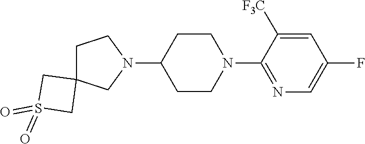 [SMILES: O=S1(=O)CC2(CCN(C3CCN(c4ncc(F)cc4C(F)(F)F)CC3)C2)C1]

### Step 1: 8-(5-Fluoro-3-(trifluoromethyl)pyridin-2-yl)-1,4-dioxa-8-azaspiro[4.5]decane

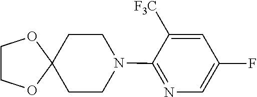 [SMILES: Fc1cnc(N2CCC3(CC2)OCCO3)c(C(F)(F)F)c1]

To a solution of 2-chloro-5-fluoro-3-(trifluoromethyl)pyridine (200 mg, 1.0 mmol), 1,4-dioxa-8-azaspiro[4.5]decane (215 mg, 1.5 mmol) and sodium tert-butoxide (289 mg, 3 mmol) in tetrahydrofuran (1 mL) was added 1,3-bis[2,6-bis(1-propylbutyl)phenyl]-4,5-dichloro-2H-imidazole, 3-chloropyridine, dichloropalladium (60 mg, 0.06 mmol). The reaction was then stirred at 60° C. under N2 for 16 h. The mixture was filtered and concentrated in vacuo. The residue was purified by silica flash chromatography to afford the title compound (287 mg, 75% yield). LCMS (ESI) [M+H]⁺=307.1.

### Step 2: 1-[5-Fluoro-3-(trifluoromethyl)-2-pyridyl] piperidin-4-one

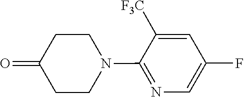 [SMILES: O=C1CCN(c2ncc(F)cc2C(F)(F)F)CC1]

To a solution of 8-[5-fluoro-3-(trifluoromethyl)-2-pyridyl]-1,4-dioxa-8-azaspiro[4.5]decane (150 mg, 0.49 mmol) was added hydrochloric acid (1.6 mL, 6.4 mmol, 4 M in 1,4-dioxane). The reaction mixture was stirred at 25° C. for 2 h. The pH of the reaction mixture was then adjusted to 11 with NaOH (2N). The aqueous phase was extracted with ethyl acetate (25 mL×3). The combined organic layers were washed with brine (15 mL×3), dried over anhydrous Na₂SO₄, filtered and concentrated in vacuo. The residue was purified by silica flash chromatography to afford the title compound (55 mg, 42.8% yield). LCMS (ESI) [M+H]⁺=263.

### Step 3: 6-(1-(5-Fluoro-3-(trifluoromethyl)pyridin-2-yl)piperidin-4-yl)-2-thia-6-azaspiro[3.4]octane 2,2-dioxide

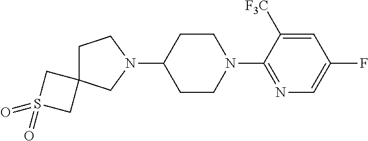 [SMILES: O=S1(=O)CC2(CCN(C3CCN(c4ncc(F)cc4C(F)(F)F)CC3)C2)C1]

To a solution of 1-[5-fluoro-3-(trifluoromethyl)-2-pyridyl]piperidin-4-one (50 mg, 0.19 mmol), 2-thia-6-azaspiro[3.4]octane 2,2-dioxide (37 mg, 0.23 mmol) and acetic acid (11 mg, 0.19 mmol) in methyl alcohol (2 mL) was added NaBH₃CN (59 mg, 0.95 mmol). The mixture was stirred at 50° C. for 3 h. The reaction mixture was diluted with dichloromethane and adjust pH to ˜9 with NaHCO₃, The resulting solution was extracted with dichloromethane. The combined organics were washed with brine, dried over anhydrous Na₂SO₄, filtered and concentrated in vacuo. The residue was purified by pre-TLC to afford the title compound (30 mg, 39% yield). LCMS (ESI) [M+H]⁺=408.2; ¹H NMR (400 MHz, CDCl₃) δ 8.33 (d, J=2.4 Hz, 1H), 7.64 (d, J=2.8, 8.0 Hz, 1H), 4.09 (s, 4H), 3.43-3.40 (m, 2H), 2.95-2.87 (m, 4H), 2.83-2.80 (m, 2H), 2.34-2.30 (m, 1H), 2.19-2.16 (m, 2H), 1.94-1.91 (m, 2H), 1.74-1.67 (m, 2H).

### Example PP: (Compounds 46A*, 46B*, 46C*, and 46D*): 7-((1R,3R)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide, 7-((1S,3R)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide, 7-((1R,3S)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide and 7-((1S,3S)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide

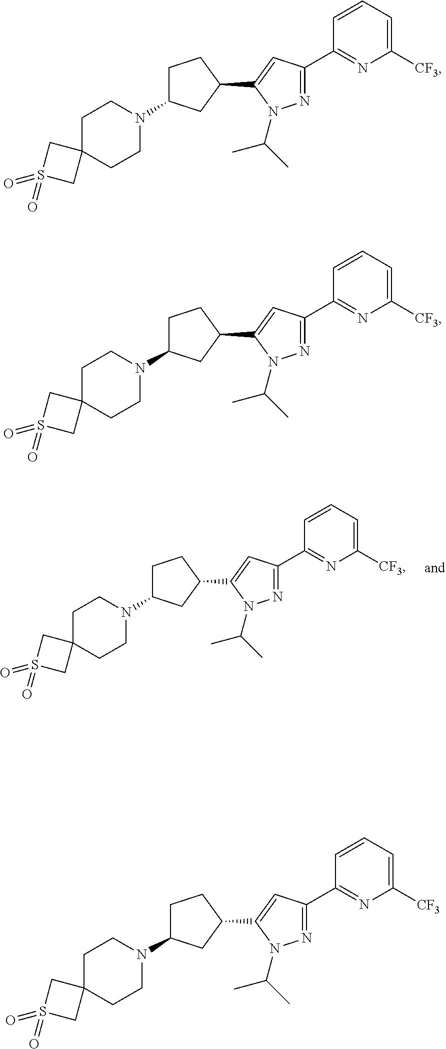 [SMILES: CC(C)n1nc(-c2cccc(C(F)(F)F)n2)cc1[C@@H]1CC[C@@H](N2CCC3(CC2)CS(=O)(=O)C3)C1 ; CC(C)n1nc(-c2cccc(C(F)(F)F)n2)cc1[C@@H]1CC[C@H](N2CCC3(CC2)CS(=O)(=O)C3)C1 ; CC(C)n1nc(-c2cccc(C(F)(F)F)n2)cc1[C@H]1CC[C@@H](N2CCC3(CC2)CS(=O)(=O)C3)C1 ; CC(C)n1nc(-c2cccc(C(F)(F)F)n2)cc1[C@H]1CC[C@H](N2CCC3(CC2)CS(=O)(=O)C3)C1]

### Step 1: 3-(1-Isopropyl-3-(6-(trifluoromethyl)pyridin-2-yl)-1H-pyrazol-5-yl)cyclopentanone

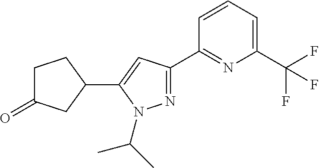 [SMILES: CC(C)n1nc(-c2cccc(C(F)(F)F)n2)cc1C1CCC(=O)C1]

To a solution of 3-(5-bromo-2-isopropyl-pyrazol-3-yl)cyclopentanone (1000 mg, 3.69 mmol), [6-(trifluoromethyl)-2-pyridyl]boronic acid (1408 mg, 7.38 mmol), bis(di-tert-butyl(4-dimethylaminophenyl)phosphine)dichloropalladium(II) (261 mg, 0.37 mmol) and K₂CO₃ (1529 mg, 11.1 mmol) in 1,4-dioxane (40 mL) and water (8 mL). The resulting mixture was stirred at 100° C. under N2 atmosphere for 3 h. The reaction mixture was diluted with water (20 mL×3) and extracted with ethyl acetate (60 mL×3). The combined organics were dried over anhydrous Na₂SO₄, filtered and the filtrate was concentrated in vacuo. The residue was purified by silica column chromatography (0-20% ethyl acetate in petroleum ether) to afford the title compound (1 g, 2.96 mmol, 80% yield). LCMS (ESI) [M+H]⁺=338.2. SFC showed two peaks.

### Step 2: (R)-3-(1-Isopropyl-3-(6-(trifluoromethyl)pyridin-2-yl)-1H-pyrazol-5-yl)cyclopentanone and (S)-3-(1-isopropyl-3-(6-(trifluoromethyl)pyridin-2-yl)-1H-pyrazol-5-yl)cyclopentanone

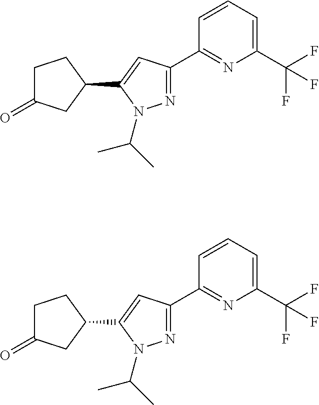 [SMILES: CC(C)n1nc(-c2cccc(C(F)(F)F)n2)cc1[C@@H]1CCC(=O)C1 ; CC(C)n1nc(-c2cccc(C(F)(F)F)n2)cc1[C@H]1CCC(=O)C1]

3-[2-Isopropyl-5-[6-(trifluoromethyl)-2-pyridyl]pyrazol-3-yl]cyclopentanone (1000 mg, 2.96 mmol) was purified by SFC (Daicel Chiralcel OD (250 mm*30 mm, 10 μm); 0.1% NH₃ in H₂O; EtOH; 45%; 60 mL/min) to afford the title compound 46A* (the first peak on SFC, 400 mg, 1.19 mmol, 40% yield) and the title compound 46B* (the second peak on SFC, 360 mg, 1.067 mmol, 36% yield). The absolute stereochemistry was assigned arbitrarily.

### Step 3: 7-((3R)-3-(1-Isopropyl-3-(6-(trifluoromethyl)pyridin-2-yl)-1H-pyrazol-5-yl)cyclopentyl)-2-thia-7-azaspiro[3.5]nonane 2,2-dioxide

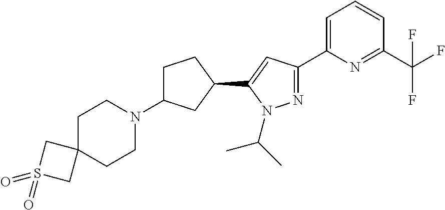 [SMILES: CC(C)n1nc(-c2cccc(C(F)(F)F)n2)cc1[C@@H]1CCC(N2CCC3(CC2)CS(=O)(=O)C3)C1]

To a solution of (3R)-3-[2-isopropyl-5-[6-(trifluoromethyl)-2-pyridyl]pyrazol-3-yl]cyclopentanone (80 mg, 0.24 mmol) in methanol (8 mL) were added 2-thia-7-azaspiro[3.5]nonane 2,2-dioxide hydrochloride (64 mg, 0.30 mmol) and acetic acid (14 mg, 0.24 mmol), then NaBH₃CN (45 mg, 0.71 mmol). The reaction mixture was stirred at 60° C. for 16 h. The reaction was diluted with NaHCO₃ (10 mL), and extracted with dichloromethane (10 mL×3). The combined organic layers were dried over anhydrous Na₂SO₄, filtered and concentrated in vacuo. The residue was purified by silica column chromatography (0-5% methyl alcohol in dichloromethane) to afford the title compound (100 mg, 0.20 mmol, 84.9% yield) as a mixture of diastereosiomers.

### Step 4: 7-((1R,3R)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide, 7-((1S,3R)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide

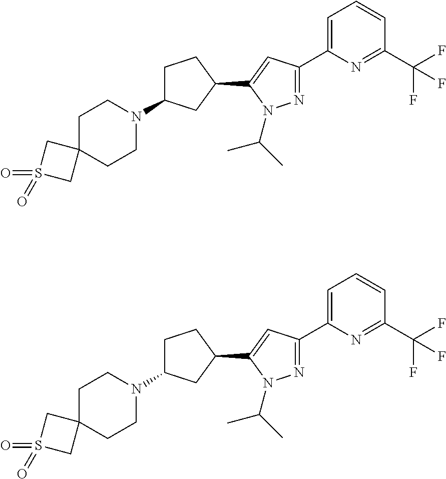 [SMILES: CC(C)n1nc(-c2cccc(C(F)(F)F)n2)cc1[C@@H]1CC[C@H](N2CCC3(CC2)CS(=O)(=O)C3)C1 ; CC(C)n1nc(-c2cccc(C(F)(F)F)n2)cc1[C@@H]1CC[C@@H](N2CCC3(CC2)CS(=O)(=O)C3)C1]

The mixture of diastereoisomers (120 mg, 0.24 mmol) was purified by SFC (Daicel Chiralcel OD (250 mm*30 mm, 10 μm), 0.1% NH₃ in water; EtOH; 45%; 60 mL/min) to provide title Compound 46A (the first peak on SFC, 73.1 mg, 0.14 mmol, 58% yield), and title Compound 46B (the second peak on SFC, 38.2 mg, 0.0722 mmol, 29.9% yield). LCMS (ESI) [M+H]⁺=497.2.

Compound 46A*: ¹H NMR (400 MHz, CD₃OD) δ 8.17 (d, J=8.0 Hz, 1H), 7.98 (t, J=8.0 Hz, 1H), 7.63 (d, J=7.2 Hz, 1H), 6.79 (s, 1H), 4.70-4.61 (m, 1H), 3.93 (s, 4H), 3.28-3.23 (m, 1H), 2.89-2.83 (m, 2H), 2.74-2.02 (m, 6H), 1.95 (t, J=5.2 Hz, 4H), 1.81-1.74 (m, 2H), 1.72-1.64 (m, 1H), 1.52 (d, J=6.4 Hz, 6H).

Compound 46B*: ¹H NMR (400 MHz, CD₃OD) δ 8.18 (d, J=8.0 Hz, 1H), 7.98 (t, J=8.0 Hz, 1H), 7.63 (d, J=7.8 Hz, 1H), 6.72 (s, 1H), 4.69-4.61 (m, 1H), 3.93 (s, 4H), 3.45-3.37 (m, 1H), 2.96-2.92 (m, 1H), 2.84-2.45 (m, 3H), 2.29-1.99 (m, 5H), 1.96 (d, J=5.2 Hz, 4H), 1.79-1.72 (m, 1H), 1.69-1.59 (m, 1H), 1.52 (d, J=6.4 Hz, 6H).

### Step 5: 7-((3S)-3-(1-Isopropyl-3-(6-(trifluoromethyl)pyridin-2-yl)-1H-pyrazol-5-yl)cyclopentyl)-2-thia-7-azaspiro[3.5]nonane 2,2-dioxide

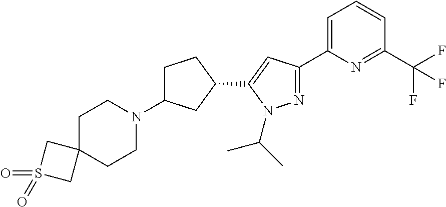 [SMILES: CC(C)n1nc(-c2cccc(C(F)(F)F)n2)cc1[C@H]1CCC(N2CCC3(CC2)CS(=O)(=O)C3)C1]

To a solution of (3S)-3-[2-isopropyl-5-[6-(trifluoromethyl)-2-pyridyl]pyrazol-3-yl]cyclopentanone (80 mg, 0.24 mmol) in methyl alcohol (8 mL) was added 2-thia-7-azaspiro[3.5]nonane 2,2-dioxide hydrochloride (64 mg, 0.30 mmol) and acetic acid (14 mg, 0.24 mmol), then NaBH₃CN (45 mg, 0.71 mmol). The reaction mixture was stirred at 60° C. for 16 h. The reaction was diluted with NaHCO₃ (10 mL), and extracted with dichloromethane (10 mL×3). The combined organic layers were dried over anhydrous Na₂SO₄, filtered and concentrated in vacuo. The residue was purified by silica column chromatography (0-5% methyl alcohol in dichloromethane) to afford the title compound (110 mg, 0.2215 mmol, 93% yield), which was obtained as a mixture of diastereosiomers.

### Step 6: 7-((1R,3S)-3-(1-Isopropyl-3-(6-(trifluoromethyl)pyridin-2-yl)-1H-pyrazol-5-yl)cyclopentyl)-2-thia-7-azaspiro[3.5]nonane 2,2-dioxide and 7-((1S,3S)-3-(1-isopropyl-3-(6-(trifluoromethyl)pyridin-2-yl)-1H-pyrazol-5-yl)cyclopentyl)-2-thia-7-azaspiro[3.5]nonane 2,2-dioxide

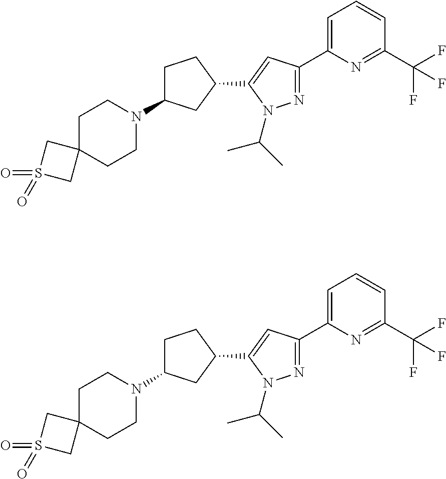 [SMILES: CC(C)n1nc(-c2cccc(C(F)(F)F)n2)cc1[C@H]1CC[C@H](N2CCC3(CC2)CS(=O)(=O)C3)C1 ; CC(C)n1nc(-c2cccc(C(F)(F)F)n2)cc1[C@H]1CC[C@@H](N2CCC3(CC2)CS(=O)(=O)C3)C1]

The mixture of diastereoisomers (120 mg, 0.24 mmol) was purified by SFC (Daicel Chiralcel OD (250 mm*30 mm, 10 μm), 0.1% NH₃ in water; EtOH; 45%; 60 mL/min) to provide the title compound 46C* (the first peak on SFC, 44.41 mg, 0.0867 mmol, 33.1% yield), and the title compound 46D* (the second peak on SFC, 81.33 mg, 0.1556 mmol, 59.4% yield). LCMS (ESI) [M+H]⁺=497.2.

Compound 46C*: ¹H NMR (400 MHz, CD₃OD) δ 8.19 (d, J=8.0 Hz, 1H), 7.99 (t, J=8.0 Hz, 1H), 7.65 (d, J=7.6 Hz, 1H), 6.73 (s, 1H), 4.68-4.62 (m, 1H), 3.93 (s, 4H), 3.45-3.37 (m, 1H), 2.94-2.90 (m, 1H), 2.60 (s, 3H), 2.30-1.99 (m, 5H), 1.96 (d, J=5.2 Hz, 4H), 1.83-1.73 (m, 1H), 1.69-1.59 (m, 1H), 1.53 (d, J=6.4 Hz, 6H).

Compound 46D*: ¹H NMR (400 MHz, CD₃OD) δ 8.19 (d, J=8.0 Hz, 1H), 7.99 (t, J=8.0 Hz, 1H), 7.64 (d, J=7.6 Hz, 1H), 6.80 (s, 1H), 4.68-4.60 (m, 1H), 3.93 (s, 4H), 3.29-3.24 (m, 1H), 2.89-2.83 (m, 2H), 2.74-2.02 (m, 6H), 1.95 (d, J=5.2 Hz, 4H), 1.85-1.75 (m, 2H), 1.70-1.65 (m, 1H), 1.53 (d, J=6.4 Hz, 6H).

### Example QQ: 6-((1s,4s)-4-(3-methyl-5-(trifluoromethyl)pyridin-2-yl)cyclohexyl)-2-thia-6-azaspiro[3.4]octane 2,2-dioxide (Compound 47A) and 6-((1r,4r)-4-(3-methyl-5-(trifluoromethyl)pyridin-2-yl)cyclohexyl)-2-thia-6-azaspiro[3.4]octane 2,2-dioxide (Compound 47B)

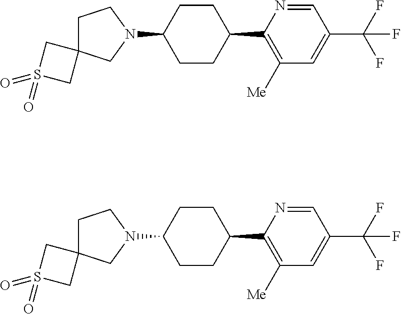 [SMILES: Cc1cc(C(F)(F)F)cnc1[C@H]1CC[C@@H](N2CCC3(C2)CS(=O)(=O)C3)CC1 ; Cc1cc(C(F)(F)F)cnc1[C@H]1CC[C@H](N2CCC3(C2)CS(=O)(=O)C3)CC1]

### Step 1: 3-Chloro-2-(1,4-dioxaspiro[4.5]dec-7-en-8-yl)-5-(trifluoromethyl)pyridine

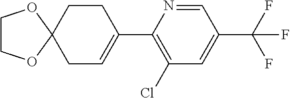 [SMILES: FC(F)(F)c1cnc(C2=CCC3(CC2)OCCO3)c(Cl)c1]

To a solution of 2-bromo-3-chloro-5-(trifluoromethyl)pyridine (2.0 g, 7.68 mmol), 1,4-dioxa-spiro[4,5]dec-7-en-8-boronic acid pinacol ester (2.04 g, 7.68 mmol) and potassium carbonate (3.18 g, 23.0 mmol) in 1,4-dioxane (24 mL) and water (6 mL) was added Pd(dppf)Cl₂ (561 mg, 0.77 mmol). The reaction mixture was stirred under N2 at 90° C. 16 h. The reaction mixture was concentrated in vacuo and the residue was purified by silica column chromatography (0-20% ethyl acetate in petroleum ether) to afford the title compound (2400 mg, 7.51 mmol, 98% yield). LCMS (ESI) [M+H]⁺=320.1.

### Step 2: 3-Methyl-2-(1,4-dioxaspiro[4.5]dec-7-en-8-yl)-5-(trifluoromethyl)pyridine

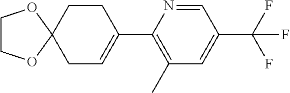 [SMILES: Cc1cc(C(F)(F)F)cnc1C1=CCC2(CC1)OCCO2]

To a solution of 3-chloro-2-(1,4-dioxaspiro[4.5]dec-7-en-8-yl)-5-(trifluoromethyl) pyridine (800.0 mg, 2.5 mmol), potassium methyltrifluoroborate (610 mg, 5 mmol) and sodium carbonate (3.75 ml, 7.50 mmol, 2N) in 1,4-dioxane (16 mL) was added RuPhos Pd G2 (389 mg, 0.50 mmol). The reaction mixture was stirred under N2 at 110° C. for 16 h. The reaction mixture was then concentrated in vacuo and the residue was purified by silica column chromatography (0-20% ethyl acetate in petroleum ether) to afford the title compound (540 mg, 1.80 mmol, 72% yield). LCMS (ESI), [M+H]+=300.2.

### Step 3: 3-Methyl-2-(1,4-dioxaspiro[4.5]decan-8-yl)-5-(trifluoromethyl)pyridine

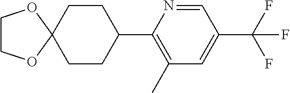 [SMILES: Cc1cc(C(F)(F)F)cnc1C1CCC2(CC1)OCCO2]

To a solution of 2-(1,4-dioxaspiro[4.5]decan-8-yl)-3-methyl-5-(trifluoromethyl)pyridine (500 mg, 1.66 mmol) in THF (10 mL) was added 10% palladium on carbon (150 mg). The reaction mixture was stirred under H₂ (15 psi) at 20° C. for 4 h. The reaction mixture was filtered through diatomite and the filtrate was concentrated in vacuo to afford the title compound (500 mg, 1.66 mmol, 100% yield), which was used directly without further purification. LCMS (ESI) [M+H]⁺=302.1.

### Step 4: 4-(3-Methyl-5-(trifluoromethyl)pyridin-2-yl)cyclohexanone

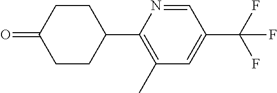 [SMILES: Cc1cc(C(F)(F)F)cnc1C1CCC(=O)CC1]

A solution of 8-[3-fluoro-5-(trifluoromethyl)-2-pyridyl]-1,4-dioxa-8-azaspiro[4.5]decane (500 mg, 1.63 mmol) in HCl (2 mL, 12 mmol, 6M) was stirred at 20° C. for 16 h. The reaction mixture was adjusted to pH=9 with saturated NaHCO₃ (10 mL) at 0° C. The resulting mixture was extracted with ethyl acetate (40 mL×2). The combined organics were washed with brine (10 mL×2), dried over anhydrous Na₂SO₄, filtered and concentrated in vacuo to afford the crude title compound (400 mg, 1.526 mmol, 93.4% yield). LCMS (ESI) [M+H]⁺=258.1.

### Step 5: 6-((cis)-4-(3-Methyl-5-(trifluoromethyl)pyridin-2-yl)cyclohexyl)-2-thia-6-azaspiro[3.4]octane 2,2-dioxide (Compound 47A) and 6-((trans)-4-(3-methyl-5-(trifluoromethyl) pyridin-2-yl)cyclohexyl)-2-thia-6-azaspiro[3.4]octane 2,2-dioxide (Compound 47B)

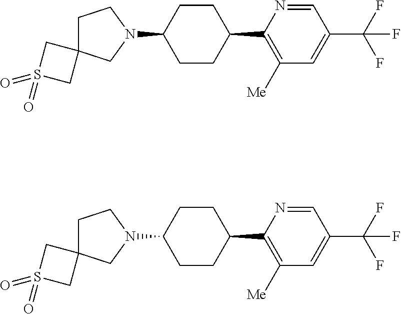 [SMILES: Cc1cc(C(F)(F)F)cnc1[C@H]1CC[C@@H](N2CCC3(C2)CS(=O)(=O)C3)CC1 ; Cc1cc(C(F)(F)F)cnc1[C@H]1CC[C@H](N2CCC3(C2)CS(=O)(=O)C3)CC1]

To a solution of 4-[3-methyl-5-(trifluoromethyl)-2-pyridyl]cyclohexanone (100 mg, 0.389 mmol), acetic acid (0.08 mL, 0.97 mmol) and 2-thia-6-azaspiro[3.4]octane 2,2-dioxide (94 mg, 0.583 mmol) in methanol (4 mL) was added sodium cyanoborohydride (73 mg, 1.18 mmol). The reaction mixture was stirred at 60° C. for 12 h. The reaction mixture was diluted with ethyl acetate (80 mL). The resulting mixture was washed with NaHCO₃ (10 mL×3). The organic phase was concentrated and the residue was purified by pre-TLC (10% dichloromethane in Methanol) to afford title Compound 47A (32.07 mg, 0.0796 mmol, 20.5% yield) and the title Compound 47B (63.48 mg, 0.157 mmol, 40.5% yield). LCMS (ESI), [M+H]⁺=403.2.

Compound 47A: ¹H NMR (400 MHz, CDCl₃) δ 8.66 (s, 1H), 7.62 (s, 1H), 4.10 (s, 4H), 3.03-2.92 (m, 1H), 2.86 (s, 2H), 2.75 (t, J=6.8 Hz, 2H), 2.44 (brs, 1H), 2.17 (t, J=7.2 Hz, 2H), 1.97-2.12-1.97 (m, 4H), 1.71 (brs, 3H), 1.65-1.47 (m, 3H), 1.26 (s, 1H).

Compound 47B: ¹H NMR (400 MHz, CDCl₃) δ 8.64 (s, 1H), 7.63 (s, 1H), 4.15-4.07 (m, 4H), 2.94 (s, 2H), 2.91-2.85 (m, 1H), 2.81 (t, J=6.8 Hz, 2H), 2.40-2.37 (m, 1H), 2.35-2.22 (m, 1H), 2.20-2.02 (m, 5H), 1.87-1.72 (m, 4H), 1.48-1.35 (m, 2H), 1.30-1.25 (m, 1H).

### Example RR: 6-((1r,4r)-4-(3-chloro-5-(trifluoromethyl)pyridin-2-yl)cyclohexyl)-2-thia-6-azaspiro[3.4]octane 2,2-dioxide (Compound 48A) and 6-((1s,4s)-4-(3-chloro-5-(trifluoromethyl)pyridin-2-yl)cyclohexyl)-2-thia-6-azaspiro[3.4]octane 2,2-dioxide (Compound 48B)

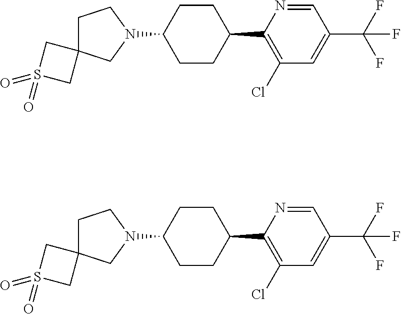 [SMILES: O=S1(=O)CC2(CCN([C@H]3CC[C@H](c4ncc(C(F)(F)F)cc4Cl)CC3)C2)C1 ; O=S1(=O)CC2(CCN([C@H]3CC[C@H](c4ncc(C(F)(F)F)cc4Cl)CC3)C2)C1]

Title compounds were synthesized following a procedure similar to Compound 47A using 2-bromo-3-chloro-5-(trifluoromethyl)pyridine.

The mixture of the diastereomers were purified by silica flash chromatography (0-10% methanol in dichloromethane) to provide Compound 48A (first peak on HPLC, 55.2 mg, 38.3% yield) and Compound 48B (second peak on HPLC, 31.4 mg, 0.0706 mmol, 21.8% yield. LCMS (ESI) [M+H]⁺=423.0

Compound 48A: ¹H NMR (400 MHz, CD₃OD) δ 8.75 (s, 1H), 8.13 (d, J=1.6 Hz, 1H), 4.15-4.08 (m, 4H), 3.28-3.27 (m, 1H), 3.03 (s, 2H), 2.87 (t, J=7.2 Hz, 2H), 2.42-2.30 (m, 1H), 2.24-2.16 (m, 4H), 1.95-1.91 (m, 2H), 1.81-1.71 (m, 2H), 1.49-1.39 (m, 2H).

Compound 48B: ¹H NMR (400 MHz, CD₃OD) δ 8.74 (s, 1H), 8.13 (d, J=1.6 Hz, 1H), 4.15-4.08 (m, 4H), 3.41-3.39 (m, 1H), 2.87 (s, 2H), 2.76 (t, J=7.2 Hz, 2H), 2.45-2.35 (m, 1H), 2.20-2.16 (m, 2H), 2.12-2.04 (m, 4H), 1.67-1.63 (m, 4H).

### Example SS: 6-((1r,4r)-4-(5-(Trifluoromethyl)pyridin-2-yl)cyclohexyl)-2-thia-6-azaspiro[3.4]octane 2,2-dioxide (Compound 49)

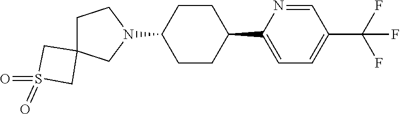 [SMILES: O=S1(=O)CC2(CCN([C@H]3CC[C@H](c4ccc(C(F)(F)F)cn4)CC3)C2)C1]

The title compound was synthesized using the same experimental procedure as Compound 50A using 2-bromo-5-(trifluoromethyl)pyridine. The relative stereochemistry was assigned based on ¹H NMR analysis. 27.4 mg (14%) of the title compound was obtained. LCMS (ESI) [M+H]⁺=389.1; ¹H NMR (400 MHz, DMSO-d₆) δ 8.86 (s, 1H), 8.10 (dd, J=8.3, 2.5 Hz, 1H), 7.52 (d, J=8.3 Hz, 1H), 4.17-4.08 (m, 4H), 2.81 (s, 2H), 2.80-2.72 (m, 1H), 2.67 (t, J=7.2 Hz, 2H), 2.23-2.11 (m, 1H), 2.07 (t, J=7.2 Hz, 2H), 2.04-1.96 (m, 2H), 1.95-1.86 (m, 2H), 1.66-1.51 (m, 2H), 1.35-1.22 (m, 2H).

### Example TT: 7-((1r,4r)-4-(5-(Trifluoromethyl)pyridin-2-yl)cyclohexyl)-2-thia-7-azaspiro[3.5]nonane 2,2-dioxide (Compound 50A) and 7-((1s,4s)-4-(5-(trifluoromethyl)pyridin-2-yl)cyclohexyl)-2-thia-7-azaspiro[3.5]nonane 2,2-dioxide (Compound 50B)

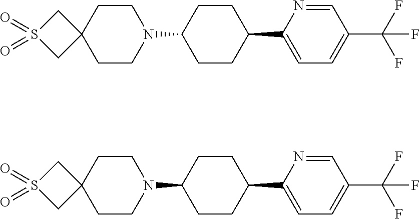 [SMILES: O=S1(=O)CC2(CCN([C@H]3CC[C@H](c4ccc(C(F)(F)F)cn4)CC3)CC2)C1 ; O=S1(=O)CC2(CCN([C@H]3CC[C@@H](c4ccc(C(F)(F)F)cn4)CC3)CC2)C1]

### Step 1: 2-(1,4-dioxaspiro[4.5]dec-7-en-8-yl)-5-(trifluoromethyl)pyridine

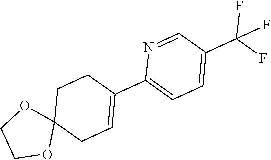 [SMILES: FC(F)(F)c1ccc(C2=CCC3(CC2)OCCO3)nc1]

A solution of 2-bromo-5-(trifluoromethyl)pyridine (2260 mg, 10.0 mmol), potassium phosphate (4245 mg, 20.0 mmol), cataCXium Pd G4 (259.8 mg, 0.35 mmol) and 4,4,5,5-tetramethyl-2-(1,4-dioxaspiro[4.5]dec-7-en-8-yl)-1,3,2-dioxaborolane (3193 mg, 12.0 mmol) in 1,4-dioxane (25.0 mL) and water (8.33 mL) was stirred at 70° C. for 18 h. The mixture was diluted with 1N aq. NH₄Cl (20 mL) and dichloromethane (60 mL). The aqueous layer was extracted with dichloromethane (2×10 mL). The combined organic layer was dried over anhydrous Na₂SO₄ and concentrated in vacuo to provide the crude title compound (4.8 g, ˜100% yield). LCMS (ESI) [M+H]⁺=286.0. ¹H NMR (400 MHz, DMSO-d₆) δ 8.89 (s, 1H), 8.13 (dd, J=8.6, 2.4 Hz, 1H), 7.76 (d, J=8.4 Hz, 1H), 6.78 (s, 1H), 3.92 (s, 4H), 2.71-2.64 (m, 2H), 2.49-2.43 (m, 2H), 1.83 (t, J=6.5 Hz, 2H).

### Step 2: 2-(1,4-dioxaspiro[4.5]decan-8-yl)-5-(trifluoromethyl)pyridine

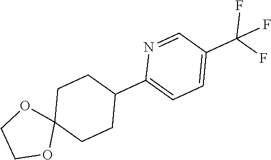 [SMILES: FC(F)(F)c1ccc(C2CCC3(CC2)OCCO3)nc1]

A solution of 2-(1,4-dioxaspiro[4.5]dec-7-en-8-yl)-5-(trifluoromethyl)pyridine (2850 mg, 10.0 mmol) and palladium hydroxide on carbon (750 mg, 5.34 mmol) in methanol (50.0 mL) was stirred at 50° C. under an atmosphere of hydrogene for 18 h. The mixture was diluted with dichloromethane (100 mL) and filtered through a pad of celite. The solid was washed with dichloromethane (2×20 mL) and the filtrate was concentrated under reduced pressure to provide the crude title compound (4170 mg, ˜100% yield). LCMS (ESI) [M+H]⁺=288.1. ¹H NMR (400 MHz, DMSO-d₆) δ 8.87 (s, 1H), 8.14-8.06 (m, 1H), 7.52 (d, J=8.2 Hz, 1H), 3.89 (s, 4H), 2.93-2.81 (m, 1H), 1.85-1.75 (m, 6H), 1.66-1.58 (m, 2H).

### Step 3: 4-(5-(trifluoromethyl)pyridin-2-yl)cyclohexan-1-one

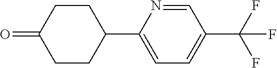 [SMILES: O=C1CCC(c2ccc(C(F)(F)F)cn2)CC1]

A solution of 2-(1,4-dioxaspiro[4.5]decan-8-yl)-5-(trifluoromethyl)pyridine (2870 mg, 10.0 mmol) in acetic acid (33.3 mL) and water (11.1 mL) was stirred at 50° C. for 18 h. The mixture was diluted with aq. sat. NaHCO₃ (50 mL), water (50 mL) and dichloromethane (50 mL). The mixture was neutralized with solid NaHCO₃ and the aqueous layer was extracted with dichloromethane (2×50 mL). The combined organic extracts was dried (Na₂SO₄) and concentrated under reduced pressure to provide the crude title compound (3360 mg, ˜100% yield). LCMS (ESI) [M+H]⁺=244.0. ¹H NMR (400 MHz, DMSO-d₆) δ 8.90 (d, J=2.4 Hz, 1H), 8.16 (dd, J=8.2, 2.5 Hz, 1H), 7.62 (d, J=8.2 Hz, 1H), 3.42-3.32 (m, 1H), 2.59 (td, J=14.0, 6.0 Hz, 2H), 2.36-2.26 (m, 2H), 2.24-2.12 (m, 2H), 2.00 (td, J=12.4, 4.3 Hz, 2H).

### Step 4: 7-((1r,4r)-4-(5-(trifluoromethyl)pyridin-2-yl)cyclohexyl)-2-thia-7-azaspiro[3.5]nonane 2,2-dioxide

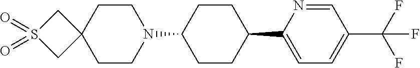 [SMILES: O=S1(=O)CC2(CCN([C@H]3CC[C@H](c4ccc(C(F)(F)F)cn4)CC3)CC2)C1]

To a solution of 2-thia-7-azaspiro[3.5]nonane 2,2-dioxide hydrochloride (138 mg, 0.65 mmol) and 4-(5-(trifluoromethyl)pyridin-2-yl)cyclohexan-1-one (122 mg, 0.50 mmol) in methanol (3.3 mL) was added N,N-diisopropylethylamine (0.261 mL, 1.50 mmol) and titanium(IV) isopropoxide (0.222 mL, 0.75 mmol). The mixture was stirred at 30° C. for 18 h. Sodium cyanoborohydride (94.3 mg, 1.50 mmol) was added and the reaction mixture was stirred at 50° C. for 18 h. The mixture was diluted with 1N aq. NH₄Cl (1.0 mL) and DMSO, filtered and purified by preparative reverse phase HPLC (acetonitrile/water gradient with 0.1% TFA) to afford the first eluting peak as a pure single stereoisomer of the title compound (Compound 50A) (39.8 mg, 20% yield). LCMS (ESI) [M+H]⁺=403.1. ¹H NMR (400 MHz, DMSO-d₆) δ 8.86 (s, 1H), 8.11 (d, J=8.4 Hz, 1H), 7.52 (d, J=8.3 Hz, 1H), 3.92 (s, 4H), 2.75 (t, J=12.3 Hz, 1H), 2.49-2.25 (m, 5H), 1.98-1.90 (m, 2H), 1.90-1.83 (m, 2H), 1.83-1.71 (m, 4H), 1.65-1.51 (m, 2H), 1.47-1.33 (m, 2H). The relative trans stereochemistry was assigned based on ¹H NMR analysis.

### 7-((1s,4s)-4-(5-(Trifluoromethyl)pyridin-2-yl)cyclohexyl)-2-thia-7-azaspiro[3.5]nonane 2,2-dioxide

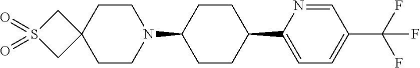 [SMILES: O=S1(=O)CC2(CCN([C@H]3CC[C@@H](c4ccc(C(F)(F)F)cn4)CC3)CC2)C1]

The crude reaction mixture from example 50A step 4 was purified by purified by preparative reverse phase HPLC (acetonitrile/water gradient with 0.1% TFA) to afford the second eluting peak as a pure single stereoisomer of the title compound (Compound 50B) (71.5 mg, 36% yield). LCMS (ESI) [M+H]⁺=403.1. ¹H NMR (400 MHz, DMSO-d₆) δ 8.89 (s, 1H), 8.10 (dd, J=8.7, 2.4 Hz, 1H), 7.53 (d, J=8.7 Hz, 1H), 3.90 (s, 4H), 3.02-2.92 (m, 1H), 2.48-2.29 (m, 4H), 2.29-2.20 (m, 1H), 2.11-1.95 (m, 2H), 1.88-1.71 (m, 6H), 1.69-1.45 (m, 4H). The relative cis stereochemistry was assigned based on ¹H NMR analysis.

### Example UU: 6-((1r,4r)-4-(2-Methyl-6-(trifluoromethyl)pyridin-3-yl)cyclohexyl)-2-thia-6-azaspiro[3.4]octane 2,2-dioxide (Compound 51A) and 6-((1s,4s)-4-(2-methyl-6-(trifluoromethyl)pyridin-3-yl)cyclohexyl)-2-thia-6-azaspiro[3.4]octane 2,2-dioxide (Compound 51B)

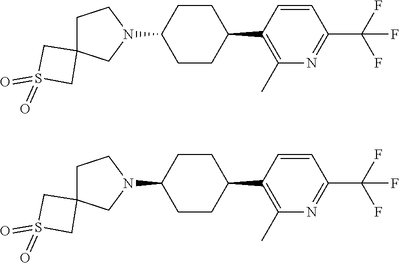 [SMILES: Cc1nc(C(F)(F)F)ccc1[C@H]1CC[C@H](N2CCC3(C2)CS(=O)(=O)C3)CC1 ; Cc1nc(C(F)(F)F)ccc1[C@H]1CC[C@@H](N2CCC3(C2)CS(=O)(=O)C3)CC1]

Title compounds were synthesized following a procedure similar to Compound 47A using 3-bromo-2-methyl-6-(trifluoromethyl)pyridine.

The mixture of the diastereomers were purified by reversed phase chromatography (water (0.05% NH₃ in water+10 mM NH₄HCO₃); ACN; 55-85%; 25 mL/min) to afford Compound 51A (second peak on SFC, 118 mg, 49% yield) and Compound 51B (first peak on SFC, 47.1 mg, 20% yield). LCMS (ESI): [M+H]⁺=403.2.

Compound 51A: ¹H NMR (400 MHz, CDCl₃) δ 7.61 (d, J=8.0 Hz, 1H), 7.48 (d, J=8.0 Hz, 1H), 4.08 (s, 4H), 2.93 (s, 2H), 2.82-2.80 (m, 2H), 2.77-2.70 (m, 1H), 2.64 (s, 3H), 2.30-2.21 (m, 1H), 2.19-2.09 (m, 4H), 1.93-1.90 (m, 2H), 1.49-1.41 (m, 4H). The relative stereochemistry was assigned based on ¹H NMR analysis.

Compound 51B: ¹H NMR (400 MHz, CDCl₃) δ 7.62 (d, J=8.0 Hz, 1H), 7.49 (d, J=8.0 Hz, 1H), 4.14-4.05 (m, 4H), 2.85-2.78 (m, 2H), 2.76-2.73 (m, 2H), 2.65 (s, 3H), 2.47 (brs, 1H), 2.18 (t, J=7.2 Hz, 2H), 2.02-1.99 (m, 2H), 1.85-1.77 (m, 3H), 1.66-1.55 (m, 4H). The relative stereochemistry was assigned based on ¹H NMR analysis.

### Example VV: 7-(1-(3-Fluoro-5-(trifluoromethyl)pyridin-2-yl)piperidin-4-yl)-2-thia-7-azaspiro[3.5]nonane 2,2-dioxide (Compound 52)

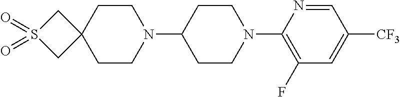 [SMILES: O=S1(=O)CC2(CCN(C3CCN(c4ncc(C(F)(F)F)cc4F)CC3)CC2)C1]

The title compound was synthesized following a procedure similar to Compound 45 using 2-bromo-3-fluoro-5-(trifluoromethyl)pyridine. The title compound was purified by pre-TLC (ethyl alcohol:ethyl acetate:petroleum ether=1:3:4) to afford the title compound (36.6 mg, 0.084 mmol, 44% yield). LCMS (ESI) [M+H]⁺=422.2. 1H NMR (400 MHz, CDCl₃) δ 8.22 (s, 1H), 7.37 (dd, J=2.0, 13.6 Hz, 1H), 4.38 (d, J=12.4 Hz, 2H), 3.85 (s, 4H), 2.94 (t, J=12.4 Hz, 2H), 2.55 (s, 5H), 1.93-1.87 (m, 6H), 1.64-1.61 (m, 2H).

### Example WW: (Compounds 53A*, 53B*, 53C*, and 53D*): 6-((1S,3S)-3-(1-Isopropyl-3-(4-(trifluoromethyl)phenyl)-1H-1,2,4-triazol-5-yl)cyclopentyl)-2-thia-6-azaspiro[3.4]octane 2,2-dioxide, 6-((1R,3S)-3-(1-isopropyl-3-(4-(trifluoromethyl)phenyl)-1H-1,2,4-triazol-5-yl)cyclopentyl)-2-thia-6-azaspiro[3.4]octane 2,2-dioxide, 6-((1S,3R)-3-(1-isopropyl-3-(4-(trifluoromethyl)phenyl)-1H-1,2,4-triazol-5-yl)cyclopentyl)-2-thia-6-azaspiro[3.4]octane 2,2-dioxide and 6-((1R,3R)-3-(1-isopropyl-3-(4-(trifluoromethyl)phenyl)-1H-1,2,4-triazol-5-yl)cyclopentyl)-2-thia-6-azaspiro[3.4]octane 2,2-dioxide

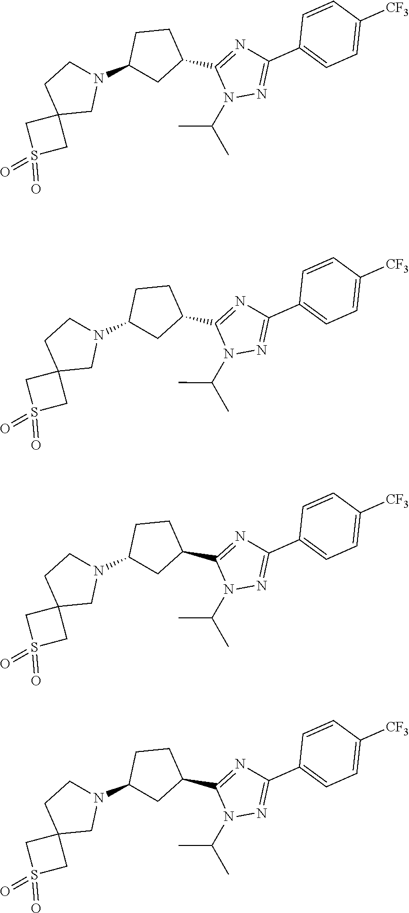 [SMILES: CC(C)n1nc(-c2ccc(C(F)(F)F)cc2)nc1[C@H]1CC[C@H](N2CCC3(C2)CS(=O)(=O)C3)C1 ; CC(C)n1nc(-c2ccc(C(F)(F)F)cc2)nc1[C@H]1CC[C@@H](N2CCC3(C2)CS(=O)(=O)C3)C1 ; CC(C)n1nc(-c2ccc(C(F)(F)F)cc2)nc1[C@@H]1CC[C@@H](N2CCC3(C2)CS(=O)(=O)C3)C1 ; CC(C)n1nc(-c2ccc(C(F)(F)F)cc2)nc1[C@@H]1CC[C@H](N2CCC3(C2)CS(=O)(=O)C3)C1]

### Step 1: (R)-3-(1-Isopropyl-3-(4-(trifluoromethyl)phenyl)-1H-1,2,4-triazol-5-yl)cyclopentanone

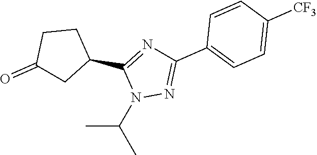 [SMILES: CC(C)n1nc(-c2ccc(C(F)(F)F)cc2)nc1[C@@H]1CCC(=O)C1]

To a solution of (R)-3-oxocyclopentanecarboxylic acid (1.0 g, 7.8 mmol) and 4-(trifluoromethyl)benzamidine hydrochloride (2.63 g, 11.71 mmol) in N,N-dimethylformamide (45 mL) was added HATU (3.26 g, 8.59 mmol) and N,N-diisopropylethylamine (3.98 mL, 23.41 mmol). The reaction mixture was stirred at 20° C. for 1 h. To the above mixture was added isopropylhydrazine hydrochloride (1.29 g, 11.7 mmol) and acetic acid (4.46 mL, 78.05 mmol) and stirred at 80° C. for another 1.5 h. The mixture was diluted with ethyl acetate (200 mL) and the resulting mixture was washed with water (50 mL) and brine (50 mL×2). The organic layer was dried over anhydrous sodium sulfate, filtered and concentrated in vacuo. The residue was purified by silica flash chromatography (0-50% ethyl acetate in petroleum ether) to afford the title compound (700 mg, 26% yield). LCMS (ESI) [M+H]⁺=338.1. SFC showed 99% ee.

### Step 2: (S)-3-(1-isopropyl-3-(4-(trifluoromethyl)phenyl)-1H-1,2,4-triazol-5-yl)cyclopentanone

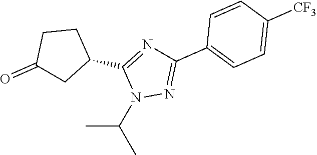 [SMILES: CC(C)n1nc(-c2ccc(C(F)(F)F)cc2)nc1[C@H]1CCC(=O)C1]

The title compound was synthesized following a procedure similar to (R)-3-(1-isopropyl-3-(4-(trifluoromethyl)phenyl)-1H-1,2,4-triazol-5-yl)cyclopentanone but instead using (S)-3-oxocyclopentanecarboxylic acid (Compound 36A, procedure, Step 1).

### Step 3A: 6-((1S,3S)-3-(1-isopropyl-3-(4-(trifluoromethyl)phenyl)-1H-1,2,4-triazol-5-yl)cyclopentyl)-2-thia-6-azaspiro[3.4]octane 2,2-dioxide and 6-((1R,3S)-3-(1-isopropyl-3-(4-(trifluoromethyl)phenyl)-1H-1,2,4-triazol-5-yl)cyclopentyl)-2-thia-6-azaspiro[3.4]octane 2,2-dioxide

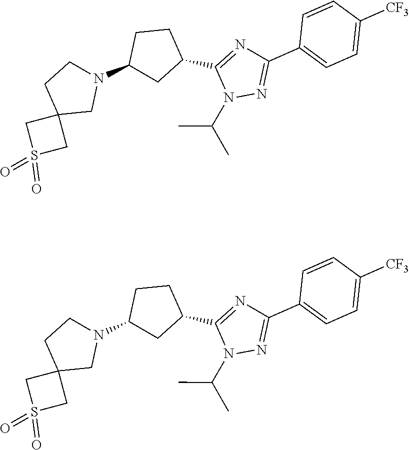 [SMILES: CC(C)n1nc(-c2ccc(C(F)(F)F)cc2)nc1[C@H]1CC[C@H](N2CCC3(C2)CS(=O)(=O)C3)C1 ; CC(C)n1nc(-c2ccc(C(F)(F)F)cc2)nc1[C@H]1CC[C@@H](N2CCC3(C2)CS(=O)(=O)C3)C1]

To a mixture of (S)-3-(1-isopropyl-3-(4-(trifluoromethyl)phenyl)-1H-1,2,4-triazol-5-yl)cyclopentanone (100 mg, 0.30 mmol) and 2-thia-6-azaspiro[3.4]octane 2,2-dioxide (57 mg, 0.36 mmol) in methanol (5 mL) was added sodium cyanoborohydride (56 mg, 0.89 mmol) and acetic acid (0.1 mL, 1.19 mmol). The reaction mixture was stirred at 60° C. for 1 h. The reaction mixture was quenched with water (10 mL) and the resulting mixture was extracted with ethyl acetate (20 mL×3). The combined organic layers were washed with brine (10 mL), dried over anhydrous sodium sulfate, filtered and concentrated in vacuo to give 120 mg of the crude product, which was separated using chiral SFC (Daicel Chiralpak (250 mm*30 mm, 10 μm); 0.1% NH₃ in water; EtOH; 25%; 60 mL/min) to afford the title Compound 53A* (first peak on SFC, 26.3 mg, 23% yield) and the title Compound 53B* (second peak on SFC, 84.8 mg, 69% yield). LCMS (ESI) [M+H]⁺=483.1.

Compound 53A*: ¹H NMR (400 MHz, CD₃OD) δ 8.20 (d, J=8.0 Hz, 2H), 7.74 (d, J=8.4 Hz, 2H), 4.80-4.75 (m, 1H), 4.20-4.08 (m, 4H), 3.64-3.53 (m, 1H), 3.08-2.99 (m, 1H), 2.94 (s, 2H), 2.80 (t, J=7.6 Hz, 2H), 2.31-1.86 (m, 7H), 1.78-1.64 (m, 1H), 1.60-1.50 (m, 6H).

Compound 53B*: ¹H NMR (400 MHz, CD₃OD) δ 8.19 (d, J=8.0 Hz, 2H), 7.75 (d, J=8.0 Hz, 2H), 4.80-4.75 (m, 1H), 4.31-4.16 (m, 4H), 3.64-3.18 (m, 1H), 2.57-2.45 (m, 1H), 2.40 (t, J=6.8 Hz, 2H), 2.31-1.85 (m, 6H), 1.59-1.46 (m, 6H).

### Step 3B: 6-((1S,3R)-3-(1-isopropyl-3-(4-(trifluoromethyl)phenyl)-1H-1,2,4-triazol-5-yl)cyclopentyl)-2-thia-6-azaspiro[3.4]octane 2,2-dioxide and 6-((1R,3R)-3-(1-isopropyl-3-(4-(trifluoromethyl)phenyl)-1H-1,2,4-triazol-5-yl)cyclopentyl)-2-thia-6-azaspiro[3.4]octane 2,2-dioxide

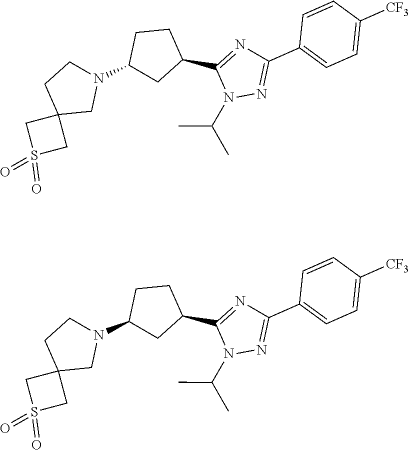 [SMILES: CC(C)n1nc(-c2ccc(C(F)(F)F)cc2)nc1[C@@H]1CC[C@@H](N2CCC3(C2)CS(=O)(=O)C3)C1 ; CC(C)n1nc(-c2ccc(C(F)(F)F)cc2)nc1[C@@H]1CC[C@H](N2CCC3(C2)CS(=O)(=O)C3)C1]

To a mixture of (R)-3-(1-isopropyl-3-(4-(trifluoromethyl)phenyl)-1H-1,2,4-triazol-5-yl)cyclopentanone (100 mg, 0.30 mmol) and 2-thia-6-azaspiro[3.4]octane 2,2-dioxide (57 mg, 0.36 mmol) in methanol (5 mL) were added sodium cyanoborohydride (56 mg, 0.89 mmol) and acetic acid (0.1 mL, 1.19 mmol) and the mixture was stirred at 60° C. for 1 h. The reaction mixture was quenched with water (10 mL) and the resulting mixture was extracted with ethyl acetate (20 mL×3). The combined organic layers were washed with brine (10 mL), dried over anhydrous sodium sulfate, filtered and the filtrate was concentrated in vacuo to give 120 mg crude product, which was separated using chiral SFC (Daicel Chiralpak OJ-H (250 mm*30 mm, 5 μm); 0.1% NH₃ in water; EtOH; 15%; 60 mL/min) to afford the title Compound 53C* (first peak on SFC, 23.6 mg, 15% yield) and the title Compound 53D* (second peak on SFC, 65.7 mg, 44% yield). LCMS (ESI), [M+H]⁺=483.1.

Compound 53C*: ¹H NMR (400 MHz, CD₃OD) δ 8.20 (d, J=8.4 Hz, 2H), 7.74 (d, J=8.0 Hz, 2H), 4.78-4.70 (m, 1H), 4.23-4.07 (m, 4H), 3.69-3.53 (m, 1H), 3.10-3.00 (m, 1H), 2.95 (s, 2H), 2.81 (t, J=7.2 Hz, 2H), 2.33-1.64 (m, 8H), 1.57-1.50 (m, 6H).

Compound 53D*: ¹H NMR (400 MHz, CD₃OD) δ 8.21 (d, J=8.0 Hz, 2H), 7.73 (d, J=8.0 Hz, 2H), 4.77-4.70 (m, 1H), 4.19-4.07 (m, 4H), 3.56-3.41 (m, 1H), 2.96 (s, 2H), 2.94-2.74 (m, 3H), 2.39-2.29 (m, 1H), 2.22 (t, J=7.2 Hz, 2H), 2.19-1.94 (m, 4H), 1.94-1.79 (m, 1H), 1.57-1.49 (m, 6H).

### Example XX: (Compounds 54A*, 54B*, 54C*, and 54D*): 6-((1R,3S)-3-(6-(Trifluoromethyl)pyridin-3-yl)cyclohexyl)-2-thia-6-azaspiro[3.4]octane 2,2-dioxide, 6-((1R,3R)-3-(6-(trifluoromethyl)pyridin-3-yl)cyclohexyl)-2-thia-6-azaspiro[3.4]octane 2,2-dioxide, 6-((1S,3R)-3-(6-(trifluoromethyl)pyridin-3-yl)cyclohexyl)-2-thia-6-azaspiro[3.4]octane 2,2-dioxide and 6-((1S,3S)-3-(6-(trifluoromethyl)pyridin-3-yl)cyclohexyl)-2-thia-6-azaspiro[3.4]octane 2,2-dioxide

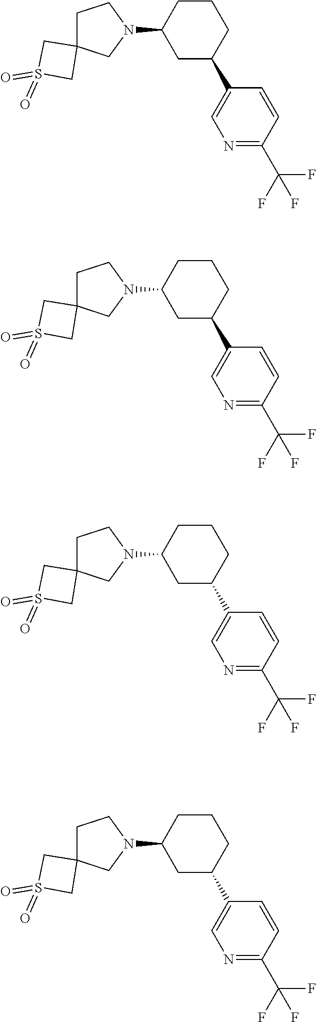 [SMILES: O=S1(=O)CC2(CCN([C@H]3CCC[C@@H](c4ccc(C(F)(F)F)nc4)C3)C2)C1 ; O=S1(=O)CC2(CCN([C@@H]3CCC[C@@H](c4ccc(C(F)(F)F)nc4)C3)C2)C1 ; O=S1(=O)CC2(CCN([C@@H]3CCC[C@H](c4ccc(C(F)(F)F)nc4)C3)C2)C1 ; O=S1(=O)CC2(CCN([C@H]3CCC[C@H](c4ccc(C(F)(F)F)nc4)C3)C2)C1]

### Step 1: 3-(6-(Trifluoromethyl)pyridin-3-yl)cyclohex-2-enone

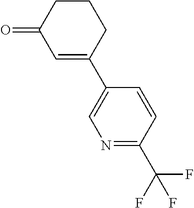 [SMILES: O=C1C=C(c2ccc(C(F)(F)F)nc2)CCC1]

To a mixture of 5-bromo-2-(trifluoromethyl)pyridine (900 mg, 3.98 mmol), 3-(4,4,5,5-tetramethyl-1,3,2-dioxaborolan-2-yl)cyclohex-2-en-1-one (1000 mg, 4.5 mmol) and K₂CO₃ (1800 mg, 13.02 mmol) in 1,4-dioxane (24 mL) and water (4 mL) was added Pd(dppf)Cl₂ (280 mg, 0.38 mmol). The resulting mixture was then stirred under nitrogen atmosphere at 90° C. for 5 h. The reaction was quenched by water (30 mL) and extracted with ethyl acetate (100 mL×3). The combined organic layers were washed with brine (30 mL×3), dried over anhydrous Na₂SO₄, filtered and concentrated in vacuo. The residue was purified by silica flash chromatography (0-40% ethyl acetate in petroleum ether) to provide the title compound (900 mg, 3.62 mmol, 91% yield). LCMS (ESI) [M+H]⁺=242.1.

### Step 2: 3-(6-(Trifluoromethyl)pyridin-3-yl)cyclohexanol

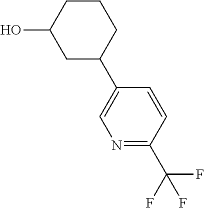 [SMILES: OC1CCCC(c2ccc(C(F)(F)F)nc2)C1]

To a solution of 3-(6-(trifluoromethyl)pyridin-3-yl)cyclohex-2-enone (900 mg, 3.73 mmol) in ethyl acetate (20 mL) was added platinum(IV) oxide (180 mg, 0.79 mmol). The suspension was then purged with H₂ for three times. The mixture was stirred under H₂ (15 psi) at 20° C. for 2 h. The reaction mixture was filtered and the filtrate was concentrated in vacuo. The residue was purified by silica column chromatography (0-50% ethyl acetate in petroleum ether) to provide the title compound (810 mg, 3.30 mmol, 88.5% yield). LCMS (ESI), [M+H]⁺=246.1.

### Step 3: 3-(6-(Trifluoromethyl)pyridin-3-yl)cyclohexanone

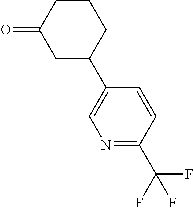 [SMILES: O=C1CCCC(c2ccc(C(F)(F)F)nc2)C1]

To a solution of 3-(6-(trifluoromethyl)pyridin-3-yl)cyclohexanol (1100 mg, 4.49 mmol) in anhydrous dichloromethane (26 mL) was added Dess-Martin periodinane (4000 mg, 9.43 mmol) at 0° C. and then the resulting mixture was stirred at 25° C. for 16 h. The mixture was then quenched by Na₂SO₃ solution (50 mL), followed by saturated NaHCO₃ solution (50 mL). The resulting mixture was extracted with dichloromethane (300 mL×3). The combined organic layers were washed with brine (100 mL), dried over anhydrous Na₂SO₄, filtered and concentrated in vacuo. The residue was purified by silica column chromatography (0-40% ethyl acetate in petroleum ether) to provide the title compound (930 mg, 3.59 mmol, 80.1% yield). LCMS (ESI) [M+H]⁺=244.1.

### Step 4: (R)-3-(6-(Trifluoromethyl)pyridin-3-yl)cyclohexanone and (S)-3-(6-(trifluoromethyl)pyridin-3-yl)cyclohexanone

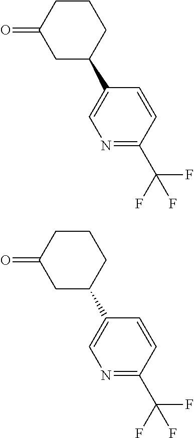 [SMILES: O=C1CCC[C@@H](c2ccc(C(F)(F)F)nc2)C1 ; O=C1CCC[C@H](c2ccc(C(F)(F)F)nc2)C1]

The mixture of enantiomers (930 mg, 3.82 mmol) was separated by chiral SFC (Daicel Chiralpak AD-H (250 mm*30 mm, 5 μm); EtOH+NH₃·H₂O=30/30; 60 mL/min) to provide the title compound 54A (first peak on SFC, 340 mg, 1.40 mmol, 36.6% yield) and the title compound 54B (second peak on SFC, 350 mg, 1.44 mmol, 38% yield). LCMS (ESI) [M+H]⁺=244.1.

### Step 5: 6-((1R,3S)-3-(6-(Trifluoromethyl)pyridin-3-yl)cyclohexyl)-2-thia-6-azaspiro[3.4]octane 2,2-dioxide, 6-((1R,3R)-3-(6-(trifluoromethyl)pyridin-3-yl)cyclohexyl)-2-thia-6-azaspiro[3.4]octane 2,2-dioxide

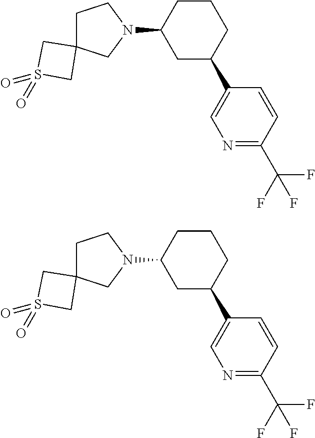 [SMILES: O=S1(=O)CC2(CCN([C@H]3CCC[C@@H](c4ccc(C(F)(F)F)nc4)C3)C2)C1 ; O=S1(=O)CC2(CCN([C@@H]3CCC[C@@H](c4ccc(C(F)(F)F)nc4)C3)C2)C1]

To a solution of (R)-3-(6-(trifluoromethyl)pyridin-3-yl)cyclohexanone (100 mg, 0.41 mmol) and 2-thia-6-azaspiro[3.4]octane 2,2-dioxide (135 mg, 0.84 mmol) in methanol (4 mL) were added NaBH₃CN (130 mg, 2.06 mmol) and acetic acid (0.1 mL, 1.75 mmol) at 20° C. The reaction mixture was then stirred at 70° C. for 4 h. The reaction mixture was quenched by saturated NaHCO₃ solution (10 mL), and extracted with dichloromethane (30 mL×2). The combined organic layers were washed with brine, dried over anhydrous Na₂SO₄, filtered and concentrated in vacuo. The resulting residue was purified by prep-TLC (10% methanol in dichloromethane) to provide title Compound 54A* (first peak, 30.37 mg, 0.077 mmol, 23.3% yield) and the title Compound 54B* (second peak, 59.11 mg, 0.14 mmol, 43.5% yield). LCMS (ESI) [M+H]⁺=389.1.

Compound 54A*: ¹H NMR (400 MHz, CDCl₃) δ 8.59 (s, 1H), 7.68 (d, J=8.0 Hz, 1H), 7.62 (d, J=8.0 Hz, 1H), 4.14-4.06 (m, 4H), 3.09 (t, J=12.4 Hz, 1H), 2.99 (d, J=9.2 Hz, 1H), 2.89-2.80 (m, 1H), 2.72 (d, J=9.2 Hz, 1H), 2.69-2.61 (m, 1H), 2.58 (brs, 1H), 2.23-2.14 (m, 2H), 2.00-1.92 (m, 3H), 1.83-1.78 (m, 1H), 1.71-1.47 (m, 4H).

Compound 54B*: H NMR (400 MHz, CDCl₃) δ 8.59 (s, 1H), 7.70 (d, J=8.4 Hz, 1H), 7.64 (d, J=8.4 Hz, 1H), 4.07 (s, 4H), 2.95-2.67 (m, 5H), 2.36-1.87 (m, 6H), 1.76-1.45 (m, 5H).

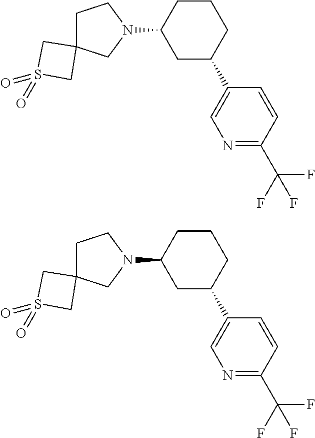 [SMILES: O=S1(=O)CC2(CCN([C@@H]3CCC[C@H](c4ccc(C(F)(F)F)nc4)C3)C2)C1 ; O=S1(=O)CC2(CCN([C@H]3CCC[C@H](c4ccc(C(F)(F)F)nc4)C3)C2)C1]

To a solution of (S)-3-(6-(trifluoromethyl)pyridin-3-yl)cyclohexanone (100 mg, 0.41 mmol) and 2-thia-6-azaspiro[3.4]octane 2,2-dioxide (135 mg, 0.84 mmol) in anhydrous methanol (4 mL) were added NaBH₃CN (130 mg, 2.06 mmol) and acetic acid (0.1 mL, 1.75 mmol) at 20° C. The resulting mixture was then stirred at 70° C. for 4 h. The reaction was quenched by saturated NaHCO₃ solution (10 mL), and extracted with dichloromethane (50 mL×2). The combined organic layers were washed with brine, dried over Na₂SO₄, filtered and concentrated under reduced pressure. The resulting residue was purified by pre-TLC (10% methanol in dichloromethane) to provide title Compound 54C* (the first peak on SFC, 33.29 mg, 0.082 mmol, 20% yield) and the title Compound 54D* (the second peak on SFC, 65.05 mg, 0.16 mmol, 38.7% yield). LCMS (ESI) [M+H]⁺=389.1.

Compound 54C*: ¹H NMR (400 MHz, CDCl₃) δ 8.59 (s, 1H), 7.68 (d, J=8.0 Hz, 1H), 7.62 (d, J=8.0 Hz, 1H), 4.14-4.06 (m, 4H), 3.09 (t, J=12.4 Hz, 1H), 2.99 (d, J=9.2 Hz, 1H), 2.89-2.80 (m, 1H), 2.73 (d, J=9.2 Hz, 1H), 2.67-2.61 (m, 1H), 2.58 (brs, 1H), 2.24-2.15 (m, 2H), 2.00-1.92 (m, 3H), 1.83-1.78 (m, 1H), 1.71-1.47 (m, 4H).

Compound 54D*: ¹H NMR (400 MHz, CDCl₃) δ 8.59 (brs, 1H), 7.69 (brs, 1H), 7.67-7.60 (m, 1H), 4.07 (brs, 4H), 2.92 (d, J=19.6 Hz, 2H), 2.81 (brs, 2H), 2.72 (d, J=11.6 Hz, 2H), 2.15 (brs, 4H), 1.99 (d, J=7.2 Hz, 1H), 1.89 (d, J=10.4 Hz, 1H), 1.54-1.36 (m, 4H).

### Example YY: (Compounds 55A and 55B): (R)-7-(1-(4-(Trifluoromethyl)phenyl)piperidin-3-yl)-2-thia-7-azaspiro[3.5]nonane 2,2-dioxide and (S)-7-(1-(4-(trifluoromethyl)phenyl)piperidin-3-yl)-2-thia-7-azaspiro[3.5]nonane 2,2-dioxide

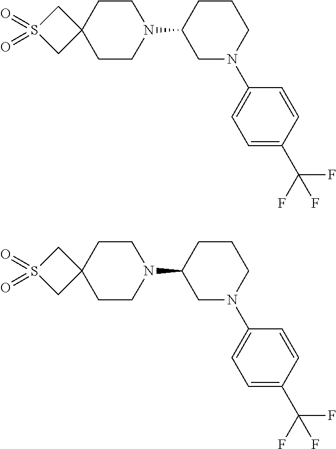 [SMILES: O=S1(=O)CC2(CCN([C@@H]3CCCN(c4ccc(C(F)(F)F)cc4)C3)CC2)C1 ; O=S1(=O)CC2(CCN([C@H]3CCCN(c4ccc(C(F)(F)F)cc4)C3)CC2)C1]

### Step 1: tert-Butyl 3-(2,2-dioxido-2-thia-7-azaspiro[3.5]nonan-7-yl)piperidine-1-carboxylate

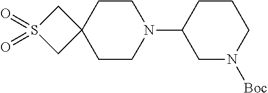 [SMILES: CC(C)(C)OC(=O)N1CCCC(N2CCC3(CC2)CS(=O)(=O)C3)C1]

To a solution of tert-butyl 3-oxopiperidine-1-carboxylate (350 mg, 1.76 mmol) and 2-thia-7-azaspiro[3.5]nonane 2,2-dioxide (400 mg, 2.28 mmol) in dichloromethane (10 mL) were added N,N-diisopropylethylamine (0.93 mL, 5.27 mmol) and 4A molecular sieves. The reaction mixture was stirred at 65° C. for 12 h. Sodium triacetoxyborohydride (1116 mg, 5.27 mmol) was then added. The mixture was stirred at 65° C. for 1 h, quenched with brine (20 mL) and extracted with dichloromethane (50 mL×3). The combined organic layer was dried over anhydrous Na₂SO₄, filtered and concentrated in vacuo. The residue was purified by silica flash chromatography (0-10% methanol in dichloromethane) to provide the title compound (300 mg, 47.6% yield). LCMS (ESI), [M+H]⁺=359.1.

### Step 2: 7-(Piperidin-3-yl)-2-thia-7-azaspiro[3.5]nonane 2,2-dioxide

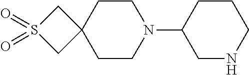 [SMILES: O=S1(=O)CC2(CCN(C3CCCNC3)CC2)C1]

tert-Butyl 3-(2,2-dioxido-2-thia-7-azaspiro[3.5]nonan-7-yl)piperidine-1-carboxylate (300.0 mg, 0.84 mmol) was dissolved in HCl/dioxane (8.0 mL, 32 mmol, 4M). The reaction mixture was stirred at 25° C. for 1 h and was concentrated in vacuo to give the title compound (220 mg, 100% yield).

### Step 3:7-(1-(4-(Trifluoromethyl)phenyl)piperidin-3-yl)-2-thia-7-azaspiro[3.5]nonane 2,2-dioxide

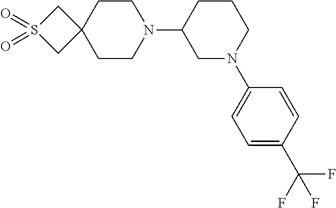 [SMILES: O=S1(=O)CC2(CCN(C3CCCN(c4ccc(C(F)(F)F)cc4)C3)CC2)C1]

To a solution of 7-(piperidin-3-yl)-2-thia-7-azaspiro[3.5]nonane 2,2-dioxide (60 mg, 0.23 mmol) and 4-bromobenzotrifluoride (190 mg, 0.84 mmol) in toluene (5.0 mL) were added (2-dicyclohexylphosphino-2′,6′-diisopropoxy-1,1′-biphenyl) [2-(2′-amino-1,1′-biphenyl)]palladium(II) methanesulfonate (70 mg, 0.08 mmol), 2-dicyclohexylphosphino-2′,6′-di-i-propoxy-1,1′-biphenyl (79 mg, 0.17 mmol) and cesium carbonate (825 mg, 2.53 mmol). The reaction mixture was stirred under N2 at 110° C. for 16 h. The mixture was filtered and concentrated in vacuo. The residue was purified by silica flash chromatography (0-2% methanol in dichloromethane). LCMS (ESI) [M+H]⁺=403.1.

### Step 4: (R)-7-(1-(4-(Trifluoromethyl)phenyl)piperidin-3-yl)-2-thia-7-azaspiro[3.5]nonane 2,2-dioxide and (S)-7-(1-(4-(trifluoromethyl)phenyl)piperidin-3-yl)-2-thia-7-azaspiro[3.5]nonane 2,2-dioxide

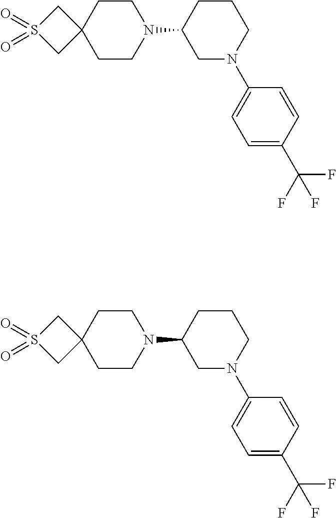 [SMILES: O=S1(=O)CC2(CCN([C@@H]3CCCN(c4ccc(C(F)(F)F)cc4)C3)CC2)C1 ; O=S1(=O)CC2(CCN([C@H]3CCCN(c4ccc(C(F)(F)F)cc4)C3)CC2)C1]

The mixture of enantiomers (110 mg, 0.27 mmol) was separated using chiral SFC (Daicel Chiralpak AD-H (250 mm*30 mm, 5 μm); 0.1% NH₃ in H₂O; MeOH; 40%; 60 mL/min) to provide the Compound 55A* (first peak on SFC, 31.45 mg, 27.2% yield) and the Compound 55B* (second peak on SFC, 36.1 mg, 31.2% yield).

Compound 55A*: ¹H NMR (400 MHz, CD₃OD) δ 7.45 (d, J=8.8 Hz, 2H), 7.02 (d, J=8.8 Hz, 2H), 3.98 (d, J=12.4 Hz, 1H), 3.92 (s, 4H), 3.81 (d, J=13.2 Hz, 1H), 2.83-2.73 (m, 2H), 2.67 (brs, 4H), 2.59-2.52 (m, 1H), 2.08-2.02 (m, 1H), 1.94-1.91 (m, 4H), 1.87-1.82 (m, 1H), 1.68-1.59 (m, 2H).

Compound 55B*: ¹H NMR (400 MHz, CD₃OD) δ 7.45 (d, J=8.8 Hz, 2H), 7.02 (d, J=8.8 Hz, 2H), 3.98 (d, J=12.4 Hz, 1H), 3.92 (s, 4H), 3.81 (d, J=12.8 Hz, 1H), 2.83-2.72 (m, 2H), 2.67 (brs, 4H), 2.59-2.52 (m, 1H), 2.08-2.02 (m, 1H), 1.94-1.91 (m, 4H), 1.87-1.83 (m, 1H), 1.66-1.45 (m, 2H).

### Example ZZ: (Compounds 56A*, 56B*, 56C*, and 56D*): 7-((1R,3S)-3-(6-(trifluoromethyl)pyridin-3-yl)cyclohexyl)-2-thia-7-azaspiro[3.5]nonane 2,2-dioxide, 7-((1S,3S)-3-(6-(trifluoromethyl)pyridin-3-yl)cyclohexyl)-2-thia-7-azaspiro[3.5]nonane 2,2-dioxide, 7-((1S,3R)-3-(6-(trifluoromethyl)pyridin-3-yl)cyclohexyl)-2-thia-7-azaspiro[3.5]nonane 2,2-dioxide and 7-((1R,3R)-3-(6-(trifluoromethyl)pyridin-3-yl)cyclohexyl)-2-thia-7-azaspiro[3.5]nonane 2,2-dioxide

### 7-((1R,3S)-3-(6-(trifluoromethyl)pyridin-3-yl)cyclohexyl)-2-thia-7-azaspiro[3.5]nonane 2,2-dioxide and 7-((1S,3S)-3-(6-(trifluoromethyl)pyridin-3-yl)cyclohexyl)-2-thia-7-azaspiro[3.5]nonane 2,2-dioxide

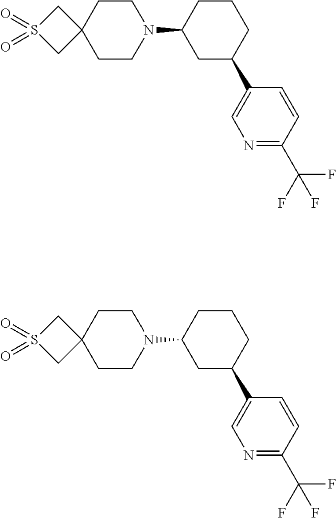 [SMILES: O=S1(=O)CC2(CCN([C@H]3CCC[C@@H](c4ccc(C(F)(F)F)nc4)C3)CC2)C1 ; O=S1(=O)CC2(CCN([C@@H]3CCC[C@@H](c4ccc(C(F)(F)F)nc4)C3)CC2)C1]

The title compounds were synthesized following a procedure similar to Compound 54A using 2-thia-7-azaspiro[3.5]nonane 2,2-dioxide. The mixture of the diastereomers were purified by prep-TLC (10% methanol in dichloromethane) to provide Compound 56A* (first peak, 40.1 mg, 0.096 mmol, 23.2% yield) and Compound 56B* (second peak, 20.6 mg, 0.049 mmol, 11.8% yield). LCMS (ESI) [M+H]⁺=403.1.

Compound 56A*: ¹H NMR (400 MHz, CDCl₃) δ 8.60 (s, 1H), 7.71 (d, J=7.8 Hz, 1H), 7.65-7.57 (m, 1H), 3.87 (s, 4H), 3.20-3.12 (m, 1H), 2.65-2.40 (m, 4H), 2.11-2.09 (m, 2H), 1.98-1.85 (m, 6H), 1.80-1.49 (m, 5H).

Compound 56B*: ¹H NMR (400 MHz, CDCl₃) δ 8.59 (s, 1H), 7.68 (dd, J=2.0, 8.4 Hz, 1H), 7.63 (d, J=8.0 Hz, 1H), 3.84 (s, 4H), 2.73-2.70 (m, 1H), 2.59-2.49 (m, 4H), 2.04-1.92 (m, 8H), 1.54-1.27 (m, 5H).

### 7-((1S,3R)-3-(6-(Trifluoromethyl)pyridin-3-yl)cyclohexyl)-2-thia-7-azaspiro[3.5]nonane 2,2-dioxide and 7-((1R,3R)-3-(6-(trifluoromethyl)pyridin-3-yl)cyclohexyl)-2-thia-7-azaspiro[3.5]nonane 2,2-dioxide

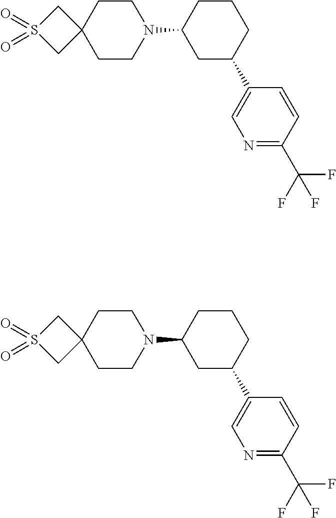 [SMILES: O=S1(=O)CC2(CCN([C@@H]3CCC[C@H](c4ccc(C(F)(F)F)nc4)C3)CC2)C1 ; O=S1(=O)CC2(CCN([C@H]3CCC[C@H](c4ccc(C(F)(F)F)nc4)C3)CC2)C1]

The title compounds were synthesized following a procedure similar to Compound 54A using 2-thia-7-azaspiro[3.5]nonane 2,2-dioxide. The mixture of the diastereomers were purified by reversed phase chromatography (acetonitrile 70-100%/(0.05% NH₃ in H₂O+10 mM NH₄HCO₃ in water) to provide Compound 56A* (second peak, 39.69 mg, 0.095 mmol, 11.5% yield) and Compound 56B* (first peak on SFC, 56.03 mg, 0.14 mmol, 16.4% yield). LCMS (ESI) [M+H]⁺=403.1.

Compound 56C*: ¹H NMR (400 MHz, CDCl₃) δ 8.59 (s, 1H), 7.68 (dd, J=2.0, 8.0 Hz, 1H), 7.63 (d, J=8.0 Hz, 1H), 3.84 (s, 4H), 2.78-2.63 (m, 1H), 2.58-2.45 (m, 5H), 2.04-1.92 (m, 8H), 1.49-1.31 (m, 4H).

Compound 56D*: ¹H NMR (400 MHz, CDCl₃) δ 8.60 (s, 1H), 7.71 (d, J=8.0 Hz, 1H), 7.62 (d, J=8.0 Hz, 1H), 3.87 (s, 4H), 3.20-3.12 (m, 1H), 2.65-2.40 (m, 4H), 2.11-2.09 (m, 2H), 1.98-1.85 (m, 6H), 1.80-1.69 (m, 2H), 1.65-1.58 (m, 1H), 1.55-1.43 (m, 2H).

### Example AAA: (Compounds 57A*, 57B*, 57C* and 57D*): 7-((1R,3R)-3-(6-(trifluoromethyl)pyridin-3-yl)cyclopentyl)-2-thia-7-azaspiro[3.5]nonane 2,2-dioxide, 7-((1S,3R)-3-(6-(trifluoromethyl)pyridin-3-yl)cyclopentyl)-2-thia-7-azaspiro[3.5]nonane 2,2-dioxide, 7-((1R,3S)-3-(6-(trifluoromethyl)pyridin-3-yl)cyclopentyl)-2-thia-7-azaspiro[3.5]nonane 2,2-dioxide and 7-((1S,3S)-3-(6-(trifluoromethyl)pyridin-3-yl)cyclopentyl)-2-thia-7-azaspiro[3.5]nonane 2,2-dioxide

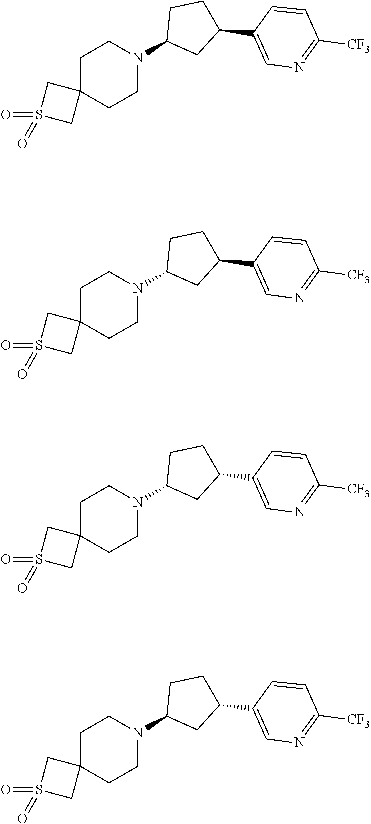 [SMILES: O=S1(=O)CC2(CCN([C@H]3CC[C@@H](c4ccc(C(F)(F)F)nc4)C3)CC2)C1 ; O=S1(=O)CC2(CCN([C@@H]3CC[C@@H](c4ccc(C(F)(F)F)nc4)C3)CC2)C1 ; O=S1(=O)CC2(CCN([C@@H]3CC[C@H](c4ccc(C(F)(F)F)nc4)C3)CC2)C1 ; O=S1(=O)CC2(CCN([C@H]3CC[C@H](c4ccc(C(F)(F)F)nc4)C3)CC2)C1]

Title compounds were synthesized following a procedure similar to Compound 54A using 3-(4,4,5,5-tetramethyl-1,3,2-dioxaborolan-2-yl)cyclopent-2-en-1-one for the first step and 2-thia-7-azaspiro[3.5]nonane 2,2-dioxide for step 5. The mixture of the diastereomers were purified by pre-TLC (dichloromethane/methanol=10/1) to provide Compound 57A* (the first peak, 31.61 mg, 0.07 mmol, 17.9% yield) and Compound 57B* (the second peak, 34.06 mg, 0.08 mmol, 19.3% yield). LCMS (ESI): [M+H]⁺=389.2.

Compound 57A*: ¹H NMR (400 MHz, CD₃OD) δ 8.62 (s, 1H), 7.97 (d, J=6.4 Hz, 1H), 7.76 (d, J=8.0 Hz, 1H), 3.94 (s, 4H), 3.28-3.23 (m, 1H), 2.92-2.37 (m, 5H), 2.35-2.03 (m, 3H), 1.98-1.95 (m, 4H), 1.85-1.76 (m, 2H), 1.65-1.62 (m, 1H).

Compound 57B*: ¹H NMR (400 MHz, CD₃OD) δ 8.61 (s, 1H), 7.95 (d, J=6.4 Hz, 1H), 7.75 (d, J=8.4 Hz, 1H), 3.95 (s, 4H), 3.43-3.35 (m, 1H), 3.20-2.39 (m, 5H), 2.32-2.05 (m, 4H), 2.05-1.95 (m, 4H), 1.78-1.68 (m, 2H).

The mixture of the diastereomers was purified by pre-TLC (dichloromethane:methanol=10:1) to provide Compound 57C* (the first peak on SFC, 41.82 mg, 0.10 mmol, 24.4% yield) and Compound 57D* (the second peak on SFC, 30.05 mg, 0.07 mmol, 17.6% yield). LCMS (ESI) [M+H]⁺=389.1.

Compound 57C*: ¹H NMR (400 MHz, CD₃OD) δ 8.64 (s, 1H), 7.99 (d, J=6.8 Hz, 1H), 7.78 (d, J=8.4 Hz, 1H), 4.01 (s, 4H), 3.30-3.24 (m, 1H), 2.95-2.51 (s, 5H), 2.37-2.15 (m, 3H), 2.12-2.08 (m, 4H), 1.97-1.74 (m, 3H).

Compound 57D*: ¹H NMR (400 MHz, CD₃OD) δ 8.60 (d, J=1.6 Hz, 1H), 7.94 (d, J=8.0 Hz, 1H), 7.75 (d, J=8.0 Hz, 1H), 3.94 (s, 4H), 3.39-3.35 (m, 1H), 3.16-2.31 (m, 5H), 2.20-2.15 (m, 4H), 2.02-1.95 (m, 4H), 1.76-1.66 (m, 2H).

### Example BBB: 7-((1R,3S)-3-(1-Isopropyl-3-(4-(trifluoromethyl)phenyl)-1H-1,2,4-triazol-5-yl)cyclopentyl)-2-thia-7-azaspiro[3.5]nonane 2,2-dioxide (Compound 58A) and 7-((1S,3S)-3-(1-isopropyl-3-(4-(trifluoromethyl)phenyl)-1H-1,2,4-triazol-5-yl)cyclopentyl)-2-thia-7-azaspiro[3.5]nonane 2,2-dioxide (Compound 58B)

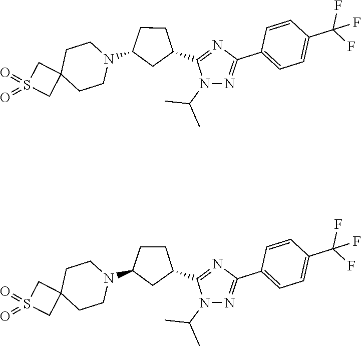 [SMILES: CC(C)n1nc(-c2ccc(C(F)(F)F)cc2)nc1[C@H]1CC[C@@H](N2CCC3(CC2)CS(=O)(=O)C3)C1 ; CC(C)n1nc(-c2ccc(C(F)(F)F)cc2)nc1[C@H]1CC[C@H](N2CCC3(CC2)CS(=O)(=O)C3)C1]

### Step 1: (S)-3-(1-Isopropyl-3-(4-(trifluoromethyl)phenyl)-1H-1,2,4-triazol-5-yl)cyclopentanone

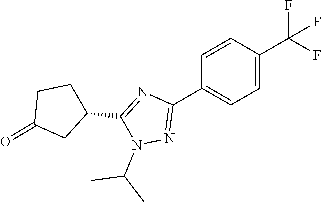 [SMILES: CC(C)n1nc(-c2ccc(C(F)(F)F)cc2)nc1[C@H]1CCC(=O)C1]

To a solution of (S)-3-oxocyclopentanecarboxylic acid (1 g, 7.8 mmol) and 4-(trifluoromethyl)benzamidine hydrochloride (2.63 g, 11.71 mmol) in DMF (45 mL) were added HATU (3.26 g, 8.59 mmol) and N,N-diisopropylethylamine (3.98 mL, 23.41 mmol). The mixture was stirred at 20° C. for 1 h. To the above mixture was added isopropylhydrazine hydrochloride (1.29 g, 11.71 mmol) and acetic acid (4.46 mL, 78.05 mmol). The reaction mixture was stirred at 80° C. for another 1.5 h. The mixture was diluted with ethyl acetate (200 mL) and the resulting mixture was washed with water (50 mL) and brine (50 mL×2). The organic layer was dried over anhydrous sodium sulfate, filtered and concentrated in vacuo. The residue was purified by silica flash chromatography (0˜50% ethyl acetate in petroleum ether) to afford the title compound (700 mg, 26% yield). LCMS (ESI) [M+H]⁺=338.1. SFC showed 99% ee.

### Step 2: 7-((1R, 3S)-3-(1-Isopropyl-3-(4-(trifluoromethyl)phenyl)-1H-1,2,4-triazol-5-yl)cyclopentyl)-2-thia-7-azaspiro[3.5]nonane 2,2-dioxide and 7-((1S, 3S)-3-(1-isopropyl-3-(4-(trifluoromethyl)phenyl)-1H-1,2,4-triazol-5-yl)cyclopentyl)-2-thia-7-azaspiro[3.5]nonane 2,2-dioxide

To a solution of (3S)-3-[2-isopropyl-5-[4-(trifluoromethyl)phenyl]-1,2,4-triazol-3-yl]cyclopentanone (100 mg, 0.2964 mmol) in methanol (4 mL) was added acetic acid (89 mg, 1.48 mmol), 2-thia-7-azaspiro[3.5]nonane 2,2-dioxide hydrochloride (82 mg, 0.386 mmol) and sodium cyanoborohydride (93 mg, 1.48 mmol). The reaction mixture was stirred at 70° C. for 4 h, then diluted with ethyl acetate (50 mL. The resulting mixture was washed with water (25 mL) and brine (50 mL×2). The organic layer was dried over anhydrous sodium sulfate, filtered and concentrated in vacuo. The residue was purified by silica flash chromatography (20-80% ethyl acetate in petroleum ether) to afford the title compound (140 mg, 95% yield). LCMS (ESI): [M+H]⁺=497.1. The mixture of the diastereomers (140 mg, 0.28 mmol) were separated using chiral SFC (Daicel Chiralcel OJ (250 mm*30 mm, 10 μm); 0.1% NH₃ in H₂O; EtOH) to afford title Compound 58A (second peak on SFC, 67.82 mg, 0.1352 mmol, 48% yield) and title Compound 58B (first peak on SFC, 23.05 mg, 0.0446 mmol, 15.8% yield). LCMS (ESI): [M+H]⁺=497.1. The relative stereochemistry was assigned based on NOE NMR analysis.

Compound 58A: ¹H NMR (400 MHz, CDCl₃) δ 8.19 (d, J=8.0 Hz, 2H), 7.66 (d, J=8.0 Hz, 2H), 4.55-4.49 (m, 1H), 3.88 (s, 4H), 3.45-3.35 (m, 1H), 2.98-2.92 (m, 1H), 2.53-2.33 (m, 3H), 2.30-2.10 (m, 3H), 2.07-1.98 (m, 6H), 1.54 (d, J=6.4 Hz, 8H).

Compound 58B: ¹H NMR (400 MHz, CDCl₃) δ 8.19 (d, J=8.0 Hz, 2H), 7.66 (d, J=8.0 Hz, 2H), 4.55-4.49 (m, 1H), 3.88 (s, 4H), 3.28-3.22 (m, 1H), 2.88-2.82 (m, 1H), 2.56-2.33 (m, 2H), 2.30-1.72 (m, 10H), 1.54 (d, J=6.4 Hz, 8H).

### Example CCC: 6-((1r,4r)-4-(3-Fluoro-5-(trifluoromethyl)pyridin-2-yl)cyclohexyl)-2-thia-6-azaspiro[3.4]octane 2,2-dioxide (Compound 59)

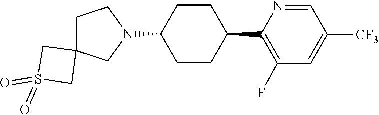 [SMILES: O=S1(=O)CC2(CCN([C@H]3CC[C@H](c4ncc(C(F)(F)F)cc4F)CC3)C2)C1]

The title compound was synthesized similarly to Compound 80A and Compound 80B using 2-bromo-3-fluoro-5-(trifluoromethyl)pyridine.

### Example DDD: 6-((1r,4r)-4-(2-(trifluoromethyl)pyridin-4-yl)cyclohexyl)-2-thia-6-azaspiro[3.4]octane 2,2-dioxide (Compound 60A) and 6-((1s,4s)-4-(2-(trifluoromethyl)pyridin-4-yl)cyclohexyl)-2-thia-6-azaspiro[3.4]octane 2,2-dioxide (Compound 60B)

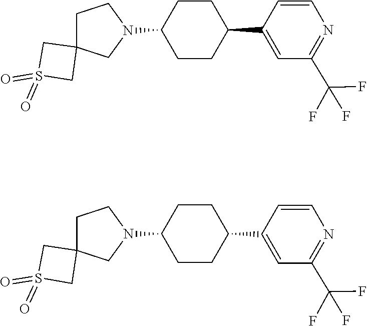 [SMILES: O=S1(=O)CC2(CCN([C@H]3CC[C@H](c4ccnc(C(F)(F)F)c4)CC3)C2)C1 ; O=S1(=O)CC2(CCN([C@H]3CC[C@@H](c4ccnc(C(F)(F)F)c4)CC3)C2)C1]

Title compounds were synthesized following a procedure similar to Compound 47A using 4-bromo-2-(trifluoromethyl)pyridine. The mixture of the diastereomers was purified by prep-HPLC (water (0.05% NH₃+10 nM NH₄HCO₃)-ACN) to afford Compound 60 (first peak on HPLC, 48.4 mg, 0.12 mmol, 41.5% yield) and Compound 96 (second peak on HPLC, 31.8 mg, 0.081 mmol, 28.2% yield). LCMS (ESI) [M+H]⁺=389.2. The relative stereochemistry was assigned based on ¹H NMR analysis.

Compound 60A: ¹H NMR (400 MHz, CDCl₃) δ 8.63 (d, J=4.8 Hz, 1H), 7.53 (s, 1H), 7.32 (d, J=3.6 Hz, 1H), 4.08 (s, 4H), 2.93 (s, 2H), 2.82-2.81 (m, 2H), 2.63-2.57 (m, 1H), 2.26-2.18 (m, 5H), 2.01-1.96 (m, 2H), 1.54-1.48 (m, 2H), 1.44-1.35 (m, 2H).

Compound 60B: ¹H NMR (400 MHz, CDCl₃) δ 8.63 (d, J=4.8 Hz, 1H), 7.52 (s, 1H), 7.33 (d, J=4.8 Hz, 1H), 4.13-4.05 (m, 4H), 2.85 (s, 2H), 2.76-2.67 (m, 3H), 2.44 (s, 1H), 2.20-2.16 (m, 2H), 2.02-1.95 (m, 2H), 1.90-1.80 (m, 2H), 1.68-1.64 (m, 4H).

### Example EEE: (Compounds 61A*, 61B*, 61C*, and 61D*): 6-((1R,3R)-3-(1-Isopropyl-3-(trifluoromethyl)-1H-pyrazol-5-yl)cyclohexyl)-2-thia-6-azaspiro[3.4]octane 2,2-dioxide, 6-((1R,3S)-3-(1-isopropyl-3-(trifluoromethyl)-1H-pyrazol-5-yl)cyclohexyl)-2-thia-6-azaspiro[3.4]octane 2,2-dioxide, 6-((1S,3R)-3-(1-isopropyl-3-(trifluoromethyl)-1H-pyrazol-5-yl)cyclohexyl)-2-thia-6-azaspiro[3.4]octane 2,2-dioxide and 6-((1S,3S)-3-(1-isopropyl-3-(trifluoromethyl)-1H-pyrazol-5-yl)cyclohexyl)-2-thia-6-azaspiro[3.4]octane 2,2-dioxide

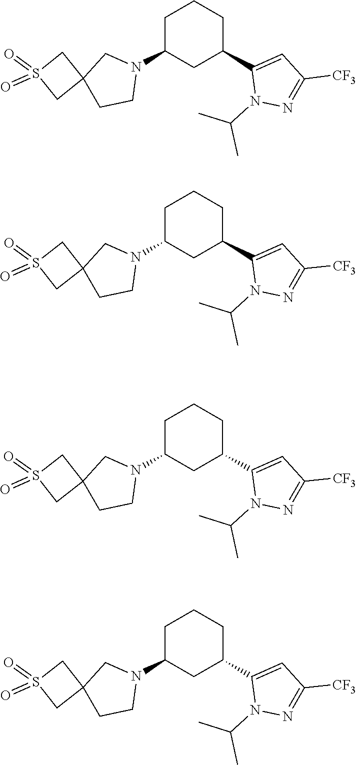 [SMILES: CC(C)n1nc(C(F)(F)F)cc1[C@@H]1CCC[C@H](N2CCC3(C2)CS(=O)(=O)C3)C1 ; CC(C)n1nc(C(F)(F)F)cc1[C@@H]1CCC[C@@H](N2CCC3(C2)CS(=O)(=O)C3)C1 ; CC(C)n1nc(C(F)(F)F)cc1[C@H]1CCC[C@@H](N2CCC3(C2)CS(=O)(=O)C3)C1 ; CC(C)n1nc(C(F)(F)F)cc1[C@H]1CCC[C@H](N2CCC3(C2)CS(=O)(=O)C3)C1]

### Step 1: 1-Isopropyl-3-(trifluoromethyl)-1H-pyrazole

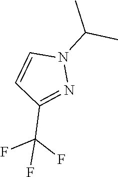

To a suspension of 3-(trifluoromethyl)pyrazole (4 g, 29.39 mmol) in acetonitrile (50 mL) were added 2-iodopropane (15 g, 88.18 mmol) and cesium carbonate (48 g, 146.97 mmol) at 25° C. The reaction mixture was stirred for 16 h. The reaction mixture was then filtered and the filtrate was diluted with MTBE (200 mL). The organic layer was washed with brine (50 mL×3), dried over anhydrous sodium sulfate, filtered and concentrated in vacuo to afford crude the title compound (4.2 g, 80% yield). ¹H NMR (400 MHz, CDCl₃) δ 7.46 (d, J=1.6 Hz, 1H), 6.50 (d, J=2.0 Hz, 1H), 4.61-4.51 (m, 1H), 1.53 (d, J=6.8 Hz, 6H).

### Step 2: 5-Bromo-1-isopropyl-3-(trifluoromethyl)-1H-pyrazole

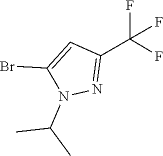

To a stirred solution of 1-isopropyl-3-(trifluoromethyl)pyrazole (3.2 g, 17.96 mmol) in tetrahydrofuran (50 mL) at −78° C. was added n-butyllithium (15 mL, 37.5 mmol, 2.3 M in THF) dropwise. The reaction mixture was stirred at −78° C. for 1 h. Then bromine (3.0 mL, 58.55 mmol) was added dropwise. The rate of addition was slow enough to allow complete decolorization of bromine prior to the next drop. After being allowed to warm to −30° C. over 1 hour, the reaction mixture was quenched with a saturated solution of NaHCO₃ (40 mL), and the reaction was allowed to warm to 25° C. The two phases were separated, and the aqueous layer was extracted with tert-butyl methyl ether (100 mL×3). The combined organic layers were washed with saturated solution of Na₂SO₃ (20 mL×2), dried over anhydrous Na₂SO₄, filtered and concentrated in vacuo to afford the title compound (3.8 g, 82% yield). LCMS (ESI): [M+H]+=258.9. ¹H NMR (400 MHz, CDCl₃) δ 6.54 (s, 1H), 4.78-4.71 (m, 1H), 1.50 (d, J=6.8 Hz, 6H).

### Step 3: 3-(1-Isopropyl-3-(trifluoromethyl)-1H-pyrazol-5-yl)cyclohex-2-enone

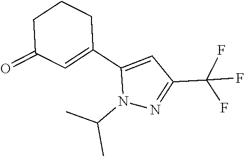 [SMILES: CC(C)n1nc(C(F)(F)F)cc1C1=CC(=O)CCC1]

To a solution of 5-bromo-1-isopropyl-3-(trifluoromethyl)pyrazole (2 g, 7.78 mmol), 3-(4,4,5,5-tetramethyl-1,3,2-dioxaborolan-2-yl)cyclohex-2-en-1-one (1728 mg, 7.78 mmol) and potassium carbonate (3226 mg, 23.34 mmol) in water (2 mL) and 1,4-dioxane (8 mL) was added Pd(dppf)Cl₂ (569 mg, 0.78 mmol). The reaction mixture was stirred under N2 at 80° C. for 4 h, concentrated in vacuo. The residue was purified by silica column chromatography (0-10% ethyl acetate in petroleum ether) to afford the title compound (1.5 g, 71% yield).

### Step 4: 3-(1-Isopropyl-3-(trifluoromethyl)-1H-pyrazol-5-yl)cyclohexanone

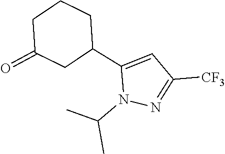 [SMILES: CC(C)n1nc(C(F)(F)F)cc1C1CCCC(=O)C1]

To a solution of 3-[2-isopropyl-5-(trifluoromethyl)pyrazol-3-yl]cyclohex-2-en-1-one (1.5 g, 5.51 mmol) in methyl alcohol (3 mL) was added 10% palladium on carbon (500 mg). The reaction mixture was stirred under H₂ (15 psi) at 25° C. for 1 h. The reaction mixture was filtered and the filtrate was concentrated in vacuo. The residue was purified by silica column chromatography (0-10% ethyl acetate in petroleum ether) to afford the title compound (830 mg, 55% yield). LCMS (ESI): [M+H]⁺=275.1.

### Step 5: (R)-3-(1-Isopropyl-3-(trifluoromethyl)-1H-pyrazol-5-yl)cyclohexanone and (S)-3-(1-isopropyl-3-(trifluoromethyl)-1H-pyrazol-5-yl)cyclohexanone

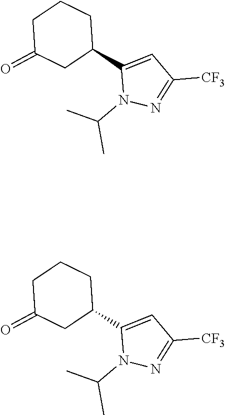 [SMILES: CC(C)n1nc(C(F)(F)F)cc1[C@@H]1CCCC(=O)C1 ; CC(C)n1nc(C(F)(F)F)cc1[C@H]1CCCC(=O)C1]

Both enantiomers of 3-(1-isopropyl-3-(trifluoromethyl)-1H-pyrazol-5-yl)cyclohexanone (960 mg, 3.5 mmol) were separated using chiral SFC (WHELK-O1; 250 mm*30 mm, 5 μm), 0.1% NH₃ in H₂O; EtOH; 10%; 60 mL/min) to afford (R)-3-(1-Isopropyl-3-(trifluoromethyl)-1H-pyrazol-5-yl)cyclohexanone (first peak on SFC, 200 mg, 21% yield) and (S)-3-(1-isopropyl-3-(trifluoromethyl)-1H-pyrazol-5-yl)cyclohexanone (second peak on SFC, 270 mg, 28% yield). LCMS (ESI): [M+H]⁺ 275.1. The absolute stereochemistry assigned arbitrarily.

Step 6A: To a solution of (R)-3-(1-isopropyl-3-(trifluoromethyl)-1H-pyrazol-5-yl)cyclohexanone (120 mg, 0.44 mmol), 2-thia-6-azaspiro[3.4]octane 2,2-dioxide hydrochloride (85 mg, 0.53 mmol) and acetic acid (26 mg, 0.44 mmol) in methyl alcohol (3 mL) was added sodium cyanoborohydride (137 mg, 2.19 mmol). The reaction mixture was stirred at 60° C. for 16 h. The pH of the resulting solution was adjusted to ˜9 with NaHCO₃ aq. The resulting mixture was extracted with ethyl acetate (20 mL×2). The combined organic layers were dried over anhydrous sodium sulfate, filtered and concentrated in vacuo. The residue was purified by reversed phase chromatography (DB, Boston Prime C18 150*30 mm*5 μm; water (0.05% NH₃H₂O+10 mM NH₄HCO₃); ACN; 55-85%) to provide a mixture of diasteromers (120 mg, 72% yield), which was further separated using chiral SFC (Daicel Chiralpak AD-H (250 mm*30 mm, 5 μm), 0.1% NH₃ in H₂O; EtOH; 10%; 60 mL/min) to afford Compound 61A* (first peak on SFC. 58.8 mg) and Compound 61B* (second peak on SFC, 41.75 mg). LCMS (ESI): [M+H]+ 420.2.

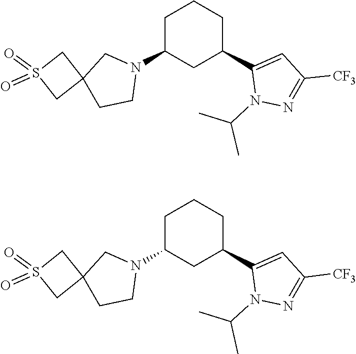 [SMILES: CC(C)n1nc(C(F)(F)F)cc1[C@@H]1CCC[C@H](N2CCC3(C2)CS(=O)(=O)C3)C1 ; CC(C)n1nc(C(F)(F)F)cc1[C@@H]1CCC[C@@H](N2CCC3(C2)CS(=O)(=O)C3)C1]

Compound 61A*: ¹H NMR (400 MHz, CD₃OD) δ 6.37 (s, 1H), 4.68-4.61 (m, 1H), 4.16-4.06 (m, 4H), 3.02-2.95 (m, 2H), 2.90-2.82 (m, 3H), 2.48-2.38 (m, 1H), 2.21-2.13 (m, 3H), 2.10-2.01 (m, 1H), 1.96-1.87 (m, 2H), 1.59-1.49 (m, 1H), 1.47 (dd, J=2.0, 6.4 Hz, 6H), 1.41-1.21 (m, 3H).

Compound 61B*: ¹H NMR (400 MHz, CD₃OD) δ 6.36 (s, 1H), 4.66-4.56 (m, 1H), 4.22-4.00 (m, 4H), 3.29-3.20 (m, 1H), 2.98-2.94 (m, 1H), 2.88-2.75 (m, 2H), 2.71-2.62 (m, 1H), 2.54 (brs, 1H), 2.24-2.13 (m, 2H), 2.07-1.99 (m, 1H), 1.96-1.93 (m, 2H), 1.89-1.78 (m, 1H), 1.68-1.50 (m, 4H), 1.48 (dd, J=2.0, 6.4 Hz, 6H).

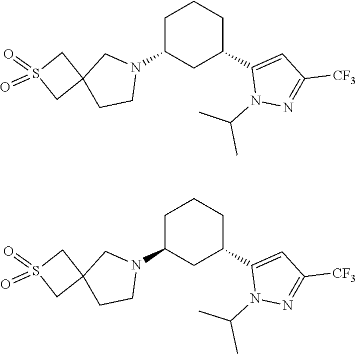 [SMILES: CC(C)n1nc(C(F)(F)F)cc1[C@H]1CCC[C@@H](N2CCC3(C2)CS(=O)(=O)C3)C1 ; CC(C)n1nc(C(F)(F)F)cc1[C@H]1CCC[C@H](N2CCC3(C2)CS(=O)(=O)C3)C1]

Step 6B: To a solution of (S)-3-(1-isopropyl-3-(trifluoromethyl)-1H-pyrazol-5-yl)cyclohexanone (150 mg, 0.55 mmol), 2-thia-6-azaspiro[3.4]octane 2,2-dioxide hydrochloride (106 mg, 0.66 mmol) and acetic acid (33 mg, 0.55 mmol) in methyl alcohol (3 mL) was sodium cyanoborohydride (172 mg, 2.73 mmol). The reaction mixture was stirred at 60° C. for 16 h. The pH of the resulting solution was adjusted to 9 with NaHCO₃ aq., then the resulting mixture was extracted with ethyl acetate (50 mL×2). The combined organic were dried over sodium sulfate, filtered and concentrated under vacuum. The residue was purified by reverse phase chromatography (DG, Phenomenex Gemini-NX 150*30 mm*5 um, water (0.05% NH₃H₂O)-ACN, 44%˜74%) to afford the mixture of diasteromers the title compound (160 mg, 68% yield), LCMS (ESI): [M+H]+ 420.2. The mixture of diasteromers were further separated using chiral SFC (SFC-22, DAICEL CHIRALPAK AD (250 mm*30 mm, 10 um) 0.1% NH₃H₂O ETOH; 10%˜10%, 60 mL/min) to afford Compound 61C* (the first peak on SFC, 51.18 mg) and Compound 61D* (second peak on SFC, 35.17 mg). LCMS (ESI) [M+H]⁺=420.2.

Compound 61C*: ¹H NMR (400 MHz, CD₃OD) δ 6.37 (s, 1H), 4.70-4.58 (m, 1H), 4.17-4.06 (m, 4H), 2.98 (d, J=0.8 Hz, 2H), 2.90-2.81 (m, 3H), 2.48-2.37 (m, 1H), 2.22-2.12 (m, 3H), 2.11-2.03 (m, 1H), 1.97-1.86 (m, 2H), 1.60-1.49 (m, 1H), 1.47 (dd, J=2.0, 6.8 Hz, 6H), 1.41-1.21 (m, 3H).

Compound 61D*: ¹H NMR (400 MHz, CD₃OD) δ 6.36 (s, 1H), 4.63-4.59 (m, 1H), 4.21-4.12 (m, 2H), 4.11-4.02 (m, 2H), 3.29-3.18 (m, 1H), 2.96 (d, J=8.8 Hz, 1H), 2.88-2.75 (m, 2H), 2.71-2.63 (m, 1H), 2.55 (brs, 1H), 2.24-2.12 (m, 2H), 2.07-1.99 (m, 1H), 1.95-1.79 (m, 3H), 1.68-1.50 (m, 4H), 1.48 (dd, J=2.0, 6.8 Hz, 6H).

### Example FFF: 6-((1s,4s)-4-(6-(trifluoromethyl)pyridin-2-yl)cyclohexyl)-2-thia-6-azaspiro[3.4]octane 2,2-dioxide (Compound 62A) and 6-((1r,4r)-4-(6-(trifluoromethyl)pyridin-2-yl)cyclohexyl)-2-thia-6-azaspiro[3.4]octane 2,2-dioxide (Compound 62B)

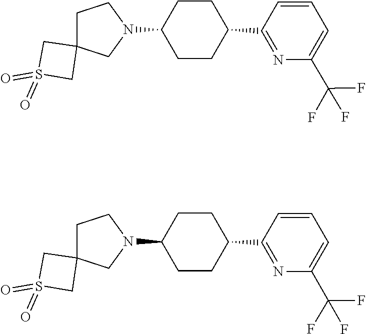 [SMILES: O=S1(=O)CC2(CCN([C@H]3CC[C@@H](c4cccc(C(F)(F)F)n4)CC3)C2)C1 ; O=S1(=O)CC2(CCN([C@H]3CC[C@H](c4cccc(C(F)(F)F)n4)CC3)C2)C1]

Title compounds were synthesized following a procedure similar to Compound 47A using 2-bromo-6-(trifluoromethyl)pyridine. The mixture of the diastereoisomers (70.0 mg, 0.18 mmol) was purified by chiral SFC (Cellulose 2; (150 mm*4.6 mm, 5 μm); 0.1% NH₃ in water; EtOH; 45%; 25 mL/min) to provide Compound 62A (first peak on SFC, 23.1 mg, 33% yield) and Compound 62B (second peak on SFC, 33.2 mg, 47.4% yield). LCMS (ESI) [M+H]⁺=389.1. The relative stereochemistry was assigned based on ¹H NMR analysis.

Compound 62A: ¹H NMR (400 MHz, CD₃OD) 7.92 (t, J=7.6 Hz, 1H), 7.57 (t, J=8.0 Hz, 2H), 4.17-4.08 (m, 4H), 3.02-2.89 (m, 3H), 2.81 (t, J=7.2 Hz, 2H), 2.47 (s, 1H), 2.20 (t, J=7.2 Hz, 2H), 2.17-2.05 (m, 2H), 1.96-1.84 (m, 2H), 1.76-1.65 (m, 4H).

Compound 62B: ¹H NMR (400 MHz, CD₃OD) 7.92 (t, J=8.0 Hz, 1H), 7.59 (d, J=7.2 Hz, 1H), 7.51 (d, J=8.0 Hz, 1H), 4.21-4.09 (m, 4H), 3.11 (s, 2H), 2.96 (t, J=7.2 Hz, 2H), 2.86-2.74 (m, 1H), 2.44-2.38 (m, 1H), 2.25 (t, J=7.2 Hz, 2H), 2.18-2.16 (m, 2H), 2.06-2.02 (m, 2H), 1.72-1.68 (m, 2H), 1.53-1.40 (m, 2H).

### Example GGG: 6-((1r,4r)-4-(5-(trifluoromethyl)pyridin-3-yl)cyclohexyl)-2-thia-6-azaspiro[3.4]octane 2,2-dioxide (Compound 63A) and 6-((1s,4s)-4-(5-(trifluoromethyl)pyridin-3-yl)cyclohexyl)-2-thia-6-azaspiro[3.4]octane 2,2-dioxide (Compound 63B)

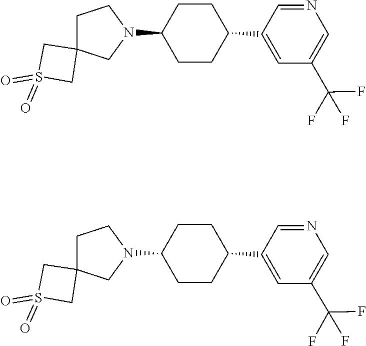 [SMILES: O=S1(=O)CC2(CCN([C@H]3CC[C@H](c4cncc(C(F)(F)F)c4)CC3)C2)C1 ; O=S1(=O)CC2(CCN([C@H]3CC[C@@H](c4cncc(C(F)(F)F)c4)CC3)C2)C1]

Title compounds were synthesized following a procedure similar to Compound 47A using 3-bromo-5-(trifluoromethyl)pyridine. The mixture of the diastereomers were purified by prep-TLC (10% methanol in dichloromethane) to provide Compound 63A (first peak, 53.3 mg, 0.13 mmol, 55% yield) and Compound 63B (second peak, 32.06 mg, 0.08 mmol, 33% yield). LCMS (ESI): [M+H]⁺=389.1. The relative stereochemistry was assigned based on ¹H NMR analysis.

Compound 63A: ¹H NMR (400 MHz, CDCl₃) δ 8.73 (s, 1H), 8.67 (d, J=1.6 Hz, 1H), 7.74 (s, 1H), 4.09 (s, 4H), 2.93 (s, 2H), 2.80 (t, J=7.2 Hz, 2H), 2.67-2.59 (m, 1H), 2.28-2.10 (m, 5H), 2.10-1.97 (m, 2H), 1.57-1.52 (m, 2H), 1.43-1.36 (m, 2H).

Compound 63B: ¹H NMR (400 MHz, CDCl₃) δ 8.73 (s, 1H), 8.67 (d, J=1.6 Hz, 1H), 7.72 (s, 1H), 4.15-4.03 (m, 4H), 2.85 (s, 2H), 2.76-2.72 (m, 3H), 2.47-2.42 (m, 1H), 2.23-2.18 (m, 2H), 2.08-2.01 (m, 2H), 1.97-1.89 (m, 2H), 1.71-1.59 (m, 4H).

### Example HHH: 6-((1r,4r)-4-(2-ethyl-6-(trifluoromethyl)pyridin-3-yl)cyclohexyl)-2-thia-6-azaspiro[3.4]octane 2,2-dioxide (Compound 64A) and 6-((1s,4s)-4-(2-ethyl-6-(trifluoromethyl)pyridin-3-yl)cyclohexyl)-2-thia-6-azaspiro[3.4]octane 2,2-dioxide (Compound 64B)

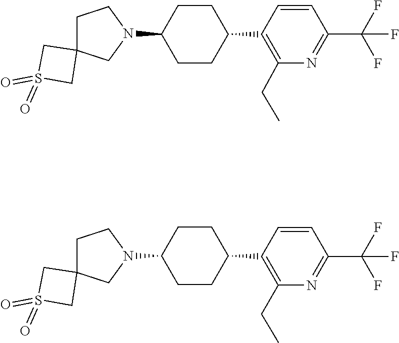 [SMILES: CCc1nc(C(F)(F)F)ccc1[C@H]1CC[C@H](N2CCC3(C2)CS(=O)(=O)C3)CC1 ; CCc1nc(C(F)(F)F)ccc1[C@H]1CC[C@@H](N2CCC3(C2)CS(=O)(=O)C3)CC1]

Title compounds were synthesized following a procedure similar to Compound 47A using 3-bromo-2-ethyl-6-(trifluoromethyl)pyridine. The mixture of the diastereomers was purified by reversed phase chromatography (water (0.05% NH₃ in H₂O+10 mM NH₄HCO₃); ACN; 60-90%) to provide Compound 64A (first peak on HPLC, 46.8 mg, 25.1% yield) and Compound 64B (second peak on HPLC, 19.2 mg, 10.3% yield). LCMS (ESI): [M+H]⁺=417.2. The relative stereochemistry was assigned based on ¹H NMR analysis.

Compound 64A: ¹H NMR (400 MHz, CDCl₃) δ 7.61 (d, J=8.0 Hz, 1H), 7.46 (d, J=8.0 Hz, 1H), 4.08 (s, 4H), 2.95-2.91 (m, 4H), 2.83-2.76 (m, 3H), 2.29-2.24 (m, 1H), 2.19-2.12 (m, 4H), 1.90-1.80 (m, 2H), 1.55-1.45 (m, 4H), 1.31 (t, J=7.6 Hz, 3H).

Compound 64B: ¹H NMR (400 MHz, CDCl₃) δ 7.61 (d, J=8.0 Hz, 1H), 7.47 (d, J=8.0 Hz, 1H), 4.14-4.06 (m, 4H), 2.96-2.90 (m, 3H), 2.85 (s, 2H), 2.75 (t, J=7.2 Hz, 2H), 2.47 (t, J=2.4 Hz, 1H), 2.19 (t, J=7.2 Hz, 2H), 2.08-1.98 (m, 2H), 1.85-1.75 (m, 2H), 1.55-1.40 (m, 4H), 1.30 (t, J=7.6 Hz, 3H).

### Example III: 6-((1r,4r)-4-(2-isopropyl-6-(trifluoromethyl)pyridin-3-yl)cyclohexyl)-2-thia-6-azaspiro[3.4]octane 2,2-dioxide (Compound 65A) and 6-((1s,4s)-4-(2-isopropyl-6-(trifluoromethyl)pyridin-3-yl)cyclohexyl)-2-thia-6-azaspiro[3.4]octane 2,2-dioxide (Compound 65B)

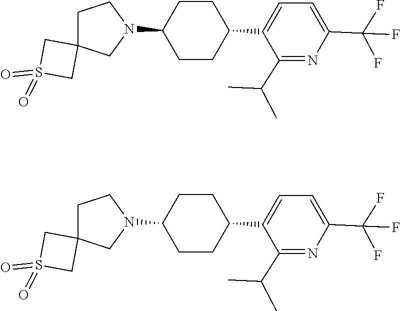 [SMILES: CC(C)c1nc(C(F)(F)F)ccc1[C@H]1CC[C@H](N2CCC3(C2)CS(=O)(=O)C3)CC1 ; CC(C)c1nc(C(F)(F)F)ccc1[C@H]1CC[C@@H](N2CCC3(C2)CS(=O)(=O)C3)CC1]

Title compounds were synthesized following a procedure similar to Compound 47A using 3-bromo-2-isopropyl-6-(trifluoromethyl)pyridine. The mixture of the diastereomers was purified by reversed phase chromatography (water (0.05% NH₃ in H₂O+10 mM NH₄HCO₃); ACN) to afford Compound 65A (first peak on HPLC, 60.18 mg, 0.13 mmol, 37.1% yield) and Compound 65B (second peak on HPLC, 16.1 mg, 0.037 mmol, 10.6% yield). LCMS (ESI) [M+H]⁺=431.2. The relative stereochemistry was assigned based on ¹H NMR analysis.

Compound 65A: ¹H NMR (400 MHz, CDCl₃) δ 7.59 (d, J=8.4 Hz, 1H), 7.42 (d, J=8.0 Hz, 1H), 4.08 (s, 4H), 3.36-3.33 (m, 1H), 2.93 (s, 2H), 2.81 (t, J=7.2 Hz, 3H), 2.29-2.23 (m, 1H), 2.17 (t, J=7.2 Hz, 2H), 2.11-2.08 (m, 2H), 1.91-1.82 (m, 2H), 1.52-1.46 (m, 4H), 1.29 (d, J=6.4 Hz, 6H).

Compound 65B: ¹H NMR (400 MHz, CDCl₃) δ 7.59 (d, J=8.4 Hz, 1H), 7.43 (d, J=8.0 Hz, 1H), 4.14-4.06 (m, 4H), 3.38-3.33 (m, 1H), 2.93-2.87 (m, 1H), 2.85 (s, 2H), 2.75 (t, J=7.2 Hz, 2H), 2.47 (s, 1H), 2.19 (t, J=7.2 Hz, 2H), 2.09-1.99 (m, 2H), 1.86-1.76 (m, 2H), 1.65-1.62 (m, 2H), 1.55-1.52 (m, 2H), 1.29 (d, J=6.4 Hz, 6H).

### Example JJJ: (Compounds 66A* and 66B*) (R)-6-(1-(6-(trifluoromethyl)pyridin-3-yl)piperidin-3-yl)-2-thia-6-azaspiro[3.4]octane 2,2-dioxide and (S)-6-(1-(6-(trifluoromethyl)pyridin-3-yl)piperidin-3-yl)-2-thia-6-azaspiro[3.4]octane 2,2-dioxide

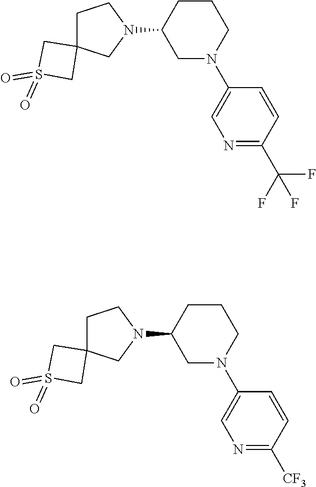 [SMILES: O=S1(=O)CC2(CCN([C@@H]3CCCN(c4ccc(C(F)(F)F)nc4)C3)C2)C1 ; O=S1(=O)CC2(CCN([C@H]3CCCN(c4ccc(C(F)(F)F)nc4)C3)C2)C1]

Title compounds were synthesized following a procedure similar to Compound 55A using 2-thia-6-azaspiro[3.4]octane 2,2-dioxide for the first step and 5-bromo-2-(trifluoromethyl)pyridine for the thirs step. The mixture (130 mg, 0.33 mmol) was separated using chiral SFC (Daicel Chiralpak AD (250 mm*30 mm, 10 μm); 0.1% NH₃ in H₂O; EtOH; 30%; 70 mL/min) to afford Compound 66A* (first peak on SFC, 46.7 mg, 36% yield) and Compound 66B* (second peak on SFC, 50 mg, 38% yield). LCMS (ESI) [M+H]⁺=390.2.

Compound 66A*: ¹H NMR (400 MHz, CD₃OD) δ 8.31 (d, J=2.8 Hz, 1H), 7.59 (d, J=8.8 Hz, 1H), 7.43 (dd, J=2.8, 8.8 Hz, 1H), 4.15-4.06 (m, 4H), 3.98-3.94 (m, 1H), 3.85-3.80 (m, 1H), 3.00 (s, 2H), 2.98-2.84 (m, 4H), 2.50-2.41 (m, 1H), 2.20 (t, J=7.2 Hz, 2H), 2.09-2.04 (m, 1H), 1.90-1.85 (m, 1H), 1.71-1.59 (m, 1H), 1.57-1.46 (m, 1H).

Compound 66B*: 1H NMR (400 MHz, CD₃OD) δ 8.31 (d, J=2.8 Hz, 1H), 7.59 (d, J=8.8 Hz, 1H), 7.43 (dd, J=2.8, 8.8 Hz, 1H), 4.15-4.05 (m, 4H), 4.01-3.95 (m, 1H), 3.85-3.80 (m, 1H), 3.00 (s, 2H), 2.98-2.84 (m, 4H), 2.48-2.39 (m, 1H), 2.19 (t, J=7.2 Hz, 2H), 2.09-2.04 (m, 1H), 1.90-1.87 (m, 1H), 1.71-1.61 (m, 1H), 1.58-1.47 (m, 1H).

### Example KKK: 6-((1r,4r)-4-(6-(difluoromethyl)pyridin-3-yl)cyclohexyl)-2-thia-6-azaspiro[3.4]octane 2,2-dioxide (Compound 67)

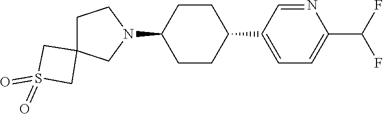 [SMILES: O=S1(=O)CC2(CCN([C@H]3CC[C@H](c4ccc(C(F)F)nc4)CC3)C2)C1]

The title compound was synthesized following a procedure similar to Compound 47A using 5-bromo-2-(difluoromethyl)pyridine. The mixture of the diastereoisomers was purified by reversed phase chromatography (DG, Phenomenex Gemini-NX 150*30 mm*5 μm, water (0.05% NH₃H₂O), ACN, 35-65%) to provide Compound 67 (the second peak on SFC, 69.68 mg, 42%). The relative stereochemistry was assigned based on ¹H NMR analysis. LCMS (ESI) [M+H]⁺=371.2: ¹H NMR (400 MHz, CD₃OD) δ 8.52 (d, J=1.6 Hz, 1H), 7.87 (dd, J=2.0, 8.0 Hz, 1H), 7.63 (d, J=8.0 Hz, 1H), 6.69 (t, J=15.6 Hz, 1H), 4.17-4.09 (m, 4H), 2.99 (s, 2H), 2.84 (t, J=7.2 Hz, 2H), 2.73-2.64 (m, 1H), 2.33-2.26 (m, 1H), 2.23-2.13 (m, 4H), 1.98-1.95 (m, 2H), 1.66-1.56 (m, 2H), 1.49-1.38 (m, 2H).

### Example LLL: (Compounds 68A*, 68B*, 68C*, and 68D*): (R)-7-((1s,4S)-4-(5-(Trifluoromethyl)pyridin-2-yl)cyclohexyl)-2-thia-7-azaspiro[4.5]decane 2,2-dioxide, (R)-7-((1r,4R)-4-(5-(trifluoromethyl)pyridin-2-yl)cyclohexyl)-2-thia-7-azaspiro[4.5]decane 2,2-dioxide, (S)-7-((1s,4R)-4-(5-(trifluoromethyl)pyridin-2-yl)cyclohexyl)-2-thia-7-azaspiro[4.5]decane 2,2-dioxide and (S)-7-((1r,4S)-4-(5-(trifluoromethyl)pyridin-2-yl)cyclohexyl)-2-thia-7-azaspiro[4.5]decane 2,2-dioxide

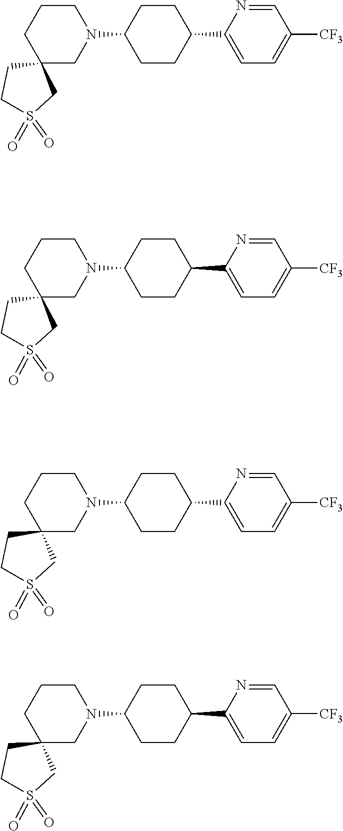 [SMILES: O=S1(=O)CC[C@@]2(CCCN([C@H]3CC[C@@H](c4ccc(C(F)(F)F)cn4)CC3)C2)C1 ; O=S1(=O)CC[C@@]2(CCCN([C@H]3CC[C@H](c4ccc(C(F)(F)F)cn4)CC3)C2)C1 ; O=S1(=O)CC[C@]2(CCCN([C@H]3CC[C@@H](c4ccc(C(F)(F)F)cn4)CC3)C2)C1 ; O=S1(=O)CC[C@]2(CCCN([C@H]3CC[C@H](c4ccc(C(F)(F)F)cn4)CC3)C2)C1]

To a solution of 2-thia-7-azaspiro[4.5]decane 2,2-dioxide hydrochloride (147 mg, 0.65 mmol) and 4-(5-(trifluoromethyl)pyridin-2-yl)cyclohexan-1-one (122 mg, 0.50 mmol) in methanol (3.3 mL) was added N,N-diisopropylethylamine (0.261 mL, 1.50 mmol) and titanium(IV) isopropoxide (0.222 mL, 0.75 mmol). The reaction mixture was stirred at 30° C. for 18 h. Sodium cyanoborohydride (94.3 mg, 1.50 mmol) was added and the reaction mixture was stirred at 50° C. for 18 h. The reaction mixture was diluted with 1N aq. NH₄Cl (2 mL) and 10% methanol in dichloromethane (5 mL). The aqueous layer was extracted with 10% methanol in dichloromethane (2×5 mL). The combined organic layer was concentrated in vacuo. The crude mixture was purified by reversed-phase HPLC (acetonitrile/water gradient with 0.1% NH₄OH) to provide two peaks constituting racemic mixtures. The first peak from the above separation was then subjected to chiral SFC purification (Chiralcel OX, 0.1% ammonium hydroxide in methanol/CO₂, 20:80) to provide the first eluting peak as a pure single stereoisomer of Compound 68A*. LCMS (ESI) [M+H]⁺=417.1. ¹H NMR (400 MHz, DMSO-d₆) δ 8.86 (s, 1H), 8.10 (dd, J=8.3, 2.5 Hz, 1H), 7.52 (d, J=1.6 Hz, 1H), 3.22-3.12 (m, 3H), 2.88 (d, J=13.6 Hz, 1H), 2.80-2.59 (m, 3H), 2.45-2.36 (m, 2H), 2.24 (d, J=11.1 Hz, 1H), 2.10-1.99 (m, 1H), 1.97-1.77 (m, 5H), 1.64-1.51 (m, 4H), 1.51-1.30 (m, 4H).

The first peak from the above separation was then subjected to chiral SFC purification (Chiralcel OX, 0.1% ammonium hydroxide in methanol/CO₂ 20:80) to provide the second eluting peak as a pure single stereoisomer of the title Compound 68B*. LCMS (ESI) [M+H]⁺=417.1. ¹H NMR (400 MHz, DMSO-d₆) δ 8.86 (s, 1H), 8.10 (dd, J=8.3, 2.5 Hz, 1H), 7.52 (d, J=1.6 Hz, 1H), 3.22-3.12 (m, 3H), 2.88 (d, J=13.6 Hz, 1H), 2.80-2.59 (m, 3H), 2.45-2.34 (m, 2H), 2.24 (d, J=11.2 Hz, 1H), 2.10-1.99 (m, 1H), 1.97-1.77 (m, 5H), 1.64-1.30 (m, 8H).

The second peak from the above separation was then subjected to chiral SFC purification (Chiralcel OX, 0.1% ammonium hydroxide in methanol/CO₂ 20:80) to provide the first eluting peak as a pure single stereoisomer of the title Compound 68C*. LCMS (ESI) [M+H]⁺=417.1. ¹H NMR (400 MHz, DMSO-d₆) δ 8.89 (s, 1H), 8.11 (dd, J=8.3, 2.5 Hz, 1H), 7.54 (d, J=8.3 Hz, 1H), 3.22-3.12 (m, 3H), 3.02-2.87 (m, 2H), 2.71-2.55 (m, 2H), 2.31-2.23 (m, 1H), 2.17-1.98 (m, 5H), 1.96-1.80 (m, 3H), 1.67-1.31 (m, 8H).

The second peak from the above separation was then subjected to chiral SFC purification (Chiralcel OX, 0.1% ammonium hydroxide in methanol/CO₂ 20:80) to provide the second eluting peak as a pure single stereoisomer of the title Compound 68D*. LCMS (ESI) [M+H]⁺=417.1. ¹H NMR (400 MHz, DMSO-d₆) δ 8.89 (s, 1H), 8.11 (dd, J=8.3, 2.5 Hz, 1H), 7.54 (d, J=8.4 Hz, 1H), 3.21-3.12 (m, 3H), 3.02-2.87 (m, 2H), 2.72-2.55 (m, 2H), 2.31-2.23 (m, 1H), 2.20-1.98 (m, 5H), 1.96-1.80 (m, 3H), 1.68-1.30 (m, 8H).

### Example MMM: (Compounds 69A*, 69B*, 69Ct*, and 69D*): (R)-7-((1s,4S)-4-(1-Cyclopropyl-3-(trifluoromethyl)-1H-pyrazol-5-yl)cyclohexyl)-2-thia-7-azaspiro[4.5]decane 2,2-dioxide; (R)-7-((1r,4R)-4-(1-cyclopropyl-3-(trifluoromethyl)-1H-pyrazol-5-yl)cyclohexyl)-2-thia-7-azaspiro[4.5]decane 2,2-dioxide; (S)-7-((1s,4R)-4-(1-cyclopropyl-3-(trifluoromethyl)-1H-pyrazol-5-yl)cyclohexyl)-2-thia-7-azaspiro[4.5]decane 2,2-dioxide and (S)-7-((1r,4S)-4-(1-cyclopropyl-3-(trifluoromethyl)-1H-pyrazol-5-yl)cyclohexyl)-2-thia-7-azaspiro[4.5]decane 2,2-dioxide

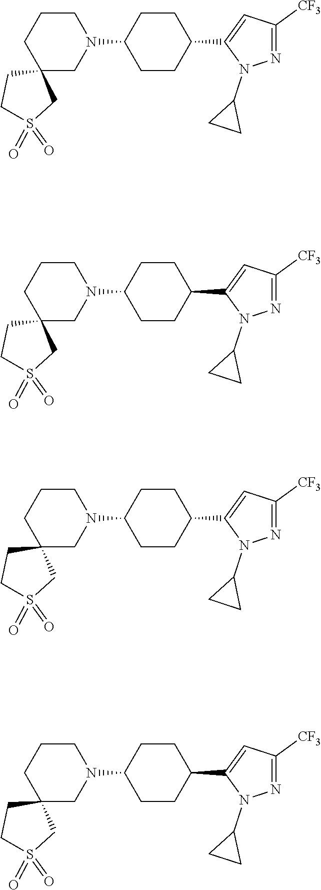 [SMILES: O=S1(=O)CC[C@@]2(CCCN([C@H]3CC[C@@H](c4cc(C(F)(F)F)nn4C4CC4)CC3)C2)C1 ; O=S1(=O)CC[C@@]2(CCCN([C@H]3CC[C@H](c4cc(C(F)(F)F)nn4C4CC4)CC3)C2)C1 ; O=S1(=O)CC[C@]2(CCCN([C@H]3CC[C@@H](c4cc(C(F)(F)F)nn4C4CC4)CC3)C2)C1 ; O=S1(=O)CC[C@]2(CCCN([C@H]3CC[C@H](c4cc(C(F)(F)F)nn4C4CC4)CC3)C2)C1]

The same procedure for Compounds 68A, 68B, 68C, and 68D was followed using 4-(1-cyclopropyl-3-(trifluoromethyl)-1H-pyrazol-5-yl)cyclohexan-1-one. The crude mixture was purified by reversed-phase HPLC (acetonitrile/water gradient with 0.1% NH₄OH) to provide two peaks constituting of racemic mixtures.

The first peak from the above separation was then subjected to chiral SFC purification (Chiralcel OX, 0.1% ammonium hydroxide in methanol/CO₂ 20:80) to provide the first eluting peak as a pure single stereoisomer of the title Compound 69A*. LCMS (ESI) [M+H]⁺=446.1. ¹H NMR (400 MHz, DMSO-d₆) δ 6.49 (s, 1H), 3.75-3.65 (m, 1H), 3.23-3.12 (m, 3H), 2.92-2.84 (m, 2H), 2.71-2.58 (m, 2H), 2.42-2.31 (m, 2H), 2.23 (d, J=11.2 Hz, 1H), 2.09-1.98 (m, 3H), 1.94-1.75 (m, 3H), 1.64-1.32 (m, 8H), 1.14-0.98 (m, 4H).

The first peak from the above separation was then subjected to chiral SFC purification (Chiralcel OX, 0.1% ammonium hydroxide in methanol/CO₂ 20:80) to provide the second eluting peak as a pure single stereoisomer of the title Compound 69B*. LCMS (ESI) [M+H]⁺=446.1. ¹H NMR (400 MHz, DMSO-d₆) δ 6.49 (s, 1H), 3.75-3.65 (m, 1H), 3.21-3.12 (m, 3H), 2.92-2.84 (m, 2H), 2.72-2.58 (m, 2H), 2.45-2.33 (m, 2H), 2.23 (d, J=11.1 Hz, 1H), 2.10-1.98 (m, 3H), 1.94-1.75 (m, 3H), 1.64-1.49 (m, 2H), 1.49-1.32 (m, 6H), 1.14-0.98 (m, 4H).

The second peak from the above separation was then subjected to chiral SFC purification (Chiralcel OX, 0.1% ammonium hydroxide in methanol/CO₂ 20:80) to provide the first eluting peak as a pure single stereoisomer of the title Compound 69C*. LCMS (ESI) [M+H]⁺=446.1. ¹H NMR (400 MHz, DMSO-d₆) δ 6.51 (s, 1H), 3.75-3.65 (m, 1H), 3.25-3.10 (m, 4H), 2.94 (d, J=13.6 Hz, 1H), 2.82-2.59 (m, 2H), 2.28-2.22 (m, 1H), 2.15-1.97 (m, 3H), 1.97-1.86 (m, 3H), 1.81-1.35 (m, 10H), 1.15-0.99 (m, 4H).

Peak 2 from the above separation was then subjected to chiral SFC purification (Chiralcel OX, 0.1% ammonium hydroxide in methanol/CO₂ 20:80) to provide the second eluting peak as a pure single stereoisomer of the title Compound 69D*. LCMS (ESI) [M+H]⁺=446.1. ¹H NMR (400 MHz, DMSO-d₆) δ 6.51 (s, 1H), 3.76-3.65 (m, 1H), 3.24-3.08 (m, 4H), 2.94 (d, J=13.6 Hz, 1H), 2.82-2.60 (m, 2H), 2.28-2.22 (m, 1H), 2.15-1.86 (m, 6H), 1.82-1.35 (m, 10H), 1.15-0.98 (m, 4H).

### Example NNN: 6-((1r,4r)-4-(3,4-difluorophenyl)cyclohexyl)-2-thia-6-azaspiro[3.4]octane 2,2-dioxide (Compound 70A) and 6-((1s,4s)-4-(3,4-difluorophenyl)cyclohexyl)-2-thia-6-azaspiro[3.4]octane 2,2-dioxide (Compound 70B)

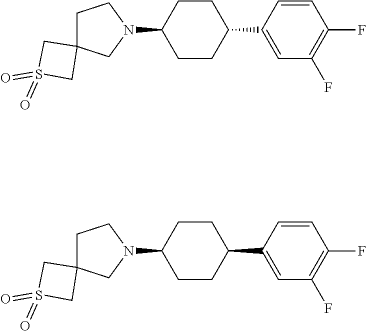 [SMILES: O=S1(=O)CC2(CCN([C@H]3CC[C@H](c4ccc(F)c(F)c4)CC3)C2)C1 ; O=S1(=O)CC2(CCN([C@H]3CC[C@@H](c4ccc(F)c(F)c4)CC3)C2)C1]

Title compounds were synthesized following a procedure similar to Compounds 77A & 77B using 4-bromo-1,2-difluorobenzene. The relative stereochemistry was assigned based on ¹H NMR analysis.

### Example OOO: (Compounds 71A*, 71B*, 71C*, and 71D*): (R)-7-((1s,4S)-4-(6-(Trifluoromethyl)pyridin-3-yl)cyclohexyl)-2-thia-7-azaspiro[4.5]decane 2,2-dioxide, (R)-7-((1r,4R)-4-(6-(trifluoromethyl)pyridin-3-yl)cyclohexyl)-2-thia-7-azaspiro[4.5]decane 2,2-dioxide, (S)-7-((1s,4R)-4-(6-(trifluoromethyl)pyridin-3-yl)cyclohexyl)-2-thia-7-azaspiro[4.5]decane 2,2-dioxide and (S)-7-((1r,4S)-4-(6-(trifluoromethyl)pyridin-3-yl)cyclohexyl)-2-thia-7-azaspiro[4.5]decane 2,2-dioxide

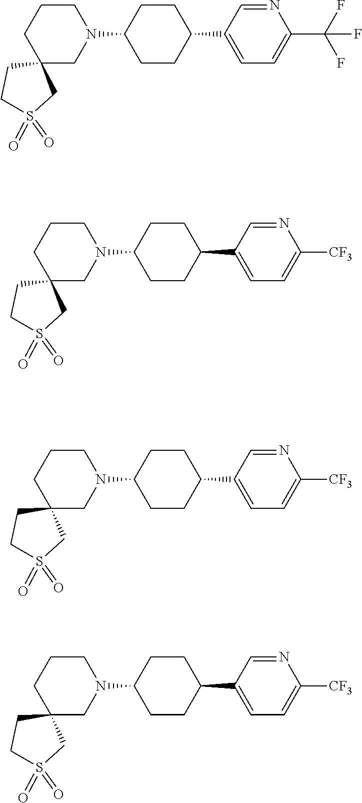 [SMILES: O=S1(=O)CC[C@@]2(CCCN([C@H]3CC[C@@H](c4ccc(C(F)(F)F)nc4)CC3)C2)C1 ; O=S1(=O)CC[C@@]2(CCCN([C@H]3CC[C@H](c4ccc(C(F)(F)F)nc4)CC3)C2)C1 ; O=S1(=O)CC[C@]2(CCCN([C@H]3CC[C@@H](c4ccc(C(F)(F)F)nc4)CC3)C2)C1 ; O=S1(=O)CC[C@]2(CCCN([C@H]3CC[C@H](c4ccc(C(F)(F)F)nc4)CC3)C2)C1]

The same procedure as that followed for compounds 68A, 68B, 68C, and 68D was followed using with 4-(6-(trifluoromethyl)pyridin-3-yl)cyclohexan-1-one. The crude mixture was purified by reversed-phase HPLC (acetonitrile/water gradient with 0.1% NH₄OH) to provide two peaks constituting racemic mixtures. The first peak from the above separation was then subjected to chiral SFC purification (Chiralcel OX, 0.1% ammonium hydroxide in methanol/CO₂ 30:70) to provide the first eluting peak as a pure single stereoisomer of the title Compound 71A*. LCMS (ESI) [M+H]⁺=417.1. ¹H NMR (400 MHz, DMSO-d₆) δ 8.66 (d, J=2.1 Hz, 1H), 7.94 (dd, J=8.1, 2.2 Hz, 1H), 7.81 (dd, J=8.2, 0.8 Hz, 1H), 3.21-3.13 (m, 3H), 2.88 (d, J=13.6 Hz, 1H), 2.72-2.58 (m, 3H), 2.45-2.33 (m, 2H), 2.24 (d, J=11.2 Hz, 1H), 2.10-1.99 (m, 1H), 1.95-1.77 (m, 5H), 1.62-1.32 (m, 8H).

The first peak from the above separation was then subjected to chiral SFC purification (Chiralcel OX, 0.1% ammonium hydroxide in methanol/CO₂ 30:70) to provide the second eluting peak as a pure single stereoisomer of the title Compound 71B*. LCMS (ESI) [M+H]⁺=417.1. ¹H NMR (400 MHz, DMSO-d₆) δ 8.66 (d, J=2.1 Hz, 1H), 7.93 (dd, J=8.1, 2.2 Hz, 1H), 7.81 (dd, J=8.1, 0.8 Hz, 1H), 3.21-3.13 (m, 3H), 2.88 (d, J=13.6 Hz, 1H), 2.72-2.59 (m, 3H), 2.46-2.33 (m, 2H), 2.24 (d, J=11.2 Hz, 1H), 2.10-1.99 (m, 1H), 1.95-1.77 (m, 5H), 1.63-1.33 (m, 8H).

The second peak from the above separation was then subjected to chiral SFC purification (Chiralcel OX, 0.1% ammonium hydroxide in methanol/CO₂ 30:70) to provide the first eluting peak as a pure single stereoisomer of the title Compound 71C*. LCMS (ESI) [M+H]⁺=417.1. ¹H NMR (400 MHz, DMSO-d₆) δ 8.67 (d, J=2.2 Hz, 1H), 7.93 (dd, J=8.2, 2.2 Hz, 1H), 7.83 (dd, J=8.1, 0.8 Hz, 1H), 3.24-3.12 (m, 3H), 2.96 (d, J=13.5 Hz, 1H), 2.89-2.81 (m, 1H), 2.81-2.61 (m, 2H), 2.27-2.22 (m, 1H), 2.10-1.77 (m, 8H), 1.64-1.45 (m, 7H), 1.41-1.30 (m, 1H).

The second peak from the above separation was then subjected to chiral SFC purification (Chiralcel OX, 0.1% ammonium hydroxide in methanol/CO₂ 30:70) to provide the second eluting peak as a pure single stereoisomer of the title Compound 71D*. LCMS (ESI) [M+H]⁺=417.1. ¹H NMR (400 MHz, DMSO-d₆) δ 8.67 (d, J=2.1 Hz, 1H), 7.93 (dd, J=8.1, 2.2 Hz, 1H), 7.83 (dd, J=8.1, 0.8 Hz, 1H), 3.25-3.15 (m, 3H), 2.96 (d, J=13.5 Hz, 1H), 2.89-2.82 (m, 1H), 2.82-2.65 (m, 2H), 2.28-2.22 (m, 1H), 2.10-1.78 (m, 8H), 1.66-1.46 (m, 7H), 1.42-1.32 (m, 1H).

### Example PPP: 6-((1r,4r)-4-(5-fluoro-6-(trifluoromethyl)pyridin-3-yl)cyclohexyl)-2-thia-6-azaspiro[3.4]octane 2,2-dioxide (Compound 72A) and 6-((1s,4s)-4-(5-fluoro-6-(trifluoromethyl)pyridin-3-yl)cyclohexyl)-2-thia-6-azaspiro[3.4]octane 2,2-dioxide (Compound 72B)

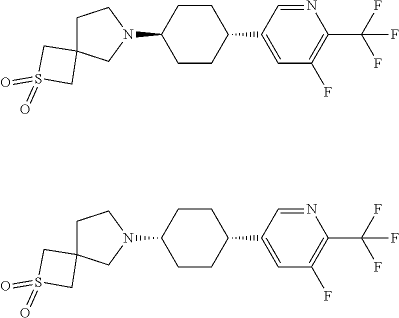 [SMILES: O=S1(=O)CC2(CCN([C@H]3CC[C@H](c4cnc(C(F)(F)F)c(F)c4)CC3)C2)C1 ; O=S1(=O)CC2(CCN([C@H]3CC[C@@H](c4cnc(C(F)(F)F)c(F)c4)CC3)C2)C1]

Title compounds were synthesized following a procedure similar to Compound 47A using 5-bromo-3-fluoro-2-(trifluoromethyl)pyridine. The mixture of the diastereomers was purified by pre-TLC (10% methanol in dichloromethane) to provide title Compound 72A (first peak on SFC, 41.66 mg, 0.10 mmol, 26% yield) and title Compound 72B (second peak on SFC, 87.27 mg, 0.20 mmol, 54% yield). LCMS (ESI) [M+H]⁺=407.1. The relative stereochemistry was assigned based on ¹H NMR analysis.

Compound 72A: ¹H NMR (400 MHz, CDCl₃) δ 8.38 (s, 1H), 7.41 (d, J=10.8 Hz, 1H), 4.08 (s, 4H), 2.92 (s, 2H), 2.80 (t, J=6.8 Hz, 2H), 2.66 (t, J=12.0 Hz, 1H), 2.19-2.10 (m, 5H), 2.02-1.99 (m, 2H), 1.53-1.39 (m, 4H).

Compound 72B: ¹H NMR (400 MHz, CDCl₃) δ 8.38 (s, 1H), 7.40 (d, J=10.8 Hz, 1H), 4.13-4.03 (m, 4H), 2.85 (s, 2H), 2.80-2.70 (m, 3H), 2.45 (s, 1H), 2.18 (t, J=7.2 Hz, 2H), 2.02-1.96 (m, 2H), 1.89-1.78 (m, 2H), 1.67-1.61 (m, 4H).

### Example QQQ: 6-((1r,4r)-4-(1-(tert-Butyl)-3-(trifluoromethyl)-1H-pyrazol-5-yl)cyclohexyl)-2-thia-6-azaspiro[3.4]octane 2,2-dioxide (Compound 73)

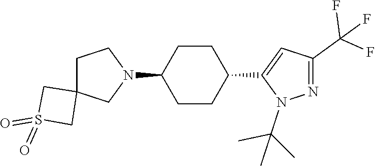 [SMILES: CC(C)(C)n1nc(C(F)(F)F)cc1[C@H]1CC[C@H](N2CCC3(C2)CS(=O)(=O)C3)CC1]

### Step 1: 1-(1,4-Dioxaspiro[4.5]decan-8-yl)ethanone

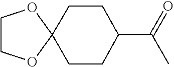

To a cooled (−20° C.) suspension mixture of N,O-dimethylhydroxylamine hydrochloride (3166 mg, 32.46 mmol) and methyl 1,4-dioxaspiro[4.5]decane-8-carboxylate (5.0 g, 24.97 mmol) in THF (150 mL) was added dropwise methyl magnesium chloride (25.8 mL, 77.41 mmol, 3 M in toluene). The reaction mixture was stirred at −20° C. for another 1 hour. Methyl magnesium chloride (43.28 mL, 129.85 mmol, 3 M in toluene) was then added, and stirred at −10° C. to 0° C. for 1.5 h. The reaction mixture was quenched with saturated ammonium chloride (100 mL), water (20 mL), and 4N aqueous HCl (5 mL). The reaction mixture was extracted with ethyl acetate (2×60 mL). The combined extracts were washed with brine, dried over anhydrous Na₂SO₄, filtered and concentrated in vacuo to give the title compound (4200 mg, 22.8 mmol, 97.7% yield). ¹H NMR (400 MHz, CDCl₃) 3.94-3.84 (m, 4H), 2.33 (m, 1H), 2.13 (s, 3H), 1.91-1.83 (m, 2H), 1.79-1.76 (m, 2H), 1.74-1.62 (m, 2H), 1.59-1.49 (m, 2H).

### Step 2: (Z)-4,4,4-Trifluoro-3-hydroxy-1-(1,4-dioxaspiro[4.5]decan-8-yl)but-2-en-1-one

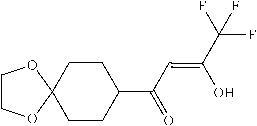 [SMILES: O=C(/C=C(\O)C(F)(F)F)C1CCC2(CC1)OCCO2]

To a stirred solution of 1-(1,4-dioxaspiro[4.5]decan-8-yl)ethanone (2200 mg, 11.94 mmol) in tetrahydrofuran (30 mL) was slowly added lithium bis (trimethylsilyl)amide (17.9 mL, 17.9 mmol, 1 M in THF) at −78° C. under nitrogen. The reaction mixture was stirred at at −78° C. for 0.5 h. Methyl trifluoroacetate (2.56 mL, 23.88 mmol) was then added and the resulting reaction mixture was allowed to warm to 20° C. and stirred for additional 6 h. The reaction mixture was quenched with saturated ammonium chloride (40 mL) and the resulting mixture was extracted with ethyl acetate (100 mL×2). The combined organic layers were washed with brine, dried over anhydrous sodium sulfate, filtered and concentrated in vacuo to give the title compound (3300 mg, 11.8 mmol, 98.6% yield). ¹H NMR (400 MHz, CDCl₃) 5.63 (s, 1H), 3.96-3.88 (m, 4H), 2.82 (brs, 2H), 2.20-2.14 (m, 1H), 1.96-1.75 (m, 3H), 1.70-1.41 (m, 5H).

### Step 3: 1-(tert-Butyl)-5-(1,4-dioxaspiro[4.5]decan-8-yl)-3-(trifluoromethyl)-1H-pyrazole and 1-(tert-butyl)-3-(1,4-dioxaspiro[4.5]decan-8-yl)-3-(trifluoromethyl)-1H-pyrazole

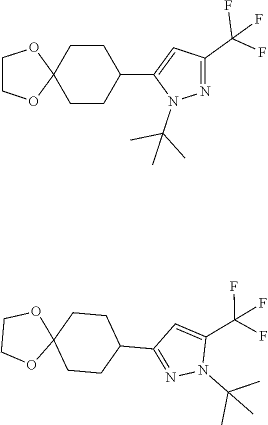 [SMILES: CC(C)(C)n1nc(C(F)(F)F)cc1C1CCC2(CC1)OCCO2 ; CC(C)(C)n1nc(C2CCC3(CC2)OCCO3)cc1C(F)(F)F]

(Z)-4-(1,4-Dioxaspiro[4.5]decan-8-yl)-1,1,1-trifluoro-4-hydroxy-but-3-en-2-one (3000.0 mg, 10.71 mmol) and 1-tert-butylhydrazine hydrochloride (1333.96 mg, 10.71 mmol) in ethanol (30 mL) was added triethylamine (1.48 mL, 10.71 mmol). The reaction mixture and stirred at 25° C. for 15 h. The mixture was concentrated in vacuo and the residue was purified by silica flash chromatography (0-10% ethyl acetate in petroleum ether) to afford 1-(tert-butyl)-5-(1,4-dioxaspiro[4.5]decan-8-yl)-3-(trifluoromethyl)-1H-pyrazole (1500 mg, 3.5654 mmol, 33.3% yield) and 1-(tert-butyl)-3-(1,4-dioxaspiro[4.5]decan-8-yl)-3-(trifluoromethyl)-1H-pyrazole (800 mg, 2.41 mmol, 22.5% yield). LCMS (ESI) [M+H]⁺=333.2.

### Step 4: 4-(1-(tert-Butyl)-3-(trifluoromethyl)-1H-pyrazol-5-yl)cyclohexanone

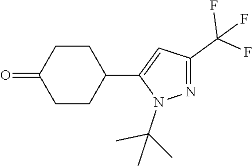 [SMILES: CC(C)(C)n1nc(C(F)(F)F)cc1C1CCC(=O)CC1]

To a solution of 1-tert-butyl-5-(1,4-dioxaspiro[4.5]decan-8-yl)-3-(trifluoromethyl) pyrazole (1500.0 mg, 4.51 mmol) in tetrahydrofuran (4 mL) was added HCl (22.57 mL, 22.57 mmol, 1M) at 25° C. The reaction mixture was stirred at 25° C. for 16 h. The pH of the mixture was adjusted to 9 with the addition of saturated aqueous Na₂CO₃. The mixture was then diluted with water (10 mL), and extracted with ethyl acetate (50 mL×2). The combined organic layers were concentrated in vacuo. The residue was purified by silica flash chromatography (0-20% ethyl acetate in petroleum ether) to afford the title compound (800 mg, 2.39 mmol, 52.9% yield). LCMS (ESI) [M+H]⁺=289.1.

### Step 5: 6-((trans)-4-(1-(tert-Butyl)-3-(Trifluoromethyl)-1H-pyrazol-5-yl)cyclohexyl)-2-thia-6-azaspiro[3.4]octane 2,2-dioxide

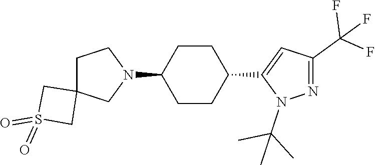 [SMILES: CC(C)(C)n1nc(C(F)(F)F)cc1[C@H]1CC[C@H](N2CCC3(C2)CS(=O)(=O)C3)CC1]

To a solution of 4-[2-tert-butyl-5-(trifluoromethyl)pyrazol-3-yl]cyclohexanone (50.0 mg, 0.17 mmol) in methanol (5 mL) were added 2-thia-6-azaspiro[3.4]octane 2,2-dioxide hydrochloride (51.42 mg, 0.26 mmol), acetic acid (0.01 mL, 0.1700 mmol) and sodium cyanoborohydride (55.0 mg, 0.88 mmol). The reaction mixture was stirred at 70° C. for 3 h. The reaction mixture was quenched with saturated Na₂CO₃ (10 mL) and extracted with ethyl acetate (20 mL×2). The combined organic layers were concentrated in vacuo. The residue was purified by reversed phase chromatography (water (0.05% NH₃ in water+10 mM NH₄HCO₃); ACN, 45-75%) to afford the title compound (30.2 mg, 0.0675 mmol, 38.9% yield). LCMS (ESI) [M+H]⁺=434.1. ¹H NMR (400 MHz, CD₃OD) δ 6.46 (s, 1H), 4.19-4.08 (m, 4H), 3.14-3.06 (m, 1H), 3.04 (s, 2H), 2.89 (t, J=7.2 Hz, 2H), 2.45-2.34 (m, 1H), 2.23 (t, J=7.2 Hz, 2H), 2.15-2.12 (m, 2H), 2.03-1.99 (m, 2H), 1.67 (s, 9H), 1.58-1.54 (m, 2H), 1.46-1.39 (m, 2H).

### Example RRR: 6-((1S,3R)-3-(1-(tert-butyl)-3-cyclopropyl-1H-1,2,4-triazol-5-yl)cyclopentyl)-2-thia-6-azaspiro[3.4]octane 2,2-dioxide (Compound 74A) and 6-((1R,3S)-3-(1-(tert-butyl)-3-cyclopropyl-1H-1,2,4-triazol-5-yl)cyclopentyl)-2-thia-6-azaspiro[3.4]octane 2,2-dioxide (Compound 74B)

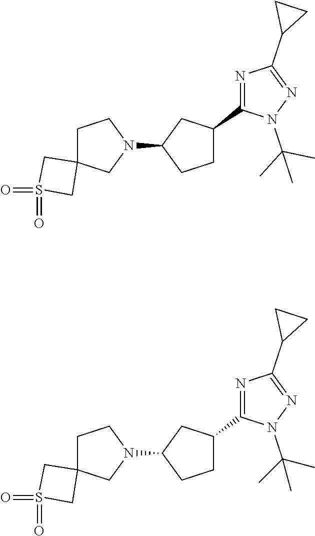 [SMILES: CC(C)(C)n1nc(C2CC2)nc1[C@H]1CC[C@@H](N2CCC3(C2)CS(=O)(=O)C3)C1 ; CC(C)(C)n1nc(C2CC2)nc1[C@@H]1CC[C@H](N2CCC3(C2)CS(=O)(=O)C3)C1]

Title compounds were synthesized following a procedure similar to Compound 53A but instead using 3-oxocyclopentanecarboxylic acid, cyclopropylcarbamidine HCl, and tert-butylhydrazine HCl in the triazole formation reaction (J. Org. Chem. 2011, 76, 1177). The mixture of the (3S)-diastereomers were purified by reversed phase chromatography (acetonitrile; 0.05% ammonia hydroxide in water; 30-60%) to afford the title Compound 74A (33.1 mg, 0.083 mmol, 29.2% yield). LCMS (ESI) [M+H]⁺=393.2. ¹H NMR (400 MHz, CDCl₃) δ 4.15-4.03 (m, 4H), 3.47-3.35 (m, 1H), 2.90-2.82 (m, 2H), 2.78-2.67 (m, 3H), 2.21-2.12 (m, 3H), 2.02-1.91 (m, 4H), 1.89-1.83 (m, 2H), 1.62 (s, 9H), 0.95-0.83 (m, 4H). The cis stereochemistry was assigned based on NOE analysis.

The mixture of the (3R)-diastereomers were purified by reversed phase chromatography (acetonitrile; 0.05% ammonia hydroxide in water; 30-60%) to afford the title Compound 74B (38.6 mg, 0.093 mmol, 21% yield). LCMS (ESI) [M+H]=393.2. ¹H NMR (400 MHz, CDCl₃) δ 4.15-4.03 (m, 4H), 3.47-3.35 (m, 1H), 2.90-2.82 (m, 2H), 2.78-2.67 (m, 3H), 2.21-2.12 (m, 3H), 2.02-1.91 (m, 4H), 1.89-1.83 (m, 2H), 1.62 (s, 9H), 0.95-0.83 (m, 4H). The cis stereochemistry was assigned based on NOE analysis.

### Example SSS: 6-((1r,4r)-4-(2-Cyclopropyl-6-(trifluoromethyl)pyridin-3-yl)cyclohexyl)-2-thia-6-azaspiro[3.4]octane 2,2-dioxide (Compound 75)

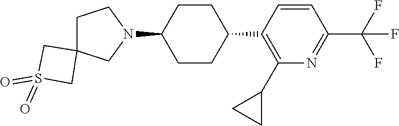 [SMILES: O=S1(=O)CC2(CCN([C@H]3CC[C@H](c4ccc(C(F)(F)F)nc4C4CC4)CC3)C2)C1]

The title compound was synthesized following a procedure similar to Compound 47A using 5-bromo-3-cyclopropyl-2-(trifluoromethyl)pyridine. The mixture of the diastereomers were purified by reversed phase chromatography (Boston Prime C18 150*30 mm*5 μm; water (0.05% NH₃ in water+10 mM NH₄HCO₃); ACN; 65-95%) to give title Compound 75 (second peak on HPLC, 26.2 mg, 0.0582 mmol, 16.5% yield). LCMS (ESI) [M+H]⁺=429.1. The relative stereochemistry was assigned based on ¹H NMR analysis.

Compound 75: ¹H NMR (400 MHz, DMSO-d₆) δ 7.78 (d, J=8.0 Hz, 1H), 7.54 (d, J=8.0 Hz, 1H), 4.23-4.09 (m, 4H), 3.16-3.13 (m, 1H), 2.76 (s, 2H), 2.63 (t, J=7.2 Hz, 2H), 2.43-2.35 (m, 2H), 2.11 (t, J=7.2 Hz, 2H), 1.95-1.91 (m, 2H), 1.87-1.79 (m, 2H), 1.67-1.51 (m, 4H), 1.04-0.92 (m, 4H).

### Example TTT: 7-((1r,4r)-4-(3,4-Difluorophenyl)cyclohexyl)-2-thia-7-azaspiro[3.5]nonane 2,2-dioxide (Compound 77A) and 7-((1s,4s)-4-(3,4-difluorophenyl)cyclohexyl)-2-thia-7-azaspiro[3.5]nonane 2,2-dioxide (Compound 77B)

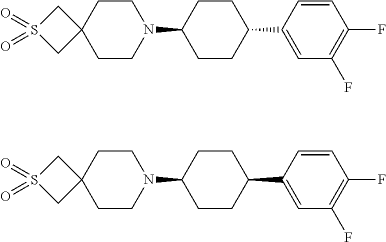 [SMILES: O=S1(=O)CC2(CCN([C@H]3CC[C@H](c4ccc(F)c(F)c4)CC3)CC2)C1 ; O=S1(=O)CC2(CCN([C@H]3CC[C@@H](c4ccc(F)c(F)c4)CC3)CC2)C1]

### Step 1: 8-(3,4-Difluorophenyl)-1,4-dioxaspiro[4.5]dec-7-ene

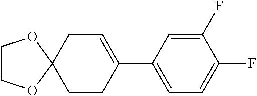 [SMILES: Fc1ccc(C2=CCC3(CC2)OCCO3)cc1F]

To a stirred solution of 4,4,5,5-tetramethyl-2-(1,4-dioxaspiro[4.5]dec-7-en-8-yl)-1,3,2-dioxaborolane (2.44 g, 9.17 mmol) in dioxane (20 mL) was added 1,2-difluoro-4-iodobenzene (2 g, 8.33 mmol), Cs₂CO₃ (8.14 g, 25 mmol) and Pd(dppf)Cl₂ (600 mg, 0.83 mmol). The reaction mixture was stirred at 95° C. overnight. The next day, the reaction mixture was filtered and purified by silica column chromatography (eluted with petroleum ether/toluene/ethyl acetate=4:5:1) to provide the title compound (800 mg, 38% yield). LCMS (ESI) [M+H]⁺=253.1.

### Step 2: 8-(3,4-Difluorophenyl)-1,4-dioxaspiro[4.5]decane

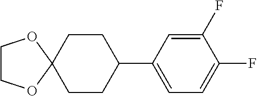 [SMILES: Fc1ccc(C2CCC3(CC2)OCCO3)cc1F]

To a stirred solution of 8-(3,4-difluorophenyl)-1,4-dioxaspiro[4.5]dec-7-ene (800 mg, 3.16 mmol) in ethyl acetate (10 mL) was added Pd/C (100 mg). The reaction mixture was flushed with H₂ and was stirred at rt for 2 h. The reaction mixture was filtered and the solvent was removed in vacuo to afford the title compound (720 mg, 89% yield). LCMS (ESI) [M+H]⁺=255.4.

### Step 3: 4-(3,4-Difluorophenyl)cyclohexan-1-one

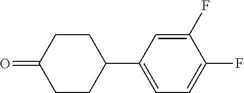 [SMILES: O=C1CCC(c2ccc(F)c(F)c2)CC1]

A solution of 8-(3,4-difluorophenyl)-1,4-dioxaspiro[4.5]decane (720 mg, 2.83 mmol) in acetic acid (8 mL) and water (8 mL) was stirred at 80° C. overnight. The solvent was removed in vacuo to afford the title compound (368 mg, 62% yield). LCMS (ESI) [M+H]⁺=211.1.

### Step 4: 7-((1r,4r)-4-(3,4-Difluorophenyl)cyclohexyl)-2-thia-7-azaspiro[3.5]nonane 2,2-dioxide and 7-((1s,4s)-4-(3,4-difluorophenyl)cyclohexyl)-2-thia-7-azaspiro[3.5]nonane 2,2-dioxide

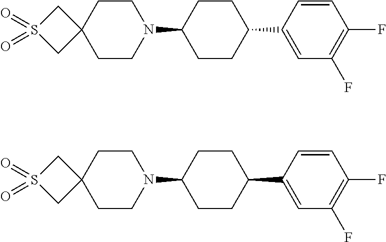 [SMILES: O=S1(=O)CC2(CCN([C@H]3CC[C@H](c4ccc(F)c(F)c4)CC3)CC2)C1 ; O=S1(=O)CC2(CCN([C@H]3CC[C@@H](c4ccc(F)c(F)c4)CC3)CC2)C1]

To a solution of 2-thia-7-azaspiro[3,5]nonane 2,2-dioxide (300 mg, 1.71 mmol) in DCM (10 mL) was added 4-(3,4-difluorophenyl)cyclohexan-1-one (555 mg, 2.64 mmol) dropwise. After stirring overnight at room temperature, NaBH(OAc)₃ (580 mg, 2.74 mmol) was then added at −5° C. The reaction mixture was stirred at −5° C. for an additional 16 h. The reaction was quenched with NaHCO₃ (20 mL), diluted by DCM, washed with brine, dried over magnesium sulfate and was removed in vacuo. The mixture of diastereoisomers was separated by Prep-HPLC (10 mM NH₄HCO₃ in water; ACN; 5%-95% 1.0 mL/min; Xbridge C18, 3.5 μm, 4.6*150 mm) to afford the title compounds (trans: 30 mg, 5% yield) and (cis: 40 mg, 7% yield). The relative cis/trans stereochemistry was assigned based on ¹H NMR analysis.

Compound 77A: ¹H NMR (400 MHz, CDCl₃) δ 7.05 (dt, J=10.3, 8.4 Hz, 1H), 6.98 (ddd, J=10.0, 7.7, 2.1 Hz, 1H), 6.92-6.85 (m, 1H), 3.85 (s, 4H), 2.55 (s, 4H), 2.41 (d, J=11.3 Hz, 2H), 2.05-1.84 (m, 8H), 1.53-1.31 (m, 4H) ppm. LCMS (ESI) [M+H]⁺=370.0

Compound 77B: ¹H NMR (400 MHz, CDCl₃) δ 7.09-7.01 (m, 2H), 6.95-6.92 (m, 1H), 3.85 (s, 4H), 2.70-2.62 (m, 1H), 2.40 (brs, 3H), 2.28-2.24 (m, 1H), 1.93-1.74 (m, 8H), 1.65-1.48 (m 5H) ppm. LCMS (ESI) [M+H]⁺=370.0.

### Example UUU: (Compounds 78A*, 78B*, 78C*, and 78D*): 7-((1R,3R)-3-(1-Isopropyl-3-(trifluoromethyl)-1H-pyrazol-5-yl)cyclohexyl)-2-thia-7-azaspiro[3.5]nonane 2,2-dioxide, 7-((1S,3R)-3-(1-isopropyl-3-(trifluoromethyl)-1H-pyrazol-5-yl)cyclohexyl)-2-thia-7-azaspiro[3.5]nonane 2,2-dioxide, 7-((1R,3S)-3-(1-isopropyl-3-(trifluoromethyl)-1H-pyrazol-5-yl)cyclohexyl)-2-thia-7-azaspiro[3.5]nonane 2,2-dioxide and 7-((1S,3S)-3-(1-isopropyl-3-(trifluoromethyl)-1H-pyrazol-5-yl)cyclohexyl)-2-thia-7-azaspiro[3.5]nonane 2,2-dioxide

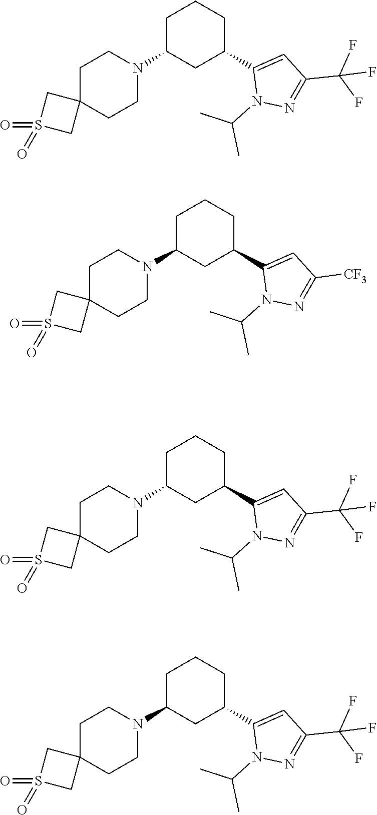 [SMILES: CC(C)n1nc(C(F)(F)F)cc1[C@H]1CCC[C@@H](N2CCC3(CC2)CS(=O)(=O)C3)C1 ; CC(C)n1nc(C(F)(F)F)cc1[C@@H]1CCC[C@H](N2CCC3(CC2)CS(=O)(=O)C3)C1 ; CC(C)n1nc(C(F)(F)F)cc1[C@@H]1CCC[C@@H](N2CCC3(CC2)CS(=O)(=O)C3)C1 ; CC(C)n1nc(C(F)(F)F)cc1[C@H]1CCC[C@H](N2CCC3(CC2)CS(=O)(=O)C3)C1]

Title compounds were synthesized following a procedure similar to Compound 61A.

To a mixture of 3-(1-isopropyl-3-(trifluoromethyl)-1H-pyrazol-5-yl)cyclohexanone (200 mg, 0.72 mmol) in dichloromethane (5 mL) were added 2-thia-7-azaspiro[3.5]nonane 2,2-dioxide hydrochloride (200 mg, 0.94 mmol) and N,N-diisopropylethylamine (282 mg, 2.18 mmol). The reaction mixture was stirred at 25° C. for 16 h. NaBH(OAc)₃ (464 mg, 2.18 mmol) was then added and the reaction mixture was stirred at 65° C. for another 16 h. The reaction mixture was diluted with water (10 mL) and the pH was adjusted to ˜9 with saturated NaHCO₃ aqueous solution. The resulting solution was extracted with ethyl acetate (20 mL×3). The combined organic layers were concentrated in vacuo. The residue was purified by prep-TLC (ethyl acetate/ethanol/petroleum ether=3:1:4). The mixture of cis diastereomers (90 mg, 0.21 mmol) were separated using chiral SFC (Daicel Chiralpak AD (250 mm*30 mm, 10 μm), 0.1% NH₃ in water, EtOH, 10%, 60 mL/min) to afford title Compound 78A* (first peak on SFC, 33.1 mg, 36% yield) and title Compound 78B* (second peak on SFC, 30.9 mg, 33% yield). LCMS (ESI): [M+H]+=434.2.

Compound 78A*: ¹H NMR (400 MHz, CD₃OD) δ 6.40 (s, 1H), 4.64-4.60 (m, 1H), 3.91 (s, 4H), 3.39-3.33 (m, 1H), 2.60-2.35 (m, 4H), 2.07-2.00 (m, 1H), 1.96-1.92 (m, 4H), 1.89-1.55 (m, 8H), 1.47 (dd, J=1.2, 6.8 Hz, 6H).

Compound 78B*: ¹H NMR (400 MHz, CD₃OD) δ 6.40 (s, 1H), 4.64-4.60 (m, 1H), 3.91 (s, 4H), 3.39-3.33 (m, 1H), 2.60-2.35 (m, 4H), 2.07-2.00 (m, 1H), 1.96-1.92 (m, 4H), 1.89-1.55 (m, 8H), 1.47 (dd, J=1.2, 6.8 Hz, 6H).

The mixture of trans diastereoisomers (90 mg, 0.21 mmol) was separated using chiral SFC (Daicel Chirapak AD (250 mm*30 mm, 10 μm) 0.1% NH₃ in water; EtOH; 15%; 60 mL/min) to afford the title compound 78C* (the first peak on SFC, 29.9 mg, 32% yield) and the title compound 78D* (the second peak on SFC, 32.4 mg, 33% yield). LCMS (ESI): [M+H]⁺=434.2.

Compound 78C*: ¹H NMR (400 MHz, CD₃OD) δ 6.38 (s, 1H), 4.69-4.61 (m, 1H), 3.91 (s, 4H), 2.89-2.82 (m, 1H), 2.72-2.64 (m, 4H), 2.10-2.02 (m, 1H), 2.00-1.85 (m, 7H), 1.69-1.19 (m, 11H).

Compound 78D*: ¹H NMR (400 MHz, CD₃OD) δ 6.38 (s, 1H), 4.69-4.61 (m, 1H), 3.91 (s, 4H), 2.89-2.82 (m, 1H), 2.72-2.64 (m, 4H), 2.10-2.02 (m, 1H), 2.00-1.85 (m, 7H), 1.69-1.19 (m, 11H).

### Example VVV: 6-((1r,4r)-4-(6-(Trifluoromethyl)pyridin-3-yl)cyclohexyl)-2-thia-6-azaspiro[3.5]nonane 2,2-dioxide (Compound 79)

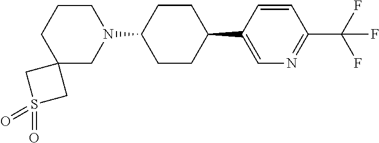 [SMILES: O=S1(=O)CC2(CCCN([C@H]3CC[C@H](c4ccc(C(F)(F)F)nc4)CC3)C2)C1]

### Step 1: 5-(1,4-Dioxaspiro[4.5]dec-7-en-8-yl)-2-(trifluoromethyl)pyridine

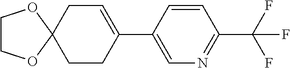 [SMILES: FC(F)(F)c1ccc(C2=CCC3(CC2)OCCO3)cn1]

To a stirred solution of 5-bromo-2-(trifluoromethyl)pyridine (1 g, 4.42 mmol) in 1,4-dioxane (15 mL) and water (3 mL) were added 1,4-dioxa-spiro[4,5]dec-7-en-8-boronic acid pinacol ester (1.41 g, 5.31 mmol), Pd(dppf)Cl₂ (324 mg, 0.44 mmol) and potassium carbonate (1.53 g, 11.06 mmol). The reaction mixture was stirred under N2 at 80° C. for 2 h. The reaction mixture was dissolved in ethyl acetate (100 mL), washed with H₂O (20 mL) and brine (20 mL). The organic layer was dried over anhydrous sodium sulfate, filtered and concentrated in vacuo. The resulting residue was purified by silica flash chromatography (0-20% ethyl acetate in petroleum ether) to afford the title compound (1.0 g, 3.50 mmol, 79% yield). LCMS (ESI) [M+H]⁺=286.1

### Step 2: 5-(1,4-Dioxaspiro[4.5]decan-8-yl)-2-(trifluoromethyl)pyridine

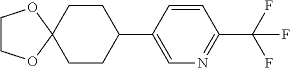 [SMILES: FC(F)(F)c1ccc(C2CCC3(CC2)OCCO3)cn1]

To a solution of 5-(1,4-dioxaspiro[4.5]dec-7-en-8-yl)-2-(trifluoromethyl)pyridine (1.0 g, 3.51 mmol) in ethanol (15 mL) was added 10% palladium on carbon (746 mg). The solution was degassed under vacuo and purged with H₂ (15 psi) three times. The mixture was stirred under H₂ (15 psi) at 25° C. for 16 h. The resulting mixture was filtered and the filtrate was concentrated in vacuo to afford the title compound (1.0 g, 3.48 mmol, 99% yield). LCMS (ESI) [M+H]⁺=288.1.

### Step 3: 4-(6-(Trifluoromethyl)pyridin-3-yl)cyclohexanone

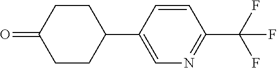 [SMILES: O=C1CCC(c2ccc(C(F)(F)F)nc2)CC1]

To a solution of 5-(1,4-dioxaspiro[4.5]decan-8-yl)-2-(trifluoromethyl)pyridine (1.0 g, 3.48 mmol) in dioxane (5 mL) was added hydrochloric acid (3.3 mL, 19.8 mmol) (6 M in water). The reaction mixture was stirred at 25° C. for 2 h. The pH of the mixture was adjusted to 7 with a saturated NaHCO₃ aqueous solution at 0° C. The reaction mixture was then extracted with ethyl acetate (50 mL×3). The combined organic layers were washed with brine (30 mL), dried over anhydrous Na₂SO₄, filtered and concentrated in vacuo. The residue was purified by silica flash chromatography (0-10% ethyl acetate in petroleum ether) to give the title compound (800 mg, 3.28 mmol, 94.5% yield).

### Step 4: 6-((trans)-4-(6-(Trifluoromethyl)pyridin-3-yl)cyclohexyl)-2-thia-6-azaspiro[3.5]nonane 2,2-dioxide

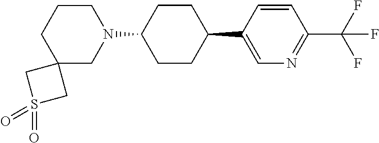 [SMILES: O=S1(=O)CC2(CCCN([C@H]3CC[C@H](c4ccc(C(F)(F)F)nc4)CC3)C2)C1]

To a solution of 4-[6-(trifluoromethyl)-3-pyridyl]cyclohexanone (50.0 mg, 0.21 mmol) in dichloromethane (2 mL) was added 2-thia-6-azaspiro[3.5]nonane 2,2-dioxide hydrochloride (48 mg, 0.23 mmol), N,N-diisopropylethylamine (0.14 mL, 0.82 mmol) and 4A& molecular sieves. The reaction mixture was stirred at 60° C. for 15 h. Sodium triacetoxyborohydride (131 mg, 0.62 mmol) was then added and stirred at 25° C. for another 2 hours. The mixture was concentrated and purified by reversed phase chromatography (DG, Phenomenex Gemini-NX 150*30 mm*5 μm; water (0.05% NH₃); ACN, 35-65%) to afford title Compound 79 (second peak, 27 mg, 32% yield). LCMS (ESI) [M+H]⁺=403.1. ¹H NMR (400 MHz, CD₃OD) δ 8.59 (s, 1H), 7.92 (d, J=8.0 Hz, 1H), 7.74 (d, J=8.0 Hz, 1H), 3.90-3.80 (m, 4H), 2.76-2.65 (m, 3H), 2.64-2.51 (m, 3H), 2.03-1.98 (m, 4H), 1.78-1.71 (m, 2H), 1.64-1.53 (m, 6H).

### Example WWW: 7-((1r,4r)-4-(3-Fluoro-5-(trifluoromethyl)pyridin-2-yl)cyclohexyl)-2-thia-7-azaspiro[3.5] nonane 2,2-dioxide (Compound 80A) and 7-((1s,4s)-4-(3-fluoro-5-(trifluoromethyl)pyridin-2-yl)cyclohexyl)-2-thia-7-azaspiro[3.5]nonane 2,2-dioxide (Compound 80B)

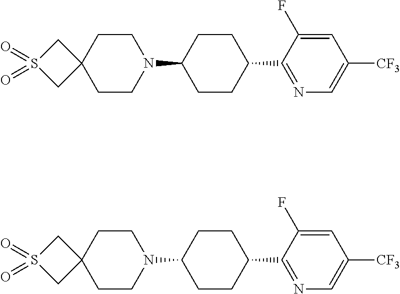 [SMILES: O=S1(=O)CC2(CCN([C@H]3CC[C@H](c4ncc(C(F)(F)F)cc4F)CC3)CC2)C1 ; O=S1(=O)CC2(CCN([C@H]3CC[C@@H](c4ncc(C(F)(F)F)cc4F)CC3)CC2)C1]

### Step 1: 3-Fluoro-2-(1,4-dioxaspiro[4.5]dec-7-en-8-yl)-5-(trifluoromethyl)pyridine

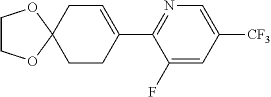 [SMILES: Fc1cc(C(F)(F)F)cnc1C1=CCC2(CC1)OCCO2]

To a stirred solution of 2-bromo-3-fluoro-5-(trifluoromethyl)pyridine (1.0 g, 4 mmol) and 4,4,5,5-tetramethyl-2-(1,4-dioxaspiro[4.5]dec-7-en-8-yl)-1,3,2-dioxaborolane (1.08 g, 4 mmol) in dioxane (20 mL) were added Cs₂CO₃ (3.9 g, 12 mmol) and 146 mg Pd(dppf)Cl₂ (146 mg, 0.2 mmol). The reaction mixture was stirred at 70° C. for 48 h. The mixture was subsequently filtrated and the solvent was evaporated in vacuo. The residue was purified by silica flash chromatography (petroleum ether/ethyl acteate, 90:10) to afford the title compound (1.21 g, 99% yield). LCMS (ESI) [M+H]⁺=304.0.

### Step 2: 3-Fluoro-2-(1,4-dioxaspiro[4.5]decan-8-yl)-5-(trifluoromethyl)pyridine

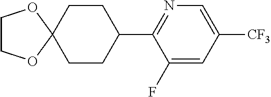 [SMILES: Fc1cc(C(F)(F)F)cnc1C1CCC2(CC1)OCCO2]

To a solution of 3-fluoro-2-(1,4-dioxaspiro[4.5]dec-7-en-8-yl)-5-(trifluoromethyl)pyridine (1.21 g, 4.0 mmol) in ethanol (15 mL) under N2 was added Pd/C (10%, 0.5 g). The nitrogen was then replaced by hydrogen. The reaction was stirred at rt for 12 h. The resultant mixture was filtered and concentrated in vacuo to provide the title compound (1.22 g, quantitative yield). LCMS (ESI) [M+H]⁺=306.1.

### Step 3: 4-(3-Fluoro-5-(trifluoromethyl)pyridin-2-yl)cyclohexan-1-one

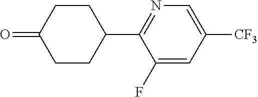 [SMILES: O=C1CCC(c2ncc(C(F)(F)F)cc2F)CC1]

3-Fluoro-2-(1,4-dioxaspiro[4.5]decan-8-yl)-5-(trifluoromethyl)pyridine (1.22 g, 4.0 mmol) was dissolved in a 1:1 mixture of AcOH/H₂O (20 mL). The reaction mixture was stirred at 50° C. for 5 h. The solvent was removed in vacuo and the residue was purified by silica flash chromatography (petroleum ether/ethyl acetate: 95/5 to 80/20) to afford the title compound (0.86 g, 82% yield). LCMS (ESI) [M+H]⁺=262.0.

### Step 4: 7-((1r,4r)-4-(3-Fluoro-5-(trifluoromethyl)pyridin-2-yl)cyclohexyl)-2-thia-7-azaspiro[3.5] nonane 2,2-dioxide and 7-((1s,4s)-4-(3-fluoro-5-(trifluoromethyl)pyridin-2-yl)cyclohexyl)-2-thia-7-azaspiro[3.5]nonane 2,2-dioxide

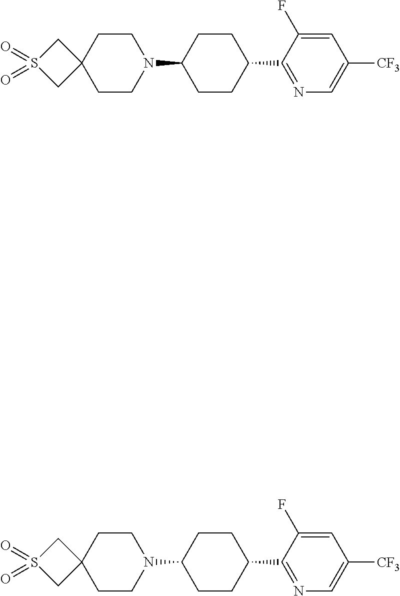 [SMILES: O=S1(=O)CC2(CCN([C@H]3CC[C@H](c4ncc(C(F)(F)F)cc4F)CC3)CC2)C1 ; O=S1(=O)CC2(CCN([C@H]3CC[C@@H](c4ncc(C(F)(F)F)cc4F)CC3)CC2)C1]

To a solution of 2-thia-7-azaspiro[3.5]nonane 2,2-dioxide hydrogen chloride (220 mg, 1.2 mmol) in DCM (10 mL) were added NaBH(OAc)₃ (4.1 mmol, 870 mg) and AcOH (200 μL, 3.2 mmol). 4-(3-Fluoro-5-(trifluoromethyl)pyridin-2-yl)cyclohexan-1-one (450 mg, 1.7 mmol) in DCE (5 mL) was added dropwise at −5° C. The reaction mixture was stirred at rt overnight. The mixture was quenched with saturated NH₄Cl solution and extracted with DCM (3×20 mL). The combined organic layers were concentrated and the residue was purified on reverse phase chromatography (0.1% TFA in water; acetonitrile; 95/5 to 5/95), followed by a separation of the diastereoisomers via prep-HPLC (water (0.01% TFA); ACN (0.01% TFA); 5% to 95%; 1.0 m/min, XBridge peptide BEH column C18, 4.6*150 mm, 3.5 μm, 130 Å) to afford the title compounds (trans: 23.5 mg, 3% yield) and (cis: 67 mg, 9% yield). The relative cis/trans stereochemistry was assigned based on ¹H NMR analysis.

Compound 80A: ¹H NMR (400 MHz, CDCl₃) δ 8.60 (s, 1H), 7.54 (dd, J=9.4, 1.6 Hz, 1H), 3.84 (s, 4H), 3.04 (t, J=11.4 Hz, 1H), 2.75-2.26 (m, 5H), 2.01-1.69 (m, 10H), 1.50-1.35 (m, 2H); LCMS (ESI) [M+H]⁺=421.1.

Compound 80B: ¹H NMR (400 MHz, CDCl₃) δ 8.64 (s, 1H), 7.54 (dd, J=9.6, 1.5 Hz, 1H), 3.85 (s, 4H), 3.29 (d, J=1.3 Hz, 1H), 2.42 (d, J=59.5 Hz, 5H), 2.19-2.06 (m, 2H), 2.06-1.97 (m, 2H), 1.95 (t, J=5.2 Hz, 4H), 1.75-1.52 (m, 4H); LCMS (ESI) [M+H]⁺=421.1.

### Example XXX: (Compounds 81A* and 81B*): 7-((1r,4r)-4-(6-(Trifluoromethyl)pyridin-3-yl)cyclohexyl)-2-thia-7-azaspiro[3.5]nonane 2,2-dioxide and 7-((1s,4s)-4-(6-(Trifluoromethyl)pyridin-3-yl)cyclohexyl)-2-thia-7-azaspiro[3.5]nonane 2,2-dioxide

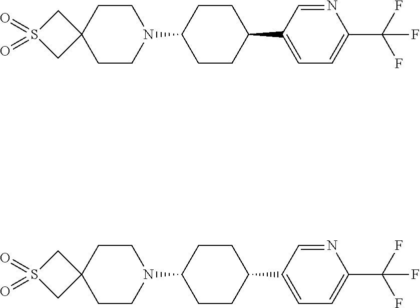 [SMILES: O=S1(=O)CC2(CCN([C@H]3CC[C@H](c4ccc(C(F)(F)F)nc4)CC3)CC2)C1 ; O=S1(=O)CC2(CCN([C@H]3CC[C@@H](c4ccc(C(F)(F)F)nc4)CC3)CC2)C1]

The title compound was synthesized using the same procedure as Compound 50A using 2-thia-7-azaspiro[3.5]nonane 2,2-dioxide for step 4 and 5-bromo-2-(trifluoromethyl)pyridine for step 1. The relative cis/trans stereochemistry was assigned based on ¹H NMR analysis.

Compound 81A* was obtained (second eluting peak, 26.8 mg, 13% yield). LCMS (ESI) [M+H]⁺=403.1, ¹H NMR (400 MHz, DMSO) δ 8.66 (d, J=2.1 Hz, 1H), 7.93 (dd, J=8.1, 2.2 Hz, 1H), 7.81 (dd, J=8.1, 0.8 Hz, 1H), 3.91 (s, 4H), 2.67-2.58 (m, 1H), 2.48-2.34 (m, 5H), 1.93-1.82 (m, 4H), 1.77 (t, J=5.3 Hz, 4H), 1.60-1.46 (m, 2H), 1.46-1.32 (m, 2H).

The title compound was synthesized using the same procedure as Compound 50B.

Compound 81B* was obtained (first eluting peak, 26.8 mg, 13% yield). LCMS (ESI) [M+H]⁺=403.1; ¹H NMR (400 MHz, DMSO) δ 8.66 (d, J=2.1 Hz, 1H), 7.92 (dd, J=8.2, 2.2 Hz, 1H), 7.82 (dd, J=8.2, 0.8 Hz, 1H), 3.92 (s, 4H), 2.91-2.80 (m, 1H), 2.47-2.26 (m, 4H), 2.25-2.20 (m, 1H), 1.98-1.83 (m, 4H), 1.80 (t, J=5.3 Hz, 4H), 1.63-1.46 (m, 4H).

### Example YYY: (Compounds 82A*, 82B*, 82C*, and 82D*): 6-((2R,4R)-2-Isopropyl-1-(6-(trifluoromethyl)pyridin-3-yl)piperidin-4-yl)-2-thia-6-azaspiro[3.4]octane 2,2-dioxide, 6-((2S,4S)-2-isopropyl-1-(6-(trifluoromethyl)pyridin-3-yl)piperidin-4-yl)-2-thia-6-azaspiro[3.4]octane 2,2-dioxide, 6-((2R,4S)-2-isopropyl-1-(6-(trifluoromethyl)pyridin-3-yl)piperidin-4-yl)-2-thia-6-azaspiro[3.4]octane 2,2-dioxide, 6-((2S,4R)-2-isopropyl-1-(6-(trifluoromethyl) pyridin-3-yl)piperidin-4-yl)-2-thia-6-azaspiro[3.4]octane 2,2-dioxide

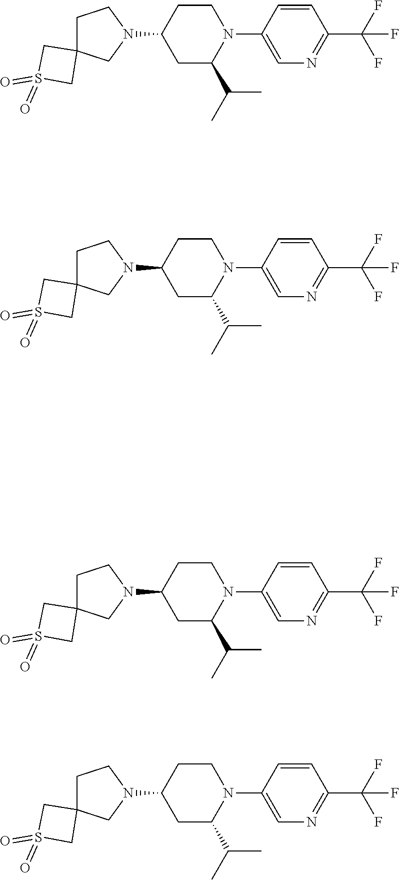 [SMILES: CC(C)[C@H]1C[C@H](N2CCC3(C2)CS(=O)(=O)C3)CCN1c1ccc(C(F)(F)F)nc1 ; CC(C)[C@@H]1C[C@@H](N2CCC3(C2)CS(=O)(=O)C3)CCN1c1ccc(C(F)(F)F)nc1 ; CC(C)[C@H]1C[C@@H](N2CCC3(C2)CS(=O)(=O)C3)CCN1c1ccc(C(F)(F)F)nc1 ; CC(C)[C@@H]1C[C@H](N2CCC3(C2)CS(=O)(=O)C3)CCN1c1ccc(C(F)(F)F)nc1]

Title compounds were synthesized following a procedure similar to Compound 55A using tert-butyl 2-isopropyl-4-oxopiperidine-1-carboxylate and 2-thia-6-azaspiro[3.4]octane 2,2-dioxide for the first step and 5-bromo-2-(trifluoromethyl)pyridine for the third step.

The mixture of isomers (160 mg, 0.371 mmol) was separated using chiral SFC (Daicel Chiralpak AD (250 mm*50 mm, 10 μm); 0.1% NH₃ in water; EtOH; 30%; 60 mL/min) to afford title Compound 82A* (15.42 mg, 0.034 mmol, 9.6% yield), title Compound 82B* (6.69 mg, 0.0146 mmol, 4.2% yield), title Compound 82C* (24.92 mg, 0.0537 mmol, 15.6% yield) and title Compound 82D* (27.06 mg, 0.0571 mmol, 16.9% yield). LCMS (ESI) [M+H]⁺=432.1.

Compound 82A*: ¹H NMR (400 MHz, CD₃OD) δ 8.24 (d, J=2.8 Hz, 1H), 7.56 (d, J=8.8 Hz, 1H), 7.38 (m, 1H), 4.15-4.06 (m, 4H), 3.67-3.58 (m, 1H), 3.56-3.49 (m, 1H), 3.43-3.35 (m, 1H), 2.96-2.87 (m, 2H), 2.77 (t, J=7.2 Hz, 2H), 2.35-2.25 (m, 1H), 2.23-2.10 (m, 4H), 2.08-1.99 (m, 1H), 1.70-1.58 (m, 2H), 1.01 (d, J=6.8 Hz, 3H), 0.95 (d, J=6.8 Hz, 3H).

Compound 82B*: ¹H NMR (400 MHz, CD₃OD) δ 8.23 (d, J=2.8 Hz, 1H), 7.56 (d, J=8.8 Hz, 1H), 7.37 (m, 1H), 4.15-4.06 (m, 4H), 3.66-3.57 (m, 1H), 3.54-3.48 (m, 1H), 3.42-3.38 (m, 1H), 2.95-2.86 (m, 2H), 2.76 (t, J=7.2 Hz, 2H), 2.34-2.24 (m, 1H), 2.22-2.10 (m, 4H), 2.07-1.99 (m, 1H), 1.70-1.57 (m, 2H), 1.01 (d, J=6.4 Hz, 3H), 0.95 (d, J=6.8 Hz, 3H).

Compound 82C*: ¹H NMR (400 MHz, CD₃OD) δ 8.28 (d, J=2.8 Hz, 1H), 7.55 (d, J=9.2 Hz, 1H), 7.40 (m, 1H), 4.16-4.07 (m, 4H), 3.95-3.84 (m, 1H), 3.79-3.72 (m, 1H), 3.30-3.20 (m, 1H), 3.00-2.91 (m, 2H), 2.88-2.75 (m, 2H), 2.72-2.63 (m, 1H), 2.38-2.29 (m, 1H), 2.26-2.16 (m, 3H), 2.03-1.95 (m, 1H), 1.54-1.38 (m, 2H), 1.08 (d, J=6.4 Hz, 3H), 0.89 (d, J=6.8 Hz, 3H).

Compound 82D*: ¹H NMR (400 MHz, CD₃OD) δ 8.26 (d, J=3.2 Hz, 1H), 7.54 (d, J=9.2 Hz, 1H), 7.38 (dd, J=2.8, 8.8 Hz, 1H), 4.15-4.04 (m, 4H), 3.95-3.87 (m, 1H), 3.79-3.73 (m 1H), 3.27-3.19 (m, 1H), 2.97-2.90 (m, 2H), 2.86-2.76 (m, 2H), 2.72-2.66 (m, 1H), 2.37-2.29 (m, 1H), 2.24-2.13 (m, 3H), 2.01-1.91 (m, 1H), 1.51-1.35 (m, 2H), 1.06 (d, J=6.4 Hz, 3H), 0.87 (d, J=6.8 Hz, 3H).

### Example ZZZ: 6-((1r,4r)-4-(6-(Trifluoromethyl)pyrimidin-4-yl)cyclohexyl)-2-thia-6-azaspiro[3.4]octane 2,2-dioxide (Compound 83)

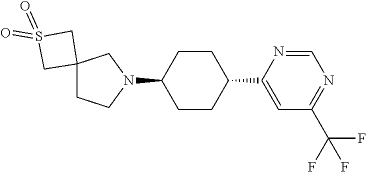 [SMILES: O=S1(=O)CC2(CCN([C@H]3CC[C@H](c4cc(C(F)(F)F)ncn4)CC3)C2)C1]

The title compound was synthesized following a procedure similar to Compound 47A using 4-bromo-6-(trifluoromethyl)pyrimidine. The mixture of the diastereoisomers was purified by reversed phase chromatography (Boston Prime C18 150*30 mm*5 μm, water (0.05% NH₃H₂O+10 mM NH₄HCO₃); ACN; 35-65%) to afford title Compound 83 (first peak on HPLC, 19.6 mg, 0.0503 mmol, 15.4% yield). The relative cis/trans stereochemistry was assigned based on ¹H NMR analysis. LCMS (ESI): [M+H]⁺=390.1. ¹H NMR (400 MHz, CDCl₃) δ 9.19 (s, 1H), 7.44 (s, 1H), 4.01 (s, 4H), 2.93 (s, 2H), 2.82-2.74 (m, 3H), 2.22-2.14 (m, 1H), 2.11-1.96 (m, 6H), 1.59-1.49 (m, 2H), 1.38-1.25 (m, 2H).

### Example AAAA: (Compounds 84A* and 84B*): 2-((1r,4r)-4-(5-(Trifluoromethyl)pyridin-2-yl)cyclohexyl)-8-thia-2-azaspiro[4.5]decane 8,8-dioxide and 2-((1s,4s)-4-(5-(Trifluoromethyl)pyridin-2-yl)cyclohexyl)-8-thia-2-azaspiro[4.5]decane 8,8-dioxide

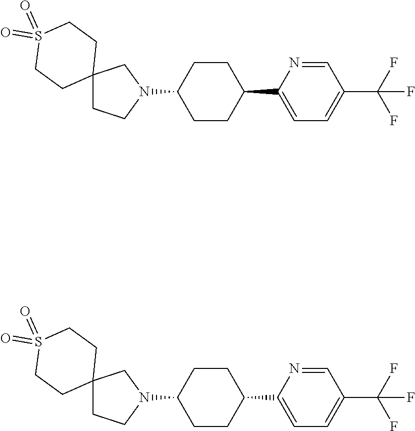 [SMILES: O=S1(=O)CCC2(CCN([C@H]3CC[C@H](c4ccc(C(F)(F)F)cn4)CC3)C2)CC1 ; O=S1(=O)CCC2(CCN([C@H]3CC[C@@H](c4ccc(C(F)(F)F)cn4)CC3)C2)CC1]

The title compounds were synthesized using the same procedure as Compound 50A using 8-thia-2-azaspiro[4.5]decane 8,8-dioxide for step 4. The mixture was purified by preparative reversed phase HPLC (acetonitrile/water gradient with 0.1% NH₄OH).

Compound 84A* was obtained as the second eluting peak (11 mg, 12% yield). LCMS (ESI) [M+H]⁺=417.1. ¹H NMR (400 MHz, DMSO-d₆) δ 8.86 (s, 1H), 8.10 (dd, J=8.3, 2.5 Hz, 1H), 7.52 (d, J=8.3 Hz, 1H), 3.03 (t, J=5.9 Hz, 4H), 2.82-2.71 (m, 1H), 2.61 (t, J=7.0 Hz, 2H), 2.11-2.00 (m, 3H), 1.97-1.82 (m, 6H), 1.67-1.48 (m, 4H), 1.36-1.16 (m, 4H).

Compound 84B* was obtained as the first eluting peak (17 mg; 19% yield). LCMS (ESI) [M+H]⁺=417.1. ¹H NMR (400 MHz, DMSO) δ 8.87 (s, 1H), 8.09 (dd, J=8.3, 2.8 Hz, 1H), 7.48 (d, J=8.3 Hz, 1H), 3.05 (t, J=5.8 Hz, 4H), 2.91-2.82 (m, 1H), 2.56 (t, J=7.1 Hz, 2H), 2.43 (s, 2H), 2.25-2.20 (m, 1H), 2.01-1.85 (m, 8H), 1.67 (t, J=7.1 Hz, 2H), 1.62-1.48 (m, 4H).

### Example BBBB: 7-((1r,4r)-4-(1-(tert-Butyl)-3-(trifluoromethyl)-1H-pyrazol-5-yl)cyclohexyl)-2-thia-7-azaspiro[3.5]nonane 2,2-dioxide (Compound 85A) and 7-((1s,4s)-4-(1-(tert-butyl)-3-(trifluoromethyl)-1H-pyrazol-5-yl)cyclohexyl)-2-thia-7-azaspiro[3.5]nonane 2,2-dioxide (Compound 85B)

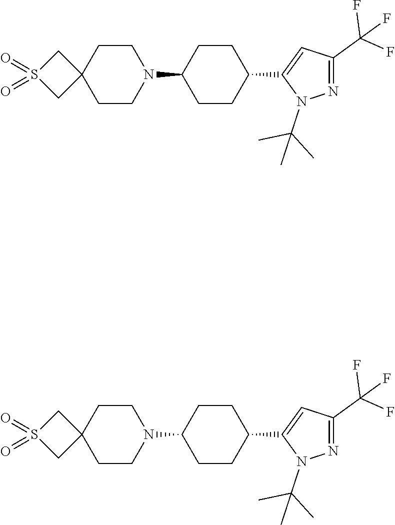 [SMILES: CC(C)(C)n1nc(C(F)(F)F)cc1[C@H]1CC[C@H](N2CCC3(CC2)CS(=O)(=O)C3)CC1 ; CC(C)(C)n1nc(C(F)(F)F)cc1[C@H]1CC[C@@H](N2CCC3(CC2)CS(=O)(=O)C3)CC1]

Title compounds were synthesized following a procedure similar to Compound 73 using 2-thia-7-azaspiro[3.5]nonane 2,2-dioxide for step 5. The mixture of the diastereoisomers was purified by reversed phase chromatography (acetonitrile; 0.05% ammonia hydroxide in water; 30% to 60%) to afford the title compound 85A (first peak on HPLC, 36.4 mg, 0.0796 mmol, 22.9% yield) and the title compound 85B (second peak on HPLC, 29.2 mg, 0.06 mmol, 17.3% yield). LCMS (ESI) [M+H]⁺=448.1. The relative cis/trans stereochemistry was assigned based on ¹H NMR analysis.

Compound 85A: ¹H NMR (400 MHz, CDCl₃) δ 6.33 (s, 1H), 3.85 (s, 4H), 2.93-2.90 (m, 1H), 2.58-2.52 (m, 3H), 2.49-2.41 (m, 1H), 2.07-1.95 (m, 4H), 1.92 (t, J=5.2 Hz, 4H), 1.66 (s, 9H), 1.51-1.32 (m, 5H).

Compound 85B: ¹H NMR (400 MHz, CDCl₃) δ 6.38 (s, 1H), 3.89 (s, 4H), 3.21-3.04 (m, 1H), 2.67-2.43 (m, 3H), 2.32-2.28 (m, 1H), 2.09-2.04 (m, 2H), 1.98 (t, J=5.2 Hz, 4H), 1.86-1.73 (m, 2H), 1.68 (s, 9H), 1.62-1.42 (m, 4H).

### Example CCCC: 6-((1r,4r)-4-(5-Chloro-2-(trifluoromethyl)pyridin-4-yl)cyclohexyl)-2-thia-6-azaspiro[3.4]octane 2,2-dioxide (Compound 87)

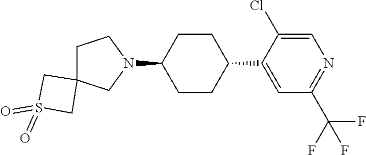 [SMILES: O=S1(=O)CC2(CCN([C@H]3CC[C@H](c4cc(C(F)(F)F)ncc4Cl)CC3)C2)C1]

The title compound was synthesized following a procedure similar to Compound 47A using 4-bromo-5-chloro-2-(trifluoromethyl)pyridine. The mixture of the diastereoisomers was purified by reversed phase chromatography (acetonitrile; 0.05% NH₃ in water+10 mM NH₄HCO₃ in water; 70-100%) to provide title Compound 87 (second peak, 21.9 mg, 0.050 mmol, 20.5% yield) LCMS (ESI) [M+H]⁺=423.1. ¹H NMR (400 MHz, CDCl₃) δ 8.63 (s, 1H), 7.54 (s, 1H), 4.08 (s, 4H), 3.10-2.99 (m, 1H), 2.93 (s, 2H), 2.81 (t, J=7.2 Hz, 2H), 2.32-2.27 (m, 1H), 2.21-2.11 (m, 4H), 2.03-1.96 (m, 2H), 1.52-1.40 (m, 4H).

### Example DDDD: 6-((1r,4r)-4-(2-(Trifluoromethyl)pyrimidin-4-yl)cyclohexyl)-2-thia-6-azaspiro[3.4]octane 2,2-dioxide (Compound 88)

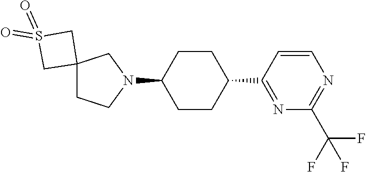 [SMILES: O=S1(=O)CC2(CCN([C@H]3CC[C@H](c4ccnc(C(F)(F)F)n4)CC3)C2)C1]

The title compound was synthesized following a procedure similar to Compound 47A using 4-bromo-2-(trifluoromethyl)pyrimidine. The mixture of the diastereomers was purified by reversed phase chromatography (water (0.05% NH₃ in water+10 mM NH₄HCO₃); ACN; 70-100%) to provide title Compound 88 (second peak, 41.2 mg, 51% yield). LCMS (ESI) [M+H]⁺=390.2. The relative cis/trans stereochemistry was assigned based on ¹H NMR analysis. ¹H NMR (400 MHz, CD₃OD) δ 8.82 (d, J=5.2 Hz, 1H), 7.60 (d, J=5.2 Hz, 1H), 7.56-7.55 (m, 1H), 4.22-4.06 (m, 4H), 3.03 (s, 2H), 2.89-2.87 (m, 2H), 2.84-2.77 (m, 1H), 2.44-2.34 (m, 1H), 2.27-2.16 (m, 4H), 2.06-2.01 (m, 2H), 1.77-1.62 (m, 2H), 1.52-1.37 (m, 2H).

### Example EEEE: 7-((1s,4s)-4-(2-(Trifluoromethyl)pyridin-4-yl)cyclohexyl)-2-thia-7-azaspiro[3.5]nonane 2,2-dioxide (Compound 90A) and 7-((1r,4r)-4-(2-(trifluoromethyl)pyridin-4-yl)cyclohexyl)-2-thia-7-azaspiro[3.5]nonane 2,2-dioxide (Compound 90B)

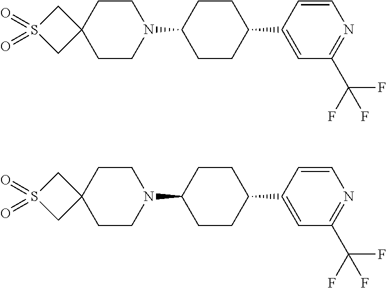 [SMILES: O=S1(=O)CC2(CCN([C@H]3CC[C@@H](c4ccnc(C(F)(F)F)c4)CC3)CC2)C1 ; O=S1(=O)CC2(CCN([C@H]3CC[C@H](c4ccnc(C(F)(F)F)c4)CC3)CC2)C1]

Title compounds were synthesized following a procedure similar to Compound 47A using 4-bromo-2-(trifluoromethyl)pyridine for step 1 and 2-thia-7-azaspiro[3.5]nonane 2,2-dioxide for step 5. The mixture of the diastereoisomers was purified by prep-TLC (25-50% ethyl alcohol in ethyl acetate) to provide title Compound 90A (first peak, 25 mg, 18.2% yield) and title Compound 90B (second peak, 34 mg, 25% yield). LCMS (ESI) [M+H]⁺=403.1. The relative cis/trans stereochemistry was assigned based on ¹H NMR analysis.

Compound 90A: ¹H NMR (400 MHz, CD₃OD) δ 8.60 (d, J=5.2 Hz, 1H), 7.75 (s, 1H), 7.61 (d, J=4.4 Hz, 1H), 3.90 (s, 4H), 2.96-2.91 (m, 1H), 2.83-2.17 (m, 5H), 2.11-2.02 (m, 2H), 1.95-1.86 (m, 6H), 1.76-1.66 (m, 4H).

Compound 90B: ¹H NMR (400 MHz, CD₃OD) δ 8.58 (d, J=5.2 Hz, 1H), 7.70 (s, 1H), 7.55 (d, J=4.8 Hz, 1H), 3.93 (s, 4H), 2.87-2.47 (m, 6H), 2.10-2.01 (m, 4H), 1.96 (t, J=5.2 Hz, 4H), 1.65-1.49 (m, 4H).

### Example FFFF: (Compounds 91A*, 91B*, 91C*, and 91D*): (R)-7-((1s,4S)-4-(1-Isopropyl-3-(trifluoromethyl)-1H-pyrazol-5-yl)cyclohexyl)-2-thia-7-azaspiro[4.5]decane 2,2-dioxide; (R)-7-((1r,4R)-4-(1-isopropyl-3-(trifluoromethyl)-1H-pyrazol-5-yl)cyclohexyl)-2-thia-7-azaspiro[4.5]decane 2,2-dioxide; (S)-7-((1s,4S)-4-(1-isopropyl-3-(trifluoromethyl)-1H-pyrazol-5-yl)cyclohexyl)-2-thia-7-azaspiro[4.5]decane 2,2-dioxide and (S)-7-((1r,4R)-4-(1-isopropyl-3-(trifluoromethyl)-1H-pyrazol-5-yl)cyclohexyl)-2-thia-7-azaspiro[4.5]decane 2,2-dioxide

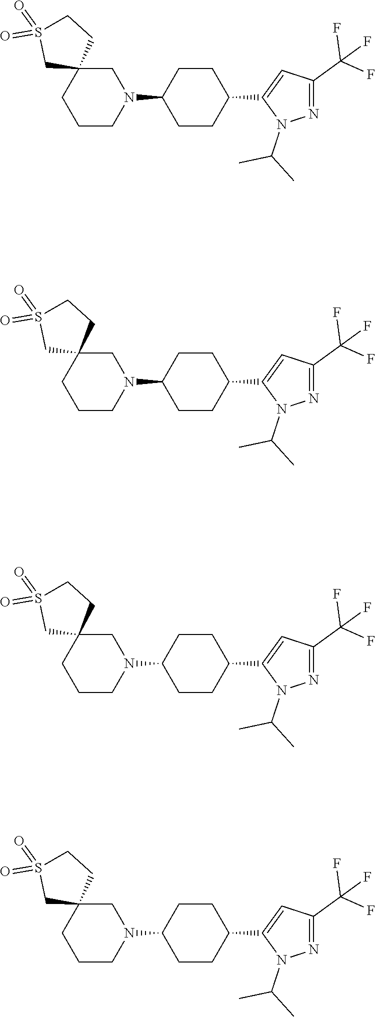 [SMILES: CC(C)n1nc(C(F)(F)F)cc1[C@H]1CC[C@H](N2CCC[C@]3(CCS(=O)(=O)C3)C2)CC1 ; CC(C)n1nc(C(F)(F)F)cc1[C@H]1CC[C@H](N2CCC[C@@]3(CCS(=O)(=O)C3)C2)CC1 ; CC(C)n1nc(C(F)(F)F)cc1[C@H]1CC[C@@H](N2CCC[C@@]3(CCS(=O)(=O)C3)C2)CC1 ; CC(C)n1nc(C(F)(F)F)cc1[C@H]1CC[C@@H](N2CCC[C@]3(CCS(=O)(=O)C3)C2)CC1]

Title compounds were synthesized following a procedure similar to Compound 47A using 5-bromo-1-isopropyl-3-(trifluoromethyl)-1H-pyrazole for step 1 and 2-thia-7-azaspiro[4.5]decane 2,2-dioxide for step 5. The mixture of the diastereoisomers was separated using chiral SFC (WHELK-O1; 25 mm*30 mm, 5 μm; 0.1% NH₃ in water; IPA, 45%; 60 mL/min) to afford title Compound 91A* (third peak on SFC, 47.3 mg, 26.3% yield), title Compound 91B* (fourth peak on SFC, 47.14 mg, 26.2% yield), title Compound 91C* (second peak on SFC, 28.8 mg, 16% yield) and title Compound 91D* (first peak on SFC, 28.7 mg, 16% yield). LCMS (ESI): [M+H]⁺=448.1.

Compound 91A*: ¹H NMR (400 MHz, CD₃OD) δ 6.33 (s, 1H), 4.68-4.61 (m, 1H), 3.21-3.13 (m, 2H), 2.93-2.89 (m, 1H), 2.82-2.66 (m, 3H), 2.58-2.48 (m, 2H), 2.38-2.34 (m, 1H), 2.21-2.11 (m, 1H), 2.06-1.89 (m, 5H), 1.74-1.63 (m, 2H), 1.61-1.51 (m, 7H), 1.46 (d, J=6.8 Hz, 6H).

Compound 91B*: ¹H NMR (400 MHz, CD₃OD) δ 6.32 (s, 1H), 4.68-4.61 (m, 1H), 3.21-3.13 (m, 2H), 2.91 (d, J=13.6 Hz, 1H), 2.82-2.66 (m, 3H), 2.55-2.41 (m, 2H), 2.39-2.33 (m, 1H), 2.21-2.11 (m, 1H), 2.06-1.89 (m, 5H), 1.74-1.66 (m, 2H), 1.61-1.50 (m, 7H), 1.46 (d, J=6.8 Hz, 6H).

Compound 91C*: ¹H NMR (400 MHz, CD₃OD) δ 6.39 (s, 1H), 4.68-4.62 (m, 1H), 3.44-3.38 (m, 1H), 3.24-3.13 (m, 2H), 2.98-2.75 (m, 4H), 2.31-2.23 (m, 1H), 2.15-2.13 (m, 2H), 2.11-1.97 (m, 4H), 1.87-1.79 (m, 2H), 1.74-1.49 (m, 8H), 1.46 (dd, J=3.6, 6.8 Hz, 6H).

Compound 91D*: ¹H NMR (400 MHz, CD₃OD) δ 6.39 (s, 1H), 4.68-4.62 (m, 1H), 3.44-3.36 (m, 1H), 3.23-3.15 (m, 2H), 2.98-2.78 (m, 4H), 2.31-2.23 (m, 1H), 2.20-2.12 (m, 2H), 2.10-2.00 (m, 4H), 1.93-1.80 (m, 2H), 1.73-1.55 (m, 8H), 1.46 (dd, J=3.6, 6.8 Hz, 6H).

### Example GGGG: 6-((1r,4r)-4-(3-Chloro-2-(trifluoromethyl)pyridin-4-yl)cyclohexyl)-2-thia-6-azaspiro[3.4]octane 2,2-dioxide (Compound 92A) and 6-((1s,4s)-4-(3-chloro-2-(trifluoromethyl)pyridin-4-yl)cyclohexyl)-2-thia-6-azaspiro[3.4]octane 2,2-dioxide (Compound 92B)

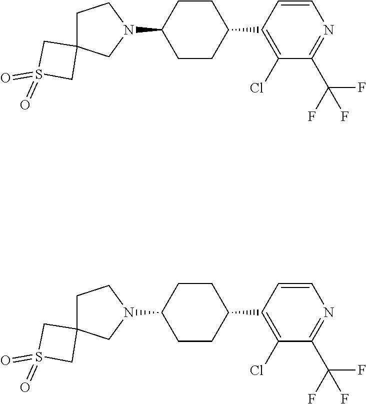 [SMILES: O=S1(=O)CC2(CCN([C@H]3CC[C@H](c4ccnc(C(F)(F)F)c4Cl)CC3)C2)C1 ; O=S1(=O)CC2(CCN([C@H]3CC[C@@H](c4ccnc(C(F)(F)F)c4Cl)CC3)C2)C1]

Title compounds were synthesized following a procedure similar to Compound 47A using 4-bromo-3-chloro-2-(trifluoromethyl)pyridine for step 1. The mixture of the diastereoisomers were purified by reversed phase chromatography (acetonitrile; 0.05% NH₃ in water+10 mM NH₄HCO₃ in water; 70-100%) to provide title Compound 92A (second peak, 55.4 mg, 0.13 mmol, 27.7% yield) and title Compound 92B (first peak on SFC, 22.2 mg, 0.050 mmol, 10.7% yield). LCMS (ESI) [M+H]⁺=423.1. The relative cis/trans stereochemistry was assigned based on ¹H NMR analysis.

Compound 92A: ¹H NMR (400 MHz, CDCl₃) δ 8.50 (d, J=4.8 Hz, 1H), 7.37 (d, J=4.8 Hz, 1H), 4.09 (s, 4H), 3.20-3.13 (m, 1H), 2.94 (s, 2H), 2.89-2.83 (m, 2H), 2.35-2.27 (m, 1H), 2.20-2.12 (m, 4H), 2.04-1.95 (m, 2H), 1.50-1.39 (m, 4H).

Compound 92B: ¹H NMR (400 MHz, CDCl₃) δ 8.51 (d, J=4.8 Hz, 1H), 7.39 (d, J=4.8 Hz, 1H), 4.14-4.05 (m, 4H), 3.26-3.16 (m, 1H), 2.86 (s, 2H), 2.75 (t, J=7.2 Hz, 2H), 2.48 (brs, 1H), 2.19 (t, J=7.2 Hz, 2H), 2.02 (d, J=13.2 Hz, 2H), 1.84-1.72 (m, 2H), 1.69-1.61 (m, 4H).

### Example HHHH: 6-((1r,4r)-4-(2-(Trifluoromethyl)pyridin-4-yl)cyclohexyl)-2-thia-6-azaspiro[3.5]nonane 2,2-dioxide (Compound 93)

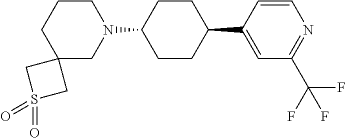 [SMILES: O=S1(=O)CC2(CCCN([C@H]3CC[C@H](c4ccnc(C(F)(F)F)c4)CC3)C2)C1]

The title compound was synthesized following a procedure similar to Compound 47A using 2-thia-6-azaspiro[3.5]nonane 2,2-dioxide for step 5 and 4-bromo-2-(trifluoromethyl)pyridine for step 1. The mixture of the diastereomers were purified by reversed phase chromatography (water (0.05% NH₃ in H₂O+10 mM NH₄HCO₃); ACN, 60-90%) to afford title Compound 93 (second peak, 30.4 mg, 18% yield). The relative cis/trans stereochemistry was assigned based on ¹H NMR analysis. LCMS (ESI) [M+H]⁺=403.1. ¹H NMR (400 MHz, CD₃OD) δ 8.57 (d, J=4.8 Hz, 1H), 7.70 (s, 1H), 7.55 (d, J=4.8 Hz, 1H), 3.89-3.80 (m, 4H), 2.72-2.50 (m, 6H), 2.01-1.98 (m, 4H), 1.74-1.65 (m, 2H), 1.62-1.50 (m, 6H).

### Example IIII: 6-((1r,4r)-4-(2-(Trifluoromethyl)pyrimidin-5-yl)cyclohexyl)-2-thia-6-azaspiro[3.4]octane 2,2-dioxide (Compound 94A) and 6-((1s,4s)-4-(2-(trifluoromethyl)pyrimidin-5-yl)cyclohexyl)-2-thia-6-azaspiro[3.4]octane 2,2-dioxide (Compound 94B)

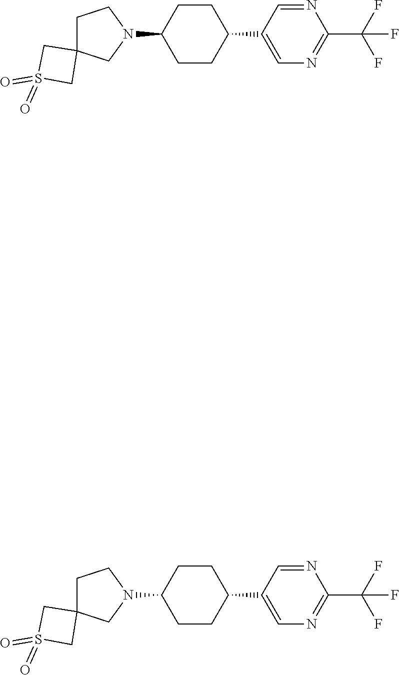 [SMILES: O=S1(=O)CC2(CCN([C@H]3CC[C@H](c4cnc(C(F)(F)F)nc4)CC3)C2)C1 ; O=S1(=O)CC2(CCN([C@H]3CC[C@@H](c4cnc(C(F)(F)F)nc4)CC3)C2)C1]

Title compounds were synthesized following a procedure similar to Compound 47A using 5-bromo-2-(trifluoromethyl)pyrimidine for step 1 and 2-thia-6-azaspiro[3.4]octane 2,2-dioxide for step 5. The mixture of the diastereomers were purified by reversed phase chromatography (DG Phenomenex Gemini-NX; 150*30 mm*5 μm, water (0.05% NH₃ in water+10 mM NH₄HCO₃)-ACN, 40-70%) to provide title Compound 94A (second peak, 80.2 mg, 50% yield) and title Compound 94B (first peak, 37.1 mg, 23% yield). LCMS (ESI) [M+H]⁺=390.2. The relative cis/trans stereochemistry was assigned based on ¹H NMR analysis.

Compound 94A: ¹H NMR (400 MHz, CD₃OD) δ 8.87 (s, 2H), 4.17-4.09 (m, 4H), 2.99 (s, 2H), 2.84 (t, J=7.6 Hz, 2H), 2.79-2.71 (m, 1H), 2.35-2.27 (m, 1H), 2.23-2.15 (m, 4H), 2.04-1.98 (m, 2H), 1.71-1.60 (m, 2H), 1.50-1.39 (m, 2H).

Compound 94B: ¹H NMR (400 MHz, CD₃OD) δ 8.87 (s, 2H), 4.13 (s, 4H), 2.89-2.82 (m, 3H), 2.76 (t, J=7.2 Hz, 2H), 2.46-2.41 (m, 1H), 2.20 (t, J=7.2 Hz, 2H), 2.04-1.94 (m, 4H), 1.72-1.63 (m, 4H).

### Example JJJJ: 2-((1r,4r)-4-(6-(Trifluoromethyl)pyridin-3-yl)cyclohexyl)-8-thia-2-azaspiro[4.5]decane 8,8-dioxide (Compound 95A) and 2-((1s,4s)-4-(6-(trifluoromethyl)pyridin-3-yl)cyclohexyl)-8-thia-2-azaspiro[4.5]decane 8,8-dioxide (Compound 95B)

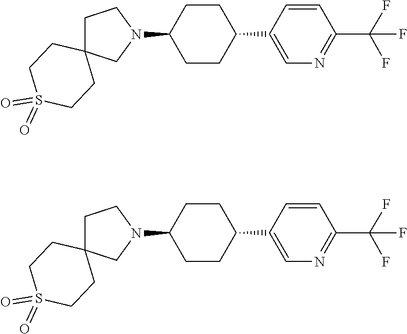 [SMILES: O=S1(=O)CCC2(CCN([C@H]3CC[C@H](c4ccc(C(F)(F)F)nc4)CC3)C2)CC1 ; O=S1(=O)CCC2(CCN([C@H]3CC[C@H](c4ccc(C(F)(F)F)nc4)CC3)C2)CC1]

### Step 1: Methyl 2-(tetrahydro-4H-thiopyran-4-ylidene) acetate

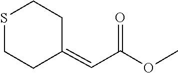

To a solution of methyl 2-(triphenyl-λ5-phosphaneylidene) acetate (7.02 g, 21 mmol) in toluene (40 mL) was added tetrahydro-4H-thiopyran-4-one (2.32 g, 20 mmol). The reaction mixture was stirred at 110° C. overnight. The solvent was removed in vacuo to obtain the crude product, which was purified by silica column chromatography (ethyl acetate/petroleum ether; 0-15%) to afford the title compound (3.19 g, 92.5% yield).

### Step 2: Methyl 2-(4-(nitromethyl) tetrahydro-2H-thiopyran-4-yl) acetate

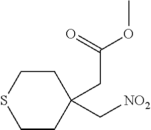

To a solution of methyl 2-(tetrahydro-4H-thiopyran-4-ylidene) acetate (3.19 g, 18.5 mmol) in ACN (10 mL) were added DBU (553 μL, 3.7 mmol) and nitromethane (4 mL, 74 mmol). The reaction mixture was stirred at 50° C. for 18 h. The solution was concentrated in vacuo, washed with 1N HCl solution (10 mL) and extracted with ethyl acetate (20 mL×3). The organic layers were dried over anydrous Na₂SO₄, filtered, concentrated in vacuo and the residue was purified by reversed phase liquid chromatography (0.1% HCl in water/ACN) to afford the title compound (4.31 g, 99.8% yield). LCMS (ESI): [M+H]⁺=234.1.

### Step 3: 8-Thia-2-azaspiro[4.5]decan-3-one

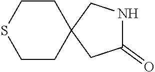 [SMILES: O=C1CC2(CCSCC2)CN1]

To a solution of methyl 2-(4-(nitromethyl)tetrahydro-2H-thiopyran-4-yl) acetate (2.14 g, 9.16 mmol) in MeOH (50 mL) was added Raney Nickel (90% Ni, 118 mg, 1.8 mmol) followed by a dropwise addition of hydrazine hydrate aqueous solution (85%, 0.04 mmol, 2.62 mL). The reaction mixture was stirred for 18 h at rt. The resulting mixture was filtered and the solvent was removed in vacuo. The residue was purified by silica column chromatography (elution with 5% MeOH in DCM) to afford the title compound (1.04 g, 66.3% yield). LCMS (ESI): [M+H]⁺=172.1.

### Step 4: 8-Thia-2-azaspiro[4.5]decane

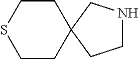

To a solution of 8-thia-2-azaspiro[4.5]decan-3-one (985 mg, 5.75 mmol) in THF (20 mL) was added LiAlH₄ (2.5 M in THF, 4.6 mL, 11.5 mmol) dropwise at 0° C. under N2. The solution was then warmed to room temperature and stirred for 16 h. The reaction was quenched with Na₂SO₄·10 H₂O and filtered. The solvent was removed in vacuo to afford the title compound (876 mg, 96% crude yield). LCMS (ESI): [M+H]⁺=158.1.

### Step 5: tert-Butyl 8-thia-2-azaspiro[4.5]decane-2-carboxylate

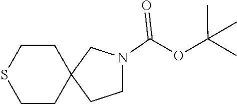 [SMILES: CC(C)(C)OC(=O)N1CCC2(CCSCC2)C1]

To a solution of 8-thia-2-azaspiro[4.5]decane (876 mg as crude, 5.57 mmol) in DCM (20 mL) were added triethylamine (564 mg, 5.57 mmol) and DMAP (34 mg, 0.28 mmol). tert-Butyldicarbonate (1.22 g, 5.57 mmol) was dissolved in a minimal amount of DCM and added into the solution dropwise. The reaction was stirred at room temperature for 15 h and the solvent was removed in vacuo. The residue was washed with 1N HCl (10 mL) and extracted with ethyl acetate (50 mL×3). The combined organic layers were dried over anhydrous Na₂SO₄, filtered and concentrated in vacuo. The resulting residue was purified by silica column chromatography (DCM) to afford the title compound (843 mg, 58.9% yield). LCMS (ESI): [M+Na]⁺=280.0.

### Step 6: tert-Butyl 8-thia-2-azaspiro[4.5]decane-2-carboxylate 8,8-dioxide

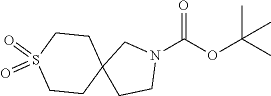 [SMILES: CC(C)(C)OC(=O)N1CCC2(CCS(=O)(=O)CC2)C1]

To a solution of tert-butyl 8-thia-2-azaspiro[4.5]decane-2-carboxylate (843 mg, 3.28 mmol) in acetone (10 mL) and water (10 mL) was added oxone (3.03 g, 4.92 mmol). The reaction mixture was stirred at rt for 5 h. The reaction mixture was then extracted with ethyl actate (30 mL×3) and the combined organic layers were dried over anhtydrous Na₂SO₄, filtered and concentrated in vacuo. The residue was purified by silica column chromatography (0% DCM in petroleum ether) to afford the title compound (585 mg, 61.6% yield). LCMS (ESI) [M+Na]⁺=312.1.

### Step 7: tert-Butyl 8-thia-2-azaspiro[4.5]decane-2-carboxylate 8,8-dioxide

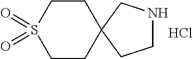 [SMILES: O=S1(=O)CCC2(CCNC2)CC1 ; Cl]

A solution of tert-butyl 8-thia-2-azaspiro[4.5]decane-2-carboxylate 8,8-dioxide (585 mg, 2.02 mmol) in HCl solution (6N, 8 mL) was stirred at room temperature for 2 h. The solvent was removed in vacuo to afford the title compound (393 mg, 86.1% yield). LCMS (ESI): [M+H]⁺=190.1.

### Step 8: 2-((1r,4r)-4-(6-(Trifluoromethyl)pyridin-3-yl)cyclohexyl)-8-thia-2-azaspiro[4.5]decane 8,8-dioxide and 2-((1s,4s)-4-(6-(trifluoromethyl)pyridin-3-yl)cyclohexyl)-8-thia-2-azaspiro[4.5]decane 8,8-dioxide

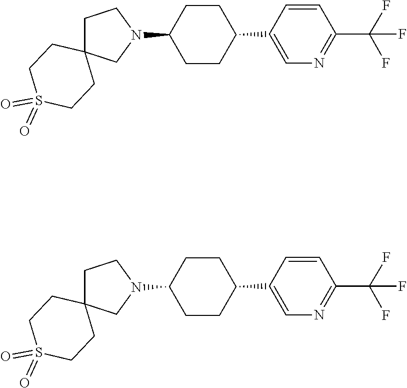 [SMILES: O=S1(=O)CCC2(CCN([C@H]3CC[C@H](c4ccc(C(F)(F)F)nc4)CC3)C2)CC1 ; O=S1(=O)CCC2(CCN([C@H]3CC[C@@H](c4ccc(C(F)(F)F)nc4)CC3)C2)CC1]

To a solution of 8-thia-2-azaspiro[4.5]decane 8,8-dioxide hydrochloride (180 mg, 0.8 mmol) in methanol (10 mL) was added N,N-diisopropylethylamine (400 μL, 2.4 mmol). The reaction mixture was stirred at 30° C. for 30 minutes prior to the addition of 4-(6-(trifluoromethyl)pyridin-3-yl)cyclohexan-1-one (219 mg, 0.9 mmol) and titanium isopropoxide (355 μL, 1.2 mmol). The reaction mixture was then stirred at 30° C. for 18 h, then NaBH(OAc)₃ (508.7 mg, 2.4 mmol) was added and the mixture was stirred at 30° C. for an additional 18 h. The reaction mixture was quenched with 1N aq. NH₄Cl (10 mL) and washed with diluted HCl solution (1N, 10 mL). The mixture was adjusted to pH to 8-9 with saturated Na₂CO₃ solution. The aqueous layer was then extracted with ethyl acetate (30 mL×3) and the combined organic layers were concentrated in vacuo. The mixture of isomers was separated by prep-TLC (5% MeOH in DCM) to afford the title compounds (trans; R_{f}=0.3, 39.6 mg, 12% yield) and (cis; R_{f}=0.6, 30.1 mg, 9.0% yield). The relative cis/trans stereochemistry was assigned based on ¹H NMR analysis.

Compound 95A: ¹H NMR (400 MHz, DMSO-d₆) δ 8.68 (s, 1H), 7.94 (d, J=6.4 Hz, 1H), 7.81 (d, J=8.0 Hz, 1H), 3.05 (t, J=5.6 Hz, 4H), 2.52-2.70 (m, 3H), 2.59 (d, J=12.8 Hz, 2H), 1.93 (q, J=6.0 Hz, 4H), 1.86 (d, J=12.8 Hz, 3H), 1.65 (t, J=6.4 Hz, 2H), 1.56 (q, J=10.4 Hz, 2H), 1.24-1.34 (m, 3H). LCMS (ESI) [M+H]⁺=417.1.

Compound 95B: ¹H NMR (400 MHz, DMSO-d₆) δ 8.64 (s, 1H), 7.88 (d, J=8.0 Hz, 1H), 7.82 (d, J=8.4 Hz, 1H), 3.06 (t, J=5.6 Hz, 4H), 2.77 (t, J=11.2 Hz, 1H), 2.57 (s, 1H), 2.44 (s, 1H), 2.25 (s, 1H), 1.92-1.98 (m, 6H), 1.3 (d, J=11.6 Hz, 2H), 1.69 (d, J=5.6 Hz, 2H), 1.55 (d, J=11.2 Hz, 4H). LCMS (ESI) [M+H]⁺=417.1.

### Example KKKK: 6-((1s,4s)-4-(5-(Trifluoromethyl)pyridin-2-yl)cyclohexyl)-2-thia-6-azaspiro[3.4]octane 2,2-dioxide (Compound 96)

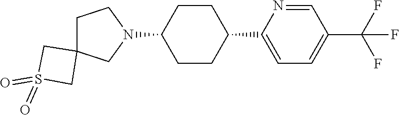 [SMILES: O=S1(=O)CC2(CCN([C@H]3CC[C@@H](c4ccc(C(F)(F)F)cn4)CC3)C2)C1]

The title compound was synthesized using the same procedure and purification as Compound 50A using 2-bromo-5-(trifluoromethyl)pyridine for step 1 and 2-thia-6-azaspiro[3.4]octane 2,2-dioxide for step 5. Compound 96 was obtained (30.6 mg, second eluting peak, 15% yield). LCMS (ESI) [M+H]⁺=389.1. ¹H NMR (400 MHz, DMSO-d₆) δ 8.87 (s, 1H), 8.09 (dd, J=8.4, 2.6 Hz, 1H), 7.51 (d, J=8.3 Hz, 1H), 4.23-4.03 (m, 4H), 2.94-2.82 (m, 1H), 2.76 (s, 2H), 2.62 (t, J=7.2 Hz, 2H), 2.37-2.31 (m, 1H), 2.10 (t, J=7.2 Hz, 2H), 2.02-1.88 (m, 2H), 1.88-1.78 (m, 2H), 1.65-1.50 (m, 4H). The relative cis stereochemistry was assigned based on ¹H NMR analysis.

### Biological Assay Examples

### Mouse OPC Preparation

To assess effects of treatments on OPCs, all treatments were assayed in two or more independent platings of epiblast stem cell-derived OPCs (EpiSC). EpiSC-derived OPCs were obtained using in vitro differentiation protocols and culture conditions described previously (Najm et al, 2011, Nature Methods). OPCs were expanded and frozen down in aliquots. OPCs were thawed into growth conditions for at least one passage before use in further assays.

### Determination of EC₅₀ Values of Compounds

### A: In Vitro Phenotypic Screening of OPCs

EpiSC-derived OPCs were grown and expanded in poly-L-ornithine (PO) and laminin-coated flasks in N2B27 media (DMEM/F12 (Gibco), N2-MAX (R&D Systems), B-27 (ThermoFisher), and GlutaMax (Gibco)) supplemented with FGF2 (10 μg/mL, R&D systems, 233-FB-025) and PDGF-AA (10 μg/mL, R&D systems, 233-AA-050) before harvesting for experiments. The cells were seeded onto poly-L-ornithine or poly-D-lysine coated CellCarrier Ultra plates (PerkinElmer) coated with laminin (Sigma, L2020) at a density of 150,000/cm² in N2B27 media without growth factors. For dose-response testing, a 1000× compound stock in dimethyl sulphoxide (DMSO) was added to assay plates, resulting in 8-point dose curves with final concentrations between 1000 nM and 0.5 nM. Positive controls and DMSO vehicle controls were included in each assay plate. Cells were incubated under standard conditions (37° C., 5% CO₂) for 3 days and fixed with 4% paraformaldehyde (PFA) in phosphate buffered saline (PBS) for 20 min. Fixed plates were washed with PBS, permeabilized with 0.1% Triton X-100, and blocked with 10% donkey serum (v/v) in PBS for 40 min. Then, cells were labelled with MBP antibodies (Abcam, ab7349; 1:200) for 2 h at room temperature, washed with PBS, and stained with Alexa Fluor conjugated secondary antibodies (1:500) for 45 min. Nuclei were visualized by DAPI staining (Sigma; 1 μg/ml), followed by further PBS washes.

### B: High-Content Imaging and Analysis

Cells and cell culture plates were imaged on the Operetta High Content Imaging and Analysis system (PerkinElmer). Analysis (PerkinElmer Harmony and Columbus software) began by identifying intact nuclei stained by DAPI. The peri-nuclear region of each cell was then cross-referenced with the mature myelin protein (MBP) stain to identify oligodendrocyte nuclei, and from this the percentage of oligodendrocytes was calculated. EC₅₀ values were calculated using The Levenberg-Marquardt algorithm to fit a Hill equation to dose-response data (0.5 nM to 1000 nM). The results are provided in Table 4 (OPC EC₅₀).

### Determination of Potency and Enzyme Target

### GC/MS-Based Sterol Profiling

Sterols were monitored using a modified Folch wash protocol (Hubler et al, 2018, Nature). EpiSC-derived OPCs were plated at 100,000 cells per well in PO- and laminin-coated 96-well plates in N2B27 media without growth factors. After 24 hours, cells were rinsed with saline and plates were frozen. Cholesterol-d7 standard was then added to each well before drying under nitrogen stream and derivatization with 55 μl of bis(trimethylsilyl) trifluoroacetamide. After derivatization, 2 μl were analyzed by gas chromatography/mass spectrometry using an Agilent 5973 Network Mass Selective Detector equipped with a 6890 gas chromatograph system and a HP-5MS capillary column (30 m×0.25 mm×0.25 mm). Samples were analyzed in full scan mode using electron impact ionization; ion fragment peaks were integrated to calculate sterol abundance, and quantitation was relative to cholesterol-d7. The following ion fragments were used to quantitate each metabolite: cholesterol-d7 (465), FF-Mas (482), cholesterol (368), zymostenol (458), zymosterol (456), Desmosterol (456, 343), 7-dehydrocholesterol (456, 325), lanosterol (393), lathosterol (458), 14-dehydrozymostenol (456, 351). For reference, Table 2 shows sterol GC-MS analytes and their relationship with inhibitors of cholesterol biosynthesis. All standards were obtained from Avanti Polar Lipids unless otherwise indicated. Calibration curves were generated by injecting varying concentrations of sterol standards and maintaining a fixed amount of cholesterol-D7. For normalized zymostenol accumulation results, the total amount of zymostenol measured after drug treatment was divided by the total amount of zymostenol accumulated after 24 hr treatment with 100 nM positive control reference. EC₅₀ values were calculated using The Levenberg-Marquardt algorithm to fit a Hill equation to dose-response data (8 doses from 0.15 nM to 333 nM). EC₅₀ values for zymostenol (Zymostenol GCMS EC₅₀) are provided in Table 4.

TABLE 2

| Analyte increased | Cholesterol enzyme inhibited |
|---|---|
| Lanosterol | CYP51 |
| FF-MAS | TM7SF2 (Sterol 14 Reductase) |
| 14-dehydrozymostenol | TM7SF2 (Sterol 14 Reductase) |
| Zymosterol | EBP |
| Zymostenol | EBP |
| 7-dehydrocholesterol | DHCR7 |

### Determination of Binding Affinity

Membrane preparation: To examine compound binding affinity to EBP, human EBP was overexpressed in human embryonic kidney 293 cells. Cell pellet was lysed in 10 times weight binding buffer (50 mM Tris, 5 mM MgCl₂, 0.1 mM EDTA, lx protease inhibitor cocktail, pH 7.5) on ice by using a dounce homogenizer. The solution was centrifuged at 25,000 g for 50 min at 4° C. The membrane pellet was re-suspended in binding buffer and run through a 25 5/8 gauge needle. After checking the concentration by Bradford assay, the whole cell membrane solution was adjusted to 20 mg/mL and stored at −80° C.

Determination of equilibrium dissociation constant Kd of radioligand: Membrane prepared as described above was pre-incubated with PVT-WGA SPA beads (Perkinelmer Cat #RPNQ0003) at a ratio of 0.3 mg beads with 5 μg membrane per 25 μL binding buffer at 20° C. for 2 hours with gental shaking. This binding solution was centrifuged at 400 g for 5 minutes to collect the bead/membrane mixture. After re-suspending the pellet in binding buffer at the same calculated volume with 0.01% BSA (Sigma A1933), the bead/membrane mixture was added in 384-well low-binding surface plate (PerkinElmer Cat #6057480) at 25 μl/well. Radioligand at different concentrations with and without the non-radio-labeled same ligand 5 uM (for nonspecific and total signal, respectively) was added to bring final volume to 50 l/well with DMSO concentration at 0.1%. At equilibrium (3 hours after ligand addition), radiometric signal CPM was counted by using a Microbeta2 microplate counter (Perkinelmer). The Kd was determined by nonlinear regression fitting of specific signal plot against the concentration of radioligand [3H]-Ifenprodil (Table 3).

TABLE 3

| Target | Radioligand | Kd | [L] |
|---|---|---|---|
| EBP | [3H]-Ifenprodil, | 15.86 nM | 10 nM |
| | (Perkinelmer Cat# NET1089250UC) | | |

[L], concentration of radioligand used in assay

Competition binding assay to determine compound affinity: The same conditions of the radioligand Kd study were used for compound single dose percentage inhibition and equilibrium dissociation constant Ki examinations, except 50 nL compound DMSO stock was pre-added in 384-well low-binding surface plate (PerkinElmer Cat #6057480) by Echo 550 (Labcyte) to reach the final concentration for single dose test at 1 uM, and dose response test from 0.06 nM to 5 uM (8 dose, 5 times dilution). A pre-incubated bead/membrane mixture was added in compound plate at 0.3 mg beads and 5 g membrane per well. Radioligand [3H]-Ifenprodil was added to reach optimized concentration [L] and bring assay volume to 50 μl. At equilibrium (3 hours after ligand addition), radiometric signal was counted as described above. The percentage inhibition of compound at each testing concentration was calculated by normalizing each condition's CPM readout to full block (5 uM non-radiolabeled ligand) and non-block (DMSO) control conditions. Compound binding inhibition IC₅₀ was determined by nonlinear regression fitting of percentage inhibition plot against compound concentration. Compound Ki was calculated from the equation Ki=IC₅₀/(1+[L]/Kd), which [L] was radioligand concentration used in assay. All tests had N bigger or equal to 2. The data from this experiment is shown in Table 4 (hEBP SPA Ki).

### Determination of Binding Affinity to EBP-7-Dehydrocholesterol Reductase

### Materials and Instruments

| Materials | Vendor | Cat# |
|---|---|---|
| (S)-6-(2-Methyl-3-(6-(trifluoromethyl) | Moravek | MT-1003106 |
| pyridin-3-yl)propyl)-2-thia-6-azaspiro | | |
| [3.4]octane 2,2-dioxide, [3H] | | |
| Ifenprodil (+)-tartrate salt | Sigma | I2892 |
| Bovine Serum Albumin (BSA) | Sigma | A1933 |
| Ethylenediaminetetraacetic acid | Sigma | E5391 |
| tetrasodium salt hydrate (EDTA) | | |
| Magnesium Chloride Hexahydrate | Sigma | M2670 |
| (MgCl2•6H2O) | | |
| Tris(hydroxymethyl)aminomethane (Tris) | Alfa Aesar | A18494 |
| Polyethylenimine, branched (PEI) | Sigma | 408727 |

### Key Instruments and Consumables

| Item | Supplier | Cat# |
|---|---|---|
| MicroBeta2 Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene Microplate | Labcyte | PP-0200 |
| 96-Well U bottom Plate | Corning | 7007 |
| Echo | LABCYTE | 550 |

### Assay Buffer Preparation

| Reagent | Concentration |
|---|---|
| Tris | 50 mM |
| MgCl₂ | 5 mM |
| EDTA | 0.1 mM |
| BSA | 0.01% (w/v) |

Adjust pH to 7.4 followed by 0.2 μM sterile filtration

Membrane Preparation: human emopamil binding protein and human 7-dehydrocholesterol reductase co-expressing cells were generated by transient transfecting host human embryonic kidney (HEK) 293 cells with 2 DNA constructs containing each protein's coding sequence. Cells were suspension cultured at 37° C. with 5% CO₂ in FREESTYLE 293 Expression Medium (Thermofisher). Whole cell membrane was prepared by harvesting the cell pellet, adding cold membrane buffer (50 mM Tris, pH7.5, lx Roche COMPLETE EDTA-free protease inhibitor cocktail) 10 times volume of the cell pellets weight, lysing cell pellet on ice by using Dounce homogenizer, spinning at 200 g 4° C. for 15 min, collecting supernatant and spinning again at 25000 g 4° C. for 50 min, transferring pellet to Dounce homogenizer, re-suspending pellet by homogenizing in membrane buffer on ice to reach ˜25 mg/mL, then keeping whole cell membrane aliquots at −80° C.

Compounds were prepared in a 96-well U bottom plate using an Echo550 machine and 10 mM compound DMSO stock solution, followed by an 8-dose 5-fold serial dilutions protocol with final testing compound concentration ranging from 0.06 to 5000 nM, with DMSO back fill to 100 nL/well and n=2. DMSO and Ifenprodil 5 uM wells were added in each plate as 0 and 100% inhibition reference controls with n=8 for each condition. The UniFilter-96 GF/B plates were pre-treated by adding 50 μl/well of 0.3% (v/v) PEI to UniFilter-96 GF/B plates. The plates were sealed and incubated at 4° C. for 3 hrs. Then, the plates were washed with ice-cold assay buffer 3 times. The radioligand binding assay was prepared by adding assay buffer diluted hEBP-DHCR7 membrane at 66.7 μg/ml×150 μl/well into the 96-well compound plate to reach 10 μg membrane per well. Then, the assay buffer diluted [3H]—(S)-6-(2-Methyl-3-(6-(trifluoromethyl)pyridin-3-yl)propyl)-2-thia-6-azaspiro[3.4] octane 2,2-dioxide was added at 25 nM×50 μl/well. Following this, the plate was centrifuged at 1000 rpm for 30 secs. The plate was then sealed and agitated at 600 rpm at 22° C. for 5 min, and then incubated at 22° C. for 3 hrs. The incubation was stopped by transferring the binding solution to the pre-treated UniFilter-96 GF/B plate, vacuum filtrated, and then washed four times with ice-cold assay buffer. Following this, the plates were dried at 37° C. for 45 min. The plates were then sealed at the bottom. 40 μl/well of scintillation cocktail was added to the plates. A MicroBeta2 microplate counter was then used to read the plate and analyze the data. For reference and test compounds, the results are expressed as % Inhibition, using the normalization equation: N=100−100×(U−C₂)/(C1−C2), where U is the unknown value, C1 is the average of high controls, and C2 is the average value of low controls. The IC50 was determined by fitting percentage of inhibition as a function of compound concentrations with Hill equation using XLfit. Results are expressed as hEBP-DHCR7 Ki (uM) in Table 4. Ki was calculated as described above. The asterix (*) indicates an isolated isomer or isolated group of isomers, but that the stereochemistry has not been assigned.

TABLE 4

| | hEBP | hEBP- | Zymostenol | OPC |
|---|---|---|---|---|
| | SPA | DHCR7 | GCMS | EC₅₀ |
| No. | Ki (μM) | Ki (μM) | EC₅₀ (μM) | (μM) |
| 1A | ND | 0.0148 | 0.0049 | 0.017 |
| 1B | 0.1 | 0.0268 | 0.0026 | 0.0018 |
| 1C | ND | 0.011 | 0.0014 | 0.0018 |
| 1D | ND | 0.015 | 0.0049 | 0.018 |
| 2A | 0.020 | 0.0445 | 0.056 | 0.240 |
| 2B | 0.020 | 0.0693 | 0.012 | 0.048 |
| 3A | 0.02 | 0.0158 | 0.0021 | 0.0051 |
| 3B | 0.062 | 0.0275 | 0.016 | 0.03 |
| 4 | 0.026 | 0.0518 | 0.0025 | 0.023 |
| 5 | 0.067 | 0.148 | 0.05 | 0.19 |
| 6 | 0.037 | 0.023 | 0.014 | 0.039 |
| 7 | 0.010 | 0.0175 | 0.034 | 0.021 |
| 8A | 0.016 | 0.034 | 0.014 | 0.071 |
| 8B | 0.021 | 0.0329 | 0.026 | 0.24 |
| 9A* | 0.034 | 0.0583 | 0.010 | 0.086 |
| 9B* | 0.17 | 0.245 | 0.394 | >1 |
| 10A* | 0.034 | 0.0187 | 0.0028 | 0.018 |
| 10B* | 0.019 | 0.0213 | 0.0029 | 0.020 |
| 10C* | 0.029 | 0.0266 | 0.003 | 0.011 |
| 10D* | 0.043 | 0.0331 | 0.044 | ND |
| 11A | 0.054 | 0.0378 | 0.03 | 0.16 |
| 11B | 0.042 | 0.0359 | 0.0073 | 0.035 |
| 11C* | 0.035 | 0.035 | 0.0041 | 0.024 |
| 11D* | 0.04 | 0.04 | 0.044 | 0.28 |
| 12A | 0.028 | 0.034 | 0.011 | 0.037 |
| 12B | 0.029 | 0.032 | 0.023 | 0.240 |
| 13A* | 0.015 | 0.0069 | 0.00091 | 0.0013 |
| 13B* | 0.025 | 0.015 | 0.0009 | 0.0013 |
| 13C* | 0.021 | 0.0235 | 0.0041 | 0.021 |
| 13D* | 0.071 | 0.019 | 0.0012 | 0.0022 |
| 14A | 0.035 | 0.059 | 0.011 | 0.016 |
| 14B | 0.03 | 0.028 | 0.043 | 0.24 |
| 15 | 0.025 | 0.056 | 0.004 | 0.044 |
| 16 | 0.036 | 0.052 | 0.012 | 0.041 |
| 17A* | 0.022 | 0.024 | 0.0021 | ND |
| 17B* | 0.031 | 0.036 | 0.004 | 0.022 |
| 17C* | 0.018 | ND | 0.0029 | 0.013 |
| 17D* | 0.020 | 0.064 | 0.0035 | 0.014 |
| 18A* | 0.31 | 0.96 | 0.072 | 0.39 |
| 18B* | 0.21 | 0.65 | 0.20 | 0.39 |
| 18C* | 0.082 | 0.811 | 0.054 | 0.24 |
| 18D* | 0.27 | 1.18 | 0.12 | 0.43 |
| 19A* | 0.027 | 0.021 | 0.023 | 0.14 |
| 19B* | 0.027 | 0.020 | 0.010 | 0.049 |
| 19C* | 0.034 | 0.018 | 0.11 | 0.27 |
| 19D* | 0.032 | 0.033 | 0.11 | 0.16 |
| 20A* | 0.28 | 0.53 | 0.24 | >1.0 |
| 20B* | 0.055 | 0.13 | 0.019 | 0.34 |
| 20C* | 0.27 | 0.42 | 0.31 | 0.66 |
| 20D* | 0.098 | 0.039 | 0.038 | 0.26 |
| 21A | 0.023 | 0.032 | 0.012 | 0.037 |
| 21B | 0.048 | 0.046 | 0.050 | 0.12 |
| 22A* | 0.034 | 0.014 | 0.002 | 0.0095 |
| 22B* | 0.070 | 0.058 | 0.035 | 0.110 |
| 23A | 0.026 | 0.021 | 0.031 | 0.100 |
| 23B | 0.021 | 0.012 | 0.009 | 0.028 |
| 24 | 0.028 | 0.011 | 0.003 | 0.006 |
| 25A | 0.110 | 0.45 | 0.043 | 0.100 |
| 25B | 0.058 | 0.27 | 0.026 | 0.087 |
| 26A* | 0.033 | 0.025 | 0.004 | 0.018 |
| 26B* | 0.021 | 0.012 | 0.038 | 0.130 |
| 27A* | 0.039 | 0.26 | 0.026 | 0.053 |
| 27B* | 0.046 | 0.18 | 0.022 | 0.130 |
| 28 | 0.033 | 0.030 | 0.0075 | 0.064 |
| 29 | 0.045 | 0.23 | 0.0071 | 0.039 |
| 30 | 0.039 | 0.013 | 0.005 | 0.01 |
| 31 | 0.059 | 0.084 | 0.032 | 0.19 |
| 32 | 0.024 | 0.011 | 0.007 | 0.06 |
| 33A | 0.037 | 0.034 | 0.0076 | 0.029 |
| 33B | 0.033 | 0.028 | 0.0098 | 0.053 |
| 35 | 0.18 | 0.28 | 0.045 | 0.21 |
| 36A* | 0.040 | 0.061 | 0.0086 | 0.073 |
| 36B* | 0.057 | 0.26 | 0.023 | 0.072 |
| 38A* | 0.027 | 0.082 | 0.049 | 0.200 |
| 38B* | 0.024 | 0.05 | 0.06 | 0.40 |
| 38C* | 0.029 | 0.26 | 0.02 | 0.05 |
| 38D* | 0.027 | 0.08 | 0.04 | 0.2 |
| 40A | 0.032 | 0.059 | 0.004 | 0.022 |
| 40B | 0.047 | 0.183 | 0.017 | 0.071 |
| 42A* | ND | 0.57 | 0.043 | 0.093 |
| 42B* | ND | 0.39 | 0.058 | 0.18 |
| 42C* | ND | 1.18 | 0.088 | 0.19 |
| 42D* | ND | 0.67 | 0.045 | 0.12 |
| 43A* | ND | 0.125 | 0.008 | 0.027 |
| 43B* | ND | 0.51 | 0.080 | 0.16 |
| 44 | ND | 0.009 | 0.004 | 0.014 |
| 45 | ND | 0.017 | 0.013 | 0.049 |
| 46A* | ND | 0.031 | 0.0013 | 0.0025 |
| 46B* | ND | 0.035 | 0.0027 | 0.0049 |
| 46C* | ND | 0.013 | 0.0017 | 0.0052 |
| 46D* | ND | 0.012 | 0.0011 | 0.0024 |
| 47A | 0.011 | 0.011 | 0.0091 | ND |
| 47B | 0.016 | 0.023 | 0.0041 | 0.020 |
| 48A | ND | 0.031 | 0.0037 | 0.0040 |
| 48B | ND | 0.0096 | 0.014 | ND |
| 49 | ND | ND | 0.0099 | 0.015 |
| 50A | ND | ND | 0.026 | 0.029 |
| 50B | ND | ND | 0.032 | 0.045 |
| 51A | ND | 0.026 | 0.0083 | 0.018 |
| 51B | ND | 0.014 | 0.0059 | ND |
| 52 | ND | 0.018 | 0.0048 | 0.0077 |
| 53A* | ND | 0.012 | 0.0012 | 0.0023 |
| 53B* | ND | 0.0099 | 0.00089 | 0.0024 |
| 53C* | ND | 0.01 | 0.0011 | ND |
| 53D* | ND | 0.096 | 0.0010 | 0.0033 |
| 54A* | ND | 0.021 | 0.061 | ND |
| 54B* | ND | 0.10 | 0.24 | 1.0 |
| 54C* | ND | 0.347 | >0.33 | ND |
| 54D* | ND | 0.068 | 0.013 | 0.035 |
| 55A* | ND | 0.0028 | 0.0023 | 0.015 |
| 55B* | ND | 0.014 | 0.025 | 0.33 |
| 56A* | ND | 0.061 | 0.030 | 0.27 |
| 56B* | ND | 0.17 | 0.32 | 0.87 |
| 56C* | ND | 0.14 | 0.019 | 0.076 |
| 56D* | ND | 0.065 | 0.15 | 0.69 |
| 57A* | ND | 0.16 | 0.024 | 0.065 |
| 57B* | ND | 0.20 | 0.044 | 0.16 |
| 57C* | ND | 0.12 | 0.016 | 0.053 |
| 57D* | ND | 0.15 | 0.018 | 0.13 |
| 58A | ND | 0.022 | 0.0044 | 0.0073 |
| 58B | ND | 0.021 | 0.0023 | 0.0034 |
| 59 | ND | 0.072 | 0.013 | 0.082 |
| 60A | ND | 0.133 | 0.010 | 0.021 |
| 60B | ND | 0.021 | 0.085 | ND |
| 61A* | ND | 0.080 | >0.33 | ND |
| 61B* | ND | 0.023 | 0.10 | ND |
| 61C* | ND | 0.031 | 0.0075 | 0.053 |
| 61D* | ND | 0.344 | >0.33 | ND |
| 62A | ND | 0.023 | 0.045 | ND |
| 62B | ND | 0.037 | 0.0064 | 0.013 |
| 63A | ND | 0.075 | 0.012 | ND |
| 63B | ND | 0.066 | 0.289 | ND |
| 64A | ND | 0.015 | 0.0037 | 0.011 |
| 64B | ND | 0.012 | 0.0032 | ND |
| 65A | ND | 0.014 | 0.0041 | 0.0089 |
| 65B | ND | 0.008 | 0.0028 | ND |
| 66A* | ND | 1.98 | >0.33 | ND |
| 66B* | ND | 0.061 | 0.012 | ND |
| 67 | ND | 0.18 | 0.030 | ND |
| 68A* | ND | ND | 0.19 | ND |
| 68B* | ND | 0.041 | 0.0058 | 0.022 |
| 68C* | ND | ND | >0.33 | ND |
| 68D* | ND | ND | 0.042 | ND |
| 69A* | ND | 0.024 | 0.0021 | 0.0075 |
| 69B* | ND | 0.035 | 0.023 | ND |
| 69C* | ND | 0.027 | 0.0060 | 0.044 |
| 69D* | ND | 0.050 | 0.058 | ND |
| 70A | ND | 0.0065 | 0.0040 | 0.018 |
| 70B | ND | 0.0067 | 0.019 | ND |
| 71A* | ND | ND | 0.006 | 0.019 |
| 71B* | ND | ND | 0.28 | ND |
| 71C* | ND | ND | 0.062 | ND |
| 71D* | ND | ND | >0.33 | ND |
| 72A | ND | ND | 0.0094 | 0.018 |
| 72B | ND | 0.026 | 0.059 | ND |
| 73 | ND | 0.021 | 0.033 | 0.11 |
| 74A | ND | 0.154 | 0.031 | 0.23 |
| 74B | ND | 0.494 | 0.017 | 0.076 |
| 75 | ND | 0.011 | 0.0032 | 0.0050 |
| 77A | ND | 0.033 | 0.0057 | 0.013 |
| 77B | ND | 0.0080 | 0.0050 | 0.015 |
| 78A* | ND | 0.071 | >0.33 | ND |
| 78B* | ND | 0.026 | 0.022 | 0.14 |
| 78C* | ND | 0.79 | 0.028 | 0.273 |
| 78D* | ND | 0.037 | 0.019 | 0.124 |
| 79 | ND | 0.022 | 0.0097 | 0.036 |
| 80A | ND | 0.067 | 0.011 | ND |
| 80B | ND | 0.028 | 0.0080 | 0.038 |
| 81A* | ND | 0.085 | 0.036 | ND |
| 81B* | ND | 0.47 | 0.042 | 0.27 |
| 82A* | ND | 0.089 | 0.28 | ND |
| 82B* | ND | 0.046 | 0.019 | ND |
| 82C* | ND | 0.036 | >0.33 | ND |
| 82D* | ND | 0.100 | >0.33 | ND |
| 83 | ND | 0.12 | 0.034 | 0.28 |
| 84A* | ND | 0.31 | 0.10 | ND |
| 84B* | ND | 0.069 | 0.027 | 0.13 |
| 85A | ND | 0.015 | 0.0081 | ND |
| 85B | ND | 0.30 | 0.017 | 0.048 |
| 87 | ND | 0.012 | 0.0088 | 0.033 |
| 88 | ND | 0.073 | 0.016 | 0.110 |
| 90A | ND | ND | 0.031 | 0.24 |
| 90B | ND | 1.29 | 0.042 | ND |
| 91A* | ND | ND | 0.0034 | 0.0094 |
| 91B* | ND | 0.31 | 0.060 | ND |
| 91C* | ND | ND | 0.0082 | ND |
| 91D* | ND | ND | 0.050 | ND |
| 92A | ND | ND | 0.036 | ND |
| 92B | ND | ND | 0.0052 | ND |
| 93 | ND | ND | 0.012 | ND |
| 94A | ND | 0.23 | 0.081 | ND |
| 94B | ND | 0.115 | 0.25 | ND |
| 95A | ND | 0.71 | 0.17 | 0.52 |
| 95B | ND | 0.17 | 0.022 | 0.043 |
| 96 | ND | ND | 0.071 | 0.11 |

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of formula I: 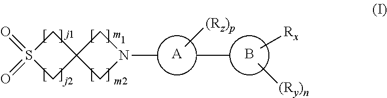
or a pharmaceutically acceptable salt thereof, wherein,
j₁, j₂, and m₁ are each independently 1, 2, or 3;
m₂ is 0, 1, 2, or 3;
wherein the sum of j₁ and j₂ and the sum of m₁ and m₂ are each no more than 5, and the total sum of j₁, j₂, m₁, and m₂ is no more than 9;
Ring A is 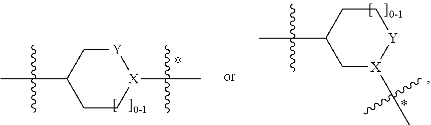
where [symbol] indicates the connection of the ring to the remainder of the molecule, wherein * indicates the connection of Ring A to Ring B;
X is N or CH, Y is O or CH₂, wherein when X is N then Y is CH₂;
R_{z} if present, in each instance is independently selected from the group consisting of halogen, hydroxy, C₁-C₆ alkyl, halo-C₁-C₆ alkyl, C₁-C₆ alkoxy, halo-C₁-C₆ alkoxy, —CN, and —NRR′;
and, two R_{z}, together with the atom on Ring A to which each is attached, may form a bridge;
p is 0, 1, 2, or 3;
Ring B is phenyl, or 5- to 6-membered heteroaryl comprising one, two, or three heteroatoms independently selected from the group consisting of O, N, and S;
R_{y} if present, in each instance is independently selected from the group consisting of halogen, C₁-C₆ alkyl, halo-C₁-C₆ alkyl, hydroxy, C₁-C₆ alkoxy, halo-C₁-C₆ alkoxy, C₃-C₇ cycloalkyl, C₃-C₇halocycloalkyl, —SO₂(C₁-C₆ alkyl), —CN, and —NRR′;
n is 0, 1, 2, 3, or 4;
Rₓ is selected from the group consisting of halogen, hydroxy, C₁-C₁₀ alkyl, halo-C₁-C₆ alkyl, C₁-C₆ alkoxy, halo-C₁-C₆ alkoxy, 5- to 7-membered heterocyclyl, C₃-C₇ cycloalkyl, C₆-C₁₀ aryl, 5- to 6-membered heteroaryl, —SO₂(C₁-C₆ alkyl), —CN, and —NRR′;
wherein said heterocyclyl, cycloalkyl, aryl, or heteroaryl is substituted with (R_{xA})_{q};
wherein q is 0, 1, 2, 3, 4, or 5; and
R_{xA}, if present, in each instance is independently selected from the group consisting of halogen, C₁-C₆ alkyl, hydroxy, C₁-C₆ alkoxy, halo-C₁-C₆ alkoxy, halo-C₁-C₆ alkyl, —SO₂(C₁-C₆ alkyl), —CN, and —NR″R′″; and
R, R′, R″, and R′″ are each independently H, C₁-C₆ alkyl, or halo-C₁-C₆ alkyl.

2.-7. (canceled)

8. The compound of claim 1, wherein the compound is of Formula Ia: 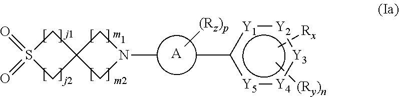
or a pharmaceutically acceptable salt thereof, wherein Y₁-Y₅ are each independently selected from the group consisting of CH, C, and N, wherein C is substituted with R_{y} or Rₓ.

9. (canceled)

10. The compound of claim 8, or a pharmaceutically acceptable salt thereof, wherein one of Y₁, Y₂, Y₃, Y₄, and Y₅ is N; one is CRₓ; and the remainder are each independently CH or CR_{y}; or
one of Y₁, Y₂, Y₃, Y₄, and Y₅ is CRₓ, and the remainder are each independently CH or CR_{y}.

11.-12. (canceled)

13. The compound of claim 10, or a pharmaceutically acceptable salt thereof, wherein Y₁, Y₂, Y₄, and Y₅ are each CH and Y₃ is C—Rₓ; or
Y₁, Y₂, and Y₅ are CH, Y₄ is N, and Y₃ is C—Rₓ.

14.-18. (canceled)

19. The compound of claim 1, wherein the compound is of Formula Ic: 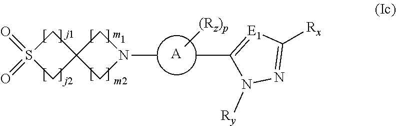
or a pharmaceutically acceptable salt thereof, wherein E₁ is N or CH.

20. The compound of claim 1, or a pharmaceutically acceptable salt thereof, wherein Rₓ is selected from the group consisting of halo-C₁-C₆ alkyl, C₃-C₅ cycloalkyl, phenyl, and 6-membered heteroaryl, wherein said cycloalkyl, phenyl, or heteroaryl is substituted with (R_{xA})_{q}.

21. (canceled)

22. The compound of claim 20, wherein Rₓ is trifluoromethyl; or cyclopropyl, and q is 0.

23. (canceled)

24. The compound of claim 19, wherein the compound is of Formula Id: 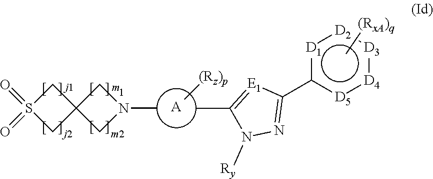
or a pharmaceutically acceptable salt thereof, wherein D₁, D₂, D₃, D₄, and D₅ are each independently N, C when bound to R_{xA}, or CH, wherein up to three of D₁, D₂, D₃, D₄, and D₅ are N; and q is 1 or 2.

25. The compound of claim 24, or a pharmaceutically acceptable salt thereof, wherein one of D₁-D₅ is N.

26. The compound of claim 24, or a pharmaceutically acceptable salt thereof, wherein D₃ or D₄ is C—R_{xA}.

27. (canceled)

28. The compound of claim 24, or a pharmaceutically acceptable salt thereof, wherein R_{xA} is halo-C₁-C₆ alkyl or C₁-C₆ halo-alkoxy.

29.-30. (canceled)

31. The compound of claim 28, or a pharmaceutically acceptable salt thereof, wherein R_{xA} is trifluoromethyl.

32. The compound of claim 1, or a pharmaceutically acceptable salt thereof, wherein R_{y} is selected from the group consisting of halogen, C₃-C₇ cycloalkyl, and C₁-C₆ alkyl.

33. The compound of claim 32, or a pharmaceutically acceptable salt thereof, wherein R_{y} is selected from the group consisting of cyclopropyl, cyclobutyl, cyclopentyl, methyl, ethyl, n-propyl, isopropyl, and butyl.

34.-36. (canceled)

37. The compound of claim 1, or a pharmaceutically acceptable salt thereof, wherein p is 0.

38. The compound of claim 1, or a pharmaceutically acceptable salt thereof, wherein Ring A is selected from the group consisting of

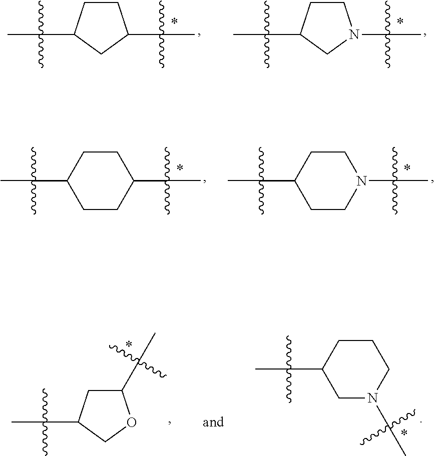

39. The compound of claim 1, or a pharmaceutically acceptable salt thereof, wherein one of m₁ and m₂ is 1 and the other is 2; or wherein m₁ and m₂ are each 2.

40.-43. (canceled)

44. The compound of claim 39, or a pharmaceutically acceptable salt thereof, wherein one of j₁ and j₂ is 1 and the other is 2.

45. (canceled)

46. The compound of claim 39, or a pharmaceutically acceptable salt thereof, wherein j₁ and j₂ are each 1.

47.-61. (canceled)

62. The compound of claim 1 wherein Ring A is:

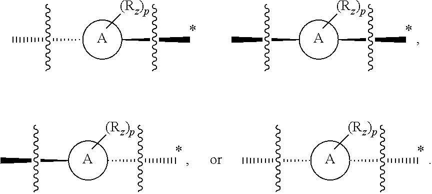

63. (canceled)

64. The compound of claim 1, wherein the compound is selected from the group consisting of: 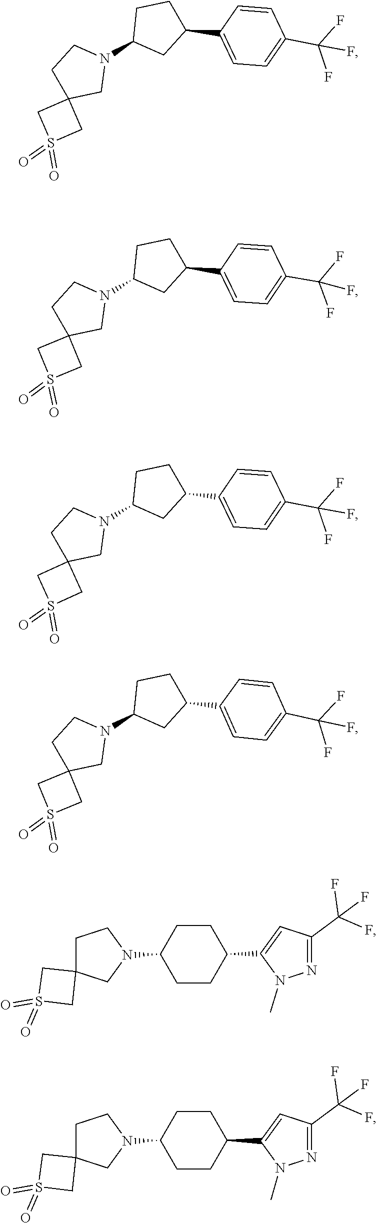 [SMILES: O=S1(=O)CC2(CCN([C@H]3CC[C@@H](c4ccc(C(F)(F)F)cc4)C3)C2)C1 ; O=S1(=O)CC2(CCN([C@@H]3CC[C@@H](c4ccc(C(F)(F)F)cc4)C3)C2)C1 ; O=S1(=O)CC2(CCN([C@@H]3CC[C@H](c4ccc(C(F)(F)F)cc4)C3)C2)C1 ; O=S1(=O)CC2(CCN([C@H]3CC[C@H](c4ccc(C(F)(F)F)cc4)C3)C2)C1 ; Cn1nc(C(F)(F)F)cc1[C@H]1CC[C@@H](N2CCC3(C2)CS(=O)(=O)C3)CC1 ; Cn1nc(C(F)(F)F)cc1[C@H]1CC[C@H](N2CCC3(C2)CS(=O)(=O)C3)CC1] 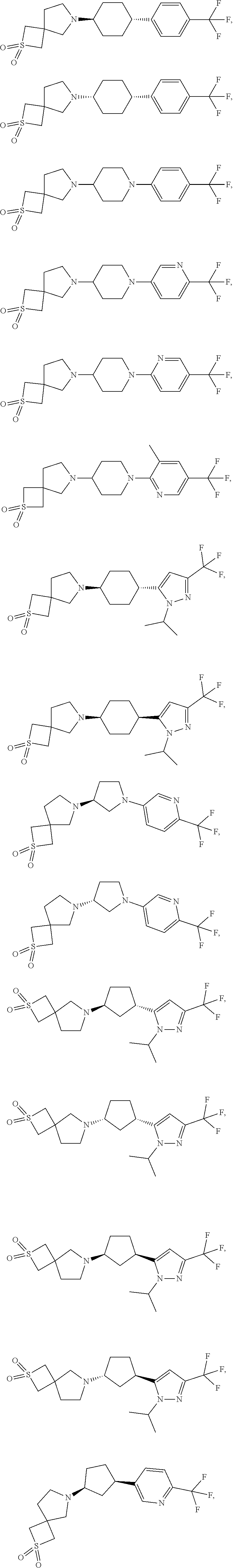 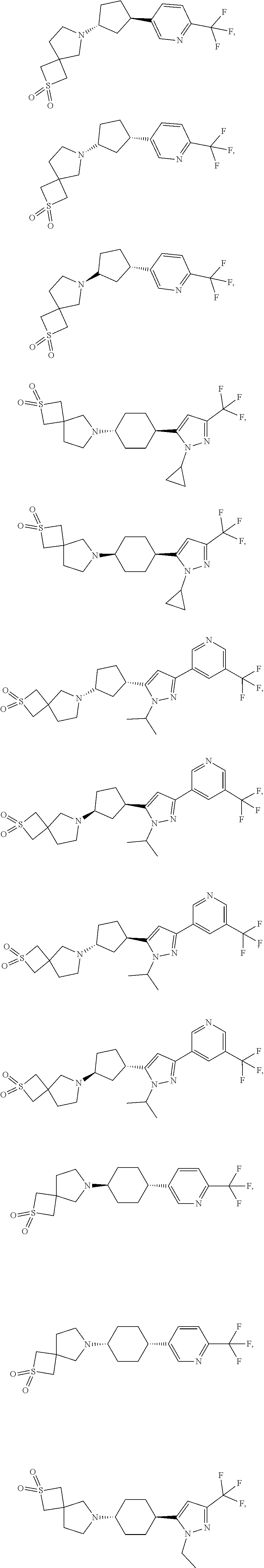 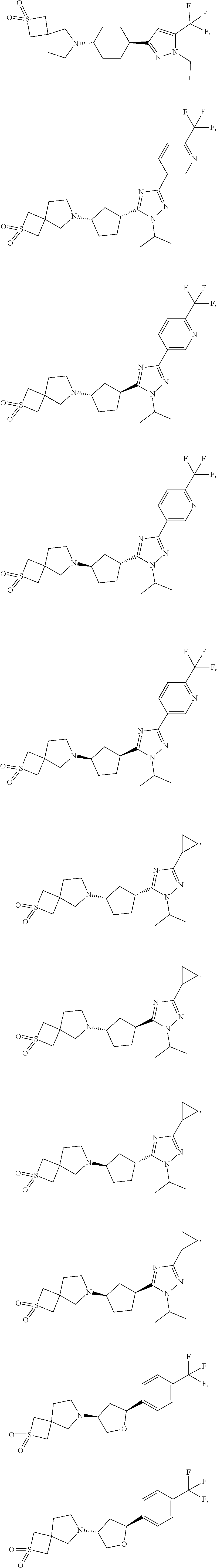 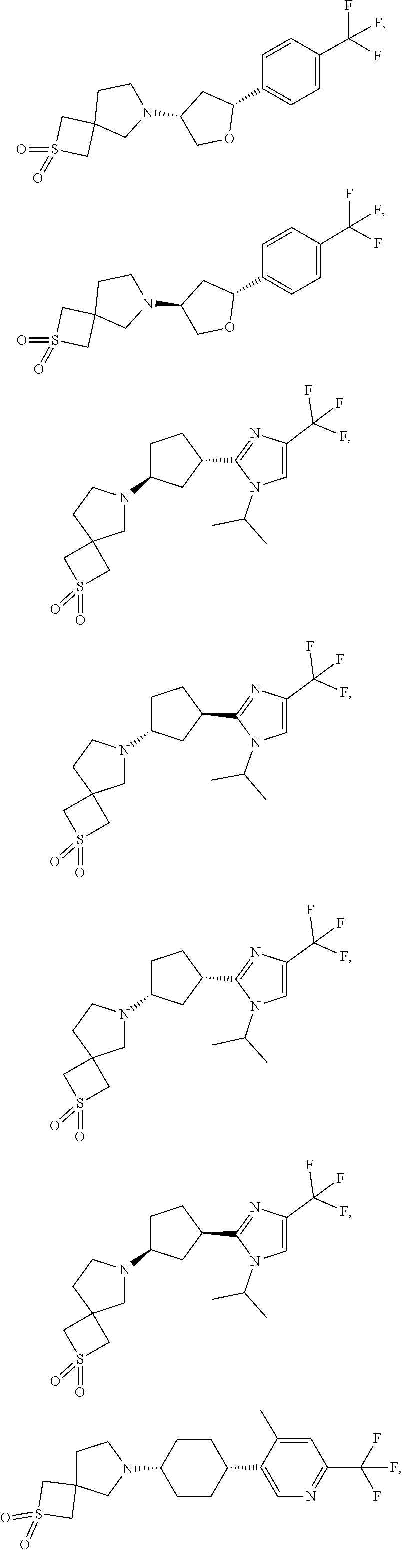 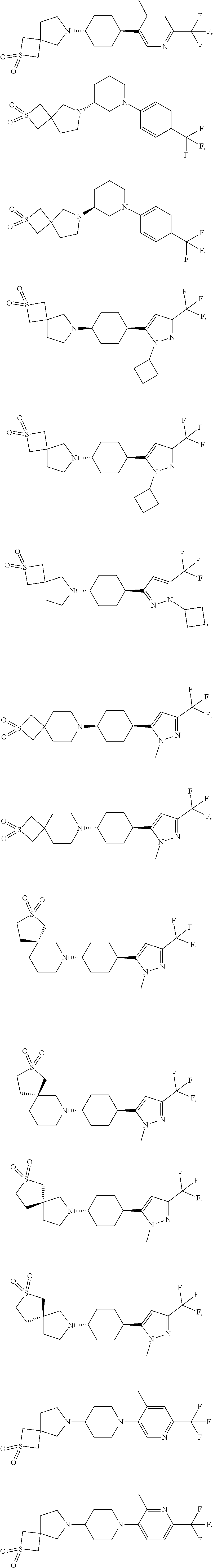 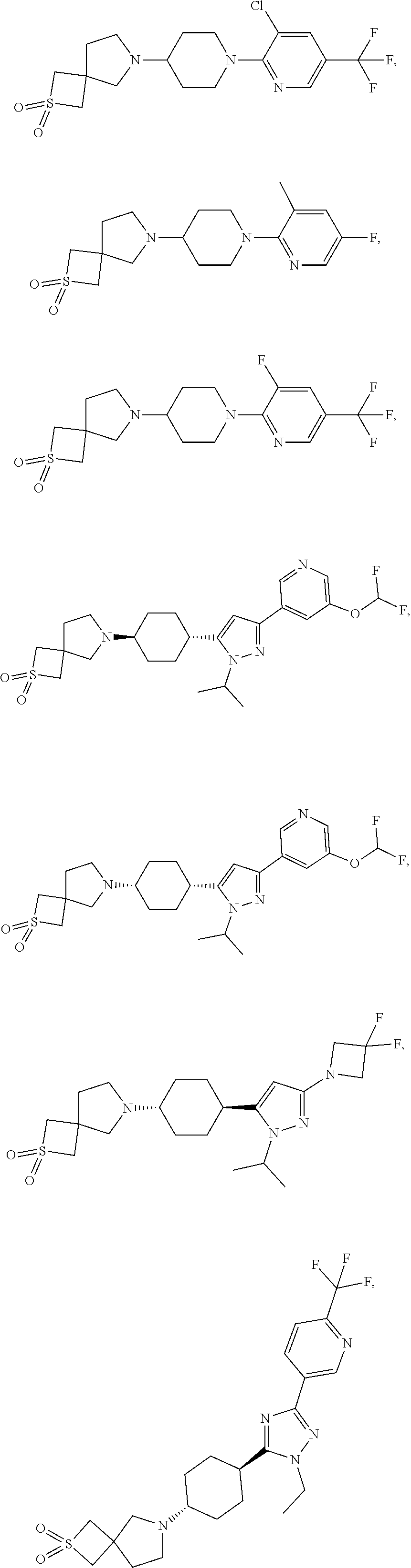 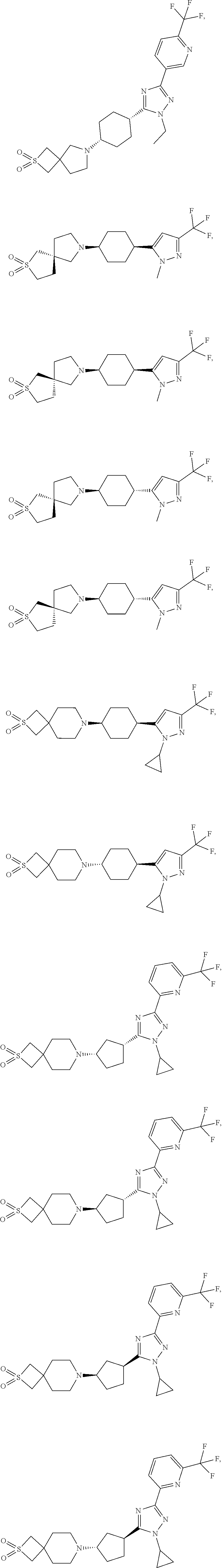 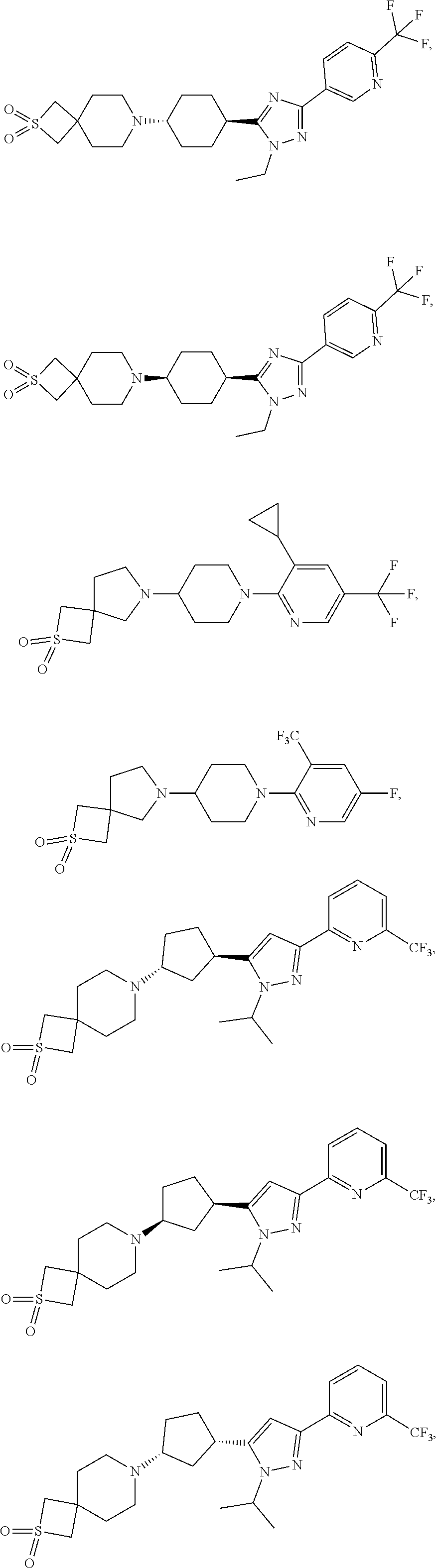 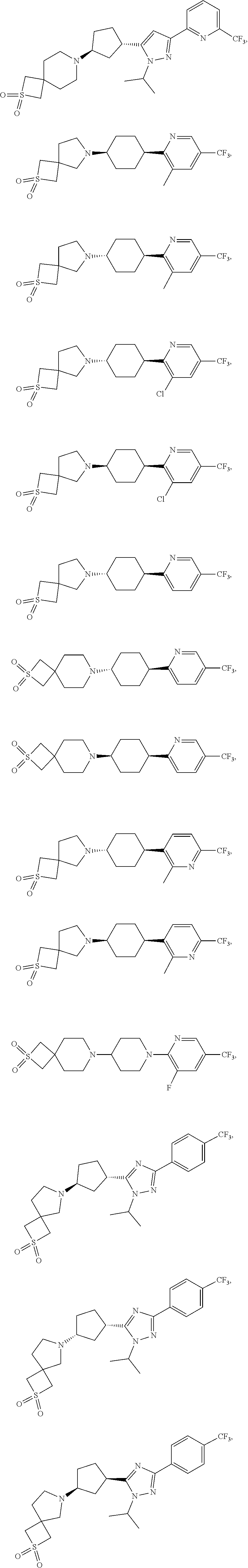 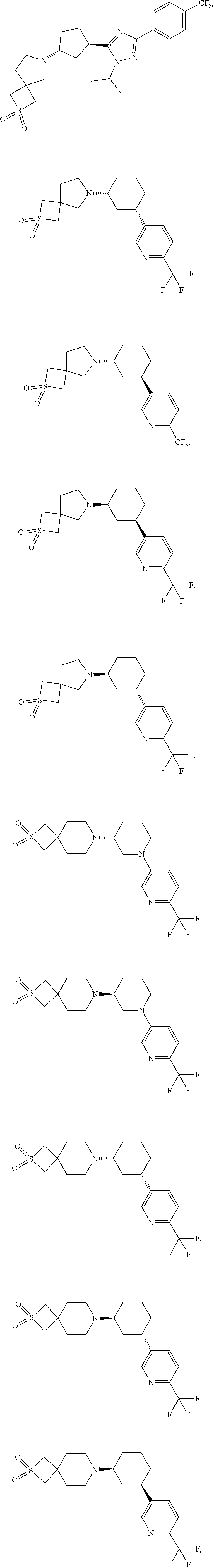 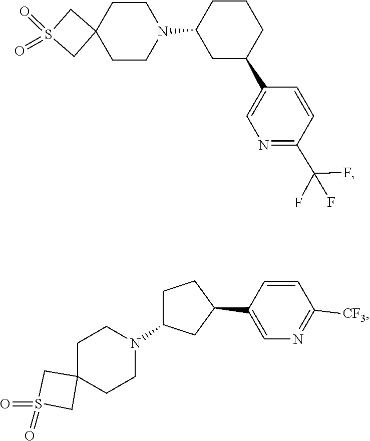 [SMILES: O=S1(=O)CC2(CCN([C@@H]3CCC[C@@H](c4ccc(C(F)(F)F)nc4)C3)CC2)C1 ; O=S1(=O)CC2(CCN([C@@H]3CC[C@@H](c4ccc(C(F)(F)F)nc4)C3)CC2)C1] 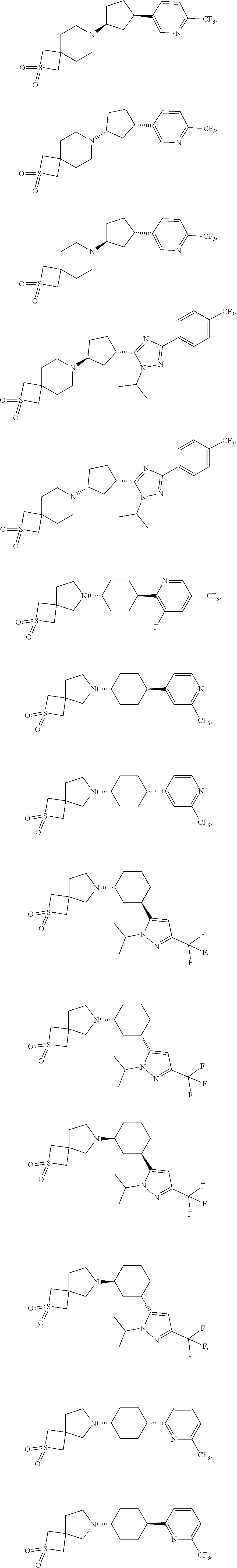 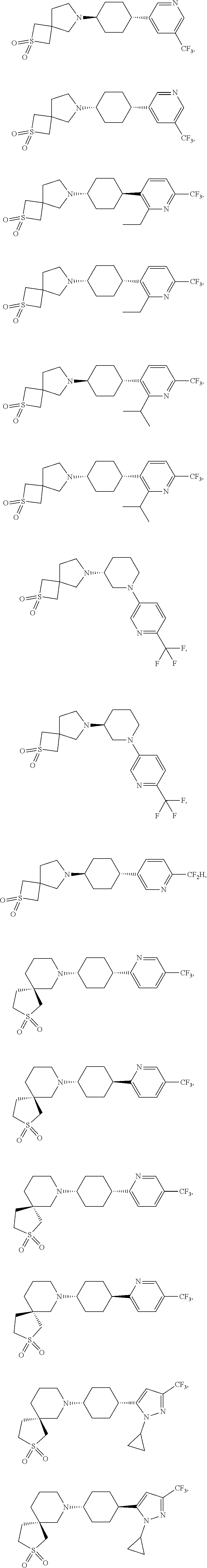 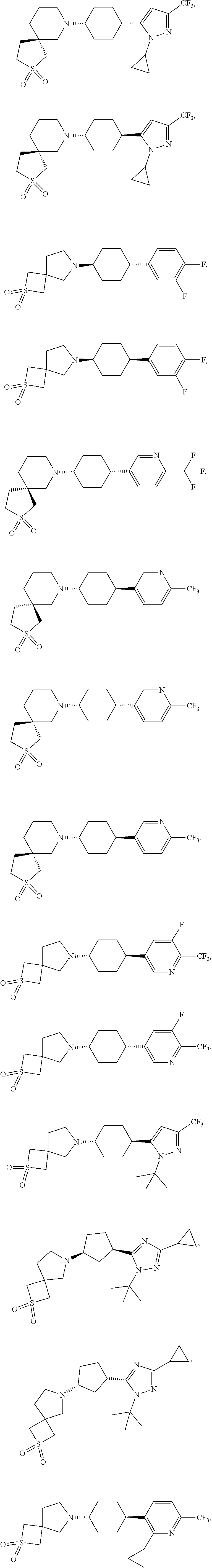 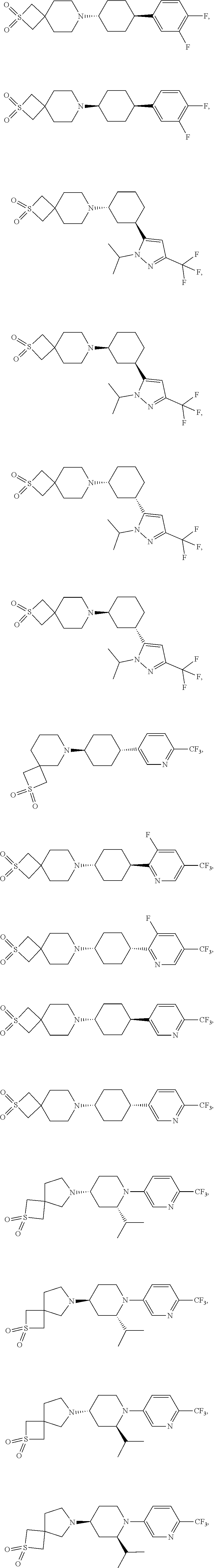 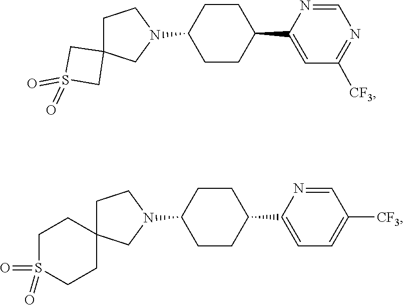 [SMILES: O=S1(=O)CC2(CCN([C@H]3CC[C@H](c4cc(C(F)(F)F)ncn4)CC3)C2)C1 ; O=S1(=O)CCC2(CCN([C@H]3CC[C@@H](c4ccc(C(F)(F)F)cn4)CC3)C2)CC1] 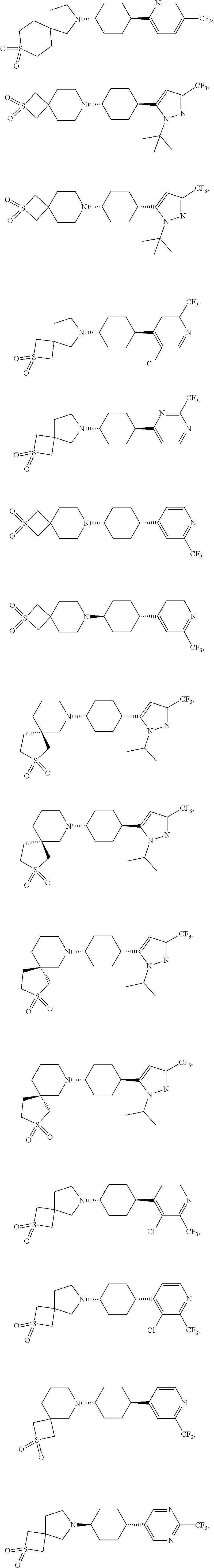 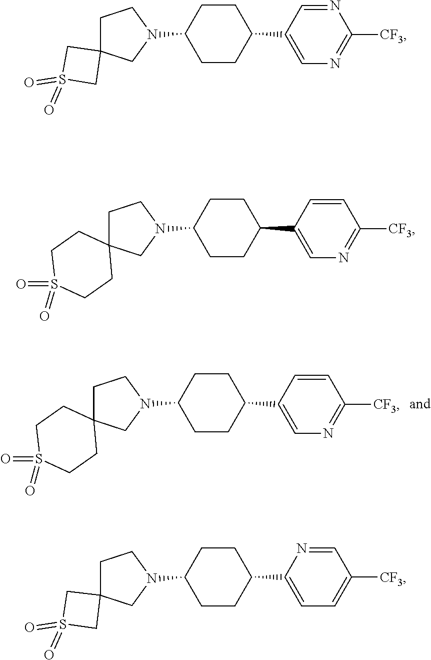 [SMILES: O=S1(=O)CC2(CCN([C@H]3CC[C@@H](c4cnc(C(F)(F)F)nc4)CC3)C2)C1 ; O=S1(=O)CCC2(CCN([C@H]3CC[C@H](c4ccc(C(F)(F)F)nc4)CC3)C2)CC1 ; O=S1(=O)CCC2(CCN([C@H]3CC[C@@H](c4ccc(C(F)(F)F)nc4)CC3)C2)CC1 ; O=S1(=O)CC2(CCN([C@H]3CC[C@@H](c4ccc(C(F)(F)F)cn4)CC3)C2)C1]
or a pharmaceutically acceptable salt thereof.

65. A pharmaceutical composition comprising a compound of claim 1 or a pharmaceutically acceptable salt thereof, and a pharmaceutically acceptable excipient.

66. A method of treating a disorder in a subject in need thereof, the method comprising administering to the subject in need thereof a therapeutically effective amount of a compound of claim 1, or a pharmaceutically acceptable salt thereof.

67.-68. (canceled)

69. A method of promoting myelination in a subject in need thereof, comprising administering to the subject a therapeutically effective amount of a compound of claim 1, or a pharmaceutically acceptable salt thereof.

70.-75. (canceled)
